(19) United States
(12) Patent Application Publication
(10) Pub. No.: US 2021/0071255 A1
(43) Pub. Date: Mar. 11, 2021

(54) METHODS FOR IDENTIFICATION OF GENES AND GENETIC VARIANTS FOR COMPLEX PHENOTYPES USING SINGLE CELL ATLASES AND USES OF THE GENES AND VARIANTS THEREOF

(71) Applicants: The Broad Institute, Inc., Cambridge, MA (US); Massachusetts Institute of Technology, Cambridge, MA (US); The General Hospital Corporation, Boston, MA (US)

(72) Inventors: Aviv Regev, Cambridge, MA (US); Karthik Jagadeesh, Cambridge, MA (US); Ramnik Xavier, Cambridge, MA (US); Christopher Smillie, Cambridge, MA (US)

(21) Appl. No.: 17/014,809

(22) Filed: Sep. 8, 2020

Related U.S. Application Data
(60) Provisional application No. 62/897,224, filed on Sep. 6, 2019.
(60) Provisional application No. 62/904,507, filed on Sep. 23, 2019.

(51) Int. Cl. C12Q 1/6883; C12N 9/22; C12N 15/113

(52) U.S. Cl. CPC C12Q 1/6883; C12N 9/22; C12N 15/113; C12Q 2600/158; C12Q 2600/112; C12N 2800/80; C12N 2310/20; C12Q 2600/156

(57) ABSTRACT

The present invention is generally directed to using a single cell atlas for identifying genes and gene programs that are associated with a phenotype (e.g., disease phenotypes or traits). The present invention is also generally directed to identifying interacting genetic variants using a single cell atlas as a prior for selecting variants for testing. The single cell atlas is used for constructing gene modules. Interactions are tested within and between modules. Applicants identified genetic variants that can be used to identify pathways and cell types important for IBD risk. Moreover, genetic variants were identified that can be used as therapeutic targets.

### CROSS-REFERENCE TO RELATED APPLICATIONS

This application claims the benefit of U.S. Provisional Application No. 62/897,224, filed Sep. 6, 2019 and U.S. Provisional Application No. 62/904,507, filed Sep. 23, 2019. The entire contents of the above-identified applications are hereby fully incorporated herein by reference.

### REFERENCE TO AN ELECTRONIC SEQUENCE LISTING

The contents of the electronic sequence listing (“BROD-4750US_ST25.txt”; Size is 12,767 bytes (16 KB on disk) and it was created on Sep. 3, 2020) is herein incorporated by reference in its entirety.

### TECHNICAL FIELD

The subject matter disclosed herein is generally directed to use of a single cell atlas to identify genes and genetic variants associated with complex phenotypes, such as disease phenotypes and traits. The methods can be used to identify pathways and therapeutic targets important for diagnosing and treating disease.

### BACKGROUND

New tools, such as single-cell genomics, have allowed for mapping single cell types in a tissue. Without maps of different cell types in a tissue and the genes they express, Applicants cannot describe all cellular activities and understand the biological networks that direct them. A comprehensive cell atlas makes it possible to catalog all cell types and even subtypes of cells in a tissue, and even distinguish different stages of differentiation and cell states, such as immune cell activation. A cell atlas has the potential to transform our approach to biomedicine. It helps to identify markers and signatures for disease phenotypes, uncover new targets for therapeutic intervention, and provides a direct view of human biology in vivo, removing the distorting aspects of cell culture. Patient cohort studies using single cell analysis allow for identifying consistent and robust features that underlie disease and response to therapy. Further uses of cell atlases remain to be elucidated.

The study of complex diseases has gradually shifted to genome-wide association studies (GWAS) (see, e.g., Li, et al., An overview of SNP interactions in genome-wide association studies. Briefings in Functional Genomics, Volume 14, Issue 2, March 2015, Pages 143-155). GWAS are mainly case-control studies that examine single-nucleotide polymorphisms (SNPs) to determine genetic factors associated with complex diseases (Id). Although GWAS have achieved a number of successes, few loci identified have a high or moderate disease risk, and some well-known genetic risk factors have been missed (Id). The relative risk of most new loci is only 1.1-1.2, which suggests that these individual SNPs have a small effect on the heritability of complex diseases, and that a large subset of SNPs associated with complex diseases has still not been identified (Id). First, pathogenic SNPs have a low population frequency, making them difficult to identify by GWAS using relatively small sample sets (Id). Another reason is that many studies use single-locus tests, in which each locus is tested independently for association with a phenotype, ignoring the combined effect of multiple loci on disease susceptibility (Id). The present invention shows that a single cell atlas can be used as a roadmap to identify disease relevant human genetic variation using combinations of genetic loci.

Genome wide association studies (GWAS) have successfully uncovered thousands of disease associated variants. Interpreting these variants to understand the biological mechanisms through which they are acting is a major unsolved challenge.

There exists several barriers to understanding the biological processes through which genetic variants are influencing disease phenotypes. This includes 1) understanding the structure of gene networks that are working together in different cellular contexts, 2) linking disease associated SNPs with causative genes in a context dependent manner and 3) aggregating signals from multiple disease associated loci that are additively working together.

Single cell RNA-seq (scRNAseq) provides an unprecedented opportunity to bridge this gap between variant and function. With scRNAseq, Applicants can generate a view into granular cell types across varying tissues and gene networks working together in cell type specific contexts. The gene expression patterns across the different cell subsets can reveal cell type specific expression signals of disease genes. Additionally, gene correlation patterns can be used to identify gene programs representing genes working together within and across cell subsets.

### SUMMARY

In one aspect, the present invention provides for a method of identifying genes associated with one or more phenotypes specific to a tissue comprising: providing one or more gene modules constructed from one or more single cell atlases for the tissue; linking genetic variants to the one or more gene modules based on enhancer-gene connections, wherein genetic variants located in enhancers predicted to regulate genes in the one or more gene modules are linked to the module; and identifying one or more phenotypes associated with the genetic variants linked to each gene module, thereby identifying genes associated with the phenotypes. In certain embodiments, linking genetic variants to the one or more gene modules comprises: calculating a gene score for genes in each module; and assigning a variant to the gene with the highest score among genes linked to that variant according to both an Activity-by-Contact (ABC) model and an epigenomic model. In certain embodiments, the epigenomic model uses chromatin state, gene expression, regulatory motif enrichment and regulator expression to predict enhancer-gene connections. In certain embodiments, gene score is based on the enrichment of each gene in each module and/or a gene level significance score based on GWAS p values of all surrounding SNPs. In certain embodiments, the phenotype is a disease phenotype and the gene modules comprise genes differentially expressed between healthy and disease states in the tissue, whereby gene programs associated with the disease phenotype are identified. In certain embodiments, the differentially expressed genes are cell type specific, whereby cell types associated with the disease phenotype are identified. In certain embodiments, the gene modules comprise transcriptomes specific for cell types in the tissue, whereby cell types associated with the phenotype are identified. In certain embodiments, the gene modules comprise biological programs indicating cell states in the tissue, whereby cell states associated with the phenotype are identified. In certain embodiments, the biological programs are determined by negative matrix factorization (NMF), topic modeling, or word embeddings.

In another aspect, the present invention provides for a method of identifying phenotypes associated with genes comprising: providing one or more gene modules comprising one or more genes of interest and one or more covarying genes constructed from one or more single cell atlases for a tissue associated with the genes of interest; linking genetic variants to the one or more gene modules based on enhancer-gene connections, wherein genetic variants located in enhancers predicted to regulate genes in the one or more gene modules are linked to the module; and identifying one or more phenotypes associated with the genetic variants linked to each gene module, thereby identifying phenotypes associated with the genes of interest. In certain embodiments, linking genetic variants to the one or more gene modules comprises: calculating a gene score for genes in each module; and assigning a variant to the gene with the highest score among genes linked to that variant according to both an Activity-by-Contact (ABC) model and an epigenomic model. In certain embodiments, the epigenomic model uses chromatin state, gene expression, regulatory motif enrichment and regulator expression to predict enhancer-gene connections. In certain embodiments, gene score is based on the enrichment of each gene in each module and/or a gene level significance score based on GWAS p values of all surrounding SNPs. In certain embodiments, the one or more genes of interest comprise one or more disease associated genes and wherein the tissue is associated with the disease, whereby phenotypes associated with disease associated genes are identified. In certain embodiments, the gene modules comprise transcriptomes specific for cell types in the tissue, whereby phenotypes associated with cell types are identified. In certain embodiments, the gene modules comprise biological programs indicating cell states in the tissue, whereby phenotypes associated with cell states are identified. In certain embodiments, the biological programs are determined by negative matrix factorization (NMF), topic modeling, or word embeddings.

In another aspect, the present invention provides for a method of determining a risk score for a disease phenotype comprising detecting in a subject two or more genetic variants associated with the disease phenotype and linked to a common gene module identified according to any embodiment herein.

In another aspect, the present invention provides for a method of determining a risk score for a disease phenotype comprising detecting in a subject one or more gene modules or cells identified according to any embodiment herein.

In certain embodiments, the gene modules are constructed using single cell RNA-seq data from the single cell atlas. In certain embodiments, the gene modules are constructed using single cell epigenetic data from the single cell atlas. In certain embodiments, the epigenetic data comprises single cell ChIP-seq data. In certain embodiments, the gene modules are constructed using single cell ATAC-seq data from the single cell atlas. In certain embodiments, the genetic variants are single nucleotide polymorphisms (SNPs). In certain embodiments, the SNPs are associated with phenotypes based on genome wide association studies (GWAS). In certain embodiments, the enhancers are specific to the tissue. In certain embodiments, identifying one or more phenotypes associated with the genetic variants linked to each gene module comprises stratified LD score regression across a set of phenotypes. In certain embodiments, the one or more single cell atlases were generated from a diseased tissue. In certain embodiments, the one or more single cell atlases were generated from a healthy tissue.

In another aspect, the present invention provides for an unbiased method of identifying interacting genetic variants associated with a phenotype comprising assigning genetic variants identified in one or more subjects having the phenotype to one or more gene modules, wherein the gene modules are derived from a single cell atlas specific for a tissue of interest associated with the phenotype, wherein the atlas comprises one or more single cell analyses of genomic loci comprising the genetic variants, and wherein a genetic variant is assigned to a gene module where the genomic loci comprising the genetic variant is transcriptionally active in the module; and determining interactions by testing the association of two or more genetic variants within the same module or between associated modules with the phenotype.

In certain embodiments, the genetic variant is present in a gene. In certain embodiments, the gene is a protein coding gene or a non-protein coding gene. In certain embodiments, the genetic variant is present in an exon or intron in the gene. In certain embodiments, the genetic variant is present in a regulatory element controlling expression of a gene.

In certain embodiments, the single cell atlas comprises one or more single cell analyses of tissues having the phenotype and tissues having a control phenotype. In certain embodiments, the single cell analyses comprise single cell RNA-seq data. In certain embodiments, the single cell analyses comprise epigenetic data. In certain embodiments, the epigenetic data comprises single cell ChIP-seq data. In certain embodiments, the single cell analyses comprise single cell ATAC-seq data.

In certain embodiments, the phenotype is a disease state. In certain embodiments, the disease state is classified by severity or subtype. In certain embodiments, the genetic variants tested are present at a higher frequency in subjects having the disease than in control subjects. In certain embodiments, the gene modules are conserved across disease states. In certain embodiments, the gene modules are non-conserved across disease states.

In certain embodiments, each gene module comprises genes or genomic loci that are transcriptionally active in a specific cell type, whereby the gene modules are cell type specific. In certain embodiments, the gene modules are constructed by: grouping one or more genes associated with the phenotype by cell type specificity; and adding one or more additional genes to each group that co-vary in each cell type with the genes associated with the phenotype. In certain embodiments, each gene module comprises genes differentially expressed in single cell types between disease and control subjects. In certain embodiments, each gene module comprises genes located in open chromatin in single cells. In certain embodiments, each gene module comprises genes located in chromatin comprising active epigenetic marks in single cells. In certain embodiments, each gene module comprises a gene program expressed across the single cells. In certain embodiments, associated gene modules comprise cell type specific modules for interacting cell types. In certain embodiments, the interacting cell types are selected from the group consisting of immune cells, stromal cells and epithelial cells.

In certain embodiments, the method further comprises identifying genetic variants in the one or more subjects. In certain embodiments, the genetic variants are identified by whole exome sequencing (WES).

In certain embodiments, the method further comprises identifying pathways associated with the phenotype, said method comprising clustering the identified genetic variants by traits associated with the tissue of interest. In certain embodiments, the genetic variants are clustered using Bayesian nonnegative matrix factorization (bNMF). In certain embodiments, the method further comprises identifying cell types associated with the phenotype, said method comprising determining the expression of genomic loci comprising the identified genetic variants in single cells in the tissue. In certain embodiments, the method further comprises determining a risk score for the phenotype for a subject, said method comprising detecting in the subject genetic variants in one or more gene modules comprising an interacting genetic variant, wherein detecting a genetic variant in the gene modules indicates increased risk for the phenotype.

In certain embodiments, the tissue of interest is colon or intestinal tissue. In certain embodiments, the disease is inflammatory bowel disease (IBD). In certain embodiments, the IBD is ulcerative colitis (UC). In certain embodiments, the disease is cancer. In certain embodiments, the cancer is colorectal cancer (CRC).

In another aspect, the present invention provides for a method of determining a risk score for a disease phenotype for a subject, said method comprising detecting in the subject genetic variants in one or more cell type specific gene modules, wherein detecting a variant in a gene module indicates increased risk for the disease phenotype, and wherein the one or more gene modules comprise one or more genes associated with the disease phenotype and one or more genes that co-vary with the disease genes in each cell type. In certain embodiments, the genes associated with the disease phenotype are determined by genome wide association studies. In certain embodiments, the genes associated with the disease phenotype are determined by the method according to any embodiment herein. In certain embodiments, the cell type specific gene expression is determined by single cell RNA sequencing one or more control and disease tissue samples. In certain embodiments, the disease is inflammatory bowel disease (IBD). In certain embodiments, the IBD is ulcerative colitis (UC). In certain embodiments, the one or more cell type specific gene modules are selected from Table 4, Table 5, Table 6, or the group consisting of myeloid cells, epithelial cells, stromal cells, cycling B cells, germinal center B cells, transit amplifying cells, macrophages, enterocytes, enterocyte progenitors, CD8+ IELs and goblet cells. In certain embodiments, the disease is cancer. In certain embodiments, the cancer is colorectal cancer (CRC).

In another aspect, the present invention provides for a method of treating inflammatory bowel disease (IBD) in a subject in need thereof comprising altering one or more genetic variants, or altering expression, activity and/or function of one or more genes comprising the one or more genetic variants in one or more cell types, wherein the one or more genetic variants are selected from Table 7 or from the group consisting of 16:50763778 (NOD2), 16:50745199 (NOD2), 19:55144141 (LILRB1), 16:50744624 (NOD2), 1:117122130 (IGSF3), 2:233659553 (GIGYF2), 11:55595018 (OR5L2) and 16:2155426 (PKD1). In certain embodiments, two or more genetic variants or genes comprising the genetic variants are altered. In certain embodiments, the one or more genetic variants are in transcriptionally active loci in the same cell type. In certain embodiments, the one or more genetic variants are in transcriptionally active loci in different cell types. In certain embodiments, the one or more genetic variants are within NOD2. In certain embodiments, the one or more genetic variants are 16:50763778 and 16:50745199.

In certain embodiments, the expression, activity and/or function of the one or more genes comprising the one or more genetic variants is reduced or abolished. In certain embodiments, the one or more genetic variants is altered using genome editing. In certain embodiments, the one or more genetic variants or genes comprising the one or more genetic variants are altered in one or more cell types in vivo. In certain embodiments, the one or more genetic variants or genes comprising the one or more genetic variants are altered in one or more cell types ex vivo and the cells are transferred to the subject. In certain embodiments, the one or more genetic variants or genes comprising the one or more genetic variants are altered in intestinal stem cells. In certain embodiments, the one or more genetic variants or genes comprising the one or more genetic variants are altered in transit-amplifying cells (TA cells).

In certain embodiments, the cells are treated with one or more agents comprising a small molecule, small molecule degrader, genetic modifying agent, antibody, antibody fragment, antibody-like protein scaffold, aptamer, protein, or any combination thereof. In certain embodiments, the genetic modifying agent comprises a CRISPR system, RNAi system, a zinc finger nuclease system, a TALE system, or a meganuclease. In certain embodiments, the CRISPR system may be a CRISPR-Cas base editing system, a prime editor system, or a CAST system.

In certain embodiments, the IBD is ulcerative colitis (UC). In certain embodiments, the genetic variants are single-nucleotide polymorphisms (SNPs).

In another aspect, the present invention provides for a method of determining a risk score for a phenotype comprising detecting in a subject altered expression of one or more gene modules in Tables 8 to 12 or altered signaling in a pathway in FIGS. 34 to 42. In certain embodiments, an altered GABA-ergic neuron cell type program indicates a risk for Major Depressive Disorder (MDD) and/or body mass index (BMI). In certain embodiments, TCF4 and/or PCLO are detected. In certain embodiments, an altered TGF-beta regulation of extracellular matrix and/or ECM-receptor interaction program indicates a risk for decreased lung capacity and/or asthma. In certain embodiments, one or more genes selected from the group consisting of ITGA1, LOX, TGFBR3, COL8A1, BAMBI and VCL are detected. In certain embodiments, an altered pericyte and/or vascular smooth muscle gene program indicates a risk for abnormal systolic and diastolic blood pressure. In certain embodiments, one or more genes selected from the group consisting of GUCY1A3, CACNA1C, PDE8A and EDNRA are detected. In certain embodiments, an altered atrial cardiomyocyte gene program indicates a risk for abnormal atrial fibrillation and cardiac rhythm. In certain embodiments, one or more genes selected from the group consisting of PKD2L2, CASQ2 and KCNN2 are detected. In certain embodiments, ‘potassium channel’ pathways are detected. In certain embodiments, an altered T Lymphocyte, enterocyte and/or ILC disease gene program indicates a risk for ulcerative colitis. In certain embodiments, IL2RA is detected.

In another aspect, the present invention provides for a method of modifying a phenotype comprising administering one or more agents to a subject in need thereof capable of altering expression of one or more gene modules in Tables 8 to 12 or altering signaling in a pathway in FIGS. 34 to 42. In certain embodiments, Major Depressive Disorder (MDD) and/or body mass index (BMI) is treated and the one or more agents alter the GABA-ergic neuron cell type program. In certain embodiments, TCF4 and/or PCLO are altered. In certain embodiments, decreased lung capacity and/or asthma is treated and the one or more agents alter the TGF-beta regulation of extracellular matrix and/or ECM-receptor interaction program. In certain embodiments, one or more genes selected from the group consisting of ITGA1, LOX, TGFBR3, COL8A1, BAMBI and VCL are altered. In certain embodiments, abnormal systolic and diastolic blood pressure is treated and the one or more agents alter the pericyte and/or vascular smooth muscle gene program. In certain embodiments, one or more genes selected from the group consisting of GUCY1A3, CACNA1C, PDE8A and EDNRA are altered. In certain embodiments, abnormal atrial fibrillation and cardiac rhythm is treated and the one or more agents alter the atrial cardiomyocyte gene program. In certain embodiments, one or more genes selected from the group consisting of PKD2L2, CASQ2 and KCNN2 are altered. In certain embodiments, ‘potassium channel’ pathways are altered. In certain embodiments, ulcerative colitis is treated and the one or more agents alter the T Lymphocyte, enterocyte and/or ILC disease gene program. In certain embodiments, IL2RA is altered.

These and other aspects, objects, features, and advantages of the example embodiments will become apparent to those having ordinary skill in the art upon consideration of the following detailed description of illustrated example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIG. 1A-1D—Genome wide association studies (GWAS) and structure underlying polygenic traits. FIG. 1A. Schematic showing that statistically significant genomic variants can be identified that are present at higher frequencies in disease cases as compared to control cases. FIG. 1B. Schematic showing that genetic risk genes organize into gene programs (see, e.g., Smillie, Biton, Ordovas-Montanes et al., Cell 2019). FIG. 1C. Schematic showing that each gene program can represent a risk module. FIG. 1D. Schematic showing disease loci can be used to identify gene programs related to biological pathways, identify therapeutic targets, and detection of high risk individuals.

FIG. 2—Plot showing GWAS over 50K exomes for IBD.

FIG. 3—Heat maps showing UKBBK phenotype clustering.

FIG. 4—Heat map showing single cell expression data for cell types by disease genes.

FIG. 5—Graph showing IBD diagnosis prediction using logistic regression and a deep neural network.

FIG. 6A-6B—FIG. 6A. Schematics showing the complexity of testing every pair of SNPs and assigning the SNPs to cell type modules based on expression of the SNPs. FIG. 6B. Diagrams showing combining an IBD exome cohort with colon single cell atlas to identify genome-wide SNP interactions.

FIG. 7—Schematic showing building modules of genes to extend beyond disease genes.

FIG. 8A-8C—FIG. 8A. Schematic and chart showing that a burden test of gene modules over all the UC patients picks up subtler effects. FIG. 8B. Chart and plot showing that a burden test of gene modules over all the UC patients picks up subtler effects. FIG. 8C. Schematic and chart showing that a burden test of gene modules over all the UC patients picks up subtler effects.

FIG. 9—Heat map showing patient stratification over modules.

FIG. 10—Chart showing interactions occurring between modules.

FIG. 11A-11B—FIG. 11A. Schematic of the genomic locus comprising the NOD2 gene (interacting SNPs are indicated by boxes). FIG. 11B. Protein structure of NOD2 and indicated domain comprising variants.

FIG. 12A-12B—FIG. 12A. Schematic showing SNP interactions within a module and between modules. FIG. 12B. Schematic showing SNP interactions within a module and between modules.

FIG. 13—Schematics showing a summary of the value of combining single cell RNA-seq and human genetics.

FIG. 14—Schematics showing determining a polygenic risk score for each individual genome using variants derived from the GWAS (left) and using variants derived from the GWAS for each module (right).

FIG. 15—An overview of SCALED (Single Cell Analysis of Linked Enhancers for Disease) (also referred to as sc-ldsc and SCONE): An schematic representation of the SCALED workflow that comprise of the following steps in sequence, (i) generating gene programs (as used in this example “gene program” is used to refer to gene modules) that are enriched in a healthy cell-type or enriched specifically in the disease state of a cell type across 10 different tissues, (ii) combining the gene score with the union of Activity-By-Contact and Roadmap Enhancer-to-gene (E2G) strategy matched to the tissue of interest to generate SNP program matrix and (iii) evaluating the resulting SNP annotations for complex trait heritability using the Stratified LD score (S-LDSC) regression method. The post-processing of the output leads to inference about the association of a gene with a disease through a cellular program.

FIG. 16A-16F—SCALED analysis of healthy cell type specific (CTS) programs (“modules”) in blood and brain: FIG. 16(A) A demo of the UMAP representation of scRNA-seq data from a tissue (here PBMC), with heatmap representations of top cell type specific (CTS) genes. These genes have high annotation value in healthy CTS gene programs. FIG. 16(B) Heritability Enrichment score (Escore) analysis of SNP annotations corresponding to 6 CTS programs, aggregated over 4 healthy scRNA-seq data (2 PBMC, 1 cordblood, 1 bonemarrow), combined with the Roadmap-U-ABC-blood E2G strategy. Results analyzed for 5 blood biomarker traits with matched CTS program marked by the dotted square. FIG. 16(C) Average Escore and average standardized effect size (τ*) of matched blood biomarkers and blood CTS programs from panel (B), combined with 100 kb, ABC-blood and Roadmap-blood S2G strategies compared to Roadmap-U-ABC-blood. FIG. 16(D) Heritability Enrichment score (Escore) analysis of the SNP annotations from Panel (B) for 11 immune diseases. FIG. 16(E) Heritability Enrichment score (Escore) analysis of 3 CTS programs aggregated over 3 healthy brain scRNA-seq data, combined with the Roadmap-U-ABC-brain E2G strategy. Results analyzed for 11 brain related traits. FIG. 16(F) Assessing Escore of blood and brain CTS programs from Panels (B) and (E) (colored along X axis), combined with either Roadmap-U-ABC-blood or Roadmap-U-ABC-brain E2G strategies (column facets), averaged over 11 brain and 11 immune traits (row facets). In Panels (B), (D) and (E), the size and the color grade of circles represent the magnitude and significance level of Escore respectively. Errors bars denote 95% confidence intervals. All results are conditional on 86 baseline-LDv2.1 model annotations.

FIG. 17A-17D—SCALED analysis of healthy cell type specific (CTS) programs (“modules”) in kidney, liver, heart, lung and colon: Applicants evaluated SNP annotations corresponding to healthy celltype specific (CTS) programs from scRNA-seq data in different tissues such as kidney, liver, heart, lung and colon, combined with Roadmap-U-ABC E2G strategy for the corresponding tissue. FIG. 17(A) Heritability Enrichment score (Escore) analysis of SNP annotations corresponding to healthy kidney and liver CTS programs, combined with Roadmap-U-ABC-kidney and Roadmap-U-ABC-liver E2G strategies. Results are analyzed for 7 urine biomarker traits (shaded blue and pink for kidney and liver related). FIG. 17(B, C, D) Escore analysis of SNP annotations corresponding to healthy heart, lung and colon tissues for 6, 2 and 6 cardiovascular, lung and colon related traits. FIG. 17(E) Correlation in the healthy CTS program for an immune celltype (e.g. B cells) across different tissues. In Panels (A)-(D), the size and the color grade of circles represent the magnitude and significance level of Escore respectively. All results are conditional on 86 baseline-LDv2.1 model annotations.

FIG. 18A-18F—SCALED analysis of differentially disease specific (DDS) programs (“modules”) for Inflammatory Bowel Disease (IBD), Multiple Sclerosis (MS) and Asthma.: FIG. 18(A) An overview of how the DDS program for a particular cell type (T cells) is constructed with an example of a gene with high annotation value in the DDS program. FIG. 18(B) Average negative log p-value of Enrichment Score (p.Escore) for DDS programs in IBD, MS and Asthma, combined with Roadmap-U-ABC strategy for gut, blood and lung respectively (rows), with respect to their corresponding matched diseases (column). Each row is scaled by the maximum value. FIG. 18(C) Heritability Enrichment score (Escore) analysis of SNP annotations corresponding to IBD DDS programs, combined with matched Roadmap-U-ABC-gut E2G strategy. FIG. 18(D) Heritability Enrichment score (Escore) analysis of SNP annotations corresponding to Multiple Sclerosis (MS) DDS programs, combined with Roadmap-U-ABC-blood E2G strategy for MS trait (shaded red) and Roadmap-U-ABC-brain E2G strategy for two schizophrenia related traits (shaded blue). FIG. 18(E) Heritability Enrichment score (Escore) analysis of SNP annotations corresponding to Asthma DDS programs, combined with Roadmap-U-ABC-lung E2G strategy. In Panels (C)-(E), results are shown only for 4, 3 and 3 celltypes (healthy CTS and DDS) with most significant DDS program signal, and the size and the color grade of circles represent the magnitude and significance level of Escore respectively FIG. 18(F) Applicants report celltypes with significant difference in composition between the healthy CTS and the DDS programs for IBD, MS and Asthma. All results are conditional on 86 baseline-LDv2.1 model annotations, and for the DDS program, also on the corresponding healthy CTS program.

FIG. 19—4 blood single cell RNAseq datasets. UMAP plots corresponding to 4 separate blood single cell RNAseq datasets. In each dataset Applicants identify the predominant cell types. There are two peripheral blood mononucleated cell datasets, one bone marrow dataset and one cord blood dataset.

FIG. 20—4 blood single cell RNAseq datasets. UMAP plots corresponding to 3 separate brain single cell RNAseq datasets. In each dataset Applicants identify the predominant cell types.

FIG. 21—Evaluation of different S2G strategies in SCONE analysis of blood biomarker traits. Heritability Enrichment score (Escore) analysis corresponding to 5 blood biomarker traits for SNP annotations corresponding to 6 CTS programs, aggregated over 4 healthy scRNA-seq data (2 PBMC, 1 cordblood, 1 bonemarrow), combined with 100 kb, ABC-blood and Roadmap-blood S2G strategies instead of the Roadmap-U-ABC-blood strategy used in FIG. 16 Panel B. The size and the color grade of circles represent the magnitude and significance level of Escore respectively. All results are conditional on 86 baseline-LDv2.1 model annotations.

FIG. 22A-22C—SCONE standardized τ* analysis of healthy cell type specific (CTS) programs (“modules”) in blood and brain. Standardized effect size (τ*) analysis of SNP annotations corresponding to FIG. 22(A, B) 6 healthy blood CTS programs combined with Roadmap-U-ABC-blood strategy for (A) 5 blood biomarker traits and (B) 11 autoimmune diseases, and corresponding to FIG. 22(C) 3 healthy brain CTS programs combined with Roadmap-U-ABC-brain strategy for 11 brain related traits. The size and the color grade of circles represent the magnitude and significance level of τ* respectively. All results are conditional on 86 baseline-LDv2.1 model annotations.

FIG. 23—Additional healthy single cell RNAseq datasets. UMAP plots corresponding to Kidney, Liver, Heart, Liver, and Colon. Each dataset contains a subset of common cell types found across varying tissues as well as context specific cell types specific to the tissue of interest.

FIG. 24—4 blood single cell RNAseq datasets. UMAP plots corresponding to Adipose and Skin single cell RNAseq datasets. In each dataset Applicants identify the predominant cell types.

FIG. 25A-25B—SCONE analysis of healthy cell type specific (CTS) programs (“modules”) in adipose and skin. Applicants evaluated SNP annotations corresponding to healthy cell type specific (CTS) programs from scRNA-seq data in adipose and skin. FIG. 25(A) Heritability Enrichment score (Escore) analysis of SNP annotations corresponding to 5 fat related traits for healthy adipose CTS programs, combined with Roadmap-U-ABC-fat strategy. FIG. 25(B) Heritability Enrichment score (Escore) analysis of SNP annotations corresponding to 2 skin related traits for healthy skin CTS programs, combined with Roadmap-U-ABC-skin strategy. The size and the color grade of circles represent the magnitude and significance level of τ* respectively. All results are conditional on 86 baseline-LDv2.1 model annotations.

FIG. 26—3 lung related disease datasets. UMAP plots corresponding to asthma, fibrosis and COVID-19.

FIG. 27—Additional disease datasets. UMAP plots for ulcerative colitis, multiple sclerosis and Alzheimer's.

FIG. 28—Correlation between healthy CTS, disease CTS and DDS programs (“modules”) in IBD, MS and Asthma. Correlation matrix of healthy cell type specific, disease cell type specific (disease CTS) and differentially disease specific (DDS) programs for three healthy plus disease scRNA-seq studies corresponding to IBD, MS and Asthma.

FIG. 29—Correlation between healthy CTS, disease CTS and DDS programs (“modules”) in Alzheimer's, Lung Fibrosis and COVID-19. Correlation matrix of healthy cell type specific, disease celltype specific (disease CTS) and differentially disease specific (DDS) programs for three healthy plus disease scRNA-seq studies corresponding to Alzheimers, Lung Fibrosis and COVID-19.

FIG. 30—Evaluating disease specificity of DDS programs (“modules”) for IBD, MS and Asthma when combined with a single E2G strategy, Roadmap-U-ABC-blood. Average negative log p-value of Enrichment Score (p.Escore) for DDS programs in IBD, MS and Asthma, combined with Roadmap-U-ABC-blood strategy (rows), with respect to their corresponding matched diseases (column). Each row is SCONE by the maximum value.

FIG. 31A-31G—SCONE analysis of healthy cell type specific (CTS) programs (“modules”) in different tissues using non-tissue-specific E2G strategy. Heritability Enrichment score (Escore) analysis of SNP annotations corresponding to healthy CTS programs for FIG. 31(A) blood, FIG. 31(B) brain, FIG. 31(C) heart, FIG. 31(D) lung, FIG. 31(E) colon, FIG. 31(F) adipose and FIG. 31(G) combined with Roadmap-U-ABC-all E2G strategy. Results reported only for traits matched to respective tissues. The size and the color grade of circles represent the magnitude and significance level of τ* respectively. All results are conditional on 86 baseline-LDv2.1 model annotations.

FIG. 32A-32D—SCONE analysis of healthy CTS and disease DDS programs (“modules”) for COVID-19. Heritability Enrichment score (Escore) analysis of SNP annotations corresponding to healthy CTS and disease DDS programs for COVID-19 scRNA-seq data, combined with Roadmap-U-ABC-lung and Roadmap-U-ABC-blood E2G strategies. The size and the color grade of circles represent the magnitude and significance level of τ* respectively. All results are conditional on 86 baseline-LDv2.1 model annotations.

FIG. 33—SCONE analysis of disease DDS programs (“modules”) for Lung Fibrosis. Heritability Enrichment score (Escore) analysis of SNP annotations corresponding to disease DDS programs in Lung Fibrosis scRNA-seq data, combined with Roadmap-U-ABC-lung and Roadmap-U-ABC-blood E2G strategies. The size and the color grade of circles represent the magnitude and significance level of τ* respectively. All results are conditional on 86 baseline-LDv2.1 model annotations and corresponding healthy CTS programs.

FIG. 34A-34B—Gene set enrichment analysis identified pathways and genes significantly altered in MS Disease Glutamatergic cells (Table 9).

FIG. 35A-35B—Gene set enrichment analysis identified pathways and genes significantly altered in MS Disease Endothelial cells (Table 9).

FIG. 36—Gene set enrichment analysis identified pathways and genes significantly altered in MS Disease Stromal cells (Table 9).

FIG. 37—Gene set enrichment analysis identified pathways and genes significantly altered in MS Disease Myeloid cells (Table 9).

FIG. 38—Gene set enrichment analysis identified pathways and genes significantly altered in UC disease (Table 9).

FIG. 39A-39B—Gene set enrichment analysis identified pathways and genes significantly altered in Healthy Celiac PBMC T lymphocytes (Table 12).

FIG. 40A-40B—Gene set enrichment analysis identified pathways and genes significantly altered in Healthy UC PBMC B lymphocytes (Table 12).

FIG. 41A-41B—Gene set enrichment analysis identified pathways and genes significantly altered in Healthy MDD GABAergic (Table 12).

FIG. 42A-42B—Gene set enrichment analysis identified pathways and genes significantly altered in Healthy Intelligence glutamatergic (Table 12).

The figures herein are for illustrative purposes only and are not necessarily drawn to scale.

### DETAILED DESCRIPTION OF THE EXAMPLE EMBODIMENTS

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4^{th} edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2nd edition 2013 (E. A. Greenfield ed.); Animal Cell Culture (1987) (R.I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2^{nd} edition (2011).

As used herein, the singular forms “a”, “an”, and “the” include both singular and plural referents unless the context clearly dictates otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of +/−10% or less, +/−5% or less, +/−1% or less, and +/−0.1% or less of and from the specified value, insofar such variations are appropriate to perform in the disclosed invention. It is to be understood that the value to which the modifier “about” or “approximately” refers is itself also specifically, and preferably, disclosed.

As used herein, a “biological sample” may contain whole cells and/or live cells and/or cell debris. The biological sample may contain (or be derived from) a “bodily fluid”. The present invention encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from a mammal organism, for example by puncture, or other collecting or sampling procedures.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

All publications, published patent documents, and patent applications cited herein are hereby incorporated by reference to the same extent as though each individual publication, published patent document, or patent application was specifically and individually indicated as being incorporated by reference.

### Overview

Single cell data provides granular information about genes and the context in which they are expressed across a range of cell types. Here, Applicants hypothesized that the information on which genes are co-varying within each cell type can serve as a prior to increase the power and ability to interpret disease relevant human genetic variation. Using single cell atlas and genetic data from inflammatory bowel disease (IBD), Applicants show that combining signals from single cells and human genetics helps identify cell types affecting disease, stratify disease subtypes by a combination of genetic and functional signals, organize casual genes into modules, determine genetic interactions within and between loci, and find disease relevant interactions between cell types and SNPs. Applicants provide a method that allows for genome wide interaction studies that were previously unfeasible due to the number of interactions to be tested. The methods allow for identifying subtle genetic associations to disease. In certain embodiments, the association of a genetic loci with disease can only be identified in combination with one or more additional genetic loci (e.g., polygenic).

Moreover, understanding the cellular mechanisms through which genetic variants influence disease outcomes remains a major biological challenge. Single cell RNAseq (scRNAseq) provides a unprecedented ability to learn the gene programs driving biological mechanisms across varied cellular contexts. Additionally, population scale GWAS studies are now pinpointing the genetic variation influencing disease. Here, Applicants introduce a new approach to link variant (human genetics from GWAS) to function (disease critical cellular programs from scRNAseq) by learning from and integrating heterogeneous information rich biological datasets including: scRNAseq, GWAS, ROADMAP epigenomic markers and Hi-C activity. Applicants analyze scRNAseq data from over 10 healthy and 5 disease tissues (including COVID-19) spanning 186 individuals and over 1.5 million single cells. Applicants then transform the gene programs into SNP annotations using tissue specific SNP-to-gene (S2G) linking strategies and evaluate the resulting annotations using stratified LD score regression across 127 complex traits and diseases. The approach showed high specificity of capturing known cell type-trait pairs in terms of excess enrichment adjusted for S2G strategy e.g. T and B Lymphocytes for lymphocyte count (2.3×, p=3×10-5). In analysis of healthy tissues, notable cell type-trait pairs with high trait enrichment included monocytes and dendritic cells for Alzheimer's, GABAergic neurons for Major Depressive Disorder, Fibroblasts for Lung capacity. In disease tissue, Applicants identified a disease specific lymphocyte activation program in T Lymphocytes for Ulcerative Colitis. Genes co-expressed with COVID-19 associated genes (ACE2, TMPRSS2) in Alveolar Type 2 cells showed excess enrichment for lung capacity (0.6×, p=4×10-6). Applicants demonstrate a novel approach integrating scRNAseq, GWAS and tissue specific S2G strategies to systematically identify disease critical cell types and programs and uncover the genes driving these disease signals.

### Linking Single Cells and Single Cell Gene Programs to Biological Function Through Genetic Variants

### Identifying Genetic Variants

In certain embodiments, genetic variants are identified for subjects having a phenotype of interest (e.g., a disease) by comparing genetic variants in subjects having the phenotype and control subjects. As used herein “genetic variants” refers to any difference in DNA among individuals. Genetic variation is caused by variation in the order of bases in the nucleotides in genomic loci. Examination of DNA has shown genetic variation in both coding regions and in the non-coding intron region of genes. Genetic variations may be present in regulatory regions (e.g., promoters, enhancers, repressors) or non-protein coding genes (e.g., lncRNA, miRNA, snRNA). In certain embodiments, the genetic variants are single-nucleotide polymorphisms (SNPs). A SNP is a substitution of a single nucleotide that occurs at a specific position in the genome, where each variation is present to some appreciable degree within a population (e.g. >1%). In certain embodiments, genetic variants are identified using a biobank or database (see, e.g., UK Biobank; Bycroft et al., The UK Biobank resource with deep phenotyping and genomic data. Nature 562, 203-209 (2018); and 1000 Genomes Project Consortium. A global reference for human genetic variation. Molecular cell, 526(7571):68-74, 2015).

Example genetic variants useful in the present invention include UC specific genes identified by GWAS (Tables 1-3).

TABLE 1
Ashkenazi Jewish GWAS

| locus | alleles | genes | p_value |
|---|---|---|---|
| 17:39340812 | [“T”,“C”] | KRTAP4-1 | 2.29E−16 |
| 16:50763778 | [“G”,“GC”] | NOD2 | 2.34E−14 |
| 17:39340826 | [“T”,“C”] | KRTAP4-1 | 2.40E−13 |
| 1:67705958 | [“G”,“A”] | IL23R | 5.37E−13 |
| 14:105416323 | [“T”,“C”] | AHNAK2 | 4.75E−12 |
| 1:117122288 | [“G”,“GTCCTCC”] | IGSF3 | 1.83E−11 |
| 16:50756540 | [“G”,“C”] | NOD2 | 1.37E−10 |
| 5:140476396 | [“G”,“T”] | PCDHB2 | 5.51E−09 |
| 5:140476395 | [“T”,“C”] | PCDHB2 | 5.77E−09 |
| 1:117122269 | [“GGTC”,“G”] | IGSF3 | 6.16E−09 |
| 6:31084034 | [“C”,“T”] | CDSN | 4.74E−08 |
| 16:50745656 | [“G”,“A”] | NOD2 | 5.76E−08 |
| 16:2155426 | [“T”,“C”] | PKD1 | 7.24E−08 |
| 16:50750842 | [“A”,“G”] | NOD2 | 8.78E−08 |
| 22:38471033 | [“GGGA”,“G”] | PICK1 | 1.80E−07 |
| 16:49671101 | [“G”,“A”] | ZNF423 | 2.00E−07 |
| 16:50259156 | [“T”,“TGTC”] | PAPD5 | 2.70E−07 |
| 6:31557836 | [“C”,“T”] | NCR3 | 8.28E−07 |
| 1:225707033 | [ATCCAGGCGTTCCTG | ENAH | 1.22E−06 |
| | CCGC”,“A”] | | |
| | (SEQ ID NO: 1) | | |
| 6:31474884 | [“G”,“A”] | MICB | 3.24E−06 |
| 14:106235654 | [“T”,“C”] | IGHG3 | 6.72E−06 |
| 1:248224451 | [“T”,“C”] | OR2L3 | 9.78E−06 |
| 11:1268481 | [“G”,“A”] | MUC5B | 1.03E−05 |
| 5:140481841 | [“T”,“C”] | PCDHB3 | 1.62E−05 |
| 6:31497622 | [“A”,“G”] | MCCD1 | 2.07E−05 |
| 6:31129642 | [“A”,“G”] | TCF19 | 2.41E−05 |
| 16:2018580 | [“G”,“A”] | RNF151 | 2.67E−05 |
| 19:619099 | [“G”,“A”] | POLRMT | 2.68E−05 |
| 1:248524937 | [“ATGGGACTCTTCA | OR2T4 | 3.40E−05 |
| | GACAATCCAAACATC | | |
| | CAATGGCCAATATCA | | |
| | CCTGGATGGCCAACC | | |
| | ACACTGGATGGTCGG | | |
| | ATTTCATCCTGT”, | | |
| | “A”] | | |
| | (SEQ ID NO: 2) | | |
| 16:50745926 | [“C”,“T”] | NOD2 | 8.27E−05 |
| 6:32363888 | [“C”,“T”] | BTNL2 | 1.06E−04 |
| 6:32369554 | [“G”,“A”] | BTNL2 | 1.10E−04 |
| 6:32370927 | [“G”,“A”] | BTNL2 | 1.33E−04 |
| 6:32370791 | [“G”,“A”] | BTNL2 | 1.34E−04 |
| 6:32363973 | [“C”,“T”] | BTNL2 | 1.52E−04 |
| 6:32362521 | [“C”,“A”] | BTNL2 | 1.56E−04 |
| 6:32370860 | [“G”,“A”] | BTNL2 | 1.71E−04 |
| 6:32364011 | [“T”,“C”] | BTNL2 | 1.77E−04 |
| 6:32364046 | [“T”,“A”] | BTNL2 | 2.03E−04 |
| 6:32364052 | [“C”,“T”] | BTNL2 | 2.26E−04 |
| 6:32364057 | [“C”,“T”] | BTNL2 | 2.68E−04 |
| 17:4837117 | [“AAGCCCGACCAC | GP1BA | 3.91E−04 |
| | CCCAGAGCCCACCT | | |
| | CAGAGCCCGCCCCC | | |
| | AGCCCGACCACCCC | | |
| | GGAGCCCACCTCAG | | |
| | AGCCCGCCCCC”, | | |
| | “A”] | | |
| | (SEQ ID NO: 3) | | |
| 6:32369586 | [“GAA”,“G”] | BTNL2 | 4.61E−04 |
| 19:55144141 | [“A”,“G”] | LILRB1 | 4.87E−04 |
| 19:49878275 | [“G”,“A”] | DKKL1 | 6.97E−04 |
| 9:139358899 | [“C”,“T”] | SEC16A | 7.71E−04 |

TABLE 2
Finnish GWAS

| locus | alleles | genes | beta | p_value |
|---|---|---|---|---|
| 1:248224451 | [“T”,“C”] | OR2L3 | −3.41E−01 | 1.00E−16 |
| 19:43031248 | [“T”,“G”] | CEACAM1 | −2.89E−01 | 1.05E−14 |
| 17:4837117 | [“AAGCCCGACCACCCCA | GP1BA | 3.34E−01 | 2.08E−12 |
| | GAGCCCACCTCAGCCCC | | | |
| | AGCCCGCAGCCCGACCA | | | |
| | CCCCGGAGCCCACCTCA | | | |
| | GAGCCCGCCCCC”, | | | |
| | “A”] (SEQ | | | |
| | ID NO: 4) | | | |
| 19:55148045 | [“G”,“A”] | LILRB1 | 2.66E−01 | 4.00E−10 |
| 19:55148043 | [“T”,“C”] | LILRB1 | 2.62E−01 | 4.71E−10 |
| 17:39340812 | [“T”,“C”] | KRTAP4-1 | −2.86E−01 | 1.42E−09 |
| 4:69202890 | [“TTCC”,“T”] | YTHDC1 | −3.88E−01 | 2.62E−09 |
| 17:55183813 | [“A”,“G”] | AKAP1 | 2.74E−01 | 1.64E−08 |
| 17:55183792 | [“G”,“A”] | AKAP1 | 2.67E−01 | 1.24E−07 |
| 10:30316500 | [“ACTG”,“A”] | KIAA1462 | −3.12E−01 | 1.43E−07 |
| 11:1651594 | [“AGTCC”,“A”] | KRTAP5-5 | 2.72E−01 | 3.59E−07 |
| 5:140482102 | [“A”,“G”] | PCDHB3 | 1.92E−01 | 1.11E−06 |
| 11:55595017 | [“G”,“T”] | OR5L2 | 2.82E−01 | 1.52E−06 |
| 11:55595018 | [“A”,“G”] | OR5L2 | 2.82E−01 | 1.59E−06 |
| 1:12921576 | [“C”,“T”] | PRAMEF2 | 1.55E−01 | 1.60E−06 |
| 19:55494612 | [“A”,“G”] | NLRP2 | 2.97E−01 | 1.78E−06 |
| 17:39340826 | [“T”,“C”] | KRTAP4-1 | −2.57E−01 | 1.83E−06 |
| 19:22939455 | [“GTTTCATAA”, | ZNF99 | 3.05E−01 | 2.22E−06 |
| | “G”] | | | |
| 19:22939464 | [“GGGTCGAGAAATTGT | ZNF99 | 3.05E−01 | 2.26E−06 |
| | TAAAACCTTTGCCACA | | | |
| | TTCTTCACATTTGTA | | | |
| | CGGTTTCTCCCC | | | |
| | AGTATGAATTAT | | | |
| | CTTATGT”,“G”] | | | |
| | (SEQ ID NO: | | | |
| | 5) | | | |
| 11:55595012 | [“A”,“T”] | OR5L2 | 2.90E−01 | 5.00E−06 |
| 1:1420527 | [“G”,“T”] | ATAD3B | 1.99E−01 | 8.25E−06 |
| 7:5327564 | [“G”,“A”] | SLC29A4 | 1.48E−01 | 9.44E−06 |
| 14:106780727 | [“T”,“C”] | IGHV4-28 | 3.23E−01 | 1.19E−05 |
| 19:20807133 | [“GGCTTTGCCACATTC | ZNF626 | 1.71E−01 | 1.41E−05 |
| | TTCACATTTGTAGAA | | | |
| | TTTCTCTCCAGTA | | | |
| | TGATTCTCTCATGT | | | |
| | GTAGTAAGGATTGA | | | |
| | GGACTGGTTGAAGG | | | |
| | CTTTGCCACATTCT | | | |
| | TCACATTTGTAGG | | | |
| | GTCTCTCTCCAGT | | | |
| | ATGAATTTTCTTA | | | |
| | TGTGTAGTAAGGTT | | | |
| | AGAGGAGCACTTAA | | | |
| | AA”,“G”] | | | |
| | (SEQ ID NO: | | | |
| | 6) | | | |
| 11:1643227 | [“AGCCACAGCCC | KRTAP5-4 | 3.16E−01 | 2.23E−05 |
| | CCACAGCCAGAGC | | | |
| | CACAGCCCCCACA | | | |
| | GCCG”,“A”] | | | |
| | (SEQ ID NO: | | | |
| | 7) | | | |
| 1:11252369 | [“G”,“A”] | ANGPTL7 | −4.77E−01 | 2.53E−05 |
| 17:76510974 | [“G”,“A”] | DNAH17 | −3.22E−01 | 3.20E−05 |
| 19:56206137 | [“G”,“C”] | EPN1 | −2.44E−01 | 3.54E−05 |
| 2:28464198 | [“C”,“T”] | BRE | −2.75E−01 | 3.64E−05 |
| 1:226075708 | [“A”,“G”] | LEFTY1 | 2.88E−01 | 4.27E−05 |
| 19:2939267 | [“CACCACCCTTACCCA | ZNF77 | 3.29E−01 | 7.09E−05 |
| | AGGAGGCA”, | | | |
| | “C”] (SEQ ID | | | |
| | NO: 8) | | | |
| 2:233273011 | [“C”,“G”] | ALPPL2 | 2.69E−01 | 1.61E−04 |
| 1:12943171 | [“T”,“C”] | PRAMEF4 | 3.24E−01 | 1.85E−04 |
| 11:1265474 | [“C”,“T”] | MUC5B | 2.82E−01 | 1.88E−04 |
| 11:1643224 | [“CGG”,“C”] | KRTAP5-4 | 2.44E−01 | 2.09E−04 |
| 11:1265481 | [“C”,“T”] | MUC5B | 2.79E−01 | 2.16E−04 |
| 21:46011718 | [“T”,“C”] | KRTAP10-6 | 3.61E−01 | 2.36E−04 |
| 14:22476138 | [“AGGT”,“A”] | TRAV19 | −1.30E−01 | 3.19E−04 |
| 11:1265450 | [“A”,“C”] | MUC5B | 2.42E−01 | 3.71E−04 |
| 16:2155426 | [“T”,“C”] | PKD1 | 1.14E−01 | 4.06E−04 |
| 19:55144141 | [“A”,“G”] | LILRB1 | −2.09E−01 | 4.95E−04 |
| 1:248458419 | [“G”,“C”] | OR2T12 | 2.18E−01 | 4.95E−04 |
| 6:29523957 | [“A”,“G”] | UBD | 1.09E−01 | 5.50E−04 |
| 1:16073524 | [“C”,“CGA”] | TMEM82 | −2.20E−01 | 5.93E−04 |
| 1:16073525 | [“C”,“T”] | TMEM82 | −2.19E−01 | 6.57E−04 |
| 22:22782210 | [“T”,“A”] | IGLV5-37 | −8.36E−02 | 1.12E−03 |
| 6:28268824 | [“A”,“G”] | PGBD1 | 1.03E−01 | 1.25E−03 |
| 1:225707033 | [“ATCCAGGCGTTCCTG | ENAH | 1.19E−01 | 1.28E−03 |
| | CCGC”,“A”] | | | |
| | (SEQ ID NO: | | | |
| | 9) | | | |
| 1:248524937 | [“ATGGGACTCTT | OR2T4 | 1.02E−01 | 1.59E−03 |
| | CAGACAATCCAAA | | | |
| | CATCCAATGGCCA | | | |
| | ATATCACCTGGAT | | | |
| | GGCCAACCACACT | | | |
| | GGATGGTCGGATT | | | |
| | TCATCCTGT”, | | | |
| | “A”] | | | |
| | (SEQ ID NO: | | | |
| | 10) | | | |

TABLE 3
Non-Finnish European GWAS

| locus | allele | gene | pvalue |
|---|---|---|---|
| 17:39340812 | [“T”,“C”] | KRTAP4-1 | 3.08E−74 |
| 16:50763778 | [“G”,“GC”] | NOD2 | 6.18E−68 |
| 1:67705958 | [“G”,“A”] | IL23R | 6.03E−44 |
| 17:39340826 | [“T”,“C”] | KRTAP4-1 | 6.14E−36 |
| 1:248224451 | [“T”,“C”] | OR2L3 | 6.12E−35 |
| 16:50745926 | [“C”,“T”] | NOD2 | 9.53E−28 |
| 6:31915614 | [“G”,“A”] | CFB | 7.81E−25 |
| 1:225707033 | [“ATCCAGGCGTTC | ENAH | 3.62E−24 |
| | CTGCCGC”,“A”] | | |
| | (SEQ ID NO: 11) | | |
| 16:50756540 | [“G”,“C”] | NOD2 | 1.41E−23 |
| 21:46011718 | [“T”,“C”] | KRTAP10-6 | 1.53E−23 |
| 16:2142083 | [“C”,“G”] | PKD1 | 1.42E−16 |
| 1:12943171 | [“T”,“C”] | PRAMEF4 | 2.24E−16 |
| 19:43031248 | [“T”,“G”] | CEACAM1 | 2.81E−16 |
| 19:55148043 | [“T”,“C”] | LILRB1 | 1.65E−15 |
| 19:55148045 | [“G”,“A”] | LILRB1 | 3.00E−15 |
| 19:2939267 | [“CACCACCCTTAC | ZNF77 | 3.13E−15 |
| | CCAAGGAGGCA”, | | |
| | “C”] (SEQ ID | | |
| | NO: 12) | | |
| 2:233712227 | [“A”,“G”] | GIGYF2 | 4.04E−15 |
| 22:22782210 | [“T”,“A”] | IGLV5-37 | 1.15E−14 |
| 17:43552812 | [“A”,“G”] | PLEKHM1 | 1.40E−14 |
| 11:55595017 | [“G”,“T”] | OR5L2 | 3.68E−14 |
| 16:2155426 | [“T”,“C”] | PKD1 | 6.09E−14 |
| 11:55595018 | [“A”,“G”] | OR5L2 | 6.28E−14 |
| 9:139259592 | [“C”,“G”] | CARD9 | 1.05E−13 |
| 17:5038533 | [“A”,“C”] | USP6 | 1.62E−13 |
| 11:55595012 | [“A”,“T”] | OR5L2 | 2.73E−13 |
| 6:32007840 | [“C”,“T”] | CYP21A2 | 8.38E−13 |
| 17:55183813 | [“A”,“G”] | AKAP1 | 2.19E−12 |
| 11:55111057 | [“G”,“A”] | OR4A16 | 2.91E−12 |
| 1:12943144 | [“A”,“G”] | PRAMEF4 | 4.85E−12 |
| 11:55111118 | [“A”,“G”] | OR4A16 | 7.48E−12 |
| 11:1651594 | [“AGTCC”,“A”] | KRTAP5-5 | 9.05E−12 |
| 17:55183792 | [“G”,“A”] | AKAP1 | 2.24E−11 |
| 15:75981972 | [“A”,“G”] | CSPG4 | 2.84E−11 |
| 1:12941832 | [“T”,“C”] | PRAMEF4 | 3.10E−11 |
| 1:16073524 | [“C”,“CGA”] | TMEM82 | 5.63E−11 |
| 1:16073525 | [“C”,“T”] | TMEM82 | 5.82E−11 |
| 19:54721090 | [“A”,“G”] | LILRA6 | 7.25E−11 |
| 19:54721090 | [“A”,“G”] | LILRB3 | 7.25E−11 |
| 22:21998280 | [“G”,“A”] | SDF2L1 | 1.08E−09 |
| 6:32370860 | [“G”,“A”] | BTNL2 | 1.41E−09 |
| 6:32362785 | [“G”,“A”] | BTNL2 | 1.93E−09 |
| 1:22310235 | [“C”,“T”] | CELA3B | 2.18E−09 |
| 6:32370927 | [“G”,“A”] | BTNL2 | 2.26E−09 |
| 6:32363888 | [“C”,“T”] | BTNL2 | 2.88E−09 |
| 6:32364052 | [“C”,“T”] | BTNL2 | 2.91E−09 |
| 6:32364011 | [“T”,“C”] | BTNL2 | 2.91E−09 |
| 6:32364057 | [“C”,“T”] | BTNL2 | 2.92E−09 |
| 6:29523957 | [“A”,“G”] | UBD | 2.95E−09 |
| 6:32363973 | [“C”,“T”] | BTNL2 | 3.91E−09 |
| 6:32370791 | [“G”,“A”] | BTNL2 | 4.46E−09 |
| 10:37438725 | [“C”,“G”] | ANKRD30A | 6.26E−09 |
| 6:32364046 | [“T”,“A”] | BTNL2 | 6.26E−09 |
| 14:22476138 | [“AGGT”,“A”] | TRAV19 | 6.63E−09 |
| 6:32362521 | [“C”,“A”] | BTNL2 | 7.90E−09 |
| 6:31084034 | [“C”,“T”] | CDSN | 8.25E−09 |
| 16:14958514 | [“A”,“G”] | NOMO1 | 1.04E−08 |
| 1:117122288 | [“G”,“GTCCTCC”] | IGSF3 | 2.71E−08 |
| 6:31557836 | [“C”,“T”] | NCR3 | 3.55E−08 |
| 6:28891176 | [“T”,“C”] | TRIM27 | 1.10E−07 |
| 11:1265450 | [“A”,“C”] | MUC5B | 1.22E−07 |
| 6:26637724 | [“T”,“C”] | ZNF322 | 1.32E−07 |
| 6:32713044 | [“C”,“T”] | HLA-DQA2 | 1.50E−07 |
| 11:1643224 | [“CGG”,“C”] | KRTAP5-4 | 1.72E−07 |
| 11:1643227 | [“AGCCACAGCCCC | KRTAP5-4 | 1.85E−07 |
| | CACAGCCAGAGCC | | |
| | ACAGCCCCCACAG | | |
| | CCG”,“A”] (SEQ | | |
| | ID NO: 13) | | |
| 12:40740686 | [“A”,“G”] | LRRK2 | 2.25E−07 |
| 19:22939455 | [“GTTTCATAA”,“G”] | ZNF99 | 2.97E−07 |
| 6:32782897 | [“C”,“T”] | HLA-DOB | 3.11E−07 |
| 6:32782897 | [“C”,“T”] | TAP2 | 3.11E−07 |
| 5:140476396 | [“G”,“T”] | PCDHB2 | 3.29E−07 |
| 6:32052216 | [“C”,“T”] | TNXB | 3.40E−07 |
| 2:233273011 | [“C”,“G”] | ALPPL2 | 3.53E−07 |
| 19:22939464 | [“GGGTCGAGAAAT | ZNF99 | 3.61E−07 |
| | TGTTAAAACCTTTG | | |
| | CCACATTCTTCACA | | |
| | TTTGTACGGTTTCT | | |
| | CCCCAGTATGAATT | | |
| | ATCTTATGT”,“G”] | | |
| | (SEQ ID NO: 14) | | |
| 6:32036822 | [“C”,“T”] | TNXB | 4.16E−07 |
| 1:161596014 | [“A”,“G”] | FCGR3B | 4.42E−07 |
| 6:32020717 | [“G”,“T”] | TNXB | 4.56E−07 |
| 6:28268824 | [“A”,“G”] | PGBD1 | 5.77E−07 |
| 6:26199903 | [“C”,“T”] | HIST1H2BF | 6.20E−07 |
| 5:140476395 | [“T”,“C”] | PCDHB2 | 6.42E−07 |
| 9:5126343 | [“G”,“A”] | JAK2 | 6.66E−07 |
| 6:32369586 | [“GAA”,“G”] | BTNL2 | 6.85E−07 |
| 6:32168996 | [“C”,“G”] | NOTCH4 | 7.18E−07 |
| 6:27879982 | [“A”,“G”] | OR2B2 | 7.56E−07 |
| 6:27879200 | [“C”,“A”] | OR2B2 | 8.72E−07 |
| 9:139358899 | [“C”,“T”] | SEC16A | 9.13E−07 |
| 1:67705900 | [“G”,“A”] | IL23R | 1.08E−06 |
| 2:227661395 | [“TTGC”,“T”] | IRS1 | 1.17E−06 |
| 6:26463574 | [“G”,“T”] | BTN2A1 | 1.35E−06 |
| 6:26463575 | [“G”,“T”] | BTN2A1 | 1.35E−06 |
| 1:248458419 | [“G”,“C”] | OR2112 | 1.74E−06 |
| 6:31474884 | [“G”,“A”] | MICB | 1.78E−06 |
| 11:65425764 | [“C”,“T”] | RELA | 1.84E−06 |
| 11:65715204 | [“G”,“A”] | TSGA10IP | 2.02E−06 |
| 6:32369554 | [“G”,“A”] | BTNL2 | 2.36E−06 |
| 6:31379990 | [“C”,“G”] | MICA | 2.44E−06 |
| 2:9661450 | [“A”,“G”] | ADAM17 | 2.59E−06 |
| 2:233273018 | [“G”,“A”] | ALPPL2 | 2.60E−06 |
| 3:49722706 | [“G”,“A”] | MST1 | 2.84E−06 |
| 22:43616565 | [“G”,“C”] | SCUBE1 | 2.89E−06 |
| 19:10464843 | [“G”,“A”] | TYK2 | 2.99E−06 |
| 6:31496949 | [“C”,“T”] | MCCD1 | 3.23E−06 |
| 5:140482102 | [“A”,“G”] | PCDHB3 | 3.26E−06 |
| 6:31379043 | [“A”,“G”] | MICA | 3.49E−06 |
| 11:1651652 | [“C”,“T”] | KRTAP5-5 | 3.95E−06 |
| 19:49910139 | [“C”,“G”] | CCDC155 | 4.00E−06 |
| 4:114294536 | [“C”,“T”] | ANK2 | 4.04E−06 |
| 19:54848145 | [“G”,“A”] | LILRA4 | 4.28E−06 |
| 19:54848144 | [“T”,“A”] | LILRA4 | 4.33E−06 |
| 14:106478531 | [“G”,“A”] | IGHV4-4 | 4.41E−06 |
| 14:105416380 | [“A”,“G”] | AHNAK2 | 4.50E−06 |
| 1:150530548 | [“C”,“G”] | ADAMTSL4 | 5.33E−06 |
| 3:58508217 | [“G”,“A”] | ACOX2 | 5.35E−06 |
| 20:18374929 | [“A”,“G”] | DZANK1 | 5.42E−06 |
| 20:55108506 | [“C”,“CAATA”] | FAM209B | 5.95E−06 |
| 20:55108507 | [“CGTGT”,“C”] | FAM209B | 5.95E−06 |
| 6:52762717 | [“T”,“C”] | GSTA3 | 7.24E−06 |
| 6:32021414 | [“C”,“T”] | TNXB | 7.42E−06 |
| 6:32261153 | [“C”,“T”] | C6orf10 | 7.71E−06 |
| 6:32006896 | [“G”,“C”] | CYP21A2 | 8.31E−06 |
| 16:81916912 | [“A”,“G”] | PLCG2 | 9.31E−06 |
| 11:1265474 | [“C”,“T”] | MUC5B | 9.47E−06 |
| 6:27835218 | [“G”,“A”] | HIST1H1B | 1.04E−05 |
| 22:32548558 | [“T”,“C”] | C22orf42 | 1.16E−05 |
| 16:2136842 | [“C”,“T”] | TSC2 | 1.21E−05 |
| 2:233271799 | [“C”,“G”] | ALPPL2 | 1.25E−05 |
| 22:42537885 | [“T”,“A”] | CYP2D7P | 1.27E−05 |
| 11:1265481 | [“C”,“T”] | MUC5B | 1.29E−05 |
| 19:49619561 | [“T”,“C”] | LIN7B | 1.32E−05 |
| 19:49878275 | [“G”,“A”] | DKKL1 | 1.33E−05 |
| 22:39439067 | [“G”,“C”] | APOBEC3F | 1.42E−05 |
| 22:42537889 | [“T”,“C”] | CYP2D7P | 1.50E−05 |
| 22:32548561 | [“C”,“T”] | C22orf42 | 1.61E−05 |
| 2:96689178 | [“G”,“A”] | GPAT2 | 1.68E−05 |
| 4:103188709 | [“C”,“T”] | SLC39A8 | 1.70E−05 |
| 14:106780727 | [“T”,“C”] | IGHV4-28 | 1.82E−05 |
| 20:46279860 | [“GCAGCAA”,“G”] | NCOA3 | 1.90E−05 |

In certain embodiments, sequencing is used to identify genetic variants. In certain embodiments, sequencing comprises high-throughput (formerly “next-generation”) technologies to generate sequencing reads. In DNA sequencing, a read is an inferred sequence of base pairs (or base pair probabilities) corresponding to all or part of a single DNA fragment. A typical sequencing experiment involves fragmentation of the genome into millions of molecules or generating complementary DNA (cDNA) fragments, which are size-selected and ligated to adapters. The set of fragments is referred to as a sequencing library, which is sequenced to produce a set of reads. Methods for constructing sequencing libraries are known in the art (see, e.g., Head et al., Library construction for next-generation sequencing: Overviews and challenges. Biotechniques. 2014; 56(2): 61-77). A “library” or “fragment library” may be a collection of nucleic acid molecules derived from one or more nucleic acid samples, in which fragments of nucleic acid have been modified, generally by incorporating terminal adapter sequences comprising one or more primer binding sites and identifiable sequence tags. In certain embodiments, the library members (e.g., genomic DNA, cDNA) may include sequencing adaptors that are compatible with use in, e.g., Illumina's reversible terminator method, long read nanopore sequencing, Roche's pyrosequencing method (454), Life Technologies' sequencing by ligation (the SOLiD platform) or Life Technologies' Ion Torrent platform. Examples of such methods are described in the following references: Margulies et al (Nature 2005 437: 376-80); Schneider and Dekker (Nat Biotechnol. 2012 Apr. 10; 30(4):326-8); Ronaghi et al (Analytical Biochemistry 1996 242: 84-9); Shendure et al (Science 2005 309: 1728-32); Imelfort et al (Brief Bioinform. 2009 10:609-18); Fox et al (Methods Mol. Biol. 2009; 553:79-108); Appleby et al (Methods Mol. Biol. 2009; 513:19-39); and Morozova et al (Genomics. 2008 92:255-64), which are incorporated by reference for the general descriptions of the methods and the particular steps of the methods, including all starting products, reagents, and final products for each of the steps.

In certain embodiments, the present invention includes whole genome sequencing. Whole genome sequencing (also known as WGS, full genome sequencing, complete genome sequencing, or entire genome sequencing) is the process of determining the complete DNA sequence of an organism's genome at a single time. This entails sequencing all of an organism's chromosomal DNA as well as DNA contained in the mitochondria and, for plants, in the chloroplast. “Whole genome amplification” (“WGA”) refers to any amplification method that aims to produce an amplification product that is representative of the genome from which it was amplified. Non-limiting WGA methods include Primer extension PCR (PEP) and improved PEP (I-PEP), Degenerated oligonucleotide primed PCR (DOP-PCR), Ligation-mediated PCR (LMP), T7-based linear amplification of DNA (TLAD), and Multiple displacement amplification (MDA).

In certain embodiments, the present invention includes whole exome sequencing. Exome sequencing, also known as whole exome sequencing (WES), is a genomic technique for sequencing all of the protein-coding genes in a genome (known as the exome) (see, e.g., Ng et al., 2009, Nature volume 461, pages 272-276). It consists of two steps: the first step is to select only the subset of DNA that encodes proteins. These regions are known as exons—humans have about 180,000 exons, constituting about 1% of the human genome, or approximately 30 million base pairs. The second step is to sequence the exonic DNA using any high-throughput DNA sequencing technology. In certain embodiments, whole exome sequencing is used to determine genetic variants in genes associated with disease (e.g., disease genes).

In certain embodiments, targeted sequencing is used in the present invention (see, e.g., Mantere et al., PLoS Genet 12 e1005816 2016; and Carneiro et al. BMC Genomics, 2012 13:375). Targeted gene sequencing panels are useful tools for analyzing specific mutations in a given sample. Focused panels contain a select set of genes or gene regions that have known or suspected associations with the disease or phenotype under study. In certain embodiments, targeted sequencing is used to detect mutations associated with a disease in a subject in need thereof. Targeted sequencing can increase the cost-effectiveness of variant discovery and detection.

In certain embodiments, multiple displacement amplification (MDA) is used to generate a sequencing library (e.g., single cell genome sequencing). Multiple displacement amplification (MDA, is a non-PCR-based isothermal method based on the annealing of random hexamers to denatured DNA, followed by strand-displacement synthesis at constant temperature (Blanco et al. J. Biol. Chem. 1989, 264, 8935-8940). It has been applied to samples with small quantities of genomic DNA, leading to the synthesis of high molecular weight DNA with limited sequence representation bias (Lizardi et al. Nature Genetics 1998, 19, 225-232; Dean et al., Proc. Natl. Acad. Sci. U.S.A 2002, 99, 5261-5266). As DNA is synthesized by strand displacement, a gradually increasing number of priming events occur, forming a network of hyper-branched DNA structures. The reaction can be catalyzed by enzymes such as the Phi29 DNA polymerase or the large fragment of the Bst DNA polymerase. The Phi29 DNA polymerase possesses a proofreading activity resulting in error rates 100 times lower than Taq polymerase (Lasken et al. Trends Biotech. 2003, 21, 531-535).

### Single Cell Atlases

A single cell atlas can be used in combination with genetics. As used herein “single cell atlas” refers to any collection of single cell data from any tissue sample of interest having a phenotype of interest (see, e.g., Rozenblatt-Rosen O, Stubbington M J T, Regev A, Teichmann S A., The Human Cell Atlas: from vision to reality, Nature. 2017 Oct. 18; 550(7677):451-453; and Regev, A. et al. The Human Cell Atlas Preprint available at bioRxiv at dx.doi.org/10.1101/121202 (2017)). In preferred embodiments, single cell data is obtained from one or more tissue samples, more preferably, one or more tissue samples from one or more subjects. The subjects preferably include one or more subjects having a phenotype and one or more control subjects. The phenotype of the tissue sample can be a diseased phenotype and the atlas can compare diseased tissue to healthy tissue. The single cell data can include, but is not limited to transcriptome, chromatin accessibility, epigenetic data, or any combination thereof. A single cell atlas can refer to any collection of single cell data from any tissue sample. The number of cells analysed in the atlas may be about 1,000, 2,000, 5,000, 10,000, 20,000, 50,000, 100,000, 500,000, or more than a million cells. The single cell atlas can also include biological and medical information for the subjects where the tissue samples were obtained.

A single cell atlas for a tissue may be constructed by measuring single cell transcriptomes. In certain embodiments, the single cell data comprises single cell RNA-seq data (scRNA-seq) or single nucleus RNA-seq data (snRNA-seq). The single cell atlas can be used as a roadmap for any phenotype present in or associated with a specific tissue (e.g., a “Google Map” of patient tissue samples). The atlas can be generated by providing: (1) biological information, including medical records, histology, single cell profiles, and genetic information, and (2) data, including multiplexed ion beam imaging (MIBI) (see, e.g., Angelo et al., Nat Med. 2014 April; 20(4): 436-442), NanoString (DSP, digital spatial profiling) (see e.g., Geiss G K, et al., Direct multiplexed measurement of gene expression with color-coded probe pairs. Nat Biotechnol. 2008 March; 26(3):317-25), microbiome, immunoprofiling, and sequencing (e.g., bulk and single cell sequencing). Pathology of tissue samples can be performed. Tissue samples can be dissociated for scRNA-seq, flow cytometry and cell culture. Tissues can also be snap frozen for analysis of DNA by WES, bulk RNA-seq, and epigenetics. Tissue can also be OCT frozen for multiplex imaging. The data obtained can be computationally analyzed.

Non-limiting examples of a single cell atlas applicable to the present invention are disclosed in U.S. patent Ser. No. 16/072,674, International Patent Publication Nos. WO 2018/191520 and WO 2018/191558, U.S. patent Ser. No. 16/348,911, International Patent Publication No. WO 2019/018440, U.S. patent Ser. No. 15/844,601, and U.S. Provisional Application No. 62/888,347. See, also, Darmanis, S. et al. Proc. Natl Acad. Sci. USA 112, 7285-7290 (2015); Lake, B. B. et al. Science 352, 1586-1590 (2016); Pollen, A. A. et al. Nature Biotechnol. 32, 1053-1058 (2014); Tasic, B. et al. Nature Neurosci. 19, 335-346 (2016); Zeisel, A. et al. Science 347, 1138-1142 (2015); Grun. D. et al Nature 525, 251-255 (2015); Shekhar, K. et al. Cell 166, 1308-1323 (2016); Villani, A. C. et al. Science 356, eaah4573 (2017); Lönnberg, T. et al. Sci. Immunol. 2, eaa12192 (2017); Tirosh, I. et al. Science 352, 189-196 (2016); Venteicher A S, et al., Decoupling genetics, lineages, and microenvironment in IDH-mutant gliomas by single-cell RNA-seq., Science. 2017 Mar. 31; 355(6332); Tirosh, I. et al. Single-cell RNA-seq supports a developmental hierarchy in human oligodendroglioma. Nature. 2016 Nov. 10; 539(7628):309-313; Drokhlyansky et al., The enteric nervous system of the human and mouse colon at a single-cell resolution. bioRxiv 746743; doi: doi.org/10.1101/746743; Smillie C S. et al., Intra- and Inter-cellular Rewiring of the Human Colon during Ulcerative Colitis. Cell. 2019 Jul. 25; 178(3):714-730.e22; Montoro D T. et al., A revised airway epithelial hierarchy includes CFTR-expressing ionocytes. Nature. 2018 August; 560(7718):319-324; Haber A L, et al., A single-cell survey of the small intestinal epithelium. Nature. 2017 Nov. 16; 551(7680):333-339; Wang, et al., The Allen Mouse Brain Common Coordinate Framework: A 3D Reference Atlas, Cell. 2020 May 14; 181(4):936-953.e20; Lein E, et al. Genome-wide atlas of gene expression in the adult mouse brain. Nature, 2007; 445:168-76; and Allen Mouse Brain Atlas: mouse.brain-map.org/. Smillie et al. shows a cell atlas of UC, a complex disease atlas. Smillie et al. further shows that the atlas can be built from involved and uninvolved tissue in patients, in comparison to the healthy reference from a human cell atlas. A relatively small number of individuals provides a robust catalog (i.e., atlas).

In certain embodiments, single cell transcriptomes are included in the cell atlas. As used herein the term “transcriptome” refers to the set of transcripts molecules. In some embodiments, transcript refers to RNA molecules, e.g., messenger RNA (mRNA) molecules, small interfering RNA (siRNA) molecules, transfer RNA (tRNA) molecules, ribosomal RNA (rRNA) molecules, and complimentary sequences, e.g., cDNA molecules. In some embodiments, a transcriptome refers to a set of mRNA molecules. In some embodiments, a transcriptome refers to a set of cDNA molecules. In some embodiments, a transcriptome refers to one or more of mRNA molecules, siRNA molecules, tRNA molecules, rRNA molecules, in a sample, for example, a single cell or a population of cells. In some embodiments, a transcriptome refers to cDNA generated from one or more of mRNA molecules, siRNA molecules, tRNA molecules, rRNA molecules, in a sample, for example, a single cell or a population of cells. In some embodiments, a transcriptome refers to 50%, 55, 60, 65, 70, 75, 80, 85, 90, 91, 92, 93, 94, 95, 96, 97, 98, 99, 99.9, or 100% of transcripts from a single cell or a population of cells. In some embodiments, transcriptome not only refers to the species of transcripts, such as mRNA species, but also the amount of each species in the sample. In some embodiments, a transcriptome includes each mRNA molecule in the sample, such as all the mRNA molecules in a single cell.

In certain embodiments, the invention involves single cell RNA sequencing (see, e.g., Kalisky, T., Blainey, P. & Quake, S. R. Genomic Analysis at the Single-Cell Level. Annual review of genetics 45, 431-445, (2011); Kalisky, T. & Quake, S. R. Single-cell genomics. Nature Methods 8, 311-314 (2011); Islam, S. et al. Characterization of the single-cell transcriptional landscape by highly multiplex RNA-seq. Genome Research, (2011); Tang, F. et al. RNA-Seq analysis to capture the transcriptome landscape of a single cell. Nature Protocols 5, 516-535, (2010); Tang, F. et al. mRNA-Seq whole-transcriptome analysis of a single cell. Nature Methods 6, 377-382, (2009); Ramskold, D. et al. Full-length mRNA-Seq from single-cell levels of RNA and individual circulating tumor cells. Nature Biotechnology 30, 777-782, (2012); and Hashimshony, T., Wagner, F., Sher, N. & Yanai, I. CEL-Seq: Single-Cell RNA-Seq by Multiplexed Linear Amplification. Cell Reports, Cell Reports, Volume 2, Issue 3, p 666-6′73, 2012).

In certain embodiments, the present invention involves single cell RNA sequencing (scRNA-seq). In certain embodiments, the invention involves plate based single cell RNA sequencing (see, e.g., Picelli, S. et al., 2014, “Full-length RNA-seq from single cells using Smart-seq2” Nature protocols 9, 171-181, doi:10.1038/nprot.2014.006).

In certain embodiments, the invention involves high-throughput single-cell RNA-seq where the RNAs from different cells are tagged individually, allowing a single library to be created while retaining the cell identity of each read. In this regard reference is made to Macosko et al., 2015, “Highly Parallel Genome-wide Expression Profiling of Individual Cells Using Nanoliter Droplets” Cell 161, 1202-1214; International Patent Application No. PCT/US2015/049178, published as WO2016/040476 on Mar. 17, 2016; Klein et al., 2015, “Droplet Barcoding for Single-Cell Transcriptomics Applied to Embryonic Stem Cells” Cell 161, 1187-1201; International Patent Application No. PCT/US2016/027734, published as WO2016168584A1 on Oct. 20, 2016; Zheng, et al., 2016, “Haplotyping germline and cancer genomes with high-throughput linked-read sequencing” Nature Biotechnology 34, 303-311; Zheng, et al., 2017, “Massively parallel digital transcriptional profiling of single cells” Nat. Commun. 8, 14049 doi: 10.1038/ncomms14049; International Patent Publication No. WO2014210353A2; Zilionis, et al., 2017, “Single-cell barcoding and sequencing using droplet microfluidics” Nat Protoc. January; 12(1):44-73; Cao et al., 2017, “Comprehensive single cell transcriptional profiling of a multicellular organism by combinatorial indexing” bioRxiv preprint first posted online Feb. 2, 2017, doi: dx.doi.org/10.1101/104844; Rosenberg et al., 2017, “Scaling single cell transcriptomics through split pool barcoding” bioRxiv preprint first posted online Feb. 2, 2017, doi: dx.doi.org/10.1101/105163; Rosenberg et al., “Single-cell profiling of the developing mouse brain and spinal cord with split-pool barcoding” Science 15 Mar. 2018; Vitak, et al., “Sequencing thousands of single-cell genomes with combinatorial indexing” Nature Methods, 14(3):302-308, 2017; Cao, et al., Comprehensive single-cell transcriptional profiling of a multicellular organism. Science, 357(6352):661-667, 2017; Gierahn et al., “Seq-Well: portable, low-cost RNA sequencing of single cells at high throughput” Nature Methods 14, 395-398 (2017); and Hughes, et al., “Highly Efficient, Massively-Parallel Single-Cell RNA-Seq Reveals Cellular States and Molecular Features of Human Skin Pathology” bioRxiv 689273; doi: doi.org/10.1101/689273, all the contents and disclosure of each of which are herein incorporated by reference in their entirety.

In certain embodiments, the invention involves single nucleus RNA sequencing. In this regard reference is made to Swiech et al., 2014, “In vivo interrogation of gene function in the mammalian brain using CRISPR-Cas9” Nature Biotechnology Vol. 33, pp. 102-106; Habib et al., 2016, “Div-Seq: Single-nucleus RNA-Seq reveals dynamics of rare adult newborn neurons” Science, Vol. 353, Issue 6302, pp. 925-928; Habib et al., 2017, “Massively parallel single-nucleus RNA-seq with DroNc-seq” Nat Methods. 2017 October; 14(10):955-958; International Patent Application No. PCT/US2016/059239, published as WO2017164936 on Sep. 28, 2017; International Patent Application No. PCT/US2018/060860, published as WO/2019/094984 on May 16, 2019; International Patent Application No. PCT/US2019/055894, published as WO/2020/077236 on Apr. 16, 2020; and Drokhlyansky, et al., “The enteric nervous system of the human and mouse colon at a single-cell resolution,” bioRxiv 746743; doi: doi.org/10.1101/746743, which are herein incorporated by reference in their entirety.

In certain embodiments, a single cell atlas includes single cell chromatin accessibility data. A single cell atlas for a tissue may include analysis of open or accessible chromatin in single cells. In certain embodiments, the invention involves the Assay for Transposase Accessible Chromatin sequencing (ATAC-seq) or single cell ATAC-seq as described (see, e.g., Buenrostro, et al., Transposition of native chromatin for fast and sensitive epigenomic profiling of open chromatin, DNA-binding proteins and nucleosome position. Nature methods 2013; 10 (12): 1213-1218; Buenrostro et al., Single-cell chromatin accessibility reveals principles of regulatory variation. Nature 523, 486-490 (2015); Cusanovich, D. A., Daza, R., Adey, A., Pliner, H., Christiansen, L., Gunderson, K. L., Steemers, F. J., Trapnell, C. & Shendure, J. Multiplex single-cell profiling of chromatin accessibility by combinatorial cellular indexing. Science. 2015 May 22; 348(6237):910-4. doi: 10.1126/science.aab1601. Epub 2015 May 7; US20160208323A1; US20160060691A1; and WO2017156336A1). The term “tagmentation” refers to a step in the Assay for Transposase Accessible Chromatin using sequencing (ATAC-seq) as described. Specifically, a hyperactive Tn5 transposase loaded in vitro with adapters for high-throughput DNA sequencing can simultaneously fragment and tag a genome with sequencing adapters. In certain embodiments, ATAC-seq is used on a bulk DNA sample to determine mitochondrial mutations.

In certain embodiments, a single cell atlas includes single cell epigenetic data. A single cell atlas for a tissue may be constructed by measuring epigenetic marks on chromatin in single cells. The epigenetic marks can indicate genomic loci that are in active or silent chromatin states (see, e.g., Epigenetics, Second Edition, 2015, Edited by C. David Allis; Marie-Laure Caparros; Thomas Jenuwein; Danny Reinberg; Associate Editor Monika Lachlan). In certain embodiments, single cell ChIP-seq can be used to determine chromatin states in single cells (see, e.g., Rotem, et al., Single-cell ChIP-seq reveals cell subpopulations defined by chromatin state. Nat Biotechnol. 2015 November; 33(11): 1165-1172). In certain embodiments, single cell ChIP-seq is used to determine genomic loci that are occupied by histone modifications, histone variants, transcription factors and/or chromatin modifying enzymes. In certain embodiments, epigenetic features can be chromatin contact domains, chromatin loops, superloops, or chromatin architecture data, such as obtained by single cell HiC (see, e.g., Rao et al., Cell. 2014 Dec. 18; 159(7):1665-80; and Ramani, et al., Sci-Hi-C: A single-cell Hi-C method for mapping 3D genome organization in large number of single cells Methods. 2020 Jan. 1; 170: 61-68).

In certain embodiments, a single cell atlas includes spatially resolved single cell data. The spatial data used in the present invention can be any spatial data. Methods of generating spatial data of varying resolution are known in the art, for example, ISS (Ke, R. et al. In situ sequencing for RNA analysis in preserved tissue and cells. Nat. Methods 10, 857-860 (2013)), MERFISH (Chen, K. H., Boettiger, A. N., Moffitt, J. R., Wang, S. & Zhuang, X. Spatially resolved, highly multiplexed RNA profiling in single cells. Science 348, (2015)), smFISH (Codeluppi, S. et al. Spatial organization of the somatosensory cortex revealed by cyclic smFISH. biorxiv.org/lookup/doi/10.1101/276097 (2018) doi:10.1101/276097), osmFISH (Codeluppi, S. et al. Spatial organization of the somatosensory cortex revealed by osmFISH. Nat. Methods 15, 932-935 (2018)), STARMap (Wang, X. et al. Three-dimensional intact-tissue sequencing of single-cell transcriptional states. Science 361, eaat5691 (2018)), Targeted ExSeq (Alon, S. et al. Expansion Sequencing: Spatially Precise In Situ Transcriptomics in Intact Biological Systems. biorxiv.org/lookup/doi/10.1101/2020.05.13.094268 (2020) doi:10.1101/2020.05.13.094268), seqFISH+(Eng, C.-H. L. et al. Transcriptome-scale super-resolved imaging in tissues by RNA seqFISH+. Nature (2019) doi:10.1038/s41586-019-1049-y.), Spatial Transcriptomics methods (e.g., Spatial Transcriptomics (ST))(St∪hl, P. L. et al. Visualization and analysis of gene expression in tissue sections by spatial transcriptomics. Science 353, 78-82 (2016)) (now available commercially as Visium), Slide-seq (Rodrigues, S. G. et al. Slide-seq: A scalable technology for measuring genome-wide expression at high spatial resolution. Science 363, 1463-1467 (2019)), or High Definition Spatial Transcriptomics (Vickovic, S. et al. High-definition spatial transcriptomics for in situ tissue profiling. Nat. Methods 16, 987-990 (2019)). In certain embodiments, proteomics and spatial patterning using antenna networks is used to spatially map a tissue specimen and this data can be further used to align single cell data to a larger tissue specimen (see, e.g., US20190285644A1). In certain embodiments, the spatial data can be immunohistochemistry data or immunofluorescence data.

In certain embodiments, a single cell atlas includes single cell proteomics data. In certain embodiments, single cell proteomics can be used to generate the single cell data. In certain embodiments, the single cell proteomics data is combined with single cell transcriptome data. Non-limiting examples include multiplex analysis of single cell constituents (U.S Patent Publication No. US20180340939A), single-cell proteomic assay using aptamers (U.S Patent Publication No. US20180320224A1), and methods of identifying multiple epitopes in cells (U. S Patent Publication No. US20170321251A1).

In certain embodiments, a single cell atlas includes single cell multimodal data. In certain embodiments, SHARE-Seq (Ma, S. et al. Chromatin potential identified by shared single cell profiling of RNA and chromatin. bioRxiv 2020.06.17.156943 (2020) doi:10.1101/2020.06.17.156943) is used to generate single cell RNA-seq and chromatin accessibility data. In certain embodiments, CITE-seq (Stoeckius, M. et al. Simultaneous epitope and transcriptome measurement in single cells. Nat. Methods 14, 865-868 (2017)) (cellular proteins) is used to generate single cell RNA-seq and proteomics data. In certain embodiments, Patch-seq (Cadwell, C. R. et al. Electrophysiological, transcriptomic and morphologic profiling of single neurons using Patch-seq. Nat. Biotechnol. 34, 199-203 (2016)) is used to generate single cell RNA-seq and patch-clamping electrophysiological recording and morphological analysis of single neurons data (e.g., for the brain or enteric nervous system (ENS)) (see, e.g., van den Hurk, et al., Patch-Seq Protocol to Analyze the Electrophysiology, Morphology and Transcriptome of Whole Single Neurons Derived From Human Pluripotent Stem Cells, Front Mol Neurosci. 2018; 11: 261).

The present invention may encompass incorporation of a unique molecular identifier (UMI) (see, e.g., Kivioja et al., 2012, Nat. Methods. 9 (1): 72-4 and Islam et al., 2014, Nat. Methods. 11 (2): 163-6) a unique sample barcode, a unique cell barcode (cell into the sequencing library, or a combination. The barcode as used herein refers to a short sequence of nucleotides (for example, DNA or RNA) that is used as an identifier for an associated molecule, such as a target molecule and/or target nucleic acid, or as an identifier of the source of an associated molecule, such as a sample or cell-of-origin. A barcode may also refer to any unique, non-naturally occurring, nucleic acid sequence that may be used to identify the originating source of a nucleic acid fragment.

Barcoding may be performed based on any of the compositions or methods disclosed in International Patent Publication No. WO 2014047561 A1, Compositions and methods for labeling of agents, incorporated herein in its entirety. In certain embodiments barcoding uses an error correcting scheme (T. K. Moon, Error Correction Coding: Mathematical Methods and Algorithms (Wiley, New York, ed. 1, 2005)). Not being bound by a theory, amplified sequences from different sources can be sequenced together and resolved based on the barcode associated with each sequencing read.

In preferred embodiments, sequencing is performed using unique molecular identifiers (UMI). The term “unique molecular identifiers” (UMI) as used herein refers to a sequencing linker or a subtype of nucleic acid barcode used in a method that uses molecular tags to detect and quantify unique amplified products. A UMI is used to distinguish effects through a single clone from multiple clones. The term “clone” as used herein may refer to a single mRNA or target nucleic acid to be sequenced. Unique Molecular Identifiers may be short (usually 4-10 bp) random barcodes added to transcripts during reverse-transcription. They enable sequencing reads to be assigned to individual transcript molecules and thus the removal of amplification noise and biases from RNA-seq data. The UMI may also be used to determine the number of transcripts that gave rise to an amplified product.

In certain embodiments, any tissue associated with a phenotype may be analysed to generate a tissue specific atlas. Exemplary tissues include, but are not limited to disease and control tissues, particularly, animal and plant tissues (e.g., tumor, intestine, colon, lungs, heart, brain, roots, stems, leaves). Tissue samples can be obtained from any organ in the subject.

In certain embodiments, the phenotype may be associated with any disease. Non-limiting diseases include immune related diseases (e.g., autoimmune, inflammation), cancer, IBD, cardiovascular disease, gastrointestinal disease, rheumatism, skin diseases and infectious diseases.

As used throughout the present specification, the terms “autoimmune disease” or “autoimmune disorder” are used interchangeably refer to diseases or disorders caused by an immune response against a self-tissue or tissue component (self-antigen) and include a self-antibody response and/or cell-mediated response. The terms encompass organ-specific autoimmune diseases, in which an autoimmune response is directed against a single tissue, as well as non-organ specific autoimmune diseases, in which an autoimmune response is directed against a component present in two or more, several or many organs throughout the body.

Examples of autoimmune diseases include, but are not limited to, acute disseminated encephalomyelitis (ADEM); Addison's disease; ankylosing spondylitis; antiphospholipid antibody syndrome (APS); aplastic anemia; autoimmune gastritis; autoimmune hepatitis; autoimmune thrombocytopenia; Behçet's disease; coeliac disease; dermatomyositis; diabetes mellitus type I; Goodpasture's syndrome; Graves' disease; Guillain-Barré syndrome (GBS); Hashimoto's disease; idiopathic thrombocytopenic purpura; inflammatory bowel disease (IBD) including Crohn's disease and ulcerative colitis; mixed connective tissue disease; multiple sclerosis (MS); myasthenia gravis; opsoclonus myoclonus syndrome (OMS); optic neuritis; Ord's thyroiditis; pemphigus; pernicious anaemia; polyarteritis nodosa; polymyositis; primary biliary cirrhosis; primary myoxedema; psoriasis; rheumatic fever; rheumatoid arthritis; Reiter's syndrome; scleroderma; Sjögren's syndrome; systemic lupus erythematosus; Takayasu's arteritis; temporal arteritis; vitiligo; warm autoimmune hemolytic anemia; or Wegener's granulomatosis.

Examples of inflammatory diseases or disorders include, but are not limited to, asthma, allergy, allergic rhinitis, allergic airway inflammation, atopic dermatitis (AD), chronic obstructive pulmonary disease (COPD), inflammatory bowel disease (IBD), multiple sclerosis, arthritis, psoriasis, eosinophilic esophagitis, eosinophilic pneumonia, eosinophilic psoriasis, hypereosinophilic syndrome, graft-versus-host disease, uveitis, cardiovascular disease, pain, multiple sclerosis, lupus, vasculitis, chronic idiopathic urticaria and Eosinophilic Granulomatosis with Polyangiitis (Churg-Strauss Syndrome).

The asthma may be allergic asthma, non-allergic asthma, severe refractory asthma, asthma exacerbations, viral-induced asthma or viral-induced asthma exacerbations, steroid resistant asthma, steroid sensitive asthma, eosinophilic asthma or non-eosinophilic asthma and other related disorders characterized by airway inflammation or airway hyperresponsiveness (AHR).

The COPD may be a disease or disorder associated in part with, or caused by, cigarette smoke, air pollution, occupational chemicals, allergy or airway hyperresponsiveness.

The allergy may be associated with foods, pollen, mold, dust mites, animals, or animal dander.

The IBD may be ulcerative colitis (UC), Crohn's Disease, collagenous colitis, lymphocytic colitis, ischemic colitis, diversion colitis, Behcet's syndrome, infective colitis, indeterminate colitis, and other disorders characterized by inflammation of the mucosal layer of the large intestine or colon.

In certain embodiments, the methods described herein are applicable to any cancer type. In preferred embodiments, the cancer is colorectal cancer (CRC). The cancer may include, without limitation, liquid tumors such as leukemia (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (e.g., Hodgkin's disease, non-Hodgkin's disease), Waldenstrom's macroglobulinemia, heavy chain disease, or multiple myeloma.

The cancer may include, without limitation, solid tumors such as sarcomas and carcinomas. Examples of solid tumors include, but are not limited to fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, epithelial carcinoma, bronchogenic carcinoma, hepatoma, colorectal cancer (e.g., colon cancer, rectal cancer), anal cancer, pancreatic cancer (e.g., pancreatic adenocarcinoma, islet cell carcinoma, neuroendocrine tumors), breast cancer (e.g., ductal carcinoma, lobular carcinoma, inflammatory breast cancer, clear cell carcinoma, mucinous carcinoma), ovarian carcinoma (e.g., ovarian epithelial carcinoma or surface epithelial-stromal tumor including serous tumor, endometrioid tumor and mucinous cystadenocarcinoma, sex-cord-stromal tumor), prostate cancer, liver and bile duct carcinoma (e.g., hepatocelluar carcinoma, cholangiocarcinoma, hemangioma), choriocarcinoma, seminoma, embryonal carcinoma, kidney cancer (e.g., renal cell carcinoma, clear cell carcinoma, Wilm's tumor, nephroblastoma), cervical cancer, uterine cancer (e.g., endometrial adenocarcinoma, uterine papillary serous carcinoma, uterine clear-cell carcinoma, uterine sarcomas and leiomyosarcomas, mixed mullerian tumors), testicular cancer, germ cell tumor, lung cancer (e.g., lung adenocarcinoma, squamous cell carcinoma, large cell carcinoma, bronchioloalveolar carcinoma, non-small-cell carcinoma, small cell carcinoma, mesothelioma), bladder carcinoma, signet ring cell carcinoma, cancer of the head and neck (e.g., squamous cell carcinomas), esophageal carcinoma (e.g., esophageal adenocarcinoma), tumors of the brain (e.g., glioma, glioblastoma, medullablastoma, astrocytoma, medulloblastoma, craniopharyngioma, ependymoma, pinealoma, hemangioblastoma, acoustic neuroma, oligodenroglioma, schwannoma, meningioma), neuroblastoma, retinoblastoma, neuroendocrine tumor, melanoma, cancer of the stomach (e.g., stomach adenocarcinoma, gastrointestinal stromal tumor), or carcinoids. Lymphoproliferative disorders are also considered to be proliferative diseases.

### Gene Modules

In certain embodiments, a single cell atlas is used to generate gene modules. As used herein, “gene module” refers to any group of genes having an association. The association may be cell type expression (e.g., genes whose expression is enriched in a cell type). The association may be gene program or biological program expression. The association may be genes differentially expressed in cell types between healthy and diseased tissues. The association may be genes that co-vary in single cells (e.g., covariation). As used herein, the term “co-vary’ refers to genes that are upregulated and downregulated together. A correlation between genes refers to genes that co-vary. The association may be expression of genes expressed in a cell type having a specific cell state. The association may be a spatial association, such that specific cell types are located in specific regions of a tissue or biological programs are expressed in specific regions of a tissue.

The association may be encompassed by any group of signature genes. In exemplary embodiments, a single cell atlas can be as simple as including a few single cells (e.g., less than 1000 cells) of a tissue type. The expression of genes in the single cells can be used to construct gene modules to be used in assigning genetic variants. In certain embodiments, including a greater number of cells can increase the number of gene modules constructed.

### Signature Genes

In certain embodiments, a gene module may include signature genes. As used herein a “signature” may encompass any gene or genes, protein or proteins, or epigenetic element(s) whose expression profile or whose occurrence is associated with a specific cell type, subtype, or cell state of a specific cell type or subtype within a population of cells. For ease of discussion, when discussing gene expression, any of gene or genes, protein or proteins, or epigenetic element(s) may be substituted. As used herein, the terms “signature”, “expression profile”, or “expression program” may be used interchangeably. As used herein, the term “biological program” or “cell program” may be a type of “signature”, “expression program” or “transcriptional program” and refers to a set of genes that share a role in a biological function (e.g., an activation program, cell differentiation program, proliferation program). Biological programs can include a pattern of gene expression that result in a corresponding physiological event or phenotypic trait. Biological programs can include up to several hundred genes that are expressed in a spatially and temporally controlled fashion. Expression of individual genes can be shared between biological programs. Expression of individual genes can be shared among different single cell types; however, expression of a biological program may be cell type specific or temporally specific (e.g., the biological program is expressed in a cell type at a specific time). Biological programs may be expressed across different cell types. In certain embodiments, a biological program includes genes that co-vary. Expression of a biological program may be regulated by a master switch, such as a nuclear receptor or transcription factor. As used herein, the term “topic” refers to a biological program. The biological program (e.g., topics) can be modeled as a distribution over expressed genes. One method to identify cell programs is non-negative matrix factorization (NMF) (see, e.g., Lee D D and Seung H S, Learning the parts of objects by non-negative matrix factorization, Nature. 1999 Oct. 21; 401(6755):788-91). Other approaches are topic models (Bielecki, Riesenfeld, Kowalczyk, et al., 2018 Skin inflammation driven by differentiation of quiescent tissue-resident ILCs into a spectrum of pathogenic effectors. bioRxiv 461228) and word embeddings. Identifying cell programs can recover cell states and bridge differences between cells. Single cell types may span a range of continuous cell states (see, e.g., Shekhar et al., Comprehensive Classification of Retinal Bipolar Neurons by Single-Cell Transcriptomics Cell. 2016 Aug. 25; 166(5):1308-1323.e30; and Bielecki, et al., 2018 bioRxiv 461228).

It is to be understood that also when referring to proteins (e.g. differentially expressed proteins), such may fall within the definition of “gene” signature. Levels of expression or activity or prevalence may be compared between different cells in order to characterize or identify for instance signatures specific for cell (sub)populations. Increased or decreased expression or activity or prevalence of signature genes may be compared between different cells in order to characterize or identify for instance specific cell (sub)populations. The detection of a signature in single cells may be used to identify and quantitate for instance specific cell (sub)populations. A signature may include a gene or genes, protein or proteins, or epigenetic element(s) whose expression or occurrence is specific to a cell (sub)population, such that expression or occurrence is exclusive to the cell (sub)population. A gene signature as used herein may thus refer to any set of up- and down-regulated genes that are representative of a cell type or subtype. A gene signature as used herein may also refer to any set of up- and down-regulated genes between different cells or cell (sub)populations derived from a gene-expression profile. For example, a gene signature may comprise a list of genes differentially expressed in a distinction of interest.

The signature as defined herein (being it a gene signature, protein signature or other genetic or epigenetic signature) can be used to indicate the presence of a cell type, a subtype of the cell type, the state of the microenvironment of a population of cells, a particular cell type population or subpopulation, and/or the overall status of the entire cell (sub)population. Furthermore, the signature may be indicative of cells within a population of cells in vivo. The signature may also be used to suggest for instance particular therapies, or to follow up treatment, or to suggest ways to modulate immune systems. The signatures of the present invention may be discovered by analysis of expression profiles of single-cells within a population of cells from isolated samples (e.g. tumor samples), thus allowing the discovery of novel cell subtypes or cell states that were previously invisible or unrecognized. The presence of subtypes or cell states may be determined by subtype specific or cell state specific signatures. The presence of these specific cell (sub)types or cell states may be determined by applying the signature genes to bulk sequencing data in a sample. Not being bound by a theory the signatures of the present invention may be microenvironment specific, such as their expression in a particular spatio-temporal context. Not being bound by a theory, signatures as discussed herein are specific to a particular pathological context. Not being bound by a theory, a combination of cell subtypes having a particular signature may indicate an outcome. Not being bound by a theory, the signatures can be used to deconvolute the network of cells present in a particular pathological condition. Not being bound by a theory the presence of specific cells and cell subtypes are indicative of a particular response to treatment, such as including increased or decreased susceptibility to treatment. The signature may indicate the presence of one particular cell type. In one embodiment, the novel signatures are used to detect multiple cell states or hierarchies that occur in subpopulations of cancer cells that are linked to particular pathological condition (e.g. cancer grade), or linked to a particular outcome or progression of the disease (e.g. metastasis), or linked to a particular response to treatment of the disease.

The signature according to certain embodiments of the present invention may comprise or consist of one or more genes, proteins and/or epigenetic elements, such as for instance 1, 2, 3, 4, 5, 6, 7, 8, 9, 10 or more. In certain embodiments, the signature may comprise or consist of two or more genes, proteins and/or epigenetic elements, such as for instance 2, 3, 4, 5, 6, 7, 8, 9, 10 or more. In certain embodiments, the signature may comprise or consist of three or more genes, proteins and/or epigenetic elements, such as for instance 3, 4, 5, 6, 7, 8, 9, 10 or more. In certain embodiments, the signature may comprise or consist of four or more genes, proteins and/or epigenetic elements, such as for instance 4, 5, 6, 7, 8, 9, 10 or more. In certain embodiments, the signature may comprise or consist of five or more genes, proteins and/or epigenetic elements, such as for instance 5, 6, 7, 8, 9, 10 or more. In certain embodiments, the signature may comprise or consist of six or more genes, proteins and/or epigenetic elements, such as for instance 6, 7, 8, 9, 10 or more. In certain embodiments, the signature may comprise or consist of seven or more genes, proteins and/or epigenetic elements, such as for instance 7, 8, 9, 10 or more. In certain embodiments, the signature may comprise or consist of eight or more genes, proteins and/or epigenetic elements, such as for instance 8, 9, 10 or more. In certain embodiments, the signature may comprise or consist of nine or more genes, proteins and/or epigenetic elements, such as for instance 9, 10 or more. In certain embodiments, the signature may comprise or consist of ten or more genes, proteins and/or epigenetic elements, such as for instance 10, 11, 12, 13, 14, 15, or more. It is to be understood that a signature according to the invention may for instance also include genes or proteins as well as epigenetic elements combined.

In certain embodiments, a signature is characterized as being specific for a particular cell or cell (sub)population if it is upregulated or only present, detected or detectable in that particular tumor cell or tumor cell (sub)population, or alternatively is downregulated or only absent, or undetectable in that particular tumor cell or tumor cell (sub)population. In this context, a signature consists of one or more differentially expressed genes/proteins or differential epigenetic elements when comparing different cells or cell (sub)populations, including comparing different cells or cell (sub)populations, as well as comparing tumor cells or tumor cell (sub)populations with non-tumor cells or non-tumor cell (sub)populations. It is to be understood that “differentially expressed” genes/proteins include genes/proteins which are up- or down-regulated as well as genes/proteins which are turned on or off. When referring to up- or down-regulation, in certain embodiments, such up- or down-regulation is preferably at least two-fold, such as two-fold, three-fold, four-fold, five-fold, or more, such as for instance at least ten-fold, at least 20-fold, at least 30-fold, at least 40-fold, at least 50-fold, or more. Alternatively, or in addition, differential expression may be determined based on common statistical tests, as is known in the art.

As discussed herein, differentially expressed genes/proteins, or differential epigenetic elements may be differentially expressed on a single cell level, or may be differentially expressed on a cell population level. Preferably, the differentially expressed genes/proteins or epigenetic elements as discussed herein, such as constituting the gene signatures as discussed herein, when as to the cell population level, refer to genes that are differentially expressed in all or substantially all cells of the population (such as at least 80%, preferably at least 90%, such as at least 95% of the individual cells). This allows one to define a particular subpopulation of tumor cells. As referred to herein, a “subpopulation” of cells preferably refers to a particular subset of cells of a particular cell type which can be distinguished or are uniquely identifiable and set apart from other cells of this cell type. The cell subpopulation may be phenotypically characterized and is preferably characterized by the signature as discussed herein. A cell (sub)population as referred to herein may constitute of a (sub)population of cells of a particular cell type characterized by a specific cell state.

When referring to induction, or alternatively suppression of a particular signature, preferably, is meant induction or alternatively suppression (or upregulation or downregulation) of at least one gene/protein and/or epigenetic element of the signature, such as for instance at least to, at least three, at least four, at least five, at least six, or all genes/proteins and/or epigenetic elements of the signature.

Signatures may be functionally validated as being uniquely associated with a particular immune responder phenotype. Induction or suppression of a particular signature may consequentially be associated with or causally drive a particular immune responder phenotype.

Various aspects and embodiments of the invention may involve analyzing gene signatures, protein signature, and/or other genetic or epigenetic signature based on single cell analyses (e.g. single cell RNA sequencing) or alternatively based on cell population analyses, as is defined herein elsewhere.

In further aspects, the invention relates to gene signatures, protein signature, and/or other genetic or epigenetic signature of particular tumor cell subpopulations, as defined herein elsewhere. The invention hereto also further relates to particular tumor cell subpopulations, which may be identified based on the methods according to the invention as discussed herein, as well as methods to obtain such cell (sub)populations and screening methods to identify agents capable of inducing or suppressing particular tumor cell (sub)populations.

The invention further relates to various uses of the gene signatures, protein signature, and/or other genetic or epigenetic signature as defined herein, as well as various uses of the tumor cells or tumor cell (sub)populations as defined herein. Particular advantageous uses include methods for identifying agents capable of inducing or suppressing particular tumor cell (sub)populations based on the gene signatures, protein signature, and/or other genetic or epigenetic signature as defined herein. The invention further relates to agents capable of inducing or suppressing particular tumor cell (sub)populations based on the gene signatures, protein signature, and/or other genetic or epigenetic signature as defined herein, as well as their use for modulating, such as inducing or repressing, a particular gene signature, protein signature, and/or other genetic or epigenetic signature. In one embodiment, genes in one population of cells may be activated or suppressed in order to affect the cells of another population. In related aspects, modulating, such as inducing or repressing, a particular a particular gene signature, protein signature, and/or other genetic or epigenetic signature may modify overall tumor composition, such as tumor cell composition, such as tumor cell subpopulation composition or distribution, or functionality.

The signature genes of the present invention were discovered by analysis of expression profiles of single-cells within a population of cells from tissues, thus allowing the discovery of novel cell subtypes that were previously invisible in a population of cells within a tissue. The presence of subtypes may be determined by subtype specific signature genes. The presence of these specific cell types may be determined by applying the signature genes to bulk sequencing data in a patient tumor. Not being bound by a theory, a tumor is a conglomeration of many cells that make up a tumor microenvironment, whereby the cells communicate and affect each other in specific ways. As such, specific cell types within this microenvironment may express signature genes specific for this microenvironment. Not being bound by a theory the signature genes of the present invention may be microenvironment specific, such as their expression in a tumor. Not being bound by a theory, signature genes determined in single cells that originated in a tumor are specific to other tumors. Not being bound by a theory, a combination of cell subtypes in a tumor may indicate an outcome. Not being bound by a theory, the signature genes can be used to deconvolute the network of cells present in a tumor based on comparing them to data from bulk analysis of a tumor sample. Not being bound by a theory, the presence of specific cells and cell subtypes may be indicative of tumor growth, invasiveness and resistance to treatment. The signature gene may indicate the presence of one particular cell type. In one embodiment, the signature genes may indicate that tumor infiltrating T-cells are present. The presence of cell types within a tumor may indicate that the tumor will be resistant to a treatment. In one embodiment, the signature genes of the present invention are applied to bulk sequencing data from a tumor sample obtained from a subject, such that information relating to disease outcome and personalized treatments is determined. In one embodiment, the novel signature genes are used to detect multiple cell states that occur in a subpopulation of tumor cells that are linked to resistance to targeted therapies and progressive tumor growth.

All gene name symbols refer to the gene as commonly known in the art. The examples described herein that refer to the mouse gene names are to be understood to also encompasses human genes, as well as genes in any other organism (e.g., homologous, orthologous genes). The term homolog may apply to the relationship between genes separated by the event of speciation (e.g., ortholog). Orthologs are genes in different species that evolved from a common ancestral gene by speciation. Normally, orthologs retain the same function in the course of evolution. Gene symbols may be those referred to by the HUGO Gene Nomenclature Committee (HGNC) or National Center for Biotechnology Information (NCBI). Any reference to the gene symbol is a reference made to the entire gene or variants of the gene. The signature as described herein may encompass any of the genes described herein.

### Genome Wide Association Studies (GWAS)

In certain embodiments, gene modules include genome wide association studies (GWAS) risk genes. Genome-wide association studies (GWAS) have identified thousands of genetic loci for hundreds of traits (see, e.g., Welter, D. et al. The NHGRI GWAS catalogue, a curated resource of SNP-trait associations. Nucleic Acids Res. 42, D1001-D1006 (2014); Wood, A. R. et al. Defining the role of common variation in the genomic and biological architecture of adult human height. Nat. Genet. 46, 1173-1186 (2014); Ripke, S. et al. Biological insights from 108 schizophrenia-associated genetic loci. Nature 511, 421-427 (2014); Okbay, A. et al. Genome-wide association study identifies 74 loci associated with educational attainment. Nature 533, 539-542 (2016); and Sudlow, C. et al. UK biobank: an open access resource for identifying the causes of a wide range of complex diseases of middle and old age. PLoS Med. 12, 1-10 (2015)). Applicants previously found that most “GWAS genes” are expressed in a specific cell subset (e.g., module) (Smillie et al., 2019). The GWAS genes fall into co-varying modules with each other and other genes, such that >50% GWAS genes map into 10 meta modules. Smillie et al. 2019, also showed that expanding the tissue coverage from mucosa to inner layers, allowed for relating nearly every gene to cell type(s). Example gene modules useful in the present invention include healthy and UC colon gene modules identified in Smillie et al., 2019 (Table 4) (see also, International Patent Publication No. WO 2019/018440). These gene modules may be augmented with additional co-varying genes.

TABLE 4
Meta-modules in healthy and UC cells that may contribute to disease onset and progression (HQ = high quality).

| | | seed | | | | |
|---|---|---|---|---|---|---|
| rank | ident | gene | health | hq_genes | putative_risk_genes | all_genes |
| 1 | Cycling | CYTH1 | UC | CD28, | CYTH1, CD5, | CYTH1, CD5, SPSB1, ZNF574, PTPN2, RAP1A, TNFAIP8, |
| | T | | | CTLA4, | PTPN2, | FUBP3, PPP1CC, RNF145, POU2F2, SMAP2, STAT3, ZNF638, |
| | | | | NFKB1, | PHACTR2, | RILPL2, ING5, PHACTR2, CD28, ZNF518B, ZNF280D, CIRBP, |
| | | | | LRBA, | CD28, STAT5A, | ETV6, UBQLN2, P2RX5, SH3KBP1, CD4, MSI2, BCL3, |
| | | | | ADAM17, | SUFU, IRF4, | ATP6V0E2,SYNJ1, TNIK, SECTM1, SIAH2, DEAF1, IL6ST, |
| | | | | TTC7A, | CTLA4, NFKB1, | STAT5A, METTL8, SUFU, IRF4, MTERFD2, MTRR, SEPT6, |
| | | | | ICOS | LRBA, | CTLA4, RBM26-AS1, AFTPH, LIMS1, SATB1, NFKB1, |
| | | | | | ADAM17, | MAPKAPK3, FAM188A, FGFR1, FAM60A, USP4, LRBA, |
| | | | | | SLC2A4RG, | ADD3, MAFG, DGKA, MYB, RP1-134E15.3, ANKRD10, |
| | | | | | CYLD, TTC7A, | MIR181A1HG, SNHG7, PIM2, GTF2H1, TRAF, CLK3, |
| | | | | | ICOS | ZNF281, AKT2, RANBP3, C19orf38, MYOM2, ADAM17, |
| | | | | | | IGBP1, UBR5, ERBB2IP, AC011841.1, SLC2A4RG, CTSB, |
| | | | | | | HSDL2, CYLD, CTD-2369P2.2, ITPKC, MAN2B1, GLCCI1, |
| | | | | | | GOSR1, PVT1, SPOCK2, LBH, RDH10, RP11-134P9.1, |
| | | | | | | LINC00963, SUPT20H, ATG9A, AP1S2, DDX19B, |
| | | | | | | TTC7A, SESN3, NSMAF, CCM2, ICOS |
| 2 | DC2 | TNFAIP3 | Healthy | TNFAIP3, | TNFAIP3, | TNFAIP3, N4BP1, IQGAP2, MGAT1, IL8, PHTF2, LDLR, |
| | | | | TNFSF15, | TNFSF15, | LINC00926, CCL8, TP53I3, TNFSF15, PLAU, FCGR1A, STX4, |
| | | | | PLAU, | PLAU, | RP11-44N11.1, FCGR1B, METTL15, MPHOSPH8, RP11-42I10.1, |
| | | | | STAT4, | STAT4, AHR, | CTSS, STAT4, FRMD4B, IRAK3, GBP3, AHR, CPM, EMR2, |
| | | | | AHR, | INO80, CRTC3, | FEZ2, INO80, CRTC3, MCOLN1, TCF7L2, CCRL2, ZNF331, |
| | | | | IL10, | CCRL2, CXCL3, | SLC36A4, PPP1R15B, MS4A7, CXCL3, ZFP57, RP3-402G11.26, |
| | | | | NDFIP1, | IL10, PDGFB, | CD300C, SCPEP1, HSBP1L1, GLUL, C2orf49, IL10, TMEM63A, |
| | | | | NCF4, | NDFIP1, NCF4, | PEPD, MGAT4A, LYRM5, CHMP1B, SETMAR, LRRC32, PYGL, |
| | | | | IL10RB | IL10RB | RP11-425D10.10, CD99, HBEGF, AGTRAP, SPATA6, |
| | | | | | | AC005306.3, SLC12A4, VAV1, ZNF821, ABL2, GBP2, CCL4L2, |
| | | | | | | RMDN3, HES4, RHOB, CDK8, PHLDA1, EVI5, PDGFB, BNIP3L, |
| | | | | | | CACNA2D4, FUS, SLC39A1, NUMA1, IFRD1, RP11-473M20.7, |
| | | | | | | SLC43A3, MRVI1, SP1, CR1, RNF135, DDX19B, ARHGAP18, |
| | | | | | | NDFIP1, NADSYN1, CD300LF, CSF1R, CLEC4E, RNF141, |
| | | | | | | ATF3, LAPTM4A, NCF4, IL10RB, CRYL1, TGFBI, CEP19 |
| 3 | Cycling | ZFP36L1 | UC | PTPRC, | ZFP36L1, | ZFP36L1, GPR18, PTPRC, CERS4, CR2, RHOH, CTA- |
| | B | | | CYBB, | PTPRC, | 250D10.23, TNF, UBAC2, CYB561A3, CD40, CXCR5, TRIM38, |
| | | | | MAP3K8, | UBAC2, | PCSK7, CATSPER2, CD22, ING4, CYBB, FAM43A, MTHFR, CR1, |
| | | | | RIPK2, | CD40, CXCR5, | ZNF230, GNB4, SESTD1, CDC40, LINC00685, SNHG7, BCL10, |
| | | | | SKAP2 | CYBB, | ZNF267, HLA-DOB, LAT2, SLC44A2, PTK2, TMEM55B, RABEP2, |
| | | | | | FAM175B, | CEPT1, ATAD2B, SCAF4, DRAM2, RP11-35G9.3, FAM175B, |
| | | | | | MAP3K8, | RNF44, FCRLA, CRYZL1, APBB1IP, MAP3K8, ERV3-1, WASF2, |
| | | | | | SOCS1, | IGHD, FCRL1, CD72, MAD1L1, CD79B, ZNF680, SOCS1, RP11- |
| | | | | | IFNGR2, REL, | 861A13.4, ARID4B, POU2F2, SNX2, RAB8B, FAM65B, SCIMP, |
| | | | | | RIPK2, SKAP2, | CD19, C5orf15, ACAP2, FKBP15, TNKS, LAPTM5, ADPGK, |
| | | | | | NFATC1 | CYFIP2, TAOK3, TRANK1, IFNGR2, SCAF11, TLR1, SEPT6, |
| | | | | | | ELK4, REL, FAM129C, MAP4K4, RIPK2, SKAP2, VPS4B, HERC4, |
| | | | | | | SIPA1, ERICH1, NFATC1, HMGA1P4, CHURC1, INPP5D, BIRC6, |
| | | | | | | LRCH4, DUSP10, TNFRSF13C, LYSMD2, STAT5B, SNX29, |
| | | | | | | LYRM7, HSPBAP1, TBC1D10C |
| 4 | CD4+ | CYLD | UC | CD28, | CYLD, CD28, | CYLD, LBH, ELMO1, ANAPC5, HADHA, CBFB, RBM26- |
| | Memory | | | PTPRC, | PTPRC, | AS1, TPR, WIPI2, TMEM243, CD28, CIRBP, ANP32B, TGOLN2, |
| | | | | NDFIP1, | NDFIP1, | TNFAIP8, PTPRC, GRB2, GIN1, CNOT7, IL27RA, RP11-902B17.1, |
| | | | | ITGB2, | CD5, ITGB2, | SERINC1, MAF1, SIKE1, UBQLN1, PGLS, HAGH, SUPT20H, |
| | | | | IL10RB, | HMHA1, | TARDBP, VPS4B, HNRNPA0, FXR1, NDFIP1, MATR3, ILKAP, |
| | | | | UBASH3A | CYTH1, | XPA, CD5, UXS1, SNW1, EIF2AK1, ACADSB, ITGB2, HLA-E, |
| | | | | | LOH12CR1, | HNRNPK, HMHA1, GPSM3, YWHAB, PPP2R5A, SFI1, RNF145, |
| | | | | | IL10RB, | BTF3L4, CYTH1, RPRD2, RIC3, SUSD3, CCNYL1, OXA1L, |
| | | | | | PMPCA, | SH3KBP1, TSN, LIF, MAFG, FKBP5, EIF3D, DHRS7B, EMC10, |
| | | | | | TNFRSF14, | KLHDC3, UNC45A, RWDD1, LOH12CR1, IL10RB, THOC5, |
| | | | | | UBASH3A | ORMDL1, MED28, RILPL2, TMC6, PMPCA, KIAA1407, |
| | | | | | | TNFRSF14, UBASH3A, PSME3, ALDH9A1, DENND6A, SRSF3, |
| | | | | | | MCM3, PHRF1, PGGT1B, SZRD1, EIF2S2, PAWR, TACC3, RHOF, |
| | | | | | | RING1, PPM1A, SCAMP4, PHAX, TMEM165, SPPL3, SERBP1, |
| | | | | | | TAX1BP1, TRIM4 |
| 5 | WNT2B+ | ERAP1 | Healthy | ERAP1, | ERAP1, | ERAPl, ARL17B, MAP3K5, CXCL3, SLAMF8, CXCL2, STAT2, |
| | Fos-lo 1 | | | SLAMF8, | CXCL3, | SOWAHC, SOD2, DUSP10, RP11-293M10.5, NR2F2, SLC9A6, |
| | | | | EGFR, | SLAMF8, | YME1L1, IRAK3, STK4, PHIP, FAM120A, ICAM4, PARP11, |
| | | | | GPR65, | ICAM4, | DPYSL2, PARD3B, ERO1LB, CYLD, ZNF559, MBD2, BCL6, |
| | | | | AHR, | CYLD, | CXCL1, XYLT1, MTHFR, FBXL4, SLC19A2, IL6ST, TAF5L, |
| | | | | NFKB2 | SLC19A2, | ADAM28, DENND2D, EGFR, WWP1, BARD1, RN7SL336P, |
| | | | | | EGFR, GPR65, | GPR124, EXOC8, MARCH3, TMEM25, AKAP12, STEAP1, GPD2, |
| | | | | | PTPRK, AHR, | ADAMTS1, PCDH7, TRIP12, GPR65, AC006994.2, EPHA7, |
| | | | | | FADS3, NFKB2 | PPTC7, VANGL2, FAM133B, SLC15A4, AR, KPNA5, ARNT, |
| | | | | | | ZBTB10, TNFSF10, SLC25A29, WDR91, MFSD6, PTPRK, AHR, |
| | | | | | | SBF2-AS1, CCDC59, NCOA7, TRIB3, SPTBN1, FADS3, ST8SIA1, |
| | | | | | | RIPK1, STIM1, GJB2, ATL1, CXCL10, ANKRD32, PIGL, |
| | | | | | | SDR42E2, RP11-102N12.3, AC116366.6, YWHAG, NFKB2, SCFD2, |
| | | | | | | POLR1E, BNC2, OFD1, LAS1L, ATP6V1A, PTP4A2, CDK17, |
| | | | | | | LETM2, TIMP2, C9orf156, PLEKHA4, ATP8B4, ZCCHC17 |
| 6 | CD4+ | ITGA4 | UC | JAZF1, | ITGA4, JAZF1, | ITGA4, ANGEL2, HSPA1B, JAZF1, UBE2Q1, SEPT11, ZNF407, |
| | Memory | | | CASP8, | CASP8, | CASP8, EPM2AIP1, ACAP2, FOXP4, ZFAND2A, MPZL3, RP11- |
| | | | | TAGAP, | TAGAP, | 212P7.2, WDTC1, PRPF8, RNF115, ADAM19, TAGAP, DCAF5, |
| | | | | COG6, | DGKE, COG6, | PDE4D, MXD1, DUSP5, LGALS8, EDEM3, PICALM, RORA, RP5- |
| | | | | TGFBR1, | REL, TGFBR1, | 1073O3.7, DGKE, COG6, GPATCH2, TCP11L2, REL, CLASP2, |
| | | | | PRDM1 | COQ10B, | BCL2L11, TRAK2, EIF4E3, MAPK14, UBL3, MIR181A1HG, |
| | | | | | CUL2, | TGFBR1, ZNF33A, RP11-174G6.5, ZFAND4, RP11-727A23.5, |
| | | | | | DAP, PRDM1 | GSPT2, SRSF7, KDM3A, CEP152, COQ10B, CERK, G3BP2, PHC1, |
| | | | | | | LPGAT1, RSRC1, RBM12B, DDHD1, IREB2, PPP1R15A, SFXN3, |
| | | | | | | ZNF606, CUL2, TRAPPC8, DIRC2, TGFBR3, DAAM1, SUN1, |
| | | | | | | CAND1, NR1D1, FAM46C, TIAM2, IVNS1ABP, BCORL1, |
| | | | | | | TOM1L2, DAP, HSPA1A, ZRANB1, MYO5A, HMGCS1, SPEN, |
| | | | | | | MYO9A, BICD1, DDX26B, RPP14, CXorf56, CCDC91, RANBP6, |
| | | | | | | CCR6, FRMD4B, PPIP5K2, AFF1, PRDM1, ARMC5, SETD2, |
| | | | | | | RNPEPL1, NIN, FAM122C, ZNF75D, AKAP10, EMB |
| 7 | DC2 | VDR | Healthy | PTPRC, | VDR, SRRT, | VDR, ETNK1, G0S2, SPIB, ZNF276, RP3- |
| | | | | REV3L, | PTPRC, REV3L, | 402G11.25, OSBPL8, PLCXD1, FAM71D, SRRT, LINC00665, |
| | | | | CASP8, | CASP8, IKZF1, | C15orf48, ARID2, TNFSF14, WDR48, MAVS, RBM34, TRAPPC8, |
| | | | | IKZF1, | LY75, PPIF, | NUP160, CCZ1, PTPRC, PDE4B, REV3L, SH2B2, SEH1L, EFNB1, |
| | | | | GPR65, | GPR65, | ULK4, CASP8, STIM2, RBBP9, OPN3, ZBTB2, CAPN2, IKZF1, |
| | | | | PRKCB | PLAGL2, | FAM117B, CD55, SLC44A2, SH2D3A, TRMT6, GPR157, PRPF4B, |
| | | | | | IRF4, PRKCB | TPRKB, POLR2D, ZNF606, MOCS3, LY75, ETV3, CD52, ADSS, |
| | | | | | | PPIF, NAB2, NR4A3, UAP1, CHURC1, RPP40, WDR37, METTL22, |
| | | | | | | GNA13, PDE12, ZC3H11A, MARCH5, CTD-2267D19.2, |
| | | | | | | ELMSAN1, GPR65, TMA7, HIVEP1, ENTPD4, PAK2, SATB1, |
| | | | | | | AVPI1, ZNF335, ELF1, MARCKSL1, TMEM8B, PLAGL2, OST4, |
| | | | | | | TIMM23, AC004069.2, VHL, DDX21, AREG, USP3, AP1S2, |
| | | | | | | AC013394.2, ZNF514, STARD4, HOTAIRM1, IRF4, CAMKMT, |
| | | | | | | EZR, FGR, RBM39, MAT2A, FLNA, SPINK1, EPM2AIP1, LCP1, |
| | | | | | | CCDC28B, PRKCB, TRIP12 |
| 8 | CD4+ | IL10RA | UC | IL10RA, | IL10RA, IPMK, | IL10RA, LETM2, KDELC2, IPMK, CD69, KLRG1, TMBIM1, |
| | Memory | | | TMBIM1, | TMBIM1, | NCOA6, ZFAND2A, TSC22D3, ADRB2, PEX13, NFKBIZ, MCM9, |
| | | | | TAGAP, | NFKBIZ, | NPIPB4, C12orf75, TAGAP, VIM-AS1, DUSP3, MAPK8IP3, JUN, |
| | | | | SLC22A5, | TAGAP, | EIF4E3, SNX30, SAMHD1, RP11-299J3.8, IGHA1, SLC27A5, |
| | | | | TNFAIP3, | SLC22A5, | ZNF787, SLC22A5, OSGIN2, GCLM, PCGF6, C9orf41, IFIT3, |
| | | | | FOS, | OSGIN2, | PRKAB2, IGJ, ITGA4, RP11-549J18.1, MTFR1, PCOLCE, |
| | | | | PTGER4 | ITGA4, | SLC2A13, PIGW, ATF3, GBP1, MBNL2, TNFAIP3, CNN3, |
| | | | | | TNFAIP3, FOS, | ARHGEF40, PPP1R15A, UFSP2, FOS, HIST1H4J, SSFA2, |
| | | | | | PTGER 4 | HIST4H4, AC079210.1, PAXBP1, ANXA1, POLR2M, SMARCD3, |
| | | | | | | PRICKLE4, TMPRSS2, RP11-290F5.1, ASUN, BBS12, ANXA2R, |
| | | | | | | PTGER4, DLK2, N4BP3, ARMC5, OSM, RP11-302B13.5, TMEM62, |
| | | | | | | DNAJB1, SGK3, LAIR1, BCYRN1, RAD54B, DUSP1, PARP8, |
| | | | | | | UBE2Q1, ZNF230, C11orf74, ZDHHC14, SGPP1, TRPV3, TMEM91, |
| | | | | | | OGFRL1, PTGER2, RP11-500C11.3, SZT2, C2, ZNF665, KLHL18, |
| | | | | | | PLCXD1, RABL2A, LINC01004, SGOL2, NAP1L6, TNFSF9, |
| | | | | | | NR4A3 |
| 9 | GC | NFATC1 | Healthy | IRF8, NCF1, | NFATC1, IRF8, | NFATC1, IRF8, RP11-277L2.3, LYSMD2, PEA15, CIITA, YTHDC2, |
| | | | | LCK, ITGB2, | FADS3, NCF1, | RFX5, PPP1R18, MAP4K4, ZNF429, LAT2, HOPX, TTC9, P2RX5, |
| | | | | PTPRC, | REL, LCK, | ITSN2, GMIP, BCAS4, PLEKHG1, SWAP70, COMMD2, |
| | | | | BACH2, | ITGB2, CD40, | MARCKSL1, GPR18, CERS4, ARHGAP25, RP11-960L18.1, FADS3, |
| | | | | WAS | PTPRC, | MFSD10, ATP2B1, HIP1R, SNAP23, MBD4, SPI1, RAB4B, SEPT6, |
| | | | | | BACH2, WAS | CAMP, PXK, TFEB, NUBP1, ACTG1, NCF1, REL, ARID4B, LCK, |
| | | | | | | TRAPPC2, CTA-250D10.23, AP1B1, ITGB2, PGLS, UCP2, CD40, |
| | | | | | | ATP2A3, LCP1, LSM6, KDM1A, TCL1A, VNN2, C1orf228, PTPRC, |
| | | | | | | BRK1, BLOC1S2, STRIP1, TMEM199, MAP4K1, CLEC2D, CD22, |
| | | | | | | ACAP2, HTT, BACH2, BLCAP, UGCG, NCOA4, SREBF2, MITD1, |
| | | | | | | POLE4, MOB1A, LAMTOR5, RCC2, MAP3K7CL, WDR11, REST, |
| | | | | | | WDFY4, WEE1, SLTM, C7orf73, SHKBP1, HNRNPK, ZNF431, |
| | | | | | | FLI1, LYRM1, GPR132, SNX29P2, GGA2, WAS, FKBP1A, DAXX, |
| | | | | | | CAPZB, MTMR14, CSK, GEN1 |
| 10 | Tregs | RORC | UC | RORC, | RORC, CCL20, | RORC, CCL20, IL23R, MXD4, TNFRSF1A, AP2B1, CPD, SKAP2, |
| | | | | CCL20, | IL23R, | KATNB1, ATG16L1, ST3GAL5, TMEM167A, RAP2A, ADAM12, |
| | | | | IL23R, | TNFRSF1A, | ARHGEF7, GFI1, SLC15A4, CEP250, INVS, MYO9B, FAM89B, |
| | | | | SKAP2, | SKAP2, | INPP5D, MRPL10, SLC26A3, POLDIP3, RRAGC, PRDM1, |
| | | | | ATG16L1, | ATG16L1, | UEVLD,COL5A3, MDM2, CBLB, RP3-428L16.2, PLEKHO2, |
| | | | | PRDM1, | SLC26A3, | SEC61A1, DPF2, RNF213, PLAA, BCL2L11, PPM1B, SH2B3, |
| | | | | SH2B3, | PRDM1, | RAP1B, CD86,RPAP2, ANGPT2, RP11-252A24.7, FOCAD, |
| | | | | TMBIM1, | SH2B3, | ADAM19, MYCBP, YWHAH, CISH, ATXN2, FAM53B, DLEU2, |
| | | | | PTPN22 | TMBIM1, | BARD1, LRRC14, HSH2D, ANAPC4, SEPN1, BRIP1, APOL3, |
| | | | | | PTPN22 | TARSL2, ATP6V1A, FAM126A, NXPE2, SNORD3A, COX15, |
| | | | | | | DNAJC17, KCTD20, NOL8, CEPT1, VPS36, MT-TP, ZDHHC24, |
| | | | | | | TMEM260, ITPRIPL1, TMBIM1, DDX52, PHF11, CMTR1, SSH1, |
| | | | | | | MAPK1, PTPN22, RBM41, APOL1, GOLGA8B, TBCD, TTC31, |
| | | | | | | ABHD17A, SEC24D, PPP2R5E, CCDC9, ZSWIM8, FAM168B, |
| | | | | | | HOXB4, P2RY11, TM4SF5, RP11-356I2.4, GSPT2, |
| | | | | | | UBALD2, IP6K2 |
| 11 | Goblet | CCL20 | Healthy | CCL20, | CCL20, EFNA1, | CCL20, TSTD2, GPR128, MPZL3, SYT8, RAI14, RP11-349K16.1, |
| | | | | EFNA1, | EGFR, | RP11-1220K2.2, CENPJ, CTD-2566J3.1, EDN1, TLR3, NPTX2, |
| | | | | EGFR, | TNFAIP3, | RP11-640M9.1, PIM1, RFK, NFKBIA, RP11-567C2.1, NAALADL1, |
| | | | | TNFAIP3, | SLC26A3, | DPP4, FKBP1A, LMBRD1, BIRC3, CLCA4, AIM1L, CDA, |
| | | | | CASP8, | CASP8, IL2RG, | PSORS1C1, GLRA4, SEMA3C, AC016683.6, SLC1A1, GADD45A, |
| | | | | IL2RG, | NFKBIZ, | C2orf54, TTC22, SSUH2, SLC5A1, PDLIM2, IFITM10, AC005550.3, |
| | | | | SMAD3 | SMAD3, | P2RY1, FCGRT, CTSA, SLC3A2, ABTB2, EFNA1, AQP7, |
| | | | | | SEPHS2 | KRTAP13-2, EGFR, KIF2A, ESPN, EMP1, PMEPA1, FAM95B1, |
| | | | | | | RP11-227H15.4, TCHP, TMEM37, POMGNT2, SLCS30A10, |
| | | | | | | EPSTI1, SCARB1, ABCG2, DAB2, RBP2, CXCR3, TCTN3, |
| | | | | | | TNFAIP3, SLC26A3, RP11-373D23.2, CASP8, IL2RG, CASP10, |
| | | | | | | SLC3A1, ERO1L, ACSS1, SLC35G1, DEPDC7, TMIGD1, TM6SF2, |
| | | | | | | RHOD, SPTSSA, ALPI, PUS10, CEACAM7, AQP11, HLA-DRB5, |
| | | | | | | MPZL2, HUS1, PID1, HHLA2, NCBP1, AC079602.1, |
| | | | | | | RP5-828H9.1, NFKBIZ, CTSD, DENND5B, SLC9A3R1, LL22NC03- |
| | | | | | | 32F9.1, SMAD3, SEPHS2, MUC20 |
| 12 | Entero- | HPS1 | Healthy | HPS1, | HPS1, TOM1, | HPS1, LRP10, ZC3H12A, JUP, SLC25A25, VPS37B, LSR, IST1, |
| | cyte | | | SMAD3, | SMAD3, | CTDSP2, RHPN2, SRSF5, HDAC5, ADM, TBC1D1, TOM1, |
| | Pro- | | | TTC7A, | TTC7A, | DHRS3,SRC, SMAD3, SLC2A1, PKP3, HLA-E, RP11-465N4.4, |
| | genitors | | | C1orf106, | PTK2B, | RRAS, ALPI, PCDH1, TTC7A, OSBPL2, SGK223, MAP3K11, |
| | | | | TMBIM1 | ICOSLG, | TAPBPL, LASP1, SUN2, SLC25A23, FAM102A, ITSN1, MUC13, |
| | | | | | PRKD2, | MICA,MOV10, TXNIP, PTPRH, SEC14L1, TLE3, ATF3, UCK2, |
| | | | | | C1orf106, | GBA,PSORS1C1, PTK2B, PLXNA2, CTD-2267D19.2, SH3BP2, |
| | | | | | FOSL2, | ARSA, FURIN, EPS8L3, ICOSLG, IRF7, NEDD4L, SOX13, DDIT3, |
| | | | | | TMBIM1 | HEXIM1, FBXW5, TMEM127, ACVRL1, PRKD2, MGAT5, |
| | | | | | | RNA5SP151, LRRC8A, SERINC1, RP11-680F8.1, CTSD, SP110, |
| | | | | | | SPSB3, FAM211A, ATG2A, AGPAT3, ADIPOR2, ACAP2, GTPBP1, |
| | | | | | | KIAA0247, C19orf25, PNPLA2, PDCD4-AS1, ARHGEF18, ASPG, |
| | | | | | | SQSTM1, EPS8L2, ZNF213, SORL1, KCNK6, PSD4, Clorf106, |
| | | | | | | FOSL2, IRF1, TMBIM1, SYNPO, RETSAT, GPRIN2, TACC2, |
| | | | | | | AKAP13, APLP2, SPECC1L |
| 13 | NKs | ITGA4 | UC | TNFAIP3, | ITGA4, | ITGA4, OSM, MCAM, RORA, TNFAIP3, TUBD1, MGAT4A, |
| | | | | NFKB2, | TNFAIP3, | ADH1B, JUN, NFKB2, CASP8, NAV1, LGALS8, PHC1, |
| | | | | CASP8, | NFKB2, | CCDC157, FOSB, MIR24-2, GFPT2, PLK3, TCP11L1, IGHV3-33, |
| | | | | AHR, | CASP8, | KIAA0368, INADL, RP11-166B2.3, DUSP5, FHL1, DCP1A, |
| | | | | TAGAP, | AHR, | C12orf68, RNF152, RP11-819C21.1, SAMD12, TMEM63A, NRL, |
| | | | | PTGER4 | DNAJB4, | CSRNP1, ARHGAP10, AHR, TUBA1A, PNPLA8, MYADM, |
| | | | | | TAGAP, | PPP1R15A, ITPR1, FRMD4B, ADAM10, NEU1, CNP, KLF6, TTL, |
| | | | | | COQ10B, | RNF149, DNAJB4, C17orf107, TSC22D2, IGLV2-8, TAGAP, |
| | | | | | PTGER4, | TEAD3, NCK2, IGHV4-61, AMD1, EPM2AIP1, XPO1, COQ10B, |
| | | | | | SLC2A4RG | DNAJB1, IGLV3-21, CD69, TNIK, IGSF6, PTGER4, SLC2A4RG, |
| | | | | | | RBM23, LMNA, AFF1, KIAA0319L, ZNF324B, RP11-356C43, |
| | | | | | | EREG, WDYHV1, USP36, JPX, MCL1, PER1, ZGPAT, IGLV3-1, |
| | | | | | | RBL2, SPATA5, JUND, GCC1, FAM122C, ZNF674-AS1, DDX6, |
| | | | | | | SORL1, BTG2, DPP4, IFFO2, DUSP1, IGHV3-7, MLLT4, ARAP2, |
| | | | | | | NFE2L2, SPOCK2, IP6K1, RP11-293M10.5 |
| 14 | CD8+ | PIK3R1 | Healthy | PIK3R1, | PIK3R1, | PIK3R1, DCTN4, JMY, SERPINB9, LDOC1, DNAJC9-AS1, |
| | IELs | | | GPR65, | DCTN4, | GPR65, DERL3, DNAJA2, CD55, GLMN, NAA50, EDEM2, |
| | | | | GPR35, | GPR65, GPR35, | GABPA, SULT2B1, WBP11, TRIM73, LITAF, RBM4, ACTR5, |
| | | | | PTPRC, | PTPRC, | CKS2, C16orf91, DNAJB6, MCPH1, MTHFD2, SYTL3, ZNF569, |
| | | | | THADA, | THADA, | MORF4L2, PPP2R2A, LSMEM1, PJA2, MRPL47, SAMSN1, DLG5, |
| | | | | BACH2, | BACH2, | SDCBP, SRGN, ETF1, PLD2, GLA, GPR35, PTPRJ, PTPRC, |
| | | | | MAP3K8, | MAP3K8, | ZDHHC3, H2AFZ, EMD, DBF4, TMED8, NR1H2, ZNF655, |
| | | | | LIG4 | SOCS1, LIG4 | THADA, CHD1, BACH2, GLYCTK, ASTE1, GPN1, MAP3K8, |
| | | | | | | YES1, CTPS2, AUTS2, ZNF644, CTCF, RPAIN, XRRA1, SNX9, |
| | | | | | | SNORA40, PTP4A1, SMG8, BTG3, SOCS1, TEX14, NGDN, |
| | | | | | | SLC25A30, EIF5, STAT3, LIG4, DDX27, HMGXB4, PRR7, |
| | | | | | | MCUR1, STK38L, KDM4C, SPCS3, RPGR, PRUNE, SMEK1, |
| | | | | | | PGBD4, ATG5, PRMT5, MPHOSPH6, EXOC4, CDK17, RP11- |
| | | | | | | 425D10.10, WDR33, DYNLT3, CTD-2574D22.4, GRB2, |
| | | | | | | GTF3C2, LYRM5, ROCK2, MYSM1 |
| 15 | Macro- | PRDM1 | Healthy | PRDM1, | PRDM1, AHR, | PRDM1, SIK1, STARD7, PRRG4, ARMCX1, PSPC1, PTP4A1, |
| | phages | | | AHR, | FOSL2, | DHX38, SLC4A7, UBE4A, PTGS2, AHR, YBX3, FOSL2, EIF3A, |
| | | | | TNFAIP3, | TNFAIP3, | CPM, SPRED1, LATS2, IL13RA1, RRAD, NAMPT, SETX, PTBP3, |
| | | | | TGFBR1, | SLC30A7, | TNFAIP3, CS, FYB, SSFA2, FGD5-AS1, SOAT1, NR4A2, PICALM, |
| | | | | MAP3K8, | CLTC, | QKI, MIDN, MAN1A2, WDR45B, SLTM, USP16, COPA, ROCK1, |
| | | | | SH2B3, | TGFBR1, | ZNF331, HNRNPU, SLC25A16, SAP30, U2SURP, TMEM123, |
| | | | | TGFBR2 | MAP3K8, | SAFB2, FBXL5, SERINC1, IFNAR1, CCNE1, TMEM106B, |
| | | | | | SH2B3, | UHMK1, TMPO, SRSF6, VPRBP, DCP1A, SLC30A7, GIGYF2, |
| | | | | | TGFBR2 | CLTC, ATG4C, CRTAP, KLHL12, CLEC7A, FUBP3, KTN1, CTSO, |
| | | | | | | SLC17A5, PHTF2, KIAA1551, STAG1, PPAT, TLR7, MBP, CKAP4, |
| | | | | | | HSPH1, TERF2, HIPK1, GLYR1, DDX17, FMR1, SPAG9, DAPK1, |
| | | | | | | RCSD1, RFC1, PAG1, FAM35A, FAM198B, APOL6, KMT2A, |
| | | | | | | NR4A3, FUS, TGFBR1, SPOPL, MAP3K8, USP53, TFEC, SH2B3, |
| | | | | | | TGFBR2, SYNRG, SURF4 |
| 16 | Macro- | PRKCB | Healthy | PRKCB, | PRKCB, | PRKCB, ROCK1, HIST1H2BN, YBX3, INSIG1, SYAP1, SOD2, |
| | phages | | | NFKB1, | NFKB1, | WSB1, DDX5, CCDC88A, TOR1AIP1, SPTY2D1, NFKB1, ARL5B, |
| | | | | GPR65, | GPR65, | IL6R, OTUD1, RCSD1, GPR65, EIF1AX, ARMCX1, NAMPT, |
| | | | | PTGER4, | TGER4, | CKS2, ADCY7, MAP2K3, HNRNPU, ATXN3, GCC2, ACLY, FLI1, |
| | | | | PTPRC, | COQ10B, | AFF4, PNPLA8, RBM39, NFE2L2, N4BP2L2, PTGER4, FYB, |
| | | | | SH2B3 | LPXN, | PTPRE, RPL22L1, RHOT1, AKAP9, SF3B1, HSPE1, UBQLN2, |
| | | | | | PTPRC, REL, | DOCK8, LATS2, ANKRD12, CREB1, NCOA7, RBPJ, FADS1, |
| | | | | | NFKBIZ, | LCORL, NR4A2, PTGS2, SNHG8, CLK1, USP16, COQ10B, |
| | | | | | SH2B3 | PICALM, BAZ2B, PPP1R10, ATXN1, RASSF5, LPXN, SBNO1, |
| | | | | | | TANK, EPOR, LTA4H, PTPRC, CMTM6, SAFB2, NUS1, GPR183, |
| | | | | | | AC026806.2, SLC38A2, OPA1, REL, SETD5-AS1, NCOA6, VPS51, |
| | | | | | | SLC2A3, NAA50, IDI1, NFKBIZ, ANKRD10, OXR1, |
| | | | | | | SET, MAN1A1, SH2B3, ZNF106, CRNKL1, WTAP, FAM114A2, |
| | | | | | | SMARCA2, HIPK1, |
| | | | | | | SLC20A1, CD83, BDP1, PANK3, ETF1, LCP2 |
| 17 | Cycling | ABI1 | Healthy | NDFIP1, | ABI1, NDFIP1, | ABI1, MICAL1, ANKRD12, USP12, ADI1, ISCU, RHBDD2, |
| | T | | | IL2RG, | RNASET2, | PRPSAP2, NDFIP1, DNAJB12, FAM226B, MTRR, RAP1A, DCAF8, |
| | | | | CDKAL1 | IL2RG, PROCR, | FBRSL1, MEI1, SESN2, GGA3, CMTM6, RNASET2, IL2RG, EEF2, |
| | | | | | CYLD, | MPRIP, DUSP18, LINC00338, APOM, CNIH1, TRAM1, KIAA1328, |
| | | | | | CDKAL1, | CORO1B, ADPGK, DEDD, BCL2L1, FOPNL, LETMD1, P4HTM, |
| | | | | | TNFRSF14, | IQCE, CD37, SELM, PEBP1, CERS5, PROCR, TRBC2, CREBL2, |
| | | | | | TOM1 | LGALS8, SUPT4H1, EIF2AK2, PGAP3, C18orf25, MIA3, RPA3- |
| | | | | | | AS1, DUS4L, PTPRE, ZBED5-AS1, M6PR, AC015691.13, CYLD, |
| | | | | | | SYNE2, DGCR2, TNIK, ARL14EP, CDKAL1, PCBP3, TTC32, |
| | | | | | | VAMP5, SLC25A45, LMBR1L, TBRG1, ANKRD13C, CTSB, |
| | | | | | | FAM174A, EEF1D, UBC, RPL8, YIPF5, CTC-428H11.2, PRPS1, |
| | | | | | | FXYD5, GMFG, PIM2, TRAC, TOM1L2, TNFRSF14, UCP2, |
| | | | | | | PPP2CA, SARDH, ATP6V1G1, TOM1, TRADD, ABHD8, LTA4H, |
| | | | | | | NPC2, CEP85L, HNRNPLL, PKP4, TNRC6C-AS1, LINC01011, |
| | | | | | | RAB3IP, PM20D1, PFDN5 |
| 18 | Entero- | FOSL2 | Healthy | C1orf106, | FOSL2, | FOSL2, C1orf106, JUP, TMBIM1, PTK2B, EHD1, RIOK3, |
| | cytes | | | TMBIM1, | C1orf106, | KIAA0247, NBR1, CDKN1A, SP2, ZC3H12D, PRKCD, PNPLA2, |
| | | | | IL2RG, | TMBIM1, | HMOX1, SLC16A3, MYO1E, CTSB, RHOU, TMEM51, |
| | | | | HPS1 | PTK2B, | SLC20A2, MAP2K3, SPINT1, BCL2L11, F11R, ACSS1, PTPRH, |
| | | | | | SP140L, | ZFP36, RBM23, ERBB3, AKAP13, ABHD12, BDKRB2, |
| | | | | | TNFRSF1A, | CPM, PRSS8, IRS2, SP140L, ZNF655, WAC, IFNLR1, MYLIP, |
| | | | | | IL2RG, HPS1, | AGPAT3, CLIC5, KIAA2013, TNFRSF1A, PER3, ABCG1, PPAP2B, |
| | | | | | IFNGR2 | TTC22, PSAP, TES, DNAJA1, RP11-244F12.3, RP11-490M8.1, |
| | | | | | | RAB11FIP1, PCDH1, FAM32A, ZC3H12A, ITPR3, CLSTN1, |
| | | | | | | APLP2, C10orf54, TJP1, RP11-30P6.6, CHIC2, LIPH, IST1, UACA, |
| | | | | | | PTTG1IP, MEP1A, GBA, SRSF5, AMACR, IL2RG, PPP1R3B, |
| | | | | | | LRRC1, SDC1, LAMP1, LYST, BAMBI, P2RX4, ACSL5, |
| | | | | | | ST6GALNAC6, PLIN3, IRF6, HPS1, MXD3, MAP3K11, INPP5K, |
| | | | | | | PVRL2, IFNGR2, ETS2, CTSA, KIAA1217, OSER1, DNMBP, |
| | | | | | | ACAP2, GPA33, NEDD9, TMEM37 |
| 19 | CD8+ | NFATC1 | UC | FOXP3, | NFATC1, | NFATC1, ICA1, ACTN1, TRIB1, MAGEH1, TNFRSF4, CD200, |
| | IELs | | | ITGB2, | FOXP3, | ETV7, ARID5B, LGMN, POU2AF1, CARM1, ANKS1B, SGPP2, |
| | | | | TNFRSF13B, | ITGB2, CXCR5, | CFP, TNFRSF8, FBLN7, PASK, ZSWIM1, GPR75-ASB3, NRP1, |
| | | | | ANKRD55, | TNFRSF13B, | ITGB2-AS1, PTGIR, LHFP, C1orf228, RP5-1028K7.2, CCDC6, |
| | | | | IL10 | CXCL3, | GNG8, FOXP3, KB-1980E6.3, ANG, GMEB2, EBI3, IL1RAP, |
| | | | | | ANKRD55, | FBXO10, PTPN14, RP11-796G6.2, SNX21, CHGB, EHD4, |
| | | | | | CD5, IL10 | IGFL2, CXCL13, NAPEPLD, MIR181A1HG, CAV1, GJB1, |
| | | | | | | ITGB2, CXCR5, DVL1, FAR2, CHST7, TNFRSF13B, ZBTB42, |
| | | | | | | FAAH2, DAPP1, TSHZ2, CXCL3, SUPT7L, KLF7, G0S2, |
| | | | | | | CCND1, CORO1B, CD79B, ANKRD55, PVALB, RASGRP4, |
| | | | | | | RP11-460N16.1, DIRAS3, TSPAN12, NPDC1, SELL, CD5, |
| | | | | | | IFRD2, SAV1, RP11-265P11.2, PKIA, FKBP5, RP11-345M22.1, |
| | | | | | | HNRNPLL, CEP112, EARS2, SMAD1, C14orf64, ETV5, |
| | | | | | | DERL3, PTHLH, RASGRP3, PABPC3, MAL, CYP7B1, DMD, |
| | | | | | | IL10, IGHV1-3, AL138764.1, CCR7, FLVCR2, CDK2AP1, |
| | | | | | | GPX7, HIST1H2BN, MAGEF1 |
| 20 | GC | PTPRC | UC | PTPRC, | PTPRC, | PTPRC, LRRFIP1, UBAC2, APBB1IP, BIRC6, SEPT9, REL, |
| | | | | BTK, | UBAC2, | NCOR1, TRIM38, ELOVL5, SEPT6, LYN, PPP1R12A, |
| | | | | RIPK2, | REL, BTK, | NELFCD, ORAI2, CDC40, SESTD1, MOB3A, ITSN2, SNX6, |
| | | | | SKAP2, | YDJC, | CELF1, SREBF2, ERICH1, CREBBP, PPM1K, SWAP70, |
| | | | | PLCG2 | RIPK2, SKAP2, | UBE2D1, RPL7L1, CTA-250D10.23, ATM, ELK4, CYFIP2, |
| | | | | | PLCG2, CXCR5 | TPR, POU2F2, MOB4, TAF7, IDI1, KIAA0247, GRB2, |
| | | | | | | CHORDC1, RNF41, BTK, WAC-AS1, NR3C1, SYNRG, |
| | | | | | | GMFB, TRIM33, ZNFX1, EGLN2, ARID4B, PPP1R18, |
| | | | | | | ACTR2, PXK, DDX27, ZFAS1, FAM49B, TAOK3, |
| | | | | | | ARFGAP2, RNMTL1, ATP2B1, CLEC2D, ATP6V1H, STAT6, |
| | | | | | | ENTHD2, DENR, LINC00685, SLC44A2, YDJC, EIF2B5, |
| | | | | | | NUP160, RIPK2, NGDN, FNBP1, BTF3L4, FDFT1, |
| | | | | | | KIAA0922, SKAP2, SYK, PTDSS1, ARHGEF1, CERS4, |
| | | | | | | MAP1LC3B, ABI3, SP140, XPO1, PLCG2, LARP7, PPIL4, |
| | | | | | | JAK1, ETS1, MCRS1, RP4-717I23.3, SRSF5, RBM5, TINF2, |
| | | | | | | PLEKHA2, ABCG1, CXCR5, WAPAL, PDCD10 |
| 21 | CD8+ | SLC2A4RG | Healthy | PRKCB, | SLC2A4RG, | SLC2A4RG, FAM159A, GTF3C1, AQP3, TNIK, OSM, |
| | IELs | | | CD6, | PRKCB, | SSBP2, PCYOX1L, IGHV3-30, FUT8, RP11-191L17.1, |
| | | | | CDKAL1, | DNAJB4, CD6, | EMP3, PRKCB, SH3KBP1, THEMIS2, C1R, SLA, FBXL2, |
| | | | | IL23R, | CDKAL1, | SH2D1A, ITK, SEMA4C, SYTL1, RP11-160O5.1, |
| | | | | NFKB2, | IL23R, NFKB2, | ANTXR2, MPZL2, USP45, RILPL2, IGLV2-8, TMEM86B, |
| | | | | ITGB2, | ITGB2, | UXS1, MEPCE, CDK5RAP2, IGKV3-15, CD82, TMEM55A, |
| | | | | SLC39A8 | SLC39A8 | NFKBIA, RP11-589C21.6, C4orf32, DNAJB4, THAP7-AS1, |
| | | | | | | C16orf54, POFUT2, RP11-383H13.1, HKR1, PBXIP1, XBP1, |
| | | | | | | LGALS3BP, SLAMF1, LST1, FXYD2, FSIP1, SIT1, |
| | | | | | | FAM53C, C1orf132, CTD-2201E18.3, LBH, RNU12, FKBP11, |
| | | | | | | CD6, CDKAL1, RND1, TNFRSF25, IFNGR1, CERK, |
| | | | | | | LDLRAP1, TUBB2A, CCDC109B, CCR2, IGLV3-16, |
| | | | | | | SLC25A4, SESN2, IL23R, KIF9, SEMA4A, NFKB2, CTSH, |
| | | | | | | LDHB, TTC13, KDSR, SULT1C2, CTC-523E23.11, CDKN1A, |
| | | | | | | HNRNPUL1, TXNL4B, POU2AF1, IGKV3D-20, TCEAL3, |
| | | | | | | SGK1, MYLIP, TOB1, CD44, AMIGO1, ITGB2, SS18L1, AIF1, |
| | | | | | | SLC39A8, AMN1, IGKV1-16, P2RY8, S100A6 |
| 22 | CD4+ | TAGAP | UC | TAGAP, | TAGAP, | TAGAP, MCLl, DNAJB1, TNFAIP3, PPP1R15A, ARL5B, |
| | Memory | | | TNFAIP3, | TNFAIP3, | YPEL5, KLHL18, TSC22D3, HCFC2, CYCS, SIK1, RGS2, |
| | | | | PTGER4, | PTGER4, | PTGER4, ZFP36, FAM46C, RP5-1073O3.7, IREB2, TUBA1A, |
| | | | | CASP8, | NFKBIZ, | TRAK2, BTG1, DYNLT3, CD69, EIF4E3, C4orf46, DNAJB9, |
| | | | | JAZF1 | ITGA4, CASP8, | PRR7, LOXL1-AS1, KLF6, PRNP, RP11-727A23.5, COL3A1, |
| | | | | | DAGLB, | NFKBIZ, CITED2, FOXJ1, PDIA5, TMEM62, OSM, |
| | | | | | JAZF1, FOSL2 | EPM2AIP1, PER1, DSE, PFKFB3, ITGA4, RGCC, TTC39B, |
| | | | | | | DNAJA1, NR4A1, IDI1, PCGF5, PDE4D, MT-TH, FOSB, |
| | | | | | | CAPN2, SRPX, SPG20, RP11-489E7.4, SRSF7, CASP8, |
| | | | | | | MTFR1, TCTN3, CD83, SNX30, DAGLB, RP11-191L17.1, |
| | | | | | | TAGLN2, JAZF1, IGLV3-1, LMNA, POLR2M, KIAA0754, |
| | | | | | | ARIH1, PARD6A, PARP8, ZNF250, CBWD3, ACAP2, AAED1, |
| | | | | | | WDTC1, ANXA1, KAT2B, IGJ, RUNX2, TC2N, OGFRL1, |
| | | | | | | IGLC7, CXCR4, HMGB2, ETV3, EMB, SYTL3, CDKN1A, |
| | | | | | | RORA, NEU1, RP11-504P24.8, FOSL2, TIPARP, AMD1, |
| | | | | | | NRBF2, TMEM91, PHC1 |
| 23 | TA 2 | TGFBR2 | UC | TGFBR2, | TGFBR2, | TGFBR2, VPS13C, AFAP1, SEC14L1, CPSF2, MED31, VEZT, |
| | | | | TGFBR1, | NFKBIZ, | SLC7A1, C5orf15, TMCC1, STK3, ACO1, KBTBD2, CALU, |
| | | | | IFIH1, | TGFBR1, IFIH1, | HIF1A, CHMP4C, HDGF, EID2, ARPP19, MAPK1IP1L, |
| | | | | ERAP1, | ERAP1, | TMTC2, MYO6, NFKBIZ, TGFBR1, IRF2, SPRED1, CRNKL1, |
| | | | | FERMT1, | FERMT1, | RAP2C, TCF12, CDC27, FAF2, CIR1, IFIH1, ACBD3, TMED8, |
| | | | | AFIR | SMURF1, AHR, | MESDC1, DIRC2, EPT1, ETV3, ERAP1, JAK1, TMX3, LMAN1, |
| | | | | | FOSL2 | TPM1, FERMT1, E2F3, TNFAIP1, IL6ST, DTX3L, SMURF1, |
| | | | | | | YOD1, ARL5B, GPCPD1, RAB22A, BMPR2, RASGEF1B, |
| | | | | | | AKIRIN1, POLK, FER, EPB41L4A, MSI2, FBXW2, PSME4, |
| | | | | | | RP11-747H7.3, MTUS1, GSPT1, WDR45B, RFFL, ATF6, |
| | | | | | | ATP2C1, FAM105B, IDE, AHR, EXOSC6, MPP3, RANBP2, |
| | | | | | | UBA6, PTPN12, PVRL4, NUP155, FAM160B1, TMEM33, |
| | | | | | | TROVE2, UBQLN1, TC2N, USO1, ZBTB18, TJP1, STAT1, |
| | | | | | | PALLD, PURB, ASPH, CDC16, FAM21B, GLUL, ITSN2, |
| | | | | | | IBTK, FOSL2, RLIM, LMBR1 |
| 24 | Imma- | TMBIM1 | UC | TMBIM1, | TMBIM1, | TMBIM1, ARHGEF5, AKAP13, KIAA0247, EHD1, EFNA2, |
| | ture | | | SMAD3, | TNFRSF1A, | CCDC68, CTNNA1, SCNN1A, DOK4, GNG12, ZMYND8, |
| | Entero- | | | IL10RB, | SMAD3, | LITAF, MIDN, LRRFIP1, MCL1, ACTN4, MXD1, TNFRSF1A, |
| | cytes2 | | | DCLRE1C, | PTK2B, | TRAK1, CFLAR, SMAD3, F11R, STK24, CARS, FURIN, |
| | | | | HNF4A, | IL10RB, | PTK2B, RIOK3, ZNFX1, CDC42EP4, REEP3, DOCK1, CASP7, |
| | | | | C1orf106 | FOSL2, | TOR1AIP2, EPS8, BCL2L1, CHMP1B, RASSF6, WIPF2, LASP1, |
| | | | | | DCLRE1C, | MAP2K3, IL1ORB, ANKS4B, IFIT2, HHLA2, KIAA1217, |
| | | | | | HNF4A, | EHBP1L1, PLEC, IFNAR2, FOSL2, EZR, TMEM8A, LMO7, |
| | | | | | C1orf106 | NRBP1, DSC2, KIF13B, DCLRE1C, MYD88, RXRA, TMEM2, |
| | | | | | | CERS2, DDX60L, CDK19, HNF4A, CHMP2B, CYP3A5, NT5C2, |
| | | | | | | ZC3H3, AHNAK, TMEM63B, SNRK, LRRC1, KCNK6, |
| | | | | | | ADIPOR2, P2RY2, VASP, IRF6, TMPRSS2, DST, PDCD6IP, |
| | | | | | | KLF6, TJP1, KIAA1671, ETV6, PTPN9, PAFAH1B1, SPTBN1, |
| | | | | | | ATXN7L3, PFKP, CDKN1A, B4GALT5, CYTH2, C1orf106, |
| | | | | | | MUC13, SUN2, SLC45A4, B3GNT3, SRC, MICAL2, RP11- |
| | | | | | | 427H3.3 |
| 25 | CD4+ | TYK2 | UC | TYK2, | TYK2, PTK2B, | TYK2, CDYL, CHMP1A, UBA7, KDELC1, DNAJB6, PTK2B, |
| | Acti- | | | HPS1, | PRK D2, HPS1, | ZFPL1, RNF10, PNPO, CAMTA2, RAD9A, TMC6, RASAL3, |
| | vated | | | IKZF1, | UBAC2, IKZF1, | ATP6V1D, GRAP2, PPP3R1, PPP1R8, ARMC8, INPP5K, PRKD2, |
| | Fos-hi | | | SKIV2L, | SKIV2L, YDJC, | TTC31, SUB1, OS9, HPS1, IPPK, DTX3, RAB8A, LAMP1, FOXO1, |
| | | | | ZNF831 | ZNF831 | SPHK2, CAP1, LCMT1, ZBTB17, DHX38, SRPK2, QARS, IVD, |
| | | | | | | IL17RA, FLI1, PKN1, CYB5R3, MYO6, UBAC2, RBM41, EIF2B5- |
| | | | | | | AS1, SEPT9, TNFSF4, RBMX2, CDV3, PITPNA, CLCN7, IKZF1, |
| | | | | | | C1orf216, ARHGAP30, AK3, ACTN4, PHF20, ZCCHC10, XPC, |
| | | | | | | HMG20B, METAP1, TBXAS1, TAF10, LMF2, MSL1, GPBP1L1, |
| | | | | | | USP14, LCP2, SKIV2L, ABHD17A, MKNK2, C19orf25, YDJC, |
| | | | | | | RPUSD4, WDR45B, UBN2, LZTS2, PTPN4, EID2, UNC13D, |
| | | | | | | MED7, SUPT5H, DFFA, BRWD1, FAM134A, MCTS1, |
| | | | | | | MAPKAPK3, ZNF831, RFOF, HELZ2, LDB1, NUP155, MED25, |
| | | | | | | DCTN2, MRFAP1L1, C2orf76, ZNF672, PSD4, GUCD1 |
| 26 | Endo- | CASP8 | | CASP8, | CASP8, ERAP1, | CASP8, GPCPD1, PPFIBP1, CBL, LIN7C, MIA3, ACSL4, TDRD7, |
| | thlial | | | ERAP1, | SLAIN2, | ATOH8, IER5L,SIN3B, PHLDB1, STT3B, SNX14, EFHC1, |
| | | | | SLAIN2, | ERAP2, | ACTN4, DIS3L, YY1AP1, LENG9, STARD13, GPBP1, APOLD1, |
| | | | | ERAP2, | REV3L, | DLC1, ATXN3, COG5, FOXJ2, DBF4, FNDC3B, SCAMP4, NIN, |
| | | | | REV3L, | ADAM17, | PBXIP1, MPRIP, ERAP1, STK24, TAF1B, SLAIN2, POLR2B, |
| | | | | ADAM17, | AKAP11, | ZNF563, TMEM41A, KIAA1430, BDNF-AS, GPRC5C, TTC32, |
| | | | | TTC37 | TTC37 | ERBB2IP, LIMD1, BTBD7, MEF2C, SBK1, GABPB1-AS1, |
| | | | | | | CDH17, ZNF658, SNTB2, MINK1, ZNF75A, GALNT16, GRAPL, |
| | | | | | | RP11-407G23.4, EIF4G3, PIEZO1, ADAR, FBXL4, ROCK2, |
| | | | | | | BRD1, ZNF677, USP7, GTF2I, ASH1L, UTRN, ERAP2, MAP3K4, |
| | | | | | | OXCT1, CHD3, SEMA4F.XRCC1, CAPN3, MEF2A, SLC26A4, |
| | | | | | | REV3L, SEMA4C, RNF14, CCDC13, ABCG1, SSBP3, CNTNAP1, |
| | | | | | | BEND4, PODNL1, FBXO18, ADAM17, RIOK3, NUCKS1, |
| | | | | | | DDX50, RP11-245J9.4, AKAP11, TTC37, PRKAB2, ABCB4, |
| | | | | | | LTN1, SLC33A1, AQP7, SLC16A1 |
| 27 | ILCs | CCL20 | UC | CCL20, | CCL20, | CCL20, PRR5, RBL2, MPZL3, RNF149, PTPN22, CPNE7, PERP, |
| | | | | PTPN22, | PTPN22, | RORC, RANBP9, TBC1D31, CERK, ZBTB16, APOL6, CPOX, |
| | | | | RORC, | RORC, STAT4, | SLA, DHRS3, DCAF5, NMRK1, GPR171, H1FX, |
| | | | | STAT4, | COQ10B, IFNG, | PDE4D, YWHAH, TMEM204, SPN, TMEM167A, PPP2R2B, |
| | | | | IFNG, | TNFRSF1A, | ABCB1, LCP2, TNFSF14, ERN1, ASB8, RORA, OSM, |
| | | | | CD40LG | CD40LG | G3BP2, NEO1, SPRY1, STAT4, MRPL10, MYO1F, |
| | | | | | | FSD1, APOL3, RAP1B, PITPNC1, HIC1, ETS1, TXK, CPD, |
| | | | | | | SMAP1, COQ10B, MGAT4A, ZFYVE28, TGFBR3, GRAMD1A, |
| | | | | | | IL22, TANGO6, GPR155, LTK, IFNG, TNFRSF1A, TBC1D2B, |
| | | | | | | HERPUD2, LAMP1, CD96, KIAA0232, NR1D1, AC092580.4, |
| | | | | | | SLC4A10, IL18RAP, AMICA1, CD40LG, PARP8, REEP3, |
| | | | | | | ZNF18, TNFSF13B, KLHL13, UGGT1, LMLN, GIMAP2, CTD- |
| | | | | | | 2196E14.4, CYB561, ABRACL, SETD8, PTPLAD2, STOM, |
| | | | | | | LPIN2, GYG1, PPP2R5A, RUNX2, TMIGD2, SLC7A6, CDK6, |
| | | | | | | ATXN3, RNF115, ABCA5, DNM2, KIAA1211L, RBPJ, RP11- |
| | | | | | | 81H14.2, NPY1R |
| 28 | CD8+ | CD6 | Hea1thy | CD6, | CD6, CD5, | CD6, TC2N, CD82, ANTXR2, CD5, S100A4, SLAMF1, PDCD1, |
| | 1ELs | | | PRKCB, | PRKCB, | P2RY8, EMP3, PRKCB, SORL1, FYB, MSC, LTB, TOB1, PAG1, |
| | | | | ITGB2, | ITGB2, CCL20, | TMEM173, AC013264.2, RORA, CCR2, THAP7-AS1, |
| | | | | CCL20, | PTGER4, | TNFRSF25, SLA, RIMKLB, ADRB2, CD44, MICAL2, ANXA1, |
| | | | | PTGER4, | IL23R, | CTSH, ITGB2, CCDC109B, CCL20, NELL2, C14orf64, CYB561, |
| | | | | IL23R, | CD40LG | DAPP1, C22orf34, F2R, ZNF252P-AS1, RP11-1399P15.1, PCF11, |
| | | | | CD40LG | | LDLRAP1, IFT57, PTGER4, SIT1, ITGB2-AS1, SEMA4A, LST1, |
| | | | | | | IGKV3-15, MYO5A, MTA3, CACNA2D4, ADAM10, CTC- |
| | | | | | | 228N24.3, RP11-143J12.2, C1orf132, RP11-326C3.11, |
| | | | | | | LINC00892, RP11-109G23.3, SH3KBP1, RP11-383H13.1, |
| | | | | | | SPOCK2, SH2D1A, MGAT4A, RP11-222K16.2, LGALS3BP, |
| | | | | | | RASGRP2, IL23R, HPSE, FAM84B, SS18L1, AC017002.1, |
| | | | | | | S100A6, CD40LG, IL7R, PARVA, HIVEP1, IGKV1-16, DDR1- |
| | | | | | | AS1, METTL4, CLU, B2M, SNAI1, USP36, RP11-333E1.1, |
| | | | | | | FBXL2, RP11-589C21.6, FKBP11, C1orf228, IL4I1, AIF1, |
| | | | | | | AC109826.1, PBXIP1, CD2, GCSAM, IGHV3-30, NR3C1, |
| | | | | | | PLCG1, RAB2B |
| 29 | MT-hi | CD6 | Healthy | CD6, IL23R, | CD6, IL23R, | CD6, BTG1, CHRAC1, TC2N, HNRNPUL1, CD44, CD82, |
| | | | | ITGB2, | ITGB2, | DNAJB9, IGLV2-8, SIAH2, A2M, RILPL2, SOCS3, IGLV3-10, |
| | | | | PTGER4, | PTGER4, | UBE2D1, SIK1, DPP4, RP11-134P9.3, IL23R, ITGB2, SLC2A3, |
| | | | | NFKB2, | NFKB2, | LUM, EMILIN2, GARS, SAT1, CSRNP1, IGLV1-40, XBP1, |
| | | | | TAGAP, | TAGAP, | FOXJ1, TMEM66, PTGER4, ARL6, IGHM, IGHV6-1, PLIN2, |
| | | | | CD28 | DNAJB4, | NAF1, TRIP13, SDC4, HAR1A, CCL8, RP11-418J17.1, |
| | | | | | CD28 | ZFAND2A, IGHV1-18, GPR15, IGLV7-43, IGHV3-30, NSG1, |
| | | | | | | SBDS, TPBG, NFKBIA, G3BP2, F3, TRAF4, HUS1B, NFKB2, |
| | | | | | | TAGAP, SERP1, PLK3, IGHV3-74, FBLN7, PLVAP, |
| | | | | | | ANKRD13C, ZNF354B, SLC31A2, AC096579.7, C4orf32, |
| | | | | | | IGLV3-9, RP11-313P13.5, IGHA2, DDX21, PCYOX1L, |
| | | | | | | DNAJB4, ILF3-AS1, IGLV3-25, RGS2, HERPUD1, ZBTB11- |
| | | | | | | AS1, CCND1, IBA57, NEK8, BEX5, RAB33B, CTD-2313N18.5, |
| | | | | | | CD28, CD47, MS4A6A, PHLDA1, CLU, C1QB, IGLV4-69, |
| | | | | | | TUBA1A, C1QA, APOC1, SSBP2, BAMBI, TMEM237, LTB, |
| | | | | | | DNAJB1, POLR2J4, HKR1 |
| 30 | Imma- | COQIOB | UC | STXBP2, | COQ10B, | COQ10B, RAPH1, F3, GBP3, TNFRSF21, SP110, TLR4, ST3GAL4, |
| | ture | | | JAK2, | ITGAV, | TNFAIP8, LRP10, KLF2, B3GALT4, ITGAV, CASP10, OASL, |
| | Goblet | | | IL2RG | TNFRSF1A, | TDP2, TSC22D1, AKIRIN2, RIMS3, XKR9, TNFRSF1A, HIGD1A, |
| | | | | | STXBP2, | SNAP23, IGLV1-47, HLA-F, PARM1, LDLRAD4, STXBP2, HK2, |
| | | | | | CPEB4, | ELOVL6, SKIL, CEACAM5, LMO7, KAZN, MAPK6, RP4- |
| | | | | | JAK2, CARD11, | 583P15.10, SGSM1, SULT1C3, HEXA-AS1, TMC5, OPTN, FCER2, |
| | | | | | IL2RG | PVRL2, SWAP70, BHLHE40, RCAN1, IFNGR1, MMAA, |
| | | | | | | SH3KBP1, C1QTNF6, CPEB4, ARID3A, C18orf8, IFIT2, RELB, |
| | | | | | | CCNYL1, LONRF3, CRABP2, IGHV1OR15-1, STAT1, IGHV2-70, |
| | | | | | | PARP9, C19orf67, B4GALT1, ZC3H12A, CTSE, RNF19B, KCTD10, |
| | | | | | | STS, CPA2, CAST, CXCL5, RP5-882C2.2, RP11-517B11.7, SMPD1, |
| | | | | | | GJB4, JAK2, MUC13, RFK, ARL4A, CARD11, CTNNA1, FRMD3, |
| | | | | | | ACER3, RPL34-AS1, CASP1, IL2RG, IL21R, AL133373.1, |
| | | | | | | TSPAN3, KCNK1, CAP1, SOWAHB, RP11-79H23.3, EXOC3L1, |
| | | | | | | CUZD1, CTB-119C2.1, NEK11, KB-1410C5.5, ZNF189 |
| 31 | Macro- | CPEB4 | Healthy | PIK3R1, | CPEB4, | CPEB4, SLC11A2, RARRES1, ATF6, MITF, GANAB, CPNE8, |
| | phages | | | LACC1, | PIK3R1, | SLC38A6, PDCD6IP, ENOSF1, PAPSS2, PIK3R1, MR1, GOLGA4, |
| | | | | SPRED2, | LACC1, | SEPT10, CERS2, MANBA, RNLS, HERPUD2, ABL1, PER3, |
| | | | | MAP3K8, | SPRED2, | TRAF3, LACC1, TFDP2, ATP1B1, RDH14, SPRED2, TCF4, |
| | | | | FCGR2A, | MAP3K8, | ATP1B3, CYFIP1, NPC1, ICAM1, NAPG, HSD17B4, IFNAR1, |
| | | | | CYBB | DCTN4, | IP6K1, SMPDL3A, NPEPL1, IRS2, GNS, CD163, TMCO3, |
| | | | | | FCGR2A, | SERINC1, MAP3K8, VPS26A, ABCC10, GPNMB, LIPA, CHD8, |
| | | | | | CYBB | MINA, LAMP1, PINX1, MSR1, SPG20, SMPD1, USP38, EV15, |
| | | | | | | P4HA1, IDH1, SLCO2B1, TOP1, HECTD1, TRAPPC10, G3BP1, |
| | | | | | | ADAM28, FAM13A, ATXN2, MRPS36, FICD, DCTN4, WDR45B, |
| | | | | | | STOM, MFSD8, RPN1, AGPAT5, MPP1, CANX, MAGT1, |
| | | | | | | TMEM248, PIGX, FCGR2A, RFC1, TECPR1, ELMOD2, AMPD3, |
| | | | | | | TMOD3, ARHGEF40, ANAPC4, RAPGEF1, TMEM127, |
| | | | | | | SLC35A4, RP11-192H23.4, CYBB, SFSWAP, IGHV3-72, NFIC, |
| | | | | | | DYNC1H1, SNX18, ZNF331, TM9SF4 |
| 32 | CD8+ | CYTH1 | UC | CD28, CD6, | CYTH1, CD28, | CYTH1, TNFRSF25, TMEM173, CD28, C14orf64, SPOCK2, |
| | IELs | | | ICOS, | CD6, ICOS, | RMND5B, CD4, LINC00861, PBXIP1, TPTEP1, RP11-493L12.4, |
| | | | | TNFRSF13B, | CD5, | PCBP3, RNF149, CD6, TNIK, ICOS, ZC3H12D, HAUS3, |
| | | | | FOXP3, | TNFRSF13B, | MGAT4A, C1orf228, C16orf87, RAB3A, FRMD4B, CTSB, |
| | | | | CD40LG | FOXP3, | TTC13, KCNA3, FBXL8, SH3KBP1, PXN, ALPK1, IL12RB1, |
| | | | | | CD40LG | SOCS3, BIRC3, REEP3, CD5, AC005003.1, BLOC1S3, PSAT1, |
| | | | | | | MAL, ATXN7L1, ARNTL, SESN3, RASGRP2, HNRNPLL, |
| | | | | | | ELOVL4, RP11-15H20.6, CAMK1D, LINC00649, TNFRSF13B, |
| | | | | | | RP11-126K1.6, SNHG11, ARID5B, FOXP3, ACTN1, ENTPD4, |
| | | | | | | S1PR1, UXS1, PLEKHG3, CFP, ST8SIA1, AP3M2, SIDT2, |
| | | | | | | STK39, SUSD4, IL1R2, OSM, ZCCHC11, GBP4, RP11-248G5.8, |
| | | | | | | GNA15, TMEM63A, TGIF2, FBLN7, RP11-119D9.1, KLF2, |
| | | | | | | DNAJC18, SLAMF1, KCTD21-AS1, HIC2, RP11-796G6.2, |
| | | | | | | PLEKHM1, MORN3, FAS, CTD-2267D19.2, ZFYVE1, |
| | | | | | | TNFSF13B, RABL2B, UBQLN2, ANK1, ADK, RP11-275I4.2, |
| | | | | | | ATF7IP2, C16orf52, CD40LG, RNF44, L3MBTL1, ANTXR2, |
| | | | | | | AC109826.1, RP11-265P11.2 |
| 33 | Cycling | DNAJB4 | UC | JAK2, | DNAJB4, JAK2, | DNAJB4, JAK2, ITGAV, RNF145, CTC- |
| | TA | | | SH2B3, | ITGAV, | 425F1.4, FGD6, C4orf33, PARM1, SGMS1, AC083900.1, DIO3, |
| | | | | PRDM1, | CPEB4, | FAM3C, PRKAR2B, C10orf118, C9orf135, RP11-408A13.3, HK2, |
| | | | | CYBB, | SH2B3, | NCEH1, RP11-747D18.1, RP1-193H18.2, BHLHE41, |
| | | | | CCL20 | PRDM1, | RP11-511B23.2, RNU4-1, SKIL, MXD1, TCF7L2, UEVLD, |
| | | | | | CYBB, CCL20 | CPEB4, FAM178B, SSPN, ANO5, MYLK, CTA-228A9.3, |
| | | | | | | PIK3AP1, ITGB6, USP38, RNF11, RP5-882C2.2, EMB, KCTD9, |
| | | | | | | DZIP3, MAPK6, TMPRSS6, ATP11B, C5orf17, NUDT4, |
| | | | | | | ZC3H12C, CSTA, PALLD, U3, CTC-365E16.1, SPIRE1, RP11- |
| | | | | | | 342K6.2, SHOC2, DOCK4, RNU5E-1, PAQR8, B3GNT5, TC2N, |
| | | | | | | STAT1, DUSP6, IL19, STEAP2, SH2B3, BHLHE40, RAPH1, |
| | | | | | | PARP8, SGMS2, B3GNT2, SLC26A4, RP11-536C5.7, DDX58, |
| | | | | | | TRIM60, MYO6, PRDM1, SEC22B, TCF12, PCDH20, PON3, |
| | | | | | | PDE4D, BAI1, RP11-95M15.1, GLRA2, RP11-79H23.3, |
| | | | | | | B4GALT1, CYBB, TMEM217, RP11-383CS.5, CXCL5, YPEL2, |
| | | | | | | AC005550.3, ITGA3, RP11-686D22.8, TTC40, TNFRSF21, |
| | | | | | | MTUS1, CCL20, RP2, RUNX2, APOL6 |
| 34 | TA 1 | FOSL2 | Healthy | HNF4A, | FOSL2, | FOSL2, SLC25A23, CARD10, MYH14, NDRG1, HNF4A, |
| | | | | MST1R, | HNF4A, | MST1R, GNA11, VDR, RXRA, TRAK1, JOSD1, C1orf106, |
| | | | | C1orf106, | MST1R, VDR, | KIAA0247, B4GALNT3, WIPF2, SYNPO, IGF2R, HSPG2, |
| | | | | XIAP, | C1orf106, | CTNND1, PLEC, ARHGAP17, ARHGAP35, SEPT8, MICAL2, |
| | | | | GSDMB | XIAP, | UBR2, ANTXR2, LIPH, KIAA0232, SIPA1L3, NEURL1B, |
| | | | | | PTK2B, | RHOU, LLGL2, JUND, CNNM4, XIAP, PTPRH, MIDN, INF2, |
| | | | | | GSDMB | VPS37B, TMPRSS2, FLNB, TMEM8A, TPRN, MTRNR2L12, |
| | | | | | | ERBB3, TMEM127, NADK, CHP1, NT5C2, TOR1AIP2, BMF, |
| | | | | | | NBPF1, MAST2, ECE1, RP11-385F7.1, NFE2L1, RP11-427H3.3, |
| | | | | | | PEX26, FBLIM1, RNF213, SEMA3B, PTK2B, GSDMB, ACTN4, |
| | | | | | | FAM83G, C1orf116, SLC39A14, GRAMD4, EHBP1L1, KCNK5, |
| | | | | | | ZNFX1, MAFG, C7orf43, SPTBN1, RP11-383J24.6, KIF13B, |
| | | | | | | ARHGEF18, ARHGAP27, EIF4G3, CAPN15, LRRK1, SEMA4B, |
| | | | | | | LETM1, HEPH, CCDC64B, NR2F6, CLSTN1, IL6R, EFNA2, |
| | | | | | | SH3BP2, ARSA, TRIM14, PDE6A, PLXNB2, PSD3, FAM102A, |
| | | | | | | KLF6, DYRK2, DNM2 |
| 35 | NKs | FOSL2 | UC | JAZF1, | FOSL2, JAZF1, | FO5L2, CCDC92, ANKRD37, CHMP1B, METRNL, SYTL3, |
| | | | | PIK3R1, | PIK3R1, ITCH, | AAED1, GINS4, HIST1H4E, CDC42EP4, DDX3Y, JAZF1, |
| | | | | ITCH, | MAP3K8, REL, | ZNF700, RBBP6, DLG5, HABP4, SCT, PFKFB3, NR4A2, |
| | | | | MAP3K8, | RPS6KA4, | CYP20A1, GDAP2, CSRNP1, PNMA1, PIK3R1, HOOK3, |
| | | | | IL10RA | IL10RA | DDHD2, ITCH, HCG18, HEXDC, VPS37B, MTFR1, FAM53C, |
| | | | | | | ZNF530, XPO1, TMEM42, AC093813.1, UAP1, CASZ1, |
| | | | | | | SH2D3A, ZNF771, EVI2A, HNRNPUL1, VIM-AS1, REPS1, |
| | | | | | | PSTPIP1, SYAP1, AARSD1, RP11-640M9.1, PRR7, ZFP36, |
| | | | | | | MAP3K8, REL, DNAJC3, TP53BP1, AC093323.3, ZFP36L2, |
| | | | | | | HIPK3, ZCCHC24, TSPYL2, MTMR12, MCL1, HMGXB4, |
| | | | | | | NFKBID, HELZ2, PRNP, RPS6KA4, PARP8, NUFIP2, NR4A1, |
| | | | | | | SERTAD1, ST8SIA4, CDKN2AIP, MED23, SOCS4, PTPRE, |
| | | | | | | PTPN23, KAT6B, RHOQ, ZNF618, HECTD1, LRRC48, |
| | | | | | | KIAA1191, IL10RA, WDTC1, TIPARP, PCMTD1, CCNT1, |
| | | | | | | MORF4L2, DNAJB6, KLHL28, TANGO6, IER3, TRAPPC2P1, |
| | | | | | | HSPA1A, ZNF669, GPC6, DYNC1H1, RP11-769O8.3, APOC2, |
| | | | | | | SRSF2 |
| 36 | Follicu- | HHEX | UC | JAZF1, | HHEX, JAZF1, | HHEX, IFNGR1, LPAR5, CYB561A3, JAZF1, ARPC5, CAT, |
| | lar | | | IKBKG, | IKBKG, IRF8, | CIITA, SHISA5, PTPN6, NUBP1, CD19, SNX1, RAB4B, |
| | | | | IRF8, WAS, | CARD11, WAS, | PARVG, CNPPD1, MRPS21, SNAP23, TBCB, PPP1CA, CAPZB, |
| | | | | ITGB2 | COMMD7, | HMGA1P4, SIDT2, ARPC1B, PPP4C, ITGB2-AS1, ALOX5AP, |
| | | | | | ITGB2 | LAT2, NLRC5, SNX3, BLNK, DBNL, PSMB8, TRAPPC1, |
| | | | | | | SCNM1, RFX5, RAE1, HLA-DOA, CBX3, NUDT7, CDKN2D, |
| | | | | | | CD53, GDI2, CNN2, CTC-378H22.1, LIMD2, SYNGR2, ELP5, |
| | | | | | | BLOC1S2, IKBKG, IRF8, GCA, RMI2, RP11-117D22.2, |
| | | | | | | CARD11, WAS, CAP1, UQCR11, HGS, VPS4B, SCIMP, SUMO3, |
| | | | | | | SH3BGRL3, TBPL1, WASF2, PTPN7, APOBEC3G, SPIB, |
| | | | | | | CARD16, PKIG, DTX3L, NOP10, FDFT1, TWF2, COMMD7, |
| | | | | | | PPP2R1A, CD72, ARPC2, YWHAB, GRAP, ATP6V1F, FLOT2, |
| | | | | | | STX7, LYRM4, SUMO1, HAUS1, PLEKHF2, CD81, ITGB2, DBI, PUS1, PSMB9, FCRLA, LGALS9, STX10, CASP1, |
| | | | | | | PLSCR1, ALKBH4, PCSK7, RGS19 |
| 37 | Cycling | ICOS | Healthy | ICOS, CD28, | ICOS, CD28, | ICOS, BIRC3, CD82, CD4, GPR183, CD28, SPOCK2, NFKBIA, |
| | T | | | CCL20, CD6, | CD5, CCL20, | CD44, ANTXR2, LTB, CRY1, FTH1, RP11-354P11.3, ZC3H12D, |
| | | | | NFKB2, | CD6, NFKB2, | CD5, SLC31A2, FYB, NR3C1, PBXIP1, CCL20, TGIF2, APOE, |
| | | | | NFKB1 | CYTH1, | PHLDA1, SOCS3, IRF2BP2, BCAS2, TNFRSF25, TOB2, ZNF841, |
| | | | | | NFKB1 | TMEM173, NFE2L2, GNG7, C14orf64, P2RY10, MYO5A, |
| | | | | | | INPP4B, IGLC3, TBC1D19, ELK3, ARNTL, SERPINF1, |
| | | | | | | AL928768.3, IGKV3-15, RNF145, FBLN7, MS4A6A, CD6, P2RY8, |
| | | | | | | ZXDC, PAG1, RORA, ALG13, LRRC8C, PPP1CB, PLK3, |
| | | | | | | ARHGAP10, BAG3, BTG1, ITGB2-AS1, IGLV2-11, IGHV1-18, |
| | | | | | | IGHA1, SF1, ADAMDEC1, S100A4, SNHG15, HPSE, PRKCDBP, |
| | | | | | | ARHGAP5, CNNM2, CD83, RP11-138A9.1, IGHV4OR15-8, |
| | | | | | | NFKB2, IGLC2, EIF3E, CYTH1, SLAMF1, ICAM2, C1QA, |
| | | | | | | FAM115C, IGKC, NFKB1, SPG20, IL23A, SELK, HBP1, IGHA2, |
| | | | | | | CNST, C1orf132, THEM4, MICAL2, TTC39B, LUM, CREBL2, |
| | | | | | | AXIN2, CTC-428H11.2, IGHM, IL8 |
| 38 | CD8+ | IL10RA | UC | IL10RA, | IL10RA, | IL10RA, KBTBD2, AC097500.2, PHLDB3, HS1BP3, SUN1, |
| | IL17+ | | | TAGAP, | TAGAP, | NUP188, TAGAP, PRKAB2, NAF1, TNFAIP3, MCL1, SRD5A1, |
| | | | | TNFAIP3, | TNFAIP3, | DTD2, ZNF230, IGKV3D-20, IGLV3-9, ZSCAN5A, MAP4K2, |
| | | | | CASP8, | FOSL2, | PTP4A1, LIN54, AREL1, ISG20L2, SERAC1, TMEM30B, |
| | | | | BANK1 | REL, CASP8, | TCP11L2, ZNF30, UBXN7-AS1, ZBTB1, FAM60A, TPT1-AS1, |
| | | | | | DAGLB, | ZFAND4, P2RY10, FOSL2, MX2, CYTH2, BRAF, ALDH5A1, |
| | | | | | BANK1 | REL, C19orf68, ZNF432, CLCC1, DPYD, STRN, DLGAP4, |
| | | | | | | KDM2A, RP11-212P7.2, DDIT3, CROCC, CASP8, DDX26B, |
| | | | | | | KIAA0226, IVNS1ABP, UFSP2, CTD-3184A7.4, FRAT1, FSCN1, |
| | | | | | | ZDBF2, DAGLB, DCBLD1, FAM46C, CLEC16A, FBXL18, |
| | | | | | | BANK1, MORC2-AS1, KDM6B, RGS1, SDE2, CA5B, OSM, |
| | | | | | | GPATCH2, LHPP, SLC39A6, SLC16A1, KIAA1715, FAM204A, |
| | | | | | | EID2B, EDEM1, ZNF33B, PPP1R15A, CSRNP1, AP3M2, |
| | | | | | | GLTSCR1, PSIP1, PRR12, VPRBP, RP5-935K16.1, CECR1, |
| | | | | | | FAM73B, CCDC125, MORF4L2, ZNF790, ARHGAP26, HOOK3, |
| | | | | | | RUNDC1, HERC1, TSPYL4, SBF1, SV2A, BAG4 |
| 39 | Tregs | IL18R1 | UC | NCF4, | IL18R1, NCF4, | IL18R1, MIR4435-1HG, ZC3H12A, GADD45A, TNIP3, RP11- |
| | | | | FOXP3, | NFKBIZ, | 353B9.1, LINC00884, LRRC32, NCF4, NFKBIZ, TNFRSF1B, |
| | | | | TNFRSF13B, | FOXP3, | OTUD5, AKIP1, OAS1, PTGIR, NPPC, POLR3F, PCBP3, GNG8, |
| | | | | CTLA4 | THAP4, | ADTRP, FOXP3, GK, THAP4, SLAMF1, AC074289.1, PIM2, |
| | | | | | TNFRSF13B, | IDH1, BCAS1, MEIG1, SRGAP1, CSF1, STAM, CRY1, ETV7, |
| | | | | | COMMD7, | RENBP, UGP2, TIFA, LRG1, ANKRD10, ABCC4, PHACTR1, |
| | | | | | CTLA4 | MGRN1, SAT1, ITGB1, FUCA2, RNF32, TNFRSF13B, C2CD4A, |
| | | | | | | GBP2, LIPH, EPSTI1, COX10, GRAMD4, TRMT10B, GSTM4, |
| | | | | | | ARNTL, RP11-803D5.4, ADAT2, ABHD13, COMMD7, AKIRIN2, |
| | | | | | | BRE, FAM149A, SLC35F2, ST6GALNAC6, FCHO2, SERPINE2, |
| | | | | | | CLEC7A, BAK1, IKZF4, SDHA, BCL10, RTP4, FLT1, C8orf82, |
| | | | | | | SNAPC3, PET100, RP11-214O1.3, SNX9, DHRSX, PCYOX1L, |
| | | | | | | FUT7, ARHGEF12, SLC22A18, RP11-483I13.5, CHST11, XPO5, |
| | | | | | | PNPT1, SIX5, FAM110C, MIAT, CTLA4, IL1R1, CREB3L3, |
| | | | | | | ANKRD27, RRAGB, IRAK2, CASP7, TPCN2, FANK1 |
| 40 | ILCs | LCK | UC | LCK, IL2RG, | LCK, IL2RG, | LCK, CD7, CD2, IL2RG, GIMAP7, DOK2, GIMAP5, GZMM, |
| | | | | ZAP70, | CD5, ZAP70, | CD3E, GALM, PRKCH, RHNO1, CD3D, CD5, ZAP70, TRAC, |
| | | | | ADA, | ADA, CD6, | ADA, FAS, FYN, C9orf142, SIRPG, GIMAP4, C19orf12, SEPT1, |
| | | | | CD6, CD23 | TRAF3IP3, | IL2RB, CTSC, IL12RB1, GPR68, SIT1, EVL, HNRNPLL, |
| | | | | | CD23 | SPOCK2, SH2D2A, USB1, HMOX2, CD247, CD6, RGL4, GBP2, |
| | | | | | | ECHDC2, ARNTL, SLAMF1, CASP1, TBC1D10C, RNF167, |
| | | | | | | TRAF1, GSS, CASP4, STOM, SLC9A3R1, EPS3L2, SURF4, |
| | | | | | | PHF19, SH2D1A, CMTM3, LAG3, LPAR2, OCIAD2, DTNB, |
| | | | | | | DENND2D, TSPAN5, BUB3, C9orf78, CDC42SE2, IDH2, |
| | | | | | | CFLAR, TPGS1, SLA, DLGAP1-AS1, IL32, GIMAP6, ISG15, |
| | | | | | | RAB27A, TNFRSF25, HENMT1, PTPLAD2, SIGIRRCISD3, |
| | | | | | | RAP1A, TRAF3IP3, NMRK1, SMCO4, RHOC, TNFRSF1B, |
| | | | | | | ZNF655, YIPF1, PMM1, DDB2, CD28, PCED1B-AS1, CCR5, |
| | | | | | | SQRDL, GIMAP2, URM1, MPRIP, CXCR6, ABCG1, ARL3, |
| | | | | | | CLEC2D, INPP5K |
| 41 | M cells | NFKBIZ | UC | SLAIN2, | NFKBIZ, | NFKBIZ, HLA-F, FAM91A1, TOP1, AP1G1, KIF3B, SHROOM3, |
| | | | | ERAP1, | ITGAV, | ITGAV, RAB22A, DYNC1LI2, CRK, STAT3, ATP11B, ARPC4, |
| | | | | PTGER4, | SLAIN2, | DNAJC3, SLAIN2, ERAP1, ENTPD4, MON1B, HNF4G, STK3, |
| | | | | TGFBR2, | ERAP1, | PTPN12, SGMS2, BCL3, AP3D1, MGAT2, MESDC1, KRAS, |
| | | | | ERAP2, | PTGER4, | STRN3, PITPNA, LPGAT1, VCL, ZCCHC6, GATAD2A, |
| | | | | CCL20 | TGFBR2, | CNEP1R1, STAT1, ETV3, TRIP12, CAPZA1, RNFT1, CMTM6, |
| | | | | | ERAP2, CCL20 | CLCN3, ZC3H12C, RSPH3, EFR3A, AZI2, NAMPT, NIPAL2, |
| | | | | | | ACTR2, COPG1, USP38, PARP8, UBE2K, JDP2, PCYT1A, |
| | | | | | | DAB2IP, EPT1, YWHAZ, FEZ2, RAB6A, CMIP, USP12, CRY1, |
| | | | | | | LYN, PAK2, KIF1C, SLC39A9, ZFAND5, TNFAIP1, PARM1, |
| | | | | | | IQGAP1, LGALS8, RFFL, VPS4B, PTBP3, FAM120AOS, ATP2C1, |
| | | | | | | DCUN1D1, PTGER4, CHUK, GLTP, RTN4, TMED7, TGFBR2, |
| | | | | | | ERAP2, MAGT1, MAPK1, UBR1, TINAG, CCL20, TMEM33, |
| | | | | | | ATP2A2, STAM2, STON2, RAB5B, TMEM102, C10orf118, CUL3, |
| | | | | | | DOCK9, PRDM10 |
| 42 | CD8+ | NOTCH2 | UC | CCL20, | NOTCH2, | NOTCH2, GAB2, RP3-325F22.5, MAF, CCL20, TRPS1, TBXAS1, |
| | IL17+ | | | ARIH2, | CCL20, | BCL2L11, STXBP4, MAST4, KIAA0319L, IL26, ZBTB17, |
| | | | | ZNF831, | TSPAN14, | ADAM12, CMTM6, SLA, PCBD2, VCPIP1, NTRK2, CHRM3-AS2, |
| | | | | ATG16L1, | ARIH2, | C2orf43, FKRP, VMAC, IP6K1, COL5A3, TSPAN14, ATP2B4, |
| | | | | TAB2, | ZNF831, | TMEM167A, RNF213, CTSH, ATF7IP2, MAP3K5, ARIH2, MAST4- |
| | | | | PRDM1 | ATG16L1, | AS1, BRD9, ADAM19, ZNF831, ITPRIPL1, CYB5D1, RFX7, |
| | | | | | TAB2, PRDM1 | APOL3, MAN1A1, MIAT, HECTD4, KLHDC2, MYPOP, GDE1, |
| | | | | | | GFI1, PRKAR2A, RUNX1, CENPB, PAXBP1-AS1, GPR27, POR, |
| | | | | | | HIVEP3, ARNTL, RP1-67K17.4, TBC1D31, TGOLN2, B3GNT3, |
| | | | | | | GPRIN3, ATG16L1, MDM2, SLC7A6, LRRC37B, MAP3K4, |
| | | | | | | KCTD6, DCP2, EML3, FAM105B, FBXL4, RP11-98I9.4, ATP2C1, |
| | | | | | | L12RB1, TAB2, PRDM1, NPHP3, MCCC1, ARF6, SLC4A10, |
| | | | | | | GPRASP1, JAK3, RP3-428L16.2, MYNN, PLEKHG3, INVS, RP4- |
| | | | | | | 569M23.4, POMT1, MANEA-AS1, CELF2, VPS8, NOD1, REEP2, |
| | | | | | | BIVM, WDR6, SLC44A2, B4GALT1, SMG7, LIMA1, MSL3 |
| 43 | Best4+ | PRKD2 | Healthy | HNF4A, | PRKD2, | PRKD2, DHRS3, EPS8L2, SH3BP2, GSDMD, ST14, MAP3K11, |
| | Entero- | | | C1orf106, | HNF4A, | TMEM184A, APLP2, PKP3, GBA, PRSS3, PINK1, H2AFJ, JUP, |
| | cytes | | | HPS1, | C1orf106, | PARP4, MKNK2, FRMD8, ZFAND2B, SLC37A1, ATG9A, |
| | | | | TMBIM1 | PTK2B, | HEXIM1, POR, KIF13B, HNF4A, C1orf106, PLXNA2, TLE3, |
| | | | | | TOM1, FOSL2, | CTSD, ZFAND3, LINC00035, BLOC1S1, C17orf62, ZER1, EPS8L3, |
| | | | | | HPS1, TMBIM1 | LRP10, PLEC, JUND, FURIN, FOXO4, POLD4, SUN2, DNM2, |
| | | | | | | PRSS36, CAMK2N1, KIAA2013, TNIP1, LRRC8A, INF2, CARD10, |
| | | | | | | ERBB3, SLC45A4, CLIP2, AGPAT2, ACTN4, VILL, ATG2A, |
| | | | | | | SH3BGRL3, UPP1, P2RX4, CTDSP2, PTK2B, GUCD1, BCL2L1, |
| | | | | | | PTPRH, MEF2D, SIRT7, MYH14, FBLIM1, CHMP1A, ELMSAN1, |
| | | | | | | CLTB, TOM1, HNF1A, CDKN1A, EZR, NDRG1, ELF4, TMPRSS2, |
| | | | | | | CORO1B, EHD1, CSNK1D, MOV10, TMEM127, ARHGAP35, |
| | | | | | | STAT6, SCNN1A, FOSL2, MARVELD3, VPS16, MIR22HG, |
| | | | | | | VPS37B, NR3C2, GMIP, EPHA2, HPS1, PARP12, TMBIM1, |
| | | | | | | ANXA11, RHOC |
| 44 | Entero- | PRKD2 | UC | SMAD3, | PRKD2, | PRKD2, IL4R, PARP4, SMAD3, SPTAN1, CEBPG, PTK2B, GCNT3, |
| | cytes | | | IL10RB, | SMAD3, | SLC35D2, SNX33, NT5C2, NR1I2, PTPRF, CEACAM1, TOLLIP, |
| | | | | TMBIM1, | PTK2B, | VASP, SNX9, MGLL, RHPN2, IL10RB, MAP1LC3B, RP11- |
| | | | | STXBP2, | IL10RB, | 356M20.3, TTC22, ARL14, JOSD1, CDKN1A, HS6ST1, CEACAM5, |
| | | | | KSR1 | TNFRSF1A, | C17orf62, GTPBP2, DNAJC5, ANXA11, PLEC, METRNL, LLGL2, |
| | | | | | TMBIM1, | HKDC1, TNFRSF1A, P2RY2, ACP2, KIAA1522, MICA, FBLIM1, |
| | | | | | STXBP2, KSR1 | SETD5-AS1, DHDDS, RXRA, FA2H, LRRC8A, MTMR3, SIRT7, |
| | | | | | | PPP1R13B, ACSL5, ITPKC, SLC44A4, MUC13, RALY, TMPRSS2, |
| | | | | | | TMBIM1, STXBP2, ARRDC2, RIPK3, CASP10, CLIC5, PPP1R14D, |
| | | | | | | GTPBP1, DENND3, ARHGEF18, HLA-E, DGKA, ACSS2, VWA5A, |
| | | | | | | NRBP1, ZNF394, PHYKPL, EPS8L3, ZFAND2A, PLAC8, RHOG, |
| | | | | | | CARHSP1, MYD88, EZR, SMPD1, PLEKHA7, CDC42BPG, IRF7, |
| | | | | | | RARA, KSR1, GBP2, TMPRSS4, ZMYND8, SLCO2A1, CAPN5, |
| | | | | | | CPAMD8, RIPK1, SMIM5, AKAP13, TMC4, ARHGAP27, MYO1D, |
| | | | | | | RASA4, LHFPL2 |
| 45 | Imma- | PTK2B | Healthy | C1orf106, | PTK2B, | PTK2B, C1orf106, PTPRH, JUP, SEMA3B, ATG2A, COL17A1, |
| | ture | | | TMBIM1, | C1orf106, | SLC25A23, EPS8L2, PSD4, LAMB3, PLXNA2, RETSAT, CTDSP2, |
| | Entero- | | | GPR35, | PRKD2, | ERBB3, SIPA1L3, VILL, EZR, MAPK7, CLCN2, INF2, DOK4, |
| | cytes | | | HPS1, | TMBIM1, | EHD1, PLEKHG6, TJP3, DNM2, LINC00035, SCNN1A, EHD4, |
| | | | | SMAD3, | GPR35, HPS1, | SLC6A8, TMEM2, CDHR5, ATG9A, PLEC, CNNM4, PYGB, |
| | | | | TTC7A | SMAD3, | SLC25A25, CLSTN1, SIRT7, EPHA2, AKAP13, NEDD4L, GPA33, |
| | | | | | TTC7A | KIAA0247, STAG1, KCNK6, JUND, PRKD2, TMBIM1, NBPF1, |
| | | | | | | LRP10, TBC1D1, GPR35, PKP3, CHMP1A, PARP4, HPS1, DHRS3, |
| | | | | | | RAB40C, CGN, C17orf62, NUB1, VAV2, HEXIM1, LRRC8A, |
| | | | | | | ZFYVE27, P2RX4, ECE1, TMEM184A, ALDHI8A1, TRIM15, |
| | | | | | | PNPLA2, ARHGEF18, RP13-15E13.1, FBLIM1, RALGDS, |
| | | | | | | PLXNA3, IST1, CTSD, STX3, ARHGAP17, RIOK3, UPP1, SLC2A1, |
| | | | | | | FAM102A, KIAA0195, MAP3K11, MIR22HG, AMACR, SMAD3, |
| | | | | | | SLC20A2, PTTG1IP, LASP1, OPTN, WIPF2, CHPF2, TTC7A, |
| | | | | | | SGK223, MEP1A, PINK1 |
| 46 | Entero- | PTK2B | UC | SMAD3, | PTK2B, | PTK2B, CNNM4, CDKN1A, CEACAM5, ACSS2, CDC42BPG, |
| | cytes | | | IL10RB, | SMAD3, | PTPRF, SMAD3, MYH14, ARHGAP17, MTMR3, CEACAM1, |
| | | | | C1orf106, | IL10RB, | NT5C2, DGCR2, RARA, TMPRSS2, ARHGEF18, CLSTN1, |
| | | | | IL2RG, | PRKD2, | IFNLR1, ZMYND8, RXRA, JOSD1, IL10RB, WWP2, PRKD2, |
| | | | | TMBIM1 | TNFRSF1A, | RP11-395P17.3, ZZEF1, LHFPL2, SPAG9, TMC4, PTTG1IP, |
| | | | | | C1orf106, | SLC16A3, IRF7, MUC13, ITM2C, TNFRSF1A, HIST1H2AC, |
| | | | | | IL2RG, | GCNT3, SLC6A8, COL17A1, LITAF, CAPN5, TMEM8A, |
| | | | | | TMBIM1 | CEACAM7, TRANK1, TNFSF10, SLCO2A1, TTC22, GDPD2, |
| | | | | | | GNA11, SMIM22, GPRC5A, ABTB2, SNX33, PRR15L, |
| | | | | | | RAP1GAP2, TMEM220, DUSP5, PARP12, C1orf106, |
| | | | | | | ARHGAP27, MBNL1-AS1, IL2RG, MS4A12, |
| | | | | | | EHD1, CLIC5, LRRK1, KLF6, BMP1, APLP2, HKDC1, AOC1, |
| | | | | | | GPA33, ZFYVE1, SRSF5, IL4R, PTK6, ZFAND2A, TMBIM1, |
| | | | | | | FUCA1, MTMR11, SGK223, RAB9A, MICA, METRNL, PLAC8, |
| | | | | | | FMO4, INF2, CHMP1B, ABHD3, RELL1, TUBAL3, PTPRH, |
| | | | | | | NEAT1, RFK, C1orf115, ZFP36, ITPKC, B3GNT3, KIAA0247 |
| 47 | Entero- | PTK2B | Healthy | C1orf106, | PTK2B, | PTK2B, CLSTN1, SPECC1L, VPS37B, GBA, DNM2, MICA, SUN2, |
| | endo- | | | HNF4A, | C1orf106, | METRNL, SLC25A23, FAM83G, ACTN4, SH3BP2, SLC39A14, |
| | crine | | | GSDMB, | HNF4A, | ITSN1, SGK223, DHRS3, INF2, CLIP2, RETSAT, FRMD1, KIF16B, |
| | | | | MST1R, | GSDMB, | GTPBP1, LMTK2, NPAS2, PLXNA2, GNA11, TMEM63B, |
| | | | | SMAD3, | FOSL2, | C1orf106, HNF4A, NDRG1, PCDH1, GSDMB, CNNM4, FRMD8, |
| | | | | HPS1 | MST1R, | FOSL2, JOSD1, CCNYL1, LRP10, RIPK1, ARHGAP27, WBP1L, |
| | | | | | SMAD3, HPS1 | EHD1, N4BP1, FOXO4, RXRA, PLXNB2, MAFK, PARP4, MST1R, |
| | | | | | | DYRK2, MKNK2, CTNND1, ARHGAP17, FAM211A, AMN, JUND, |
| | | | | | | STAT6, IL17RA, SMAD3, DENND1A, STK24, EPHA2, NT5C2, |
| | | | | | | ZDHHC18, TMEM8A, ZFAND2B, PRSS36, GRAMD4, SPTBN1, |
| | | | | | | CDH1, SEMA4B, ST14, MIDN, DNAJC5, BCL2L11, KIF13B, |
| | | | | | | ARHGAP35, ASPG, SPTAN1, ARHGEF16, HPS1, MAST2, AMFR, |
| | | | | | | WWP2, ZNFX1, CHPF2, TRIM14, MON1B, TRAK1, JUP, DUSP3, |
| | | | | | | ACVRL1, ZBTB7B, KIAA2013, APLP2, NFE2L1, SLC26A6, |
| | | | | | | CSNK1D, KLF6 |
| 48 | CD+ | PTPN2 | UC | ARIH2, | PTPN2, ARIH2, | PTPN2, ATF6B, SMCO4, RNF145, OTUD5, ASCC2, ARID5A, |
| | Acti- | | | TAB2, | TAB2, CD5, | DENR, PPP1CC, POMZP3, ARIH2, TAB2, TOMM34, VOPP1, CD5, |
| | vated | | | UBASH3A, | UBASH3A, | ABTB1, EEPD1, STARD3, PPHLN1, TDP1, SPPL3, FIG4, ADCK4, |
| | Fos-hi | | | ZAP70 | TRAF3IP3, | SMARCAL1, BTBD10, ARL5A, RP3-340N1.5, CCNI2, PBXIP1, |
| | | | | | SUFU, ZAP70 | CSTF2T, TRIB2, KIAA1324, RMND5B, AP1B1, ZNF786, TSPAN5, |
| | | | | | | SLC44A2, MRPL42, CREBL2, RILPL2, TMEM194B, VASH2, |
| | | | | | | UBASH3A, GOLPH3, PIK3IP1, SPOCK2, TRAF3IP3, RAP1A, |
| | | | | | | SEC14L1, SUFU, FBXW11, MAP2K7, NFE2L1, TRAF7, C21orf33, |
| | | | | | | ZFP57, MT1X, STAM, TRMT2B, GBP7, OXLD1, TAF11, POMT1, |
| | | | | | | TFE3, RAD1, FCER2, HMCES, C19orf38, B3GAT3, SRRD, IFI16, |
| | | | | | | PSMD5, SPSB1, WIPI2, MUS81, CPSF7, GLCCI1, USP48, |
| | | | | | | METTL3, HBP1, PWP2, SMAP2, RABGAP1L, ZAP70, SRP68, |
| | | | | | | JAK3, PIM2, SIRT7, TNFRSF25, CARHSP1, FKRP, SYT11, |
| | | | | | | ATP2A2, CLEC2D, SUGP1, CD59, ZNRF1, TACO1, DAZAP1, |
| | | | | | | KLHL2 |
| 49 | Cycling | REL | Healthy | PTPRC, | REL, PTPRC, | REL, SYAP1, GPR183, RNF139, CREB1, YPEL5, BAZ1A, STK38, |
| | Mono- | | | PTGER4, | PTGER4, | RBPJ, AKAP9, HCG18, GK, DOCK8, INSIG1, NFE2L2, LTA4H, |
| | cytes | | | RIPK2, | RIPK2, | KBTBD2, PHACTR1, GTF2B, PCBP1, HS3ST3B1, TGIF1, GTF2A1, |
| | | | | IL2RG, | IL2RG, | PTPRC, CSRNP1, SFPQ, CMTM6, HOTAIRM1, ARL5B, STK4, |
| | | | | PRKCB, | PRKCB, | GZF1, HNRNPLL, STX11, CD83, MCL1, ZNF562, IL1R1, CCNH, |
| | | | | NFKB2, | NFKB2, WAS | SPN, CDC42SE2, PTGER4, RIPK2, RILPL2, DR1, PIM1, MAP2K1, |
| | | | | WAS | | ZNF672, CREB3L4, ZNF207, EIF4A3, CCDC88A, MCCC1, |
| | | | | | | FAM110A, SGK1, ASCC2, IL2RG, DDX18, C10orf118, KDM6B, |
| | | | | | | RNF10, IFNGR1, NUMB, RNF166, PRKCB, GRSF1, MNDA, |
| | | | | | | MEMO1, NFKB2, AKIRIN1, TXLNG, MAP2K3, ATXN7, SPOP, |
| | | | | | | DDX21, PLSCR1, WSB1, TPPP3, SCAF11, BCLAF1, SNHG5, |
| | | | | | | SIAH2, FAM69A, SPOPL, MAN1A1, MAPK1IP1L, CD48, |
| | | | | | | ZFAND5, GOLPH3, CDKN1B, PPP6C, TRIM26, WAS, SRSF3, |
| | | | | | | SNX10, GRWD1, CAMK1D, ZNF385A, TFAM, AVPI1, SPTY2D1 |
| 50 | Imma- | SMAD3 | Healthy | SMAD3, | SMAD3, | SMAD3, RIOK3, KIAA1217, RELB, AQP7, MPP5, SNX9, TMEM2, |
| | ture | | | C1orf106, | C1orf106, | KIAA0247, RHOU, CDH1, PARP12, C15orf39, JOSD1, KIAA2013, |
| | Entero- | | | EFNA1, | FOSL2, | RAB11FIP1, LPIN2, C1orf106, STK24, CTDSP2, TMCC3, |
| | cytes 1 | | | HPS1, | PTK2B, EFNA1, | LINC00704, LRP10, EDN1, SLC25A23, STK17B, PDLIM5, |
| | | | | TMBIM1 | IFNGR2, HPS1, | C1orf115, JUP, RP11-680F8.1, VPS37B, MARVELD3, RMND5A, |
| | | | | | TMBIM1 | BDKRB2, TRANK1, ZC3H12A, F11R, MYO1E, SUN2, TMEM236, |
| | | | | | | ACVRL1, FOSL2, SORL1, CDKN1A, SLC20A2, CNKSR3, DHRS3, |
| | | | | | | UPP1, TAPBP, PTK2B, EPS8, EFNA1, PNPLA2, GLRA4, LMO7, |
| | | | | | | TLDC1, TRAFD1, PCDH1, RP11-465N4.4, IFNGR2, PLAUR, |
| | | | | | | CLSTN1, CLDN23, COL17A1, HMOX1, PLIN3, RP11-134L10.1, |
| | | | | | | SCNN1B, LSR, PTPRH, BCL2L11, HPS1, TICAM1, DTX3L, |
| | | | | | | TMBIM1, ARL14, HS6ST1, TNFRSF21, POLD4, NBR1, RHOF, |
| | | | | | | PAG1, GPA33, LASP1, INF2, CCDC68, PEX26, TMC5, PDCD6IP, |
| | | | | | | DSC2, TNFSF10, SPINT1, LITAF, GPRC5A, SMPD1, ASS1, TJP1, |
| | | | | | | AVL9, FLVCR1-AS1, ABTB2 |
| 51 | Imma- | SP140L | UC | SMAD3, | SP140L, | SP140L, APOL2, PVRL2, GSN, LAMC2, C19orf66, B4GALT1, |
| | ture | | | CASP8, | SMAD3, | IL15, MUC13, RHPN2, MOV10, VEGFA, OGFR, PLEC, |
| | Entero- | | | TNFAIP3, | TNFRSF1A, | RN7SL368P, TNFRSF1B, TNFSF10, TYMP, SLCO4A1, APOL1, |
| | cytes 2 | | | KSR1, | CASP8, | HLA-E, RIPK3, TCIRG1, CARD10, IRF9, RALGDS, SMAD3, IRF7, |
| | | | | PRDM1, | TNFAIP3, | LRP10, NT5C2, CXCL16, JOSD1, CEACAM5, CASP10, LAMA3, |
| | | | | NFKB2 | KSR1, PRDM1, | MAPKBP1, GABRE, BIRC3, SRC, DDX58, TMPRSS2, LPIN2, |
| | | | | | NFKB2 | PARP14, ZMYND15, VAMP5, RIPK1, WWC1, LMO7, TCHP, |
| | | | | | | GTPBP1, TNFRSF1A, NEAT1, EPS8L1, FHL2, MED15, B4GALT4, |
| | | | | | | SEC14L2, DAPK2, SAP30BP, PLEKHS1, ASS1, TAP2, CLIC5, |
| | | | | | | DEDD2, CSNK1D, CASP8, RP11-356M20.3, TMEM234, ARL14, |
| | | | | | | C17orf62, TNFAIP3, RGL1, RP11-425D10.10, MYO1E, HSH2D, |
| | | | | | | TRIM15, RHBDF1, MIR210HG, MAP7D1, RP11-448G15.3, |
| | | | | | | HS6ST1, POU5F1, KIF13B, ARHGEF18, RND1, ANGPTL4, CNST, |
| | | | | | | SLC3A2, DENND3, IRAK2, KSR1, PLXNB2, EZR, EHD4, JUP, |
| | | | | | | PRDM1, PLAUR, NABP1, ZNFX1, NFKB2 |
| 52 | Imma- | TNFAIP3 | UC | TNFAIP3, | TNFAIP3, | TNFAIP3, VEGFA, SMAD3, DDX58, IFIT2, TNFRSF1A, BIRC3, |
| | ture | | | SMAD3, | SMAD3, | NT5C2, ZC3H12D, CASP10, TMPRSS2, LMO7, MXD1, |
| | Entero- | | | IL2RG, | TNFRSF1A, | CEACAM5, OGFR, TNFRSF1B, DDX60, B4GALT1, TNFRSF21, |
| | cytes 2 | | | PRDM1, | IL2RG, | ABCD1, IFNAR2, PVRL2, KIAA0247, MUC13, CEACAM6, |
| | | | | TMBIM1, | PRDM1, | CCDC68, WWC3, CEACAM7, DDX60L, RIPK3, ZNFX1, |
| | | | | IL10RB | TMBIM1, | CHMP1B, SESTD1, IL2RG, HS6ST1, JOSD1, PARP14, SAMD9, |
| | | | | | ERRFI1, | EHD1, MAP2K3, CMPK2, PRDM1, CXCL16, SORBS1, ABHD3, |
| | | | | | IL10RB | F11R, RFK, CDKN1A, LRP10, RGL1, IL15, PFKP, PELI2, GSN, |
| | | | | | | RHBDF1, ASS1, TOR1AIP2, TMBIM1, ADM, NFKBIA, FLCN, |
| | | | | | | LPIN2, HLA-E, HUS1, LITAF, LAMC2, ERRFI1, APOL2, |
| | | | | | | PLEKHG5, LMOD3, PLEC, FHL2, HHLA2, MOV10, CASP7, |
| | | | | | | CYP3A5, C19orf66, KCNK1, MCL1, EHD4, BCL2L1, GCNT3, |
| | | | | | | SRC, B3GNT3, RALGPS2, FOXO3, IL10RB, GTPBP2, FHDC1, |
| | | | | | | GPRC5A, RP11-356M20.3, SLC16A3, SLC45A4, STK24, TLR3, |
| | | | | | | C6orf222, LRRFIP1, CYTH2, XRN1, SCNN1A |
| 53 | Best4+ | TNFRSF1A | UC | C1orf106, | TNFRSF1A, | TNFR5F1A, LRP10, HIST1H2BD, TTC22, OPTN, SPATS2L, |
| | Entero- | | | TMBIM1, | C1orf106, | JOSD1, C1orf106, C1orf115, SLC16A3, B4GALT1, KIAA0247, |
| | cytes | | | GPR35, | IFNGR2, | FAM102A, SNX9, TNIP1, LMO7, GPRC5A, PCDH1, ABTB2, |
| | | | | TTC7A | TOM1, | EHD1, MAX, CCDC68, VPS37B, STX3, CTDSP2, IFNGR2, |
| | | | | | TMBIM1, | MUC13, GINM1, RIPK3, SERINC2, LHFPL2, LPIN2, PEX26, |
| | | | | | PTK2B, GPR35, | SLC20A2, FAM83G, IFNLR1, PPAP2A, ARHGEF18, ABHD3, |
| | | | | | TTC7A | TAX1BP3, GABARAPL1, CTSA, MAP1LC3B, DOK4, DHRS3, |
| | | | | | | SLC9A3R1, GPA33, TOM1, PRSS8, MXI1, RHOG, APPL2, |
| | | | | | | TMPRSS2, RFK, NT5C2, PFKP, TMBIM1, LRRC1, CEACAM5, |
| | | | | | | ZC3H12D, MEF2D, C17orf62, GDA, EPS8L3, CLIP2, PARP4, |
| | | | | | | IL15, SMPD1, EPS8L2, PTTG1IP, RAB9A, EZR, PARP12, MEP1A, |
| | | | | | | LINC00035, TP53INP2, PTK2B, LAMA1, GPR35, SFXN1, |
| | | | | | | PDLIM2, LAMC2, CEACAM6, LRCH4, ARHGAP17, MISP, |
| | | | | | | ANK3, MOV10, TTC7A, HPGD, SLC6A8, TNFSF10, CARD10, |
| | | | | | | CA13, CDKN1A, IL6R, HLA-A, MXD1, GTPBP2, SPINT1 |
| 54 | Secre- | ERGIC1 | Healthy | ERGIC1, | ERGIC1, | ERGIC1, TRPT1, ZG16B, DOPEY2, FAM3D, QSOX1, TCEA3, |
| | tory TA | | | MMEL1, | CYTH1, | SLC50A1, CCDC125, CYTH1, MMEL1, CANT1, SLC39A11, |
| | | | | SLC39A11, | MMEL1, | URAD, SLC22A23, STARD10, RP11-545E17.3, SH3BGRL3, CD63, |
| | | | | SLC22A23 | SLC39A11, | SH3PXD2A, MCF2L, CST3, FKBP2, RP11-775D22.2, KAZALD1, |
| | | | | | SLC22A23, | RBBP8NL, B4GALT4, MLPH, ERN2, TAGLN, SGSM3, GOLGA2, |
| | | | | | THAP4, PRKD2 | CCL15, FAM53B, TPGS1, C2orf82, NUDT16, GALNT5, DNAJB2, |
| | | | | | | RABAC1, RPL36AL, TMEM191A, TSTD1, CDC42EP5, PNPLA7, |
| | | | | | | HES2, PIK3C2B, ZBTB7C, FAM114A1, FFAR4, OST4, SLC39A7, |
| | | | | | | CAMTA2, FERMT3, OAF, KDELR2, MADD, TTC39A, SLC17A5, |
| | | | | | | EPS8L1, BAIAP2L2, RRBP1, MXD4, CREB3L1, KCNK6, |
| | | | | | | KANSL1-AS1, SSR4, TMEM181, ATP13A2, REG4, MBD6, |
| | | | | | | CCDC60, FAM189A1, PPP1R9B, CTD-2196E14.5, GNB1, ERCC5, |
| | | | | | | MUC2, THAP4, MAP3K14, KIAA0319L, MARVELD1, UBXN6, |
| | | | | | | PRKD2, ESRP2, RASSF7, HIP1R, HLA-E, KCTD11, TBC1D2, |
| | | | | | | NOXO1, RP11-386I14.4, DAGLA, ADAP1, PPIC, SLC1A5, |
| | | | | | | UNC13B, EFCAB4A, JHDM1D-AS1, CAPN9 |
| 55 | Entero- | IFIH1 | UC | IFIH1, | IFIH1, SLAIN2, | IFIH1, SCYL2, TRMT1L, FAM91A1, SPTLC2, ANKIB1, TINAG, |
| | cyte | | | SLAIN2, | AHR, ERAP1, | HDGF, DCUN1D1, CNOT2, KCMF1, IDE, SENP6, PRDM10, |
| | Pro- | | | AHR, | NFKB1, | SOS1, C11orf35, C5orf24, RAB3IP, MTUS1, EID2, UBE2H, LIN7C, |
| | genitors | | | ERAP1, | FERMT1, | GRHL2, PPP4R1, TES, AHCYL1, NUP214, CDC42, SLAIN2, |
| | | | | NFKB1, | CLTC | GM2A, CCNY, CCDC24, KIAA1033, ENPP4, RBM43, SPAST, |
| | | | | FERMT1 | | ARPC4, OSBP, ACBD3, MKLN1, YES1, MIER1, PPP1R12A, |
| | | | | | | IMPAD1, AHR, SOD2, TSPYL1, ARFGEF2, IQGAP1, HMGCR, |
| | | | | | | ORC3, ELOVL6, SEPT11, SUV420H1, TRMT10A, OSBPL8, |
| | | | | | | UBE2M, UBE2K, NET1, ATP6V1A, ADAM10, RAB5B, ATF6, |
| | | | | | | WDR45B, DNAJC3, ITGA6, UGT8, ZC3H13, RAB21, FBXL17, |
| | | | | | | USP9X, RYBP, AP1G1, ERAP1, ADNP2, NOXO1, TRIM2, RAM1, |
| | | | | | | PCDH20, NIPAL2, PTP4A2, ACTR2, NFKB1, NCKAP1, OPA1, |
| | | | | | | TMOD3, SULF2, RAP2A, AGFG1, PAK2, MTPN, UBXN4, |
| | | | | | | ASCC3, DENR, UBR1, FERMT1, CLTC, YBX3, CTBS, IPO8 |
| 56 | CF8+ | IL2RA | UC | IL2RA, | IL2RA, | IL2RA, ZC2HC1A, CXorf21, GK, RHNO1, RP11-316P17.2, |
| | IELs | | | SLC37A4, | SLC37A4, | STRADB, RP11-295G20.2, RASGRP4, SLC37A4, ISOC1, PIM3, |
| | | | | NDFIP1, | NDFIP1, | VANGL2, NUPL1, MAGEH1, PMAIP1, MAT2B, BCAS3, |
| | | | | SLC39A8, | SLC39A8, | C18orf25, PLEKHM2, CTSB, SLC25A40, IL1R2, PTPLA, HN1L, |
| | | | | KSR1, | KSR1, FOXP3, | SREBF1, NAB1, EBI3, NPPC, EEPD1, CD80, ITPR1, NDFIP1, |
| | | | | FOXP3, | F5 | CA11, GNG8, SLC16A1, ZNF681, RP11-455F5.5, CNIH1, |
| | | | | F5 | | PARPBP, TMEM38B, ATG5, HIVEP1, ATF7, VOPP1, ZHX1- |
| | | | | | | C8ORF76, WSB2, TOX2, DCP1B, FANCM, NFE2L3, |
| | | | | | | MIR155HG, DOHH, SLC39A8, CCNH, LZTFL1, IGFL2, TACC3, |
| | | | | | | DDX28, TTBK1, KSR1, PRKCDBP, EPFIX3, PMVK, SNHG11, |
| | | | | | | CDCA7, TBC1D15, GSTZ1, POU2AF1, DIRAS3, ZNF287, |
| | | | | | | KCNK1, FOXP3, TMEM199, AC018816.3, RDH11, MSI2, |
| | | | | | | XXYLT1, DPH3, RP5-1112D6.8, LRR1, MTMR6, CD83, RP11- |
| | | | | | | 345M22.1, CREBL2, C2orf81, ATP6V0E2, HOXB2, TNFRSF8, |
| | | | | | | SLC39A13, KLHL22, NOP14AS1, NDUFV3, CLPTM1, PKP4, |
| | | | | | | F5, DNTTIP1, SMS, CDC25B, AACS |
| 57 | TA 1 | ZBTB38 | UC | ZBTB38, | ZBTB38, | ZBTB38, CTBP2, NFIB, FERMT1, IRF2BP2, PDZD8, AUTS2, |
| | | | | FERMT1, | FERMT1, | RANBP2, PUM1, ITGA6, ZNF827, NEO1, SEZ6L2, PROSER1, |
| | | | | LRBA, | ZFP36L1, | RPS6KA3, STRBP, ADNP2, ZMYM4, ARID1A, MYCBP2, |
| | | | | EGFR, | DOCK7, LRBA, | SLC9A2, FAM171A1, AL592183.1, SECISBP2, PBX1, SSBP3, |
| | | | | NRIP1 | EGFR, NRIP1 | ARID1B, TRIM2, OS9, URI1, AKAP1, ARSD, MBP, HNRNPUL2, |
| | | | | | | FAM115A, C5orf24, VPS51, CS, PRRC2A, RBM39, GATA6, |
| | | | | | | SATB1, TM2D1, PDS5A, GS1-251I9.4, BRWD3, CDHR1, |
| | | | | | | TMEM245, PHF3, FOXK1, ZBED5, WRNIP1, AMMECR1L, |
| | | | | | | PRKAR2A, GPBP1L1, MBTD1, PURB, MFHAS1, KIAA1147, |
| | | | | | | ZFR, SUDS3, AGFG1, POGK, FAM168B, IRF2BP1, ZFP36L1, |
| | | | | | | NFIA, SMEK2, LARS, YME1L1, CACUL1, KRI1, GNS, DOCK7, |
| | | | | | | LRBA, PPP2R5C, OSBP, SOS1, AGAP1, TNFRSF11A, IWS1, |
| | | | | | | BTBD2, CERS6, ZC3H13, SVIL, KDM5C, EGFR, HP1BP3, |
| | | | | | | CREB1, CCDC6, NEK9, ZNF148, RNF169, KIAA0430, NRIP1, |
| | | | | | | SMAD4, ZBTB4, SUZ12, CAMK1D, BCL11A |
| 58 | TA 1 | TAB2 | Healthy | TAB2, | TAB2, NRIP1, | TAB2, NRIP1, ANKRD13A, SETX, USP33, ATP10B, SHROOM3, |
| | | | | NRIP1, | LPP, ITGAV, | SLC38A1, PDE8A, LPP, ITGAV, XIST, CCP110, RP11-485G4.2, |
| | | | | LPP, XIAP | TET2, XIAP | PJA2, INADL, SLMAP, OTUD4, RC3H1, FRYL, HIATL1, ZNF677, |
| | | | | | | MKLN1, HIPK2, NXPE2, RP11-349A22.5, SLC35E1, TET2, SPG11, |
| | | | | | | MGEA5, UBR2, MAPK1, LRCH3, PPARGC1A, TBL1XR1, NUFIP2, |
| | | | | | | SPPL3, LGR4, STAG2, ZFX, LDLR, ZNF785, MGAT4A, NFAT5, |
| | | | | | | TRIM33, RDH10, UBP1, ARHGEF12, CTSK, GPR155, ATXN2, |
| | | | | | | SUZ12, PKN2, FAM63B, NPIPB5, DDX17, KIAA1551, XIAP, |
| | | | | | | CREBRF, MTUS1, CHD1, PDLIM5, HNRNPH1, ZNF844, BMPR2, |
| | | | | | | SLK, USP54, MON1B, C4orf32, PDPK1, TOR1AIP2, CUL3, |
| | | | | | | CTNND1, DDX3X, MSI2, FNIP2, ATP6V0A1, RAB11FIP2, |
| | | | | | | OTUD7B, RYBP, SLC25A37, NCKAP1, TOB2, GKAP1, TNRC6B, |
| | | | | | | RP11-761144, PNISR, KPNA4, USP42, snoU13, PPTC7, |
| | | | | | | AC104532.4, ZC3H13, SYTL4, GAB1, IGHA2, ZNF292, TOB1, |
| | | | | | | ROCK2, CBWD6 |
| 59 | CD69+ | HLA- | UC | ADA, NCF2, | HLA- | HLA-DQA1, ZNF385A, RAMP1, HCK, ADA, JHDM1D-AS1, |
| | Mast | DQA1 | | NLRC4 | DQA1, ADA, | YPEL1, NCF2, CTC-425F1.4, TIFAB, AURKC, FZD2, DDHD2, |
| | | | | | NCF2, NLRC4 | COQ2, C1orf54, DHRS3, AZU1, PQLC2, TFEB, EMR3, A4GALT, |
| | | | | | | GBA2, CDH17, MORN4, ZFYVE26, CLEC4A, BCAS1, CPNE8, |
| | | | | | | NLRC4, AC079767.4, ZNF526, RASSF4, RP13-20L14.4, CDC42EP1, |
| | | | | | | MMD, HECTD3, FAM3B, HLA-DQB1, GSDMA, POP1, DHX35, |
| | | | | | | RP11-110I1.12, FUT7, RP11-73M18.8, ZNF585B, BATF3, RP11- |
| | | | | | | 334C17.5, RP11-705C15.5, DNASE1L3, RP11-252A24.3, LGR4, |
| | | | | | | TJP3, ACACA, AIM2, ITGA6, XRCC3, MRM1, APOOL, DHCR7, |
| | | | | | | HLA-DPB1, EHF, DEAF1, FAM65A, CYP27B1, CTB-138E5.1, |
| | | | | | | HLA-DRA, IPO13, CD244, ATP2C2, MCOLN2, FTCDNL1, |
| | | | | | | CLEC10A, MDFIC, CD1D, NRG1, IGHV2-5, KIAA1598, C12orf5, |
| | | | | | | CTNNAL1, LEPR, PPAPDC3, SEPT10, SDC4, RP11-65J3.1, TLR8, |
| | | | | | | PLD4, DYSF, ME1, OPN3, CEBPA, CTD-2319I12.2, IL21R |
| 60 | Tregs | RGS14 | UC | DCLRE1C, | RGS14, | RGS14, ESD, CABIN1, SCAF11, EIF3E, ESYT1, THUMPD1, |
| | | | | C11orf30, | DCLRE1C, | SFT2D2, SEPT9, CSNK1G2, ZNF518A, FAM208A, DCLRE1C, |
| | | | | CD3G | C11orf30, | PPIG, KDM5A, KRI1, THOC2, LRRFIP1, C12orf65, LYRM5, |
| | | | | | CD3G | LYRM7, NKTR, PNN, KLHL36, FYB, C6orf62, RALBP1, PNISR, |
| | | | | | | BBX, WAC, MZT2B, GTF2H5, EIF3A, RPL23, SLC36A4, |
| | | | | | | C11orf30, TESPA1, DDX46, PITPNC1, CCNI, NLRP1, ITSN2, |
| | | | | | | RASSF2, NIPBL, FNBP1, NCDN, SH3GL1, ASB8, TTF1, TRIM56, |
| | | | | | | NAP1L1, ILF3, CD3G, ACAP1, SLC38A1, RIF1, AQP3, FAM217B, |
| | | | | | | ROCK1, SPTAN1, RAPGEF6, VPS35, PRPF38B, ANKZF1, |
| | | | | | | SYMPK, ZNF75A, CTR9, CCSER2, TATDIN1, SAFB, TGS1, |
| | | | | | | FNBP4, RBMX2, MFHAS1, SEC62, ARMCX4, TAF1D, ARL17A, |
| | | | | | | MTDH, RP11-94L15.2, TTC28-AS1, ARHGEF6, WASF2, MACF1, |
| | | | | | | RP11-367G6.3, FBXW7, BCLAF1, MGEA5, EMC2, AKIRIN1, |
| | | | | | | HELZ, KIAA1033, PABPC1L, RABGGTA, PPP3R1, DPH5, |
| | | | | | | SRRM2, SMARCD2, COX7B, GON4L |

### Linking Variants (SNPs) to Function (Gene Modules) and Genes to Phenotypes (Complex Traits)

In certain embodiments, genetic variants associated with complex traits (e.g., phenotypes, heritability) are linked to gene modules. Heritability is a statistic used in genetics that estimates the degree of variation in a phenotypic trait in a population that is due to genetic variation between individuals in that population. Thus, the phenotypes or heritability can be linked to the specific expression of genes and cell types. In certain embodiments, the identified cell types and biological programs can be used for detection of subjects at risk for or having a particular phenotype (e.g., a disease, intelligence, athletic ability). In certain embodiments, the identified cell types and biological programs can be used for identifying therapeutic targets. In certain embodiments, the identified cell types and biological programs can be targeted to treat disease.

In certain embodiments, linking the variants to gene modules (gene programs) includes generating or constructing gene modules, as discussed herein. The gene modules can be enriched in a healthy cell-type, enriched specifically in the disease state of a cell type, or enriched across cell types in tissues. More than one module can be generated for a tissue. The modules can include modules for every cell type. The modules can include biological programs expressed across cells in the tissues. The gene modules can include biological programs that are spatially resolved, such as programs expressed in specific regions of cells.

In certain embodiments, linking the variants to gene modules includes generating a gene score or weight for each gene in each module. In certain embodiments, a gene score is determined by calculating the expression of each gene in a module. In certain embodiments, the gene score is determined by enrichment of gene expression in a module. In certain embodiments, the gene score for a gene in a module is highest for genes with the most enrichment in that module as compared to the gene in all other modules. Enrichment can refer to genes or proteins whose expression is over-represented in a large set of genes or proteins. In certain embodiments, the gene score for a gene in a module is determined using a significance score based on GWAS p values of all surrounding SNPs (e.g., MAGMA) (see, e.g., de Leeuw C A, Mooij J M, Heskes T, Posthuma D. MAGMA: generalized gene-set analysis of GWAS data. PLoS Comput Biol. 2015; 11(4):e1004219; and ctg.cncr.nl/software/magma). Surrounding SNPs may include SNPs within a window of 500, 200, 100 kb or less. In certain embodiments a gene score is determined by using a combination of enrichment and p values.

In certain embodiments, linking the variants to gene modules includes combining the gene score or weight with a score determined by enhancer contacts with each gene (Enhancer-to-gene (E2G) strategy). In preferred embodiments, the enhancers are matched to the tissue of interest (e.g., enhancers active in the tissue of interest). For example, brain enhancers are used to link variants to gene modules constructed using brain tissues and blood enhancers are used to link variants to gene modules constructed using blood tissues.

In certain embodiments, an Activity-by-Contact (ABC) model is used to link variants to gene modules. This model is based on the simple biochemical notion that an element's quantitative effect on a gene should depend on its strength as an enhancer (“Activity”) weighted by how often it comes into 3D contact with the promoter of the gene (“Contact”), and that the relative contribution of an element on a gene's expression should depend on the element's effect divided by the total effect of all elements (see, e.g., Fulco, et al. Activity-by-contact model of enhancer-promoter regulation from thousands of CRISPR perturbations. Nat Genet. 2019; 51(12):1664-1669. doi:10.1038/s41588-019-0538-0; and Moonen, et al., 2020, KLF4 Recruits SWI/SNF to Increase Chromatin Accessibility and Reprogram the Endothelial Enhancer Landscape under Laminar Shear Stress. bioRxiv 2020.07.10.195768, doi.org/10.1101/2020.07.10.195768).

In certain embodiments, an epigenome model is used to link variants to gene modules. Previous studies showed that disease-associated variants are enriched in specific regulatory chromatin states (Ernst, J. et al. Mapping and analysis of chromatin state dynamics in nine human cell types. Nature 473, 43-49 (2011)), evolutionarily conserved elements (Lindblad-Toh, K. et al. A high-resolution map of human evolutionary constraint using 29 mammals. Nature 478, 476-482 (2011)), histone marks (Trynka, G. et al. Chromatin marks identify critical cell types for fine mapping complex trait variants. Nature Genet. 45, 124-130 (2013)) and accessible regions (Maurano, M. T. et al. Systematic localization of common disease-associated variation in regulatory DNA. Science 337, 1190-1195 (2012)). In certain embodiments, the epigenome model used to predict enhancer-gene connections is Roadmap (see, e.g., Ernst, J., Kheradpour, P., Mikkelsen, T. et al. Mapping and analysis of chromatin state dynamics in nine human cell types. Nature 473, 43-49 (2011); Kundaje, A., Meuleman, W., Ernst, J. et al. Integrative analysis of 111 reference human epigenomes. Nature 518, 317-330 (2015); and egg2.wustl.edu/roadmap/webportal/index.html).

In certain preferred embodiments, the Enhancer-to-gene (E2G) strategy is a combined union of Activity-By-Contact and Roadmap Enhancer-to-gene (E2G) strategy (Roadmap-U-ABC E2G strategy). In more preferred embodiments, the Roadmap-U-ABC E2G strategy is matched to the tissue of interest.

In certain embodiments, the variant gene modules are evaluated for complex trait heritability. In certain embodiments, linkage disequilibrium score regression is used to link the phenotypes to gene modules (e.g., function). Linkage disequilibrium score regression (LDSR or LDSC) is a technique that aims to quantify the separate contributions of polygenic effects and various confounding factors, such as population stratification, based on summary statistics from genome-wide association studies (GWASs) (see, e.g., Levinson, et al., (2018). Genetic Correlation Profile of Schizophrenia Mirrors Epidemiological Results and Suggests Link Between Polygenic and Rare Variant (22q11.2) Cases of Schizophrenia. Schizophrenia Bulletin. 44 (6): 1350-1361; and Ni, et al., (2018). Estimation of Genetic Correlation via Linkage Disequilibrium Score Regression and Genomic Restricted Maximum Likelihood”. The American Journal of Human Genetics. 102 (6): 1185-1194). In certain embodiments, the Stratified LD score (S-LDSC) regression method is used to link the phenotypes to gene modules (see, e.g., Finucane, et al., 2015, Partitioning heritability by functional annotation using genome-wide association summary statistics. Nature genetics, 47:1228-1235). In certain embodiments, the output provides an inference about the association of a gene with a disease through a cellular program (e.g., module).

### Testing Genetic Interactions

In certain embodiments, gene modules are used to determine variants for testing genetic interactions. As used herein the term “genetic interaction” refers to the total effect of non-linear interactions of multiple genetic variants associated with a phenotype (e.g., SNPs) (see, e.g., Li, et al., An overview of SNP interactions in genome-wide association studies. Briefings in Functional Genomics, Volume 14, Issue 2, March 2015, Pages 143-155). In certain embodiments, interacting genetic variants contribute to increased risk for a phenotype. If one SNP has a marginal effect on a phenotype, it is known as an SNP interaction displaying marginal effects. In some cases, however, each individual SNP has no effect on the phenotype, but the combination has a strong effect; this is known as SNP interactions displaying no marginal effects (INME) (Id.). In certain embodiments, the marginal effect is difficult to identify. In certain embodiments, the present invention allows identification of SNPs having a marginal effect on a phenotype.

In certain embodiments, interactions are tested for two or more genetic loci present in the same gene module or between gene modules constructed using a single cell atlas. Prior methods do not use single cell analysis to guide selection of genetic variants to test (see, e.g., Herold, Steffens, Brockschmidt, Baur, Becker (2009), “INTERSNP: genome-wide interaction analysis guided by a priori information”, Bioinformatics, 25(24):3275-3281). Genetic loci tested for between gene modules may comprise gene modules having an association (e.g., cell type specific gene modules derived from cell types having an association, or covarying modules within a cell type). An association between gene modules of different cell types may be based on the cell types interacting. Interacting cell types may be based on the identification of ligand receptor pairs expressed in each cell type (e.g., as determined by single cell analysis). In certain embodiments, genetic interactions are tested between genetic variants present in the same gene.

### Analysis of Interacting Genetic Variants

In certain embodiments, genetic variants identified according to the present invention are clustered to determine pathways important for the phenotype (see, e.g., Udler, et al., Type 2 diabetes genetic loci informed by multi-trait associations point to disease mechanisms and subtypes: A soft clustering analysis. PLoS Med. 2018 Sep. 21; 15(9):e1002654. doi: 10.1371/journal.pmed.1002654).

In certain embodiments, genetic variants identified by testing for interactions of two or more genetic variants are used to determine cell types associated with a phenotype. Using a single cell atlas, expression of genomic loci comprising the genetic variants can be determined. Genetic variants expressed in the same cell types or interacting cell types can be identified.

### Diagnostic, Prognostic and Therapeutic Methods

In certain embodiments, the present invention provides for methods of identifying biomarkers and therapeutic targets. The invention provides biomarkers for the identification, diagnosis, prognosis and manipulation of disease phenotypes, for use in a variety of diagnostic and/or therapeutic indications. Biomarkers in the context of the present invention encompasses, without limitation nucleic acids, proteins, reaction products, and metabolites, together with their polymorphisms, mutations, variants, modifications, subunits, fragments, and other analytes or sample-derived measures. In certain embodiments, biomarkers include genes, gene programs (modules), signature gene products, and/or cells as described herein. In certain embodiments, the biomarkers are the genetic variants. In certain embodiments, the biomarkers are genes in a gene module comprising genetic variants. In certain embodiments, the biomarkers are the entire signatures in the gene modules (e.g., including co-varying genes). In certain embodiments, interacting genetic variants or combinations of interacting genetic variants are used in a polygenic risk score for a phenotype.

In certain embodiments, the invention provides uses of the biomarkers for predicting risk for a certain phenotype. In certain embodiments, the invention provides uses of the biomarkers for selecting a treatment. In certain embodiments, a subject having a disease can be classified based on severity of the disease.

The terms “diagnosis” and “monitoring” are commonplace and well-understood in medical practice. By means of further explanation and without limitation the term “diagnosis” generally refers to the process or act of recognising, deciding on or concluding on a disease or condition in a subject on the basis of symptoms and signs and/or from results of various diagnostic procedures (such as, for example, from knowing the presence, absence and/or quantity of one or more biomarkers characteristic of the diagnosed disease or condition).

The terms “prognosing” or “prognosis” generally refer to an anticipation on the progression of a disease or condition and the prospect (e.g., the probability, duration, and/or extent) of recovery. A good prognosis of the diseases or conditions taught herein may generally encompass anticipation of a satisfactory partial or complete recovery from the diseases or conditions, preferably within an acceptable time period. A good prognosis of such may more commonly encompass anticipation of not further worsening or aggravating of such, preferably within a given time period. A poor prognosis of the diseases or conditions as taught herein may generally encompass anticipation of a substandard recovery and/or unsatisfactorily slow recovery, or to substantially no recovery or even further worsening of such.

The biomarkers of the present invention are useful in methods of identifying specific patient populations based on a detected level of expression, activity and/or function of one or more biomarkers. These biomarkers are also useful in monitoring subjects undergoing treatments and therapies for suitable or aberrant response(s) to determine efficaciousness of the treatment or therapy and for selecting or modifying therapies and treatments that would be efficacious in treating, delaying the progression of or otherwise ameliorating a symptom. The biomarkers provided herein are useful for selecting a group of patients at a specific state of a disease with accuracy that facilitates selection of treatments.

The term “monitoring” generally refers to the follow-up of a disease or a condition in a subject for any changes which may occur over time.

The terms also encompass prediction of a disease. The terms “predicting” or “prediction” generally refer to an advance declaration, indication or foretelling of a disease or condition in a subject not (yet) having said disease or condition. For example, a prediction of a disease or condition in a subject may indicate a probability, chance or risk that the subject will develop said disease or condition, for example within a certain time period or by a certain age. Said probability, chance or risk may be indicated inter alia as an absolute value, range or statistics, or may be indicated relative to a suitable control subject or subject population (such as, e.g., relative to a general, normal or healthy subject or subject population). Hence, the probability, chance or risk that a subject will develop a disease or condition may be advantageously indicated as increased or decreased, or as fold-increased or fold-decreased relative to a suitable control subject or subject population. As used herein, the term “prediction” of the conditions or diseases as taught herein in a subject may also particularly mean that the subject has a ‘positive’ prediction of such, i.e., that the subject is at risk of having such (e.g., the risk is significantly increased vis-à-vis a control subject or subject population). The term “prediction of no” diseases or conditions as taught herein as described herein in a subject may particularly mean that the subject has a ‘negative’ prediction of such, i.e., that the subject's risk of having such is not significantly increased vis-à-vis a control subject or subject population.

Hence, the methods may rely on comparing the quantity of biomarkers, or gene or gene product signatures measured in samples from patients with reference values, wherein said reference values represent known predictions, diagnoses and/or prognoses of diseases or conditions as taught herein.

For example, distinct reference values may represent the prediction of a risk (e.g., an abnormally elevated risk) of having a given disease or condition as taught herein vs. the prediction of no or normal risk of having said disease or condition. In another example, distinct reference values may represent predictions of differing degrees of risk of having such disease or condition.

In a further example, distinct reference values can represent the diagnosis of a given disease or condition as taught herein vs. the diagnosis of no such disease or condition (such as, e.g., the diagnosis of healthy, or recovered from said disease or condition, etc.). In another example, distinct reference values may represent the diagnosis of such disease or condition of varying severity.

In yet another example, distinct reference values may represent a good prognosis for a given disease or condition as taught herein vs. a poor prognosis for said disease or condition. In a further example, distinct reference values may represent varyingly favourable or unfavourable prognoses for such disease or condition.

Such comparison may generally include any means to determine the presence or absence of at least one difference and optionally of the size of such difference between values being compared. A comparison may include a visual inspection, an arithmetical or statistical comparison of measurements. Such statistical comparisons include, but are not limited to, applying a rule.

Reference values may be established according to known procedures previously employed for other cell populations, biomarkers and gene or gene product signatures. For example, a reference value may be established in an individual or a population of individuals characterised by a particular diagnosis, prediction and/or prognosis of said disease or condition (i.e., for whom said diagnosis, prediction and/or prognosis of the disease or condition holds true). Such population may comprise without limitation 2 or more, 10 or more, 100 or more, or even several hundred or more individuals.

A “deviation” of a first value from a second value may generally encompass any direction (e.g., increase: first value>second value; or decrease: first value<second value) and any extent of alteration.

For example, a deviation may encompass a decrease in a first value by, without limitation, at least about 10% (about 0.9-fold or less), or by at least about 20% (about 0.8-fold or less), or by at least about 30% (about 0.7-fold or less), or by at least about 40% (about 0.6-fold or less), or by at least about 50% (about 0.5-fold or less), or by at least about 60% (about 0.4-fold or less), or by at least about 70% (about 0.3-fold or less), or by at least about 80% (about 0.2-fold or less), or by at least about 90% (about 0.1-fold or less), relative to a second value with which a comparison is being made.

For example, a deviation may encompass an increase of a first value by, without limitation, at least about 10% (about 1.1-fold or more), or by at least about 20% (about 1.2-fold or more), or by at least about 30% (about 1.3-fold or more), or by at least about 40% (about 1.4-fold or more), or by at least about 50% (about 1.5-fold or more), or by at least about 60% (about 1.6-fold or more), or by at least about 70% (about 1.7-fold or more), or by at least about 80% (about 1.8-fold or more), or by at least about 90% (about 1.9-fold or more), or by at least about 100% (about 2-fold or more), or by at least about 150% (about 2.5-fold or more), or by at least about 200% (about 3-fold or more), or by at least about 500% (about 6-fold or more), or by at least about 700% (about 8-fold or more), or like, relative to a second value with which a comparison is being made.

Preferably, a deviation may refer to a statistically significant observed alteration. For example, a deviation may refer to an observed alteration which falls outside of error margins of reference values in a given population (as expressed, for example, by standard deviation or standard error, or by a predetermined multiple thereof, e.g., ±1×SD or ±2×SD or ±3×SD, or ±1×SE or ±2×SE or ±3×SE). Deviation may also refer to a value falling outside of a reference range defined by values in a given population (for example, outside of a range which comprises ≥40%, ≥50%, ≥60%, ≥70%, ≥75% or ≥80% or ≥85% or ≥90% or ≥95% or even ≥100% of values in said population).

In a further embodiment, a deviation may be concluded if an observed alteration is beyond a given threshold or cut-off. Such threshold or cut-off may be selected as generally known in the art to provide for a chosen sensitivity and/or specificity of the prediction methods, e.g., sensitivity and/or specificity of at least 50%, or at least 60%, or at least 70%, or at least 80%, or at least 85%, or at least 90%, or at least 95%.

For example, receiver-operating characteristic (ROC) curve analysis can be used to select an optimal cut-off value of the quantity of a given immune cell population, biomarker or gene or gene product signatures, for clinical use of the present diagnostic tests, based on acceptable sensitivity and specificity, or related performance measures which are well-known per se, such as positive predictive value (PPV), negative predictive value (NPV), positive likelihood ratio (LR+), negative likelihood ratio (LR−), Youden index, or similar.

### Detection of Biomarkers

In one embodiment, the signature genes, biomarkers, and/or cells expressing biomarkers may be detected or isolated by immunofluorescence, immunohistochemistry (IHC), fluorescence activated cell sorting (FACS), mass spectrometry (MS), mass cytometry (CyTOF), sequencing, WGS (described herein), WES (described herein), RNA-seq, single cell RNA-seq (described herein), quantitative RT-PCR, single cell qPCR, FISH, RNA-FISH, MERFISH (multiplex (in situ) RNA FISH) and/or by in situ hybridization. Other methods including absorbance assays and colorimetric assays are known in the art and may be used herein. Detection may comprise primers and/or probes or fluorescently bar-coded oligonucleotide probes for hybridization to RNA (see e.g., Geiss G K, et al., Direct multiplexed measurement of gene expression with color-coded probe pairs. Nat Biotechnol. 2008 March; 26(3):317-25). In certain embodiments, cancer is diagnosed, prognosed, or monitored. For example, a tissue sample may be obtained and analyzed for specific cell markers (IHC) or specific transcripts (e.g., RNA-FISH). In one embodiment, tumor cells are stained for cell subtype specific signature genes. In one embodiment, the cells are fixed. In another embodiment, the cells are formalin fixed and paraffin embedded. Not being bound by a theory, the presence of the tumor subtypes indicate outcome and personalized treatments.

The present invention also may comprise a kit with a detection reagent that binds to one or more biomarkers or can be used to detect one or more biomarkers.

### MS Methods

Biomarker detection may also be evaluated using mass spectrometry methods. A variety of configurations of mass spectrometers can be used to detect biomarker values. Several types of mass spectrometers are available or can be produced with various configurations. In general, a mass spectrometer has the following major components: a sample inlet, an ion source, a mass analyzer, a detector, a vacuum system, and instrument-control system, and a data system. Difference in the sample inlet, ion source, and mass analyzer generally define the type of instrument and its capabilities. For example, an inlet can be a capillary-column liquid chromatography source or can be a direct probe or stage such as used in matrix-assisted laser desorption. Common ion sources are, for example, electrospray, including nanospray and microspray or matrix-assisted laser desorption. Common mass analyzers include a quadrupole mass filter, ion trap mass analyzer and time-of-flight mass analyzer. Additional mass spectrometry methods are well known in the art (see Burlingame et al., Anal. Chem. 70:647 R-716R (1998); Kinter and Sherman, New York (2000)).

Protein biomarkers and biomarker values can be detected and measured by any of the following: electrospray ionization mass spectrometry (ESI-MS), ESI-MS/MS, ESI-MS/(MS)n, matrix-assisted laser desorption ionization time-of-flight mass spectrometry (MALDI-TOF-MS), surface-enhanced laser desorption/ionization time-of-flight mass spectrometry (SELDI-TOF-MS), desorption/ionization on silicon (DIOS), secondary ion mass spectrometry (SIMS), quadrupole time-of-flight (Q-TOF), tandem time-of-flight (TOF/TOF) technology, called ultraflex III TOF/TOF, atmospheric pressure chemical ionization mass spectrometry (APCI-MS), APCI-MS/MS, APCI-(MS).sup.N, atmospheric pressure photoionization mass spectrometry (APPI-MS), APPI-MS/MS, and APPI-(MS).sup.N, quadrupole mass spectrometry, Fourier transform mass spectrometry (FTMS), quantitative mass spectrometry, and ion trap mass spectrometry.

Sample preparation strategies are used to label and enrich samples before mass spectroscopic characterization of protein biomarkers and determination biomarker values. Labeling methods include but are not limited to isobaric tag for relative and absolute quantitation (iTRAQ) and stable isotope labeling with amino acids in cell culture (SILAC). Capture reagents used to selectively enrich samples for candidate biomarker proteins prior to mass spectroscopic analysis include but are not limited to aptamers, antibodies, nucleic acid probes, chimeras, small molecules, an F(ab′)2 fragment, a single chain antibody fragment, an Fv fragment, a single chain Fv fragment, a nucleic acid, a lectin, a ligand-binding receptor, affybodies, nanobodies, ankyrins, domain antibodies, alternative antibody scaffolds (e.g. diabodies etc.) imprinted polymers, avimers, peptidomimetics, peptoids, peptide nucleic acids, threose nucleic acid, a hormone receptor, a cytokine receptor, and synthetic receptors, and modifications and fragments of these.

### Immunoassays

Immunoassay methods are based on the reaction of an antibody to its corresponding target or analyte and can detect the analyte in a sample depending on the specific assay format. To improve specificity and sensitivity of an assay method based on immunoreactivity, monoclonal antibodies are often used because of their specific epitope recognition. Polyclonal antibodies have also been successfully used in various immunoassays because of their increased affinity for the target as compared to monoclonal antibodies Immunoassays have been designed for use with a wide range of biological sample matrices Immunoassay formats have been designed to provide qualitative, semi-quantitative, and quantitative results.

Quantitative results may be generated through the use of a standard curve created with known concentrations of the specific analyte to be detected. The response or signal from an unknown sample is plotted onto the standard curve, and a quantity or value corresponding to the target in the unknown sample is established.

Numerous immunoassay formats have been designed. ELISA or EIA can be quantitative for the detection of an analyte/biomarker. This method relies on attachment of a label to either the analyte or the antibody and the label component includes, either directly or indirectly, an enzyme. ELISA tests may be formatted for direct, indirect, competitive, or sandwich detection of the analyte. Other methods rely on labels such as, for example, radioisotopes (I¹²⁵) or fluorescence. Additional techniques include, for example, agglutination, nephelometry, turbidimetry, Western blot, immunoprecipitation, immunocytochemistry, immunohistochemistry, flow cytometry, Luminex assay, and others (see ImmunoAssay: A Practical Guide, edited by Brian Law, published by Taylor & Francis, Ltd., 2005 edition).

Exemplary assay formats include enzyme-linked immunosorbent assay (ELISA), radioimmunoassay, fluorescent, chemiluminescence, and fluorescence resonance energy transfer (FRET) or time resolved-FRET (TR-FRET) immunoassays. Examples of procedures for detecting biomarkers include biomarker immunoprecipitation followed by quantitative methods that allow size and peptide level discrimination, such as gel electrophoresis, capillary electrophoresis, planar electrochromatography, and the like.

Methods of detecting and/or quantifying a detectable label or signal generating material depend on the nature of the label. The products of reactions catalyzed by appropriate enzymes (where the detectable label is an enzyme; see above) can be, without limitation, fluorescent, luminescent, or radioactive or they may absorb visible or ultraviolet light. Examples of detectors suitable for detecting such detectable labels include, without limitation, x-ray film, radioactivity counters, scintillation counters, spectrophotometers, colorimeters, fluorometers, luminometers, and densitometers.

Any of the methods for detection can be performed in any format that allows for any suitable preparation, processing, and analysis of the reactions. This can be, for example, in multi-well assay plates (e.g., 96 wells or 384 wells) or using any suitable array or microarray. Stock solutions for various agents can be made manually or robotically, and all subsequent pipetting, diluting, mixing, distribution, washing, incubating, sample readout, data collection and analysis can be done robotically using commercially available analysis software, robotics, and detection instrumentation capable of detecting a detectable label.

### Hybridization Assays

Such applications are hybridization assays in which a nucleic acid that displays “probe” nucleic acids for each of the genes to be assayed/profiled in the profile to be generated is employed. In these assays, a sample of target nucleic acids is first prepared from the initial nucleic acid sample being assayed, where preparation may include labeling of the target nucleic acids with a label, e.g., a member of a signal producing system. Following target nucleic acid sample preparation, the sample is contacted with the array under hybridization conditions, whereby complexes are formed between target nucleic acids that are complementary to probe sequences attached to the array surface. The presence of hybridized complexes is then detected, either qualitatively or quantitatively. Specific hybridization technology which may be practiced to generate the expression profiles employed in the subject methods includes the technology described in U.S. Pat. Nos. 5,143,854; 5,288,644; 5,324,633; 5,432,049; 5,470,710; 5,492,806; 5,503,980; 5,510,270; 5,525,464; 5,547,839; 5,580,732; 5,661,028; 5,800,992; the disclosures of which are herein incorporated by reference; as well as WO 95/21265; WO 96/31622; WO 97/10365; WO 97/27317; EP 373 203; and EP 785 280. In these methods, an array of “probe” nucleic acids that includes a probe for each of the biomarkers whose expression is being assayed is contacted with target nucleic acids as described above. Contact is carried out under hybridization conditions, e.g., stringent hybridization conditions as described above, and unbound nucleic acid is then removed. The resultant pattern of hybridized nucleic acids provides information regarding expression for each of the biomarkers that have been probed, where the expression information is in terms of whether or not the gene is expressed and, typically, at what level, where the expression data, i.e., expression profile, may be both qualitative and quantitative.

Optimal hybridization conditions will depend on the length (e.g., oligomer vs. polynucleotide greater than 200 bases) and type (e.g., RNA, DNA, PNA) of labeled probe and immobilized polynucleotide or oligonucleotide. General parameters for specific (i.e., stringent) hybridization conditions for nucleic acids are described in Sambrook et al., supra, and in Ausubel et al., “Current Protocols in Molecular Biology”, Greene Publishing and Wiley-interscience, NY (1987), which is incorporated in its entirety for all purposes. When the cDNA microarrays are used, typical hybridization conditions are hybridization in 5×SSC plus 0.2% SDS at 65 C for 4 hours followed by washes at 25° C. in low stringency wash buffer (1×SSC plus 0.2% SDS) followed by 10 minutes at 25° C. in high stringency wash buffer (0.1SSC plus 0.2% SDS) (see Shena et al., Proc. Natl. Acad. Sci. USA, Vol. 93, p. 10614 (1996)). Useful hybridization conditions are also provided in, e.g., Tijessen, Hybridization With Nucleic Acid Probes”, Elsevier Science Publishers B.V. (1993) and Kricka, “Nonisotopic DNA Probe Techniques”, Academic Press, San Diego, Calif. (1992).

In certain embodiments, a subject can be categorized based on signature genes or gene programs expressed by a tissue sample obtained from the subject. In certain embodiments, the tissue sample is analyzed by bulk sequencing. In certain embodiments, subtypes can be determined by determining the percentage of specific cell subtypes expressing the identified interacting genetic variants in the sample that contribute to the phenotype. In certain embodiments, gene expression associated with the cells are determined from bulk sequencing reads by deconvolution of the sample. For example, deconvoluting bulk gene expression data obtained from a tumor containing both malignant and non-malignant cells can include defining the relative frequency of a set of cell types in the tumor from the bulk gene expression data using cell type specific gene expression (e.g., cell types may be T cells, fibroblasts, macrophages, mast cells, B/plasma cells, endothelial cells, myocytes and dendritic cells); and defining a linear relationship between the frequency of the non-malignant cell types and the expression of a set of genes, wherein the set of genes comprises genes highly expressed by malignant cells and at most two non-malignant cell types, wherein the set of genes are derived from gene expression analysis of single cells in the tumor or the same tumor type, and wherein the residual of the linear relationship defines the malignant cell-specific (MCS) expression profile (see, e.g., WO 2018/191553; and Puram et al., Cell. 2017 Dec. 14; 171(7):1611-1624.e24).

### Therapeutic Agents

In certain embodiments, the present invention provides for one or more therapeutic agents to treat any disease phenotype described herein. Targeting the identified genetic variants (i.e., including associated genes) may provide for enhanced or otherwise previously unknown activity in the treatment of disease. In certain embodiments, targeting combinations of genetic variants or genes comprising genetic variants may require less of an agent as compared to the current standard of care targeting the variant and provide for less toxicity and improved treatment. In certain embodiments, the agents are used to modulate cell types (e.g., shifting signatures). In certain embodiments, the one or more agents comprises a small molecule inhibitor, small molecule degrader (e.g., PROTAC), genetic modifying agent, antibody, antibody fragment, antibody-like protein scaffold, aptamer, protein, or any combination thereof.

The terms “therapeutic agent”, “therapeutic capable agent” or “treatment agent” are used interchangeably and refer to a molecule or compound that confers some beneficial effect upon administration to a subject. The beneficial effect includes enablement of diagnostic determinations; amelioration of a disease, symptom, disorder, or pathological condition; reducing or preventing the onset of a disease, symptom, disorder or condition; and generally counteracting a disease, symptom, disorder or pathological condition.

As used herein, “treatment” or “treating,” or “palliating” or “ameliorating” are used interchangeably. These terms refer to an approach for obtaining beneficial or desired results including, but not limited to, a therapeutic benefit and/or a prophylactic benefit. By therapeutic benefit is meant any therapeutically relevant improvement in or effect on one or more diseases, conditions, or symptoms under treatment. For prophylactic benefit, the compositions may be administered to a subject at risk of developing a particular disease, condition, or symptom, or to a subject reporting one or more of the physiological symptoms of a disease, even though the disease, condition, or symptom may not have yet been manifested. As used herein “treating” includes ameliorating, curing, preventing it from becoming worse, slowing the rate of progression, or preventing the disorder from re-occurring (i.e., to prevent a relapse).

The term “effective amount” or “therapeutically effective amount” refers to the amount of an agent that is sufficient to effect beneficial or desired results. The therapeutically effective amount may vary depending upon one or more of: the subject and disease condition being treated, the weight and age of the subject, the severity of the disease condition, the manner of administration and the like, which can readily be determined by one of ordinary skill in the art. The term also applies to a dose that will provide an image for detection by any one of the imaging methods described herein. The specific dose may vary depending on one or more of: the particular agent chosen, the dosing regimen to be followed, whether it is administered in combination with other compounds, timing of administration, the tissue to be imaged, and the physical delivery system in which it is carried.

For example, in methods for treating cancer in a subject, an effective amount of a combination of agents is any amount that provides an anti-cancer effect, such as reduces or prevents proliferation of a cancer cell or makes a cancer cell responsive to an immunotherapy.

### Standard of Care

Aspects of the invention involve modifying the therapy within a standard of care based on the detection of any of the biomarkers as described herein. In one embodiment, therapy comprising an agent is administered within a standard of care where addition of the agent is synergistic within the steps of the standard of care. In one embodiment, the agent targets and/or shifts a tumor to an immunotherapy responder phenotype. In one embodiment, the agent inhibits expression or activity of one or more transcription factors capable of regulating a gene program. In one embodiment, the agent targets tumor cells expressing a gene program. The term “standard of care” as used herein refers to the current treatment that is accepted by medical experts as a proper treatment for a certain type of disease and that is widely used by healthcare professionals. Standard of care is also called best practice, standard medical care, and standard therapy. Standards of care for cancer generally include surgery, lymph node removal, radiation, chemotherapy, targeted therapies, antibodies targeting the tumor, and immunotherapy. Immunotherapy can include checkpoint blockers (CBP), chimeric antigen receptors (CARs), and adoptive T-cell therapy. The standards of care for the most common cancers can be found on the website of National Cancer Institute (www.cancer.gov/cancertopics). A treatment clinical trial is a research study meant to help improve current treatments or obtain information on new treatments for patients with cancer. When clinical trials show that a new treatment is better than the standard treatment, the new treatment may be considered the new standard treatment.

The term “Adjuvant therapy” as used herein refers to any treatment given after primary therapy to increase the chance of long-term disease-free survival. The term “Neoadjuvant therapy” as used herein refers to any treatment given before primary therapy. The term “Primary therapy” as used herein refers to the main treatment used to reduce or eliminate the cancer. In certain embodiments, an agent that shifts a tumor to a responder phenotype are provided as a neoadjuvant before CPB therapy.

### Checkpoint Blockade Therapy

Immunotherapy can include checkpoint blockers (CBP), chimeric antigen receptors (CARs), and adoptive T-cell therapy. Antibodies that block the activity of checkpoint receptors, including CTLA-4, PD-1, Tim-3, Lag-3, and TIGIT, either alone or in combination, have been associated with improved effector CD8⁺ T cell responses in multiple pre-clinical cancer models (Johnston et al., 2014. The immunoreceptor TIGIT regulates antitumor and antiviral CD8(+) T cell effector function. Cancer cell 26, 923-937; Ngiow et al., 2011. Anti-TIM3 antibody promotes T cell IFN-gamma-mediated antitumor immunity and suppresses established tumors. Cancer research 71, 3540-3551; Sakuishi et al., 2010. Targeting Tim-3 and PD-1 pathways to reverse T cell exhaustion and restore anti-tumor immunity. The Journal of experimental medicine 207, 2187-2194; and Woo et al., 2012. Immune inhibitory molecules LAG-3 and PD-1 synergistically regulate T-cell function to promote tumoral immune escape. Cancer research 72, 917-927). Similarly, blockade of CTLA-4 and PD-1 in patients (Brahmer et al., 2012. Safety and activity of anti-PD-L1 antibody in patients with advanced cancer. The New England journal of medicine 366, 2455-2465; Hodi et al., 2010. Improved survival with ipilimumab in patients with metastatic melanoma. The New England journal of medicine 363, 711-723; Schadendorf et al., 2015. Pooled Analysis of Long-Term Survival Data From Phase II and Phase III Trials of Ipilimumab in Unresectable or Metastatic Melanoma. Journal of clinical oncology: official journal of the American Society of Clinical Oncology 33, 1889-1894; Topalian et al., 2012. Safety, activity, and immune correlates of anti-PD-1 antibody in cancer. The New England journal of medicine 366, 2443-2454; and Wolchok et al., 2017. Overall Survival with Combined Nivolumab and Ipilimumab in Advanced Melanoma. The New England journal of medicine 377, 1345-1356) has shown increased frequencies of proliferating T cells, often with specificity for tumor antigens, as well as increased CD8⁺ T cell effector function (Ayers et al., 2017. IFN-gamma-related mRNA profile predicts clinical response to PD-1 blockade. The Journal of clinical investigation 127, 2930-2940; Das et al., 2015. Combination therapy with anti-CTLA-4 and anti-PD-1 leads to distinct immunologic changes in vivo. Journal of immunology 194, 950-959; Gubin et al., 2014. Checkpoint blockade cancer immunotherapy targets tumour-specific mutant antigens. Nature 515, 577-581; Huang et al., 2017. T-cell invigoration to tumour burden ratio associated with anti-PD-1 response. Nature 545, 60-65; Kamphorst et al., 2017. Proliferation of PD-1+CD8 T cells in peripheral blood after PD-1-targeted therapy in lung cancer patients. Proceedings of the National Academy of Sciences of the United States of America 114, 4993-4998; Kvistborg et al., 2014. Anti-CTLA-4 therapy broadens the melanoma-reactive CD8+ T cell response. Science translational medicine 6, 254ra128; van Rooij et al., 2013. Tumor exome analysis reveals neoantigen-specific T-cell reactivity in an ipilimumab-responsive melanoma. Journal of clinical oncology: official journal of the American Society of Clinical Oncology 31, e439-442; and Yuan et al., 2008. CTLA-4 blockade enhances polyfunctional NY-ESO-1 specific T cell responses in metastatic melanoma patients with clinical benefit. Proceedings of the National Academy of Sciences of the United States of America 105, 20410-20415). Accordingly, the success of checkpoint receptor blockade has been attributed to the binding of blocking antibodies to checkpoint receptors expressed on dysfunctional CD8⁺ T cells and restoring effector function in these cells. The check point blockade therapy may be an inhibitor of any check point protein described herein. The checkpoint blockade therapy may comprise anti-TIM3, anti-CTLA4, anti-PD-L1, anti-PD1, anti-TIGIT, anti-LAG3, or combinations thereof. Anti-PD1 antibodies are disclosed in U.S. Pat. No. 8,735,553. Antibodies to LAG-3 are disclosed in U.S. Pat. No. 9,132,281. Anti-CTLA4 antibodies are disclosed in U.S. Pat. Nos. 9,327,014; 9,320,811; and 9,062,111. Specific check point inhibitors include, but are not limited to anti-CTLA4 antibodies (e.g., Ipilimumab and tremelimumab), anti-PD-1 antibodies (e.g., Nivolumab, Pembrolizumab), and anti-PD-L1 antibodies (e.g., Atezolizumab).

### Small Molecules

In certain embodiments, the one or more agents is a small molecule. The term “small molecule” refers to compounds, preferably organic compounds, with a size comparable to those organic molecules generally used in pharmaceuticals. The term excludes biological macromolecules (e.g., proteins, peptides, nucleic acids, etc.). Preferred small organic molecules range in size up to about 5000 Da, e.g., up to about 4000, preferably up to 3000 Da, more preferably up to 2000 Da, even more preferably up to about 1000 Da, e.g., up to about 900, 800, 700, 600 or up to about 500 Da. In certain embodiments, the small molecule may act as an antagonist or agonist (e.g., blocking an enzyme active site or activating a receptor by binding to a ligand binding site).

One type of small molecule applicable to the present invention is a degrader molecule. Proteolysis Targeting Chimera (PROTAC) technology is a rapidly emerging alternative therapeutic strategy with the potential to address many of the challenges currently faced in modern drug development programs. PROTAC technology employs small molecules that recruit target proteins for ubiquitination and removal by the proteasome (see, e.g., Zhou et al., Discovery of a Small-Molecule Degrader of Bromodomain and Extra-Terminal (BET) Proteins with Picomolar Cellular Potencies and Capable of Achieving Tumor Regression. J. Med. Chem. 2018, 61, 462-481; Bondeson and Crews, Targeted Protein Degradation by Small Molecules, Annu Rev Pharmacol Toxicol. 2017 Jan. 6; 57: 107-123; and Lai et al., Modular PROTAC Design for the Degradation of Oncogenic BCR-ABL Angew Chem Int Ed Engl. 2016 Jan. 11; 55(2): 807-810).

### Genetic Modifying Agents

In certain embodiments, the one or more modulating agents may be a genetic modifying agent (e.g., modifies a transcription factor). The genetic modifying agent may comprise a CRISPR system, a zinc finger nuclease system, a TALEN, a meganuclease or RNAi system. In certain embodiments, a target gene is genetically modified. In certain embodiments, a target gene RNA is modified, such that the modification is temporary. Methods of modifying RNA is discussed further herein.

### CRISPR-Cas Modification

In some embodiments, a polynucleotide of the present invention described elsewhere herein can be modified using a CRISPR-Cas and/or Cas-based system (e.g., genomic DNA or mRNA, preferably, for a disease gene). The nucleotide sequence may be or encode one or more components of a CRISPR-Cas system. For example, the nucleotide sequences may be or encode guide RNAs. The nucleotide sequences may also encode CRISPR proteins, variants thereof, or fragments thereof.

In general, a CRISPR-Cas or CRISPR system as used herein and in other documents, such as WO 2014/093622 (PCT/US2013/074667), refers collectively to transcripts and other elements involved in the expression of or directing the activity of CRISPR-associated (“Cas”) genes, including sequences encoding a Cas gene, a tracr (trans-activating CRISPR) sequence (e.g., tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or “RNA(s)” as that term is herein used (e.g., RNA(s) to guide Cas, such as Cas9, e.g., CRISPR RNA and transactivating (tracr) RNA or a single guide RNA (sgRNA) (chimeric RNA)) or other sequences and transcripts from a CRISPR locus. In general, a CRISPR system is characterized by elements that promote the formation of a CRISPR complex at the site of a target sequence (also referred to as a protospacer in the context of an endogenous CRISPR system). See, e.g., Shmakov et al. (2015) “Discovery and Functional Characterization of Diverse Class 2 CRISPR-Cas Systems”, Molecular Cell, DOI: dx.doi.org/10.1016/j.molcel.2015.10.008.

CRISPR-Cas systems can generally fall into two classes based on their architectures of their effector molecules, which are each further subdivided by type and subtype. The two class are Class 1 and Class 2. Class 1 CRISPR-Cas systems have effector modules composed of multiple Cas proteins, some of which form crRNA-binding complexes, while Class 2 CRISPR-Cas systems include a single, multi-domain crRNA-binding protein.

In some embodiments, the CRISPR-Cas system that can be used to modify a polynucleotide of the present invention described herein can be a Class 1 CRISPR-Cas system. In some embodiments, the CRISPR-Cas system that can be used to modify a polynucleotide of the present invention described herein can be a Class 2 CRISPR-Cas system.

### Class 1 CRISPR-Cas Systems

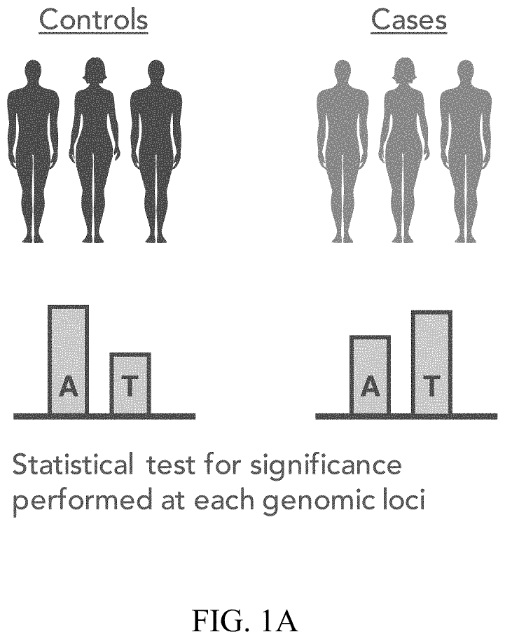

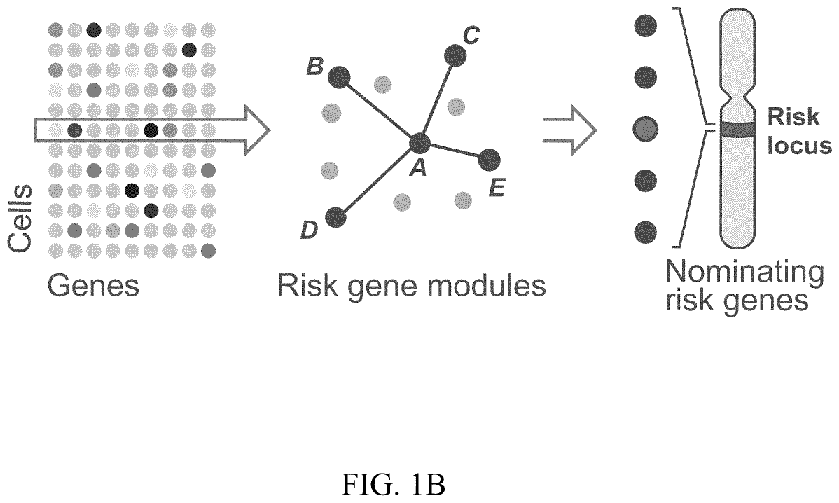

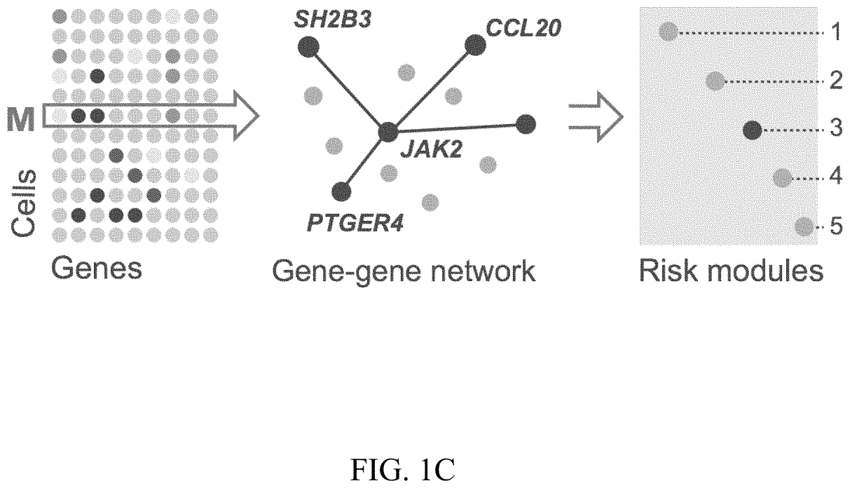

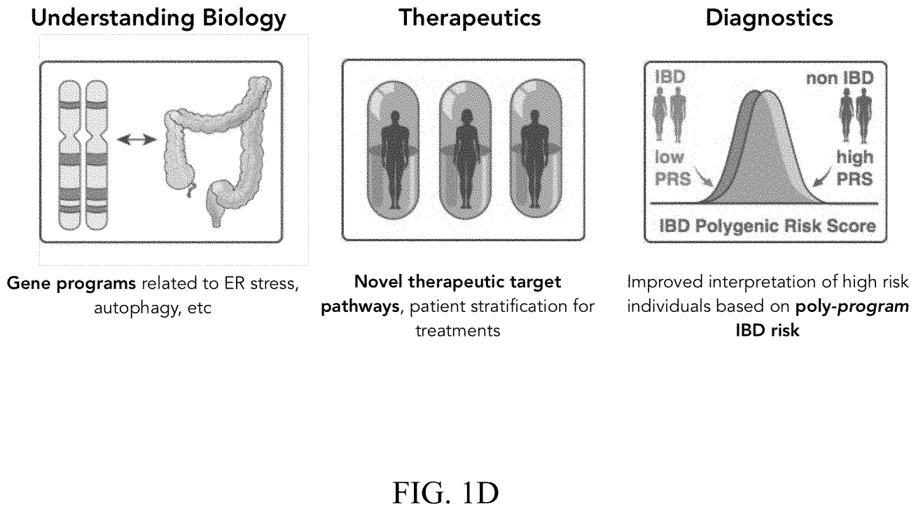

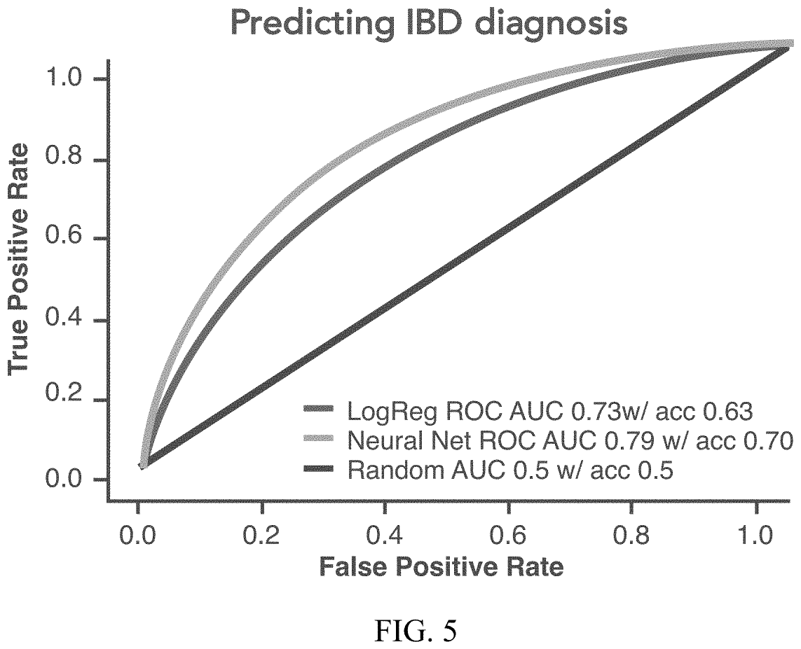

In some embodiments, the CRISPR-Cas system that can be used to modify a polynucleotide of the present invention described herein can be a Class 1 CRISPR-Cas system. Class 1 CRISPR-Cas systems are divided into types I, II, and IV. Makarova et al. 2020. Nat. Rev. 18: 67-83., particularly as described in FIG. 1. Type I CRISPR-Cas systems are divided into 9 subtypes (I-A, I-B, I-C, I-D, I-E, I-F1, I-F2, I-F3, and IG). Makarova et al., 2020. Class 1, Type I CRISPR-Cas systems can contain a Cas3 protein that can have helicase activity. Type III CRISPR-Cas systems are divided into 6 subtypes (III-A, III-B, III-E, and III-F). Type III CRISPR-Cas systems can contain a Cas10 that can include an RNA recognition motif called Palm and a cyclase domain that can cleave polynucleotides. Makarova et al., 2020. Type IV CRISPR-Cas systems are divided into 3 subtypes. (IV-A, IV-B, and IV-C). Makarova et al., 2020. Class 1 systems also include CRISPR-Cas variants, including Type I-A, I-B, I-E, I-F and I-U variants, which can include variants carried by transposons and plasmids, including versions of subtype I-F encoded by a large family of Tn7-like transposon and smaller groups of Tn7-like transposons that encode similarly degraded subtype I-B systems. Peters et al., PNAS 114 (35) (2017); DOI: 10.1073/pnas.1709035114; see also, Makarova et al. 2018. The CRISPR Journal, v. 1, n5, FIG. 5.

The Class 1 systems typically use a multi-protein effector complex, which can, in some embodiments, include ancillary proteins, such as one or more proteins in a complex referred to as a CRISPR-associated complex for antiviral defense (Cascade), one or more adaptation proteins (e.g., Cas1, Cas2, RNA nuclease), and/or one or more accessory proteins (e.g., Cas 4, DNA nuclease), CRISPR associated Rossman fold (CARF) domain containing proteins, and/or RNA transcriptase.

The backbone of the Class 1 CRISPR-Cas system effector complexes can be formed by RNA recognition motif domain-containing protein(s) of the repeat-associated mysterious proteins (RAMPs) family subunits (e.g., Cas 5, Cas6, and/or Cas7). RAMP proteins are characterized by having one or more RNA recognition motif domains. In some embodiments, multiple copies of RAMPs can be present. In some embodiments, the Class I CRISPR-Cas system can include 1, 2, 3, 4, 5, 6, 7, 8, 9, 10, 11, 12 or more Cas5, Cas6, and/or Cas 7 proteins. In some embodiments, the Cas6 protein is an RNAse, which can be responsible for pre-crRNA processing. When present in a Class 1 CRISPR-Cas system, Cas6 can be optionally physically associated with the effector complex.

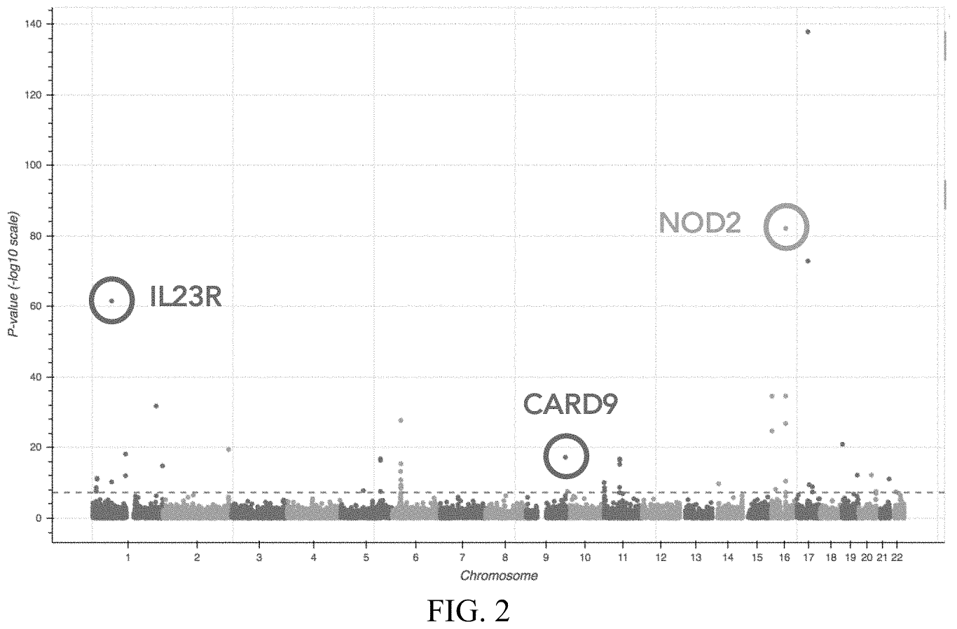

Class 1 CRISPR-Cas system effector complexes can, in some embodiments, also include a large subunit. The large subunit can be composed of or include a Cas8 and/or Cas10 protein. See, e.g., FIGS. 1 and 2. Koonin E V, Makarova K S. 2019. Phil. Trans. R. Soc. B 374: 20180087, DOI: 10.1098/rstb.2018.0087 and Makarova et al. 2020.

Class 1 CRISPR-Cas system effector complexes can, in some embodiments, include a small subunit (for example, Cash 1). See, e.g., FIGS. 1 and 2. Koonin E V, Makarova K S. 2019 Origins and Evolution of CRISPR-Cas systems. Phil. Trans. R. Soc. B 374: 20180087, DOI: 10.1098/rstb.2018.0087.

In some embodiments, the Class 1 CRISPR-Cas system can be a Type I CRISPR-Cas system. In some embodiments, the Type I CRISPR-Cas system can be a subtype I-A CRISPR-Cas system. In some embodiments, the Type I CRISPR-Cas system can be a subtype I-B CRISPR-Cas system. In some embodiments, the Type I CRISPR-Cas system can be a subtype I-C CRISPR-Cas system. In some embodiments, the Type I CRISPR-Cas system can be a subtype I-D CRISPR-Cas system. In some embodiments, the Type I CRISPR-Cas system can be a subtype I-E CRISPR-Cas system. In some embodiments, the Type I CRISPR-Cas system can be a subtype I-F1 CRISPR-Cas system. In some embodiments, the Type I CRISPR-Cas system can be a subtype I-F2 CRISPR-Cas system. In some embodiments, the Type I CRISPR-Cas system can be a subtype I-F3 CRISPR-Cas system. In some embodiments, the Type I CRISPR-Cas system can be a subtype I-G CRISPR-Cas system. In some embodiments, the Type I CRISPR-Cas system can be a CRISPR Cas variant, such as a Type I-A, I-B, I-E, I-F and I-U variants, which can include variants carried by transposons and plasmids, including versions of subtype I-F encoded by a large family of Tn7-like transposon and smaller groups of Tn7-like transposons that encode similarly degraded subtype I-B systems as previously described.

In some embodiments, the Class 1 CRISPR-Cas system can be a Type III CRISPR-Cas system. In some embodiments, the Type III CRISPR-Cas system can be a subtype III-A CRISPR-Cas system. In some embodiments, the Type III CRISPR-Cas system can be a subtype III-B CRISPR-Cas system. In some embodiments, the Type III CRISPR-Cas system can be a subtype III-C CRISPR-Cas system. In some embodiments, the Type III CRISPR-Cas system can be a subtype III-D CRISPR-Cas system. In some embodiments, the Type III CRISPR-Cas system can be a subtype III-E CRISPR-Cas system. In some embodiments, the Type III CRISPR-Cas system can be a subtype III-F CRISPR-Cas system.

In some embodiments, the Class 1 CRISPR-Cas system can be a Type IV CRISPR-Cas-system. In some embodiments, the Type IV CRISPR-Cas system can be a subtype IV-A CRISPR-Cas system. In some embodiments, the Type IV CRISPR-Cas system can be a subtype IV-B CRISPR-Cas system. In some embodiments, the Type IV CRISPR-Cas system can be a subtype IV-C CRISPR-Cas system.

The effector complex of a Class 1 CRISPR-Cas system can, in some embodiments, include a Cas3 protein that is optionally fused to a Cas2 protein, a Cas4, a Cas5, a Cash, a Cas7, a Cas8, a Cas10, a Cas11, or a combination thereof. In some embodiments, the effector complex of a Class 1 CRISPR-Cas system can have multiple copies, such as 1, 2, 3, 4, 5, 6, 7, 8, 9, 10, 11, 12, 13, or 14, of any one or more Cas proteins.

### Class 2 CRISPR-Cas Systems

The compositions, systems, and methods described in greater detail elsewhere herein can be designed and adapted for use with Class 2 CRISPR-Cas systems. Thus, in some embodiments, the CRISPR-Cas system is a Class 2 CRISPR-Cas system. Class 2 systems are distinguished from Class 1 systems in that they have a single, large, multi-domain effector protein. In certain example embodiments, the Class 2 system can be a Type II, Type V, or Type VI system, which are described in Makarova et al. “Evolutionary classification of CRISPR-Cas systems: a burst of class 2 and derived variants” Nature Reviews Microbiology, 18:67-81 (February 2020), incorporated herein by reference. Each type of Class 2 system is further divided into subtypes. See Markova et al. 2020, particularly at Figure. 2. Class 2, Type II systems can be divided into 4 subtypes: II-A, II-B, II-C1, and II-C2. Class 2, Type V systems can be divided into 17 subtypes: V-A, V-B1, V-B2, V-C, V-D, V-E, V-F1, V-F1(V-U3), V-F2, V-F3, V-G, V-H, V-I, V-K (V-U5), V-U1, V-U2, and V-U4. Class 2, Type IV systems can be divided into 5 subtypes: VI-A, VI-B1, VI-B2, VI-C, and VI-D.

The distinguishing feature of these types is that their effector complexes consist of a single, large, multi-domain protein. Type V systems differ from Type II effectors (e.g., Cas9), which contain two nuclear domains that are each responsible for the cleavage of one strand of the target DNA, with the HNH nuclease inserted inside the Ruv-C like nuclease domain sequence. The Type V systems (e.g., Cas12) only contain a RuvC-like nuclease domain that cleaves both strands. Type VI (Cas13) are unrelated to the effectors of Type II and V systems and contain two HEPN domains and target RNA. Cas13 proteins also display collateral activity that is triggered by target recognition. Some Type V systems have also been found to possess this collateral activity with two single-stranded DNA in in vitro contexts.

In some embodiments, the Class 2 system is a Type II system. In some embodiments, the Type II CRISPR-Cas system is a II-A CRISPR-Cas system. In some embodiments, the Type II CRISPR-Cas system is a II-B CRISPR-Cas system. In some embodiments, the Type II CRISPR-Cas system is a II-C1 CRISPR-Cas system. In some embodiments, the Type II CRISPR-Cas system is a II-C2 CRISPR-Cas system. In some embodiments, the Type II system is a Cas9 system. In some embodiments, the Type II system includes a Cas9.

In some embodiments, the Class 2 system is a Type V system. In some embodiments, the Type V CRISPR-Cas system is a V-A CRISPR-Cas system. In some embodiments, the Type V CRISPR-Cas system is a V-B 1 CRISPR-Cas system. In some embodiments, the Type V CRISPR-Cas system is a V-B2 CRISPR-Cas system. In some embodiments, the Type V CRISPR-Cas system is a V-C CRISPR-Cas system. In some embodiments, the Type V CRISPR-Cas system is a V-D CRISPR-Cas system. In some embodiments, the Type V CRISPR-Cas system is a V-E CRISPR-Cas system. In some embodiments, the Type V CRISPR-Cas system is a V-F1 CRISPR-Cas system. In some embodiments, the Type V CRISPR-Cas system is a V-F1 (V-U3) CRISPR-Cas system. In some embodiments, the Type V CRISPR-Cas system is a V-F2 CRISPR-Cas system. In some embodiments, the Type V CRISPR-Cas system is a V-F3 CRISPR-Cas system. In some embodiments, the Type V CRISPR-Cas system is a V-G CRISPR-Cas system. In some embodiments, the Type V CRISPR-Cas system is a V-H CRISPR-Cas system. In some embodiments, the Type V CRISPR-Cas system is a V-I CRISPR-Cas system. In some embodiments, the Type V CRISPR-Cas system is a V-K (V-U5) CRISPR-Cas system. In some embodiments, the Type V CRISPR-Cas system is a V-U1 CRISPR-Cas system. In some embodiments, the Type V CRISPR-Cas system is a V-U2 CRISPR-Cas system. In some embodiments, the Type V CRISPR-Cas system is a V-U4 CRISPR-Cas system. In some embodiments, the Type V CRISPR-Cas system includes a Cas12a (Cpfl), Cas12b (C2c1), Cas12c (C2c3), CasX, and/or Cas14.

In some embodiments the Class 2 system is a Type VI system. In some embodiments, the Type VI CRISPR-Cas system is a VI-A CRISPR-Cas system. In some embodiments, the Type VI CRISPR-Cas system is a VI-B1 CRISPR-Cas system. In some embodiments, the Type VI CRISPR-Cas system is a VI-B2 CRISPR-Cas system. In some embodiments, the Type VI CRISPR-Cas system is a VI-C CRISPR-Cas system. In some embodiments, the Type VI CRISPR-Cas system is a VI-D CRISPR-Cas system. In some embodiments, the Type VI CRISPR-Cas system includes a Cas13a (C2c2), Cas13b (Group 29/30), Cas13c, and/or Cas13d.

### Specialized Cas-Based Systems

In some embodiments, the system is a Cas-based system that is capable of performing a specialized function or activity. For example, the Cas protein may be fused, operably coupled to, or otherwise associated with one or more functionals domains. In certain example embodiments, the Cas protein may be a catalytically dead Cas protein (“dCas”) and/or have nickase activity. A nickase is a Cas protein that cuts only one strand of a double stranded target. In such embodiments, the dCas or nickase provide a sequence specific targeting functionality that delivers the functional domain to or proximate a target sequence. Example functional domains that may be fused to, operably coupled to, or otherwise associated with a Cas protein can be or include, but are not limited to a nuclear localization signal (NLS) domain, a nuclear export signal (NES) domain, a translational activation domain, a transcriptional activation domain (e.g. VP64, p 65, MyoD1, HSF1, RTA, and SETT/9), a translation initiation domain, a transcriptional repression domain (e.g., a KRAB domain, NuE domain, NcoR domain, and a SID domain such as a SID4X domain), a nuclease domain (e.g., Fold), a histone modification domain (e.g., a histone acetyltransferase), a light inducible/controllable domain, a chemically inducible/controllable domain, a transposase domain, a homologous recombination machinery domain, a recombinase domain, an integrase domain, and combinations thereof. Methods for generating catalytically dead Cas9 or a nickase Cas9 (WO 2014/204725, Ran et al. Cell. 2013 Sep. 12; 154(6):1380-1389), Cas12 (Liu et al. Nature Communications, 8, 2095 (2017), and Cas13 (WO 2019/005884, WO2019/060746) are known in the art and incorporated herein by reference.

In some embodiments, the functional domains can have one or more of the following activities: methylase activity, demethylase activity, translation activation activity, translation initiation activity, translation repression activity, transcription activation activity, transcription repression activity, transcription release factor activity, histone modification activity, nuclease activity, single-strand RNA cleavage activity, double-strand RNA cleavage activity, single-strand DNA cleavage activity, double-strand DNA cleavage activity, molecular switch activity, chemical inducibility, light inducibility, and nucleic acid binding activity. In some embodiments, the one or more functional domains may comprise epitope tags or reporters. Non-limiting examples of epitope tags include histidine (His) tags, V5 tags, FLAG tags, influenza hemagglutinin (HA) tags, Myc tags, VSV-G tags, and thioredoxin (Trx) tags. Examples of reporters include, but are not limited to, glutathione-S-transferase (GST), horseradish peroxidase (HRP), chloramphenicol acetyltransferase (CAT) beta-galactosidase, beta-glucuronidase, luciferase, green fluorescent protein (GFP), HcRed, DsRed, cyan fluorescent protein (CFP), yellow fluorescent protein (YFP), and auto-fluorescent proteins including blue fluorescent protein (BFP).

The one or more functional domain(s) may be positioned at, near, and/or in proximity to a terminus of the effector protein (e.g., a Cas protein). In embodiments having two or more functional domains, each of the two can be positioned at or near or in proximity to a terminus of the effector protein (e.g., a Cas protein). In some embodiments, such as those where the functional domain is operably coupled to the effector protein, the one or more functional domains can be tethered or linked via a suitable linker (including, but not limited to, GlySer linkers) to the effector protein (e.g., a Cas protein). When there is more than one functional domain, the functional domains can be same or different. In some embodiments, all the functional domains are the same. In some embodiments, all of the functional domains are different from each other. In some embodiments, at least two of the functional domains are different from each other. In some embodiments, at least two of the functional domains are the same as each other.

Other suitable functional domains can be found, for example, in International Patent Publication No. WO 2019/018423.

### Split CRISPR-Cas Systems

In some embodiments, the CRISPR-Cas system is a split CRISPR-Cas system. See e.g., Zetche et al., 2015. Nat. Biotechnol. 33(2): 139-142 and WO 2019/018423, the compositions and techniques of which can be used in and/or adapted for use with the present invention. Split CRISPR-Cas proteins are set forth herein and in documents incorporated herein by reference in further detail herein. In certain embodiments, each part of a split CRISPR protein are attached to a member of a specific binding pair, and when bound with each other, the members of the specific binding pair maintain the parts of the CRISPR protein in proximity. In certain embodiments, each part of a split CRISPR protein is associated with an inducible binding pair. An inducible binding pair is one which is capable of being switched “on” or “off” by a protein or small molecule that binds to both members of the inducible binding pair. In some embodiments, CRISPR proteins may preferably split between domains, leaving domains intact. In particular embodiments, said Cas split domains (e.g., RuvC and HNH domains in the case of Cas9) can be simultaneously or sequentially introduced into the cell such that said split Cas domain(s) process the target nucleic acid sequence in the algae cell. The reduced size of the split Cas compared to the wild type Cas allows other methods of delivery of the systems to the cells, such as the use of cell penetrating peptides as described herein.

### DNA and RNA Base Editing

In some embodiments, a polynucleotide of the present invention described elsewhere herein can be modified using a base editing system. In some embodiments, a Cas protein is connected or fused to a nucleotide deaminase. Thus, in some embodiments the Cas-based system can be a base editing system. As used herein “base editing” refers generally to the process of polynucleotide modification via a CRISPR-Cas-based or Cas-based system that does not include excising nucleotides to make the modification. Base editing can convert base pairs at precise locations without generating excess undesired editing byproducts that can be made using traditional CRISPR-Cas systems.

In certain example embodiments, the nucleotide deaminase may be a DNA base editor used in combination with a DNA binding Cas protein such as, but not limited to, Class 2 Type II and Type V systems. Two classes of DNA base editors are generally known: cytosine base editors (CBEs) and adenine base editors (ABEs). CBEs convert a C•G base pair into a T•A base pair (Komor et al. 2016. Nature. 533:420-424; Nishida et al. 2016. Science. 353; and Li et al. Nat. Biotech. 36:324-327) and ABEs convert an A•T base pair to a G•C base pair. Collectively, CBEs and ABEs can mediate all four possible transition mutations (C to T, A to G, T to C, and G to A). Rees and Liu. 2018.Nat. Rev. Genet. 19(12): 770-788, particularly at FIGS. 1b, 2a-2c, 3a-3f, and Table 1. In some embodiments, the base editing system includes a CBE and/or an ABE. In some embodiments, a polynucleotide of the present invention described elsewhere herein can be modified using a base editing system. Rees and Liu. 2018. Nat. Rev. Gent. 19(12):770-788. Base editors also generally do not need a DNA donor template and/or rely on homology-directed repair. Komor et al. 2016. Nature. 533:420-424; Nishida et al. 2016. Science. 353; and Gaudeli et al. 2017. Nature. 551:464-471. Upon binding to a target locus in the DNA, base pairing between the guide RNA of the system and the target DNA strand leads to displacement of a small segment of ssDNA in an “R-loop”. Nishimasu et al. Cell. 156:935-949. DNA bases within the ssDNA bubble are modified by the enzyme component, such as a deaminase. In some systems, the catalytically disabled Cas protein can be a variant or modified Cas can have nickase functionality and can generate a nick in the non-edited DNA strand to induce cells to repair the non-edited strand using the edited strand as a template. Komor et al. 2016. Nature. 533:420-424; Nishida et al. 2016. Science. 353; and Gaudeli et al. 2017. Nature. 551:464-471. Base editors may be further engineered to optimize conversion of nucleotides (e.g. A:T to G:C). Richter et al. 2020. Nature Biotechnology. doi.org/10.1038/s41587-020-0453-z.

Other Example Type V base editing systems are described in WO 2018/213708, WO 2018/213726, PCT/US2018/067207, PCT/US2018/067225, and PCT/US2018/067307 which are incorporated by referenced herein.

In certain example embodiments, the base editing system may be a RNA base editing system. As with DNA base editors, a nucleotide deaminase capable of converting nucleotide bases may be fused to a Cas protein. However, in these embodiments, the Cas protein will need to be capable of binding RNA. Example RNA binding Cas proteins include, but are not limited to, RNA-binding Cas9s such as Francisella novicida Cas9 (“FnCas9”), and Class 2 Type VI Cas systems. The nucleotide deaminase may be a cytidine deaminase or an adenosine deaminase, or an adenosine deaminase engineered to have cytidine deaminase activity. In certain example embodiments, the RNA based editor may be used to delete or introduce a post-translation modification site in the expressed mRNA. In contrast to DNA base editors, whose edits are permanent in the modified cell, RNA base editors can provide edits where finer temporal control may be needed, for example in modulating a particular immune response. Example Type VI RNA-base editing systems are described in Cox et al. 2017. Science 358: 1019-1027, WO 2019/005884, WO 2019/005886, WO 2019/071048, PCT/US20018/05179, PCT/US2018/067207, which are incorporated herein by reference. An example FnCas9 system that may be adapted for RNA base editing purposes is described in WO 2016/106236, which is incorporated herein by reference.

An example method for delivery of base-editing systems, including use of a split-intein approach to divide CBE and ABE into reconstitutable halves, is described in Levy et al. Nature Biomedical Engineering doi.org/10.1038/s41441-019-0505-5 (2019), which is incorporated herein by reference.

### Prime Editors

In some embodiments, a polynucleotide of the present invention described elsewhere herein can be modified using a prime editing system (See e.g. Anzalone et al. 2019. Nature. 576: 149-157). Like base editing systems, prime editing systems can be capable of targeted modification of a polynucleotide without generating double stranded breaks and does not require donor templates. Further prime editing systems can be capable of all 12 possible combination swaps. Prime editing can operate via a “search-and-replace” methodology and can mediate targeted insertions, deletions, all 12 possible base-to-base conversion, and combinations thereof. Generally, a prime editing system, as exemplified by PE1, PE2, and PE3 (Id.), can include a reverse transcriptase fused or otherwise coupled or associated with an RNA-programmable nickase, and a prime-editing extended guide RNA (pegRNA) to facility direct copying of genetic information from the extension on the pegRNA into the target polynucleotide. Embodiments that can be used with the present invention include these and variants thereof. Prime editing can have the advantage of lower off-target activity than traditional CRIPSR-Cas systems along with few byproducts and greater or similar efficiency as compared to traditional CRISPR-Cas systems.

In some embodiments, the prime editing guide molecule can specify both the target polynucleotide information (e.g. sequence) and contain a new polynucleotide cargo that replaces target polynucleotides. To initiate transfer from the guide molecule to the target polynucleotide, the PE system can nick the target polynucleotide at a target side to expose a 3′hydroxyl group, which can prime reverse transcription of an edit-encoding extension region of the guide molecule (e.g. a prime editing guide molecule or peg guide molecule) directly into the target site in the target polynucleotide. See e.g. Anzalone et al. 2019. Nature. 576: 149-157, particularly at FIGS. 1b, 1c, related discussion, and Supplementary discussion.

In some embodiments, a prime editing system can be composed of a Cas polypeptide having nickase activity, a reverse transcriptase, and a guide molecule. The Cas polypeptide can lack nuclease activity. The guide molecule can include a target binding sequence as well as a primer binding sequence and a template containing the edited polynucleotide sequence. The guide molecule, Cas polypeptide, and/or reverse transcriptase can be coupled together or otherwise associate with each other to form an effector complex and edit a target sequence. In some embodiments, the Cas polypeptide is a Class 2, Type V Cas polypeptide. In some embodiments, the Cas polypeptide is a Cas9 polypeptide (e.g. is a Cas9 nickase). In some embodiments, the Cas polypeptide is fused to the reverse transcriptase. In some embodiments, the Cas polypeptide is linked to the reverse transcriptase.

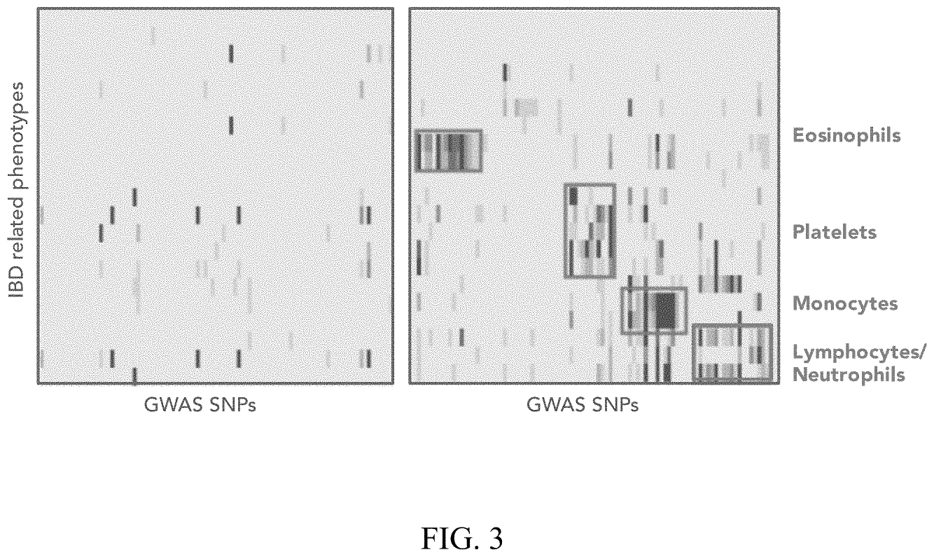

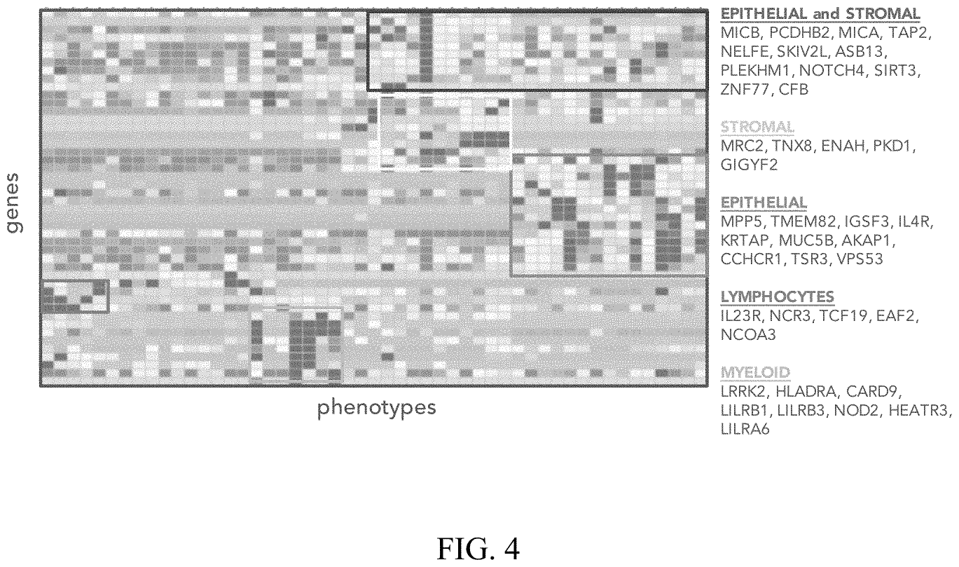

In some embodiments, the prime editing system can be a PE1 system or variant thereof, a PE2 system or variant thereof, or a PE3 (e.g. PE3, PE3b) system. See e.g., Anzalone et al. 2019. Nature. 576: 149-157, particularly at pgs. 2-3, FIGS. 2a, 3a-3f, 4a-4b, Extended data FIGS. 3a-3b, 4,

The peg guide molecule can be about 10 to about 200 or more nucleotides in length, such as 10 to/or 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, or 200 or more nucleotides in length. Optimization of the peg guide molecule can be accomplished as described in Anzalone et al. 2019. Nature. 576: 149-157, particularly at pg. 3, FIG. 2a-2b, and Extended Data FIGS. 5a-c.

### CRISPR Associated Transposase (CAST) Systems

In some embodiments, a polynucleotide of the present invention described elsewhere herein can be modified using a CRISPR Associated Transposase (“CAST”) system. CAST system can include a Cas protein that is catalytically inactive, or engineered to be catalytically active, and further comprises a transposase (or subunits thereof) that catalyze RNA-guided DNA transposition. Such systems are able to insert DNA sequences at a target site in a DNA molecule without relying on host cell repair machinery. CAST systems can be Class1 or Class 2 CAST systems. An example Class 1 system is described in Klompe et al. Nature, doi:10.1038/s41586-019-1323, which is in incorporated herein by reference. An example Class 2 system is described in Strecker et al. Science. 10/1126/science.aax9181 (2019), and PCT/US2019/066835 which are incorporated herein by reference.

### Guide Molecules

The CRISPR-Cas or Cas-Based system described herein can, in some embodiments, include one or more guide molecules. The terms guide molecule, guide sequence and guide polynucleotide, refer to polynucleotides capable of guiding Cas to a target genomic locus and are used interchangeably as in foregoing cited documents such as WO 2014/093622 (PCT/US2013/074667). In general, a guide sequence is any polynucleotide sequence having sufficient complementarity with a target polynucleotide sequence to hybridize with the target sequence and direct sequence-specific binding of a CRISPR complex to the target sequence. The guide molecule can be a polynucleotide.

The ability of a guide sequence (within a nucleic acid-targeting guide RNA) to direct sequence-specific binding of a nucleic acid-targeting complex to a target nucleic acid sequence may be assessed by any suitable assay. For example, the components of a nucleic acid-targeting CRISPR system sufficient to form a nucleic acid-targeting complex, including the guide sequence to be tested, may be provided to a host cell having the corresponding target nucleic acid sequence, such as by transfection with vectors encoding the components of the nucleic acid-targeting complex, followed by an assessment of preferential targeting (e.g., cleavage) within the target nucleic acid sequence, such as by Surveyor assay (Qui et al. 2004. BioTechniques. 36(4)702-707). Similarly, cleavage of a target nucleic acid sequence may be evaluated in a test tube by providing the target nucleic acid sequence, components of a nucleic acid-targeting complex, including the guide sequence to be tested and a control guide sequence different from the test guide sequence, and comparing binding or rate of cleavage at the target sequence between the test and control guide sequence reactions. Other assays are possible and will occur to those skilled in the art.

In some embodiments, the guide molecule is an RNA. The guide molecule(s) (also referred to interchangeably herein as guide polynucleotide and guide sequence) that are included in the CRISPR-Cas or Cas based system can be any polynucleotide sequence having sufficient complementarity with a target nucleic acid sequence to hybridize with the target nucleic acid sequence and direct sequence-specific binding of a nucleic acid-targeting complex to the target nucleic acid sequence. In some embodiments, the degree of complementarity, when optimally aligned using a suitable alignment algorithm, can be about or more than about 50%, 60%, 75%, 80%, 85%, 90%, 95%, 97.5%, 99%, or more. Optimal alignment may be determined with the use of any suitable algorithm for aligning sequences, non-limiting examples of which include the Smith-Waterman algorithm, the Needleman-Wunsch algorithm, algorithms based on the Burrows-Wheeler Transform (e.g., the Burrows Wheeler Aligner), Clustal W, Clustal X, BLAT, Novoalign (Novocraft Technologies; available at www.novocraft.com), ELAND (Illumina, San Diego, Calif.), SOAP (available at soap.genomics.org.cn), and Maq (available at maq.sourceforge.net).

A guide sequence, and hence a nucleic acid-targeting guide may be selected to target any target nucleic acid sequence. The target sequence may be DNA. The target sequence may be any RNA sequence. In some embodiments, the target sequence may be a sequence within an RNA molecule selected from the group consisting of messenger RNA (mRNA), pre-mRNA, ribosomal RNA (rRNA), transfer RNA (tRNA), micro-RNA (miRNA), small interfering RNA (siRNA), small nuclear RNA (snRNA), small nucleolar RNA (snoRNA), double stranded RNA (dsRNA), non-coding RNA (ncRNA), long non-coding RNA (lncRNA), and small cytoplasmatic RNA (scRNA). In some preferred embodiments, the target sequence may be a sequence within an RNA molecule selected from the group consisting of mRNA, pre-mRNA, and rRNA. In some preferred embodiments, the target sequence may be a sequence within an RNA molecule selected from the group consisting of ncRNA, and lncRNA. In some more preferred embodiments, the target sequence may be a sequence within an mRNA molecule or a pre-mRNA molecule.

In some embodiments, a nucleic acid-targeting guide is selected to reduce the degree secondary structure within the nucleic acid-targeting guide. In some embodiments, about or less than about 75%, 50%, 40%, 30%, 25%, 20%, 15%, 10%, 5%, 1%, or fewer of the nucleotides of the nucleic acid-targeting guide participate in self-complementary base pairing when optimally folded. Optimal folding may be determined by any suitable polynucleotide folding algorithm. Some programs are based on calculating the minimal Gibbs free energy. An example of one such algorithm is mFold, as described by Zuker and Stiegler (Nucleic Acids Res. 9 (1981), 133-148). Another example folding algorithm is the online webserver RNAfold, developed at Institute for Theoretical Chemistry at the University of Vienna, using the centroid structure prediction algorithm (see e.g., A. R. Gruber et al., 2008, Cell 106(1): 23-24; and PA Carr and GM Church, 2009, Nature Biotechnology 27(12): 1151-62).

In certain embodiments, a guide RNA or crRNA may comprise, consist essentially of, or consist of a direct repeat (DR) sequence and a guide sequence or spacer sequence. In certain embodiments, the guide RNA or crRNA may comprise, consist essentially of, or consist of a direct repeat sequence fused or linked to a guide sequence or spacer sequence. In certain embodiments, the direct repeat sequence may be located upstream (i.e., 5′) from the guide sequence or spacer sequence. In other embodiments, the direct repeat sequence may be located downstream (i.e., 3′) from the guide sequence or spacer sequence.

In certain embodiments, the crRNA comprises a stem loop, preferably a single stem loop. In certain embodiments, the direct repeat sequence forms a stem loop, preferably a single stem loop.

In certain embodiments, the spacer length of the guide RNA is from 15 to 35 nt. In certain embodiments, the spacer length of the guide RNA is at least 15 nucleotides. In certain embodiments, the spacer length is from 15 to 17 nt, e.g., 15, 16, or 17 nt, from 17 to 20 nt, e.g., 17, 18, 19, or 20 nt, from 20 to 24 nt, e.g., 20, 21, 22, 23, or 24 nt, from 23 to 25 nt, e.g., 23, 24, or 25 nt, from 24 to 27 nt, e.g., 24, 25, 26, or 27 nt, from 27 to 30 nt, e.g., 27, 28, 29, or 30 nt, from 30 to 35 nt, e.g., 30, 31, 32, 33, 34, or 35 nt, or 35 nt or longer.

The “tracrRNA” sequence or analogous terms includes any polynucleotide sequence that has sufficient complementarity with a crRNA sequence to hybridize. In some embodiments, the degree of complementarity between the tracrRNA sequence and crRNA sequence along the length of the shorter of the two when optimally aligned is about or more than about 25%, 30%, 40%, 50%, 60%, 70%, 80%, 90%, 95%, 97.5%, 99%, or higher. In some embodiments, the tracr sequence is about or more than about 5, 6, 7, 8, 9, 10, 11, 12, 13, 14, 15, 16, 17, 18, 19, 20, 25, 30, 40, 50, or more nucleotides in length. In some embodiments, the tracr sequence and crRNA sequence are contained within a single transcript, such that hybridization between the two produces a transcript having a secondary structure, such as a hairpin.

In general, degree of complementarity is with reference to the optimal alignment of the sca sequence and tracr sequence, along the length of the shorter of the two sequences. Optimal alignment may be determined by any suitable alignment algorithm, and may further account for secondary structures, such as self-complementarity within either the sca sequence or tracr sequence. In some embodiments, the degree of complementarity between the tracr sequence and sca sequence along the length of the shorter of the two when optimally aligned is about or more than about 25%, 30%, 40%, 50%, 60%, 70%, 80%, 90%, 95%, 97.5%, 99%, or higher.

In some embodiments, the degree of complementarity between a guide sequence and its corresponding target sequence can be about or more than about 50%, 60%, 75%, 80%, 85%, 90%, 95%, 97.5%, 99%, or 100%; a guide or RNA or sgRNA can be about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length; or guide or RNA or sgRNA can be less than about 75, 50, 45, 40, 35, 30, 25, 20, 15, 12, or fewer nucleotides in length; and tracr RNA can be 30 or 50 nucleotides in length. In some embodiments, the degree of complementarity between a guide sequence and its corresponding target sequence is greater than 94.5% or 95% or 95.5% or 96% or 96.5% or 97% or 97.5% or 98% or 98.5% or 99% or 99.5% or 99.9%, or 100%. Off target is less than 100% or 99.9% or 99.5% or 99% or 99% or 98.5% or 98% or 97.5% or 97% or 96.5% or 96% or 95.5% or 95% or 94.5% or 94% or 93% or 92% or 91% or 90% or 89% or 88% or 87% or 86% or 85% or 84% or 83% or 82% or 81% or 80% complementarity between the sequence and the guide, with it advantageous that off target is 100% or 99.9% or 99.5% or 99% or 99% or 98.5% or 98% or 97.5% or 97% or 96.5% or 96% or 95.5% or 95% or 94.5% complementarity between the sequence and the guide.

In some embodiments according to the invention, the guide RNA (capable of guiding Cas to a target locus) may comprise (1) a guide sequence capable of hybridizing to a genomic target locus in the eukaryotic cell; (2) a tracr sequence; and (3) a tracr mate sequence. All (1) to (3) may reside in a single RNA, i.e., an sgRNA (arranged in a 5′ to 3′ orientation), or the tracr RNA may be a different RNA than the RNA containing the guide and tracr sequence. The tracr hybridizes to the tracr mate sequence and directs the CRISPR/Cas complex to the target sequence. Where the tracr RNA is on a different RNA than the RNA containing the guide and tracr sequence, the length of each RNA may be optimized to be shortened from their respective native lengths, and each may be independently chemically modified to protect from degradation by cellular RNase or otherwise increase stability.

Many modifications to guide sequences are known in the art and are further contemplated within the context of this invention. Various modifications may be used to increase the specificity of binding to the target sequence and/or increase the activity of the Cas protein and/or reduce off-target effects. Example guide sequence modifications are described in PCT US2019/045582, specifically paragraphs [0178]-[0333]. which is incorporated herein by reference.

### Target Sequences, PAMs, and PFSs

### Target Sequences

In the context of formation of a CRISPR complex, “target sequence” refers to a sequence to which a guide sequence is designed to have complementarity, where hybridization between a target sequence and a guide sequence promotes the formation of a CRISPR complex. A target sequence may comprise RNA polynucleotides. The term “target RNA” refers to an RNA polynucleotide being or comprising the target sequence. In other words, the target polynucleotide can be a polynucleotide or a part of a polynucleotide to which a part of the guide sequence is designed to have complementarity with and to which the effector function mediated by the complex comprising the CRISPR effector protein and a guide molecule is to be directed. In some embodiments, a target sequence is located in the nucleus or cytoplasm of a cell.

The guide sequence can specifically bind a target sequence in a target polynucleotide. The target polynucleotide may be DNA. The target polynucleotide may be RNA. The target polynucleotide can have one or more (e.g., 1, 2, 3, 4, 5, 6, 7, 8, 9, 10, etc. or more) target sequences. The target polynucleotide can be on a vector. The target polynucleotide can be genomic DNA. The target polynucleotide can be episomal. Other forms of the target polynucleotide are described elsewhere herein.

The target sequence may be DNA. The target sequence may be any RNA sequence. In some embodiments, the target sequence may be a sequence within an RNA molecule selected from the group consisting of messenger RNA (mRNA), pre-mRNA, ribosomal RNA (rRNA), transfer RNA (tRNA), micro-RNA (miRNA), small interfering RNA (siRNA), small nuclear RNA (snRNA), small nucleolar RNA (snoRNA), double stranded RNA (dsRNA), non-coding RNA (ncRNA), long non-coding RNA (lncRNA), and small cytoplasmatic RNA (scRNA). In some preferred embodiments, the target sequence (also referred to herein as a target polynucleotide) may be a sequence within an RNA molecule selected from the group consisting of mRNA, pre-mRNA, and rRNA. In some preferred embodiments, the target sequence may be a sequence within an RNA molecule selected from the group consisting of ncRNA, and lncRNA. In some more preferred embodiments, the target sequence may be a sequence within an mRNA molecule or a pre-mRNA molecule.

### PAM and PFS Elements

PAM elements are sequences that can be recognized and bound by Cas proteins. Cas proteins/effector complexes can then unwind the dsDNA at a position adjacent to the PAM element. It will be appreciated that Cas proteins and systems that include them that target RNA do not require PAM sequences (Marraffini et al. 2010. Nature. 463:568-571). Instead, many rely on PFSs, which are discussed elsewhere herein. In certain embodiments, the target sequence should be associated with a PAM (protospacer adjacent motif) or PFS (protospacer flanking sequence or site), that is, a short sequence recognized by the CRISPR complex. Depending on the nature of the CRISPR-Cas protein, the target sequence should be selected, such that its complementary sequence in the DNA duplex (also referred to herein as the non-target sequence) is upstream or downstream of the PAM. In the embodiments, the complementary sequence of the target sequence is downstream or 3′ of the PAM or upstream or 5′ of the PAM. The precise sequence and length requirements for the PAM differ depending on the Cas protein used, but PAMs are typically 2-5 base pair sequences adjacent the protospacer (that is, the target sequence). Examples of the natural PAM sequences for different Cas proteins are provided herein below and the skilled person will be able to identify further PAM sequences for use with a given Cas protein.

The ability to recognize different PAM sequences depends on the Cas polypeptide(s) included in the system. See e.g., Gleditzsch et al. 2019. RNA Biology. 16(4):504-517. Table A below shows several Cas polypeptides and the PAM sequence they recognize.

Table A
Example PAM Sequences

| Cas Protein | PAM Sequence |
|---|---|
| SpCas9 | NGG/NRG |
| SaCas9 | NGRRT or NGRRN |
| NmeCas9 | NNNNGATT |
| CjCas9 | NNNNRYAC |
| StCas9 | NNAGAAW |
| Cas12a Cpf1 (including LbCpf1 | TTTV |
| and AsCpf1) | |
| Cas12b (C2c1) | TTT, TTA, and TTC |
| Cas12c (C2c3) | TA |
| Cas12d (CasY) | TA |
| Cas12e (CasX) | 5′-TTCN-3′ |

In a preferred embodiment, the CRISPR effector protein may recognize a 3′ PAM. In certain embodiments, the CRISPR effector protein may recognize a 3′ PAM which is 5′H, wherein H is A, C or U.

Further, engineering of the PAM Interacting (PI) domain on the Cas protein may allow programing of PAM specificity, improve target site recognition fidelity, and increase the versatility of the CRISPR-Cas protein, for example as described for Cas9 in Kleinstiver B P et al. Engineered CRISPR-Cas9 nucleases with altered PAM specificities. Nature. 2015 Jul. 23; 523(7561):481-5. doi: 10.1038/nature14592. As further detailed herein, the skilled person will understand that Cas13 proteins may be modified analogously. Gao et al, “Engineered Cpfl Enzymes with Altered PAM Specificities,” bioRxiv 091611; doi: http://dx.doi.org/10.1101/091611 (Dec. 4, 2016). Doench et al. created a pool of sgRNAs, tiling across all possible target sites of a panel of six endogenous mouse and three endogenous human genes and quantitatively assessed their ability to produce null alleles of their target gene by antibody staining and flow cytometry. The authors showed that optimization of the PAM improved activity and also provided an on-line tool for designing sgRNAs.

PAM sequences can be identified in a polynucleotide using an appropriate design tool, which are commercially available as well as online. Such freely available tools include, but are not limited to, CRISPRFinder and CRISPRTarget. Mojica et al. 2009. Microbiol. 155(Pt. 3):733-740; Atschul et al. 1990. J. Mol. Biol. 215:403-410; Biswass et al. 2013 RNA Biol. 10:817-827; and Grissa et al. 2007. Nucleic Acid Res. 35:W52-57. Experimental approaches to PAM identification can include, but are not limited to, plasmid depletion assays (Jiang et al. 2013. Nat. Biotechnol. 31:233-239; Esvelt et al. 2013. Nat. Methods. 10:1116-1121; Kleinstiver et al. 2015. Nature. 523:481-485), screened by a high-throughput in vivo model called PAM-SCNAR (Pattanayak et al. 2013. Nat. Biotechnol. 31:839-843 and Leenay et al. 2016.Mol. Cell. 16:253), and negative screening (Zetsche et al. 2015. Cell. 163:759-771).

As previously mentioned, CRISPR-Cas systems that target RNA do not typically rely on PAM sequences. Instead such systems typically recognize protospacer flanking sites (PFSs) instead of PAMs Thus, Type VI CRISPR-Cas systems typically recognize protospacer flanking sites (PFSs) instead of PAMs. PFSs represents an analogue to PAMs for RNA targets. Type VI CRISPR-Cas systems employ a Cas13. Some Cas13 proteins analyzed to date, such as Cas13a (C2c2) identified from Leptotrichia shahii (LShCAs13a) have a specific discrimination against G at the 3′ end of the target RNA. The presence of a C at the corresponding crRNA repeat site can indicate that nucleotide pairing at this position is rejected. However, some Cas13 proteins (e.g., LwaCAs13a and PspCas13b) do not seem to have a PFS preference. See e.g., Gleditzsch et al. 2019. RNA Biology. 16(4):504-517.

Some Type VI proteins, such as subtype B, have 5′-recognition of D (G, T, A) and a 3′-motif requirement of NAN or NNA. One example is the Cas13b protein identified in Bergeyella zoohelcum (BzCas13b). See e.g., Gleditzsch et al. 2019. RNA Biology. 16(4):504-517.

Overall Type VI CRISPR-Cas systems appear to have less restrictive rules for substrate (e.g., target sequence) recognition than those that target DNA (e.g., Type V and type II).

### Zinc Finger Nucleases

In some embodiments, the polynucleotide is modified using a Zinc Finger nuclease or system thereof. One type of programmable DNA-binding domain is provided by artificial zinc-finger (ZF) technology, which involves arrays of ZF modules to target new DNA-binding sites in the genome. Each finger module in a ZF array targets three DNA bases. A customized array of individual zinc finger domains is assembled into a ZF protein (ZFP).

ZFPs can comprise a functional domain. The first synthetic zinc finger nucleases (ZFNs) were developed by fusing a ZF protein to the catalytic domain of the Type IIS restriction enzyme FokI. (Kim, Y. G. et al., 1994, Chimeric restriction endonuclease, Proc. Natl. Acad. Sci. U.S.A. 91, 883-887; Kim, Y. G. et al., 1996, Hybrid restriction enzymes: zinc finger fusions to FokI cleavage domain. Proc. Natl. Acad. Sci. U.S.A. 93, 1156-1160). Increased cleavage specificity can be attained with decreased off target activity by use of paired ZFN heterodimers, each targeting different nucleotide sequences separated by a short spacer. (Doyon, Y. et al., 2011, Enhancing zinc-finger-nuclease activity with improved obligate heterodimeric architectures. Nat. Methods 8, 74-79). ZFPs can also be designed as transcription activators and repressors and have been used to target many genes in a wide variety of organisms. Exemplary methods of genome editing using ZFNs can be found for example in U.S. Pat. Nos. 6,534,261, 6,607,882, 6,746,838, 6,794,136, 6,824,978, 6,866,997, 6,933,113, 6,979,539, 7,013,219, 7,030,215, 7,220,719, 7,241,573, 7,241,574, 7,585,849, 7,595,376, 6,903,185, and 6,479,626, all of which are specifically incorporated by reference.

### TALE Nucleases

In some embodiments, a TALE nuclease or TALE nuclease system can be used to modify a polynucleotide. In some embodiments, the methods provided herein use isolated, non-naturally occurring, recombinant or engineered DNA binding proteins that comprise TALE monomers or TALE monomers or half monomers as a part of their organizational structure that enable the targeting of nucleic acid sequences with improved efficiency and expanded specificity.

Naturally occurring TALEs or “wild type TALEs” are nucleic acid binding proteins secreted by numerous species of proteobacteria. TALE polypeptides contain a nucleic acid binding domain composed of tandem repeats of highly conserved monomer polypeptides that are predominantly 33, 34 or 35 amino acids in length and that differ from each other mainly in amino acid positions 12 and 13. In advantageous embodiments the nucleic acid is DNA. As used herein, the term “polypeptide monomers”, “TALE monomers” or “monomers” will be used to refer to the highly conserved repetitive polypeptide sequences within the TALE nucleic acid binding domain and the term “repeat variable di-residues” or “RVD” will be used to refer to the highly variable amino acids at positions 12 and 13 of the polypeptide monomers. As provided throughout the disclosure, the amino acid residues of the RVD are depicted using the IUPAC single letter code for amino acids. A general representation of a TALE monomer which is comprised within the DNA binding domain is X₁₋₁₁-(X₁₂X₁₃)-X₁₄₋₃₃ or 34 or 35, where the subscript indicates the amino acid position and X represents any amino acid. X₁₂X₁₃ indicate the RVDs. In some polypeptide monomers, the variable amino acid at position 13 is missing or absent and in such monomers, the RVD consists of a single amino acid. In such cases the RVD may be alternatively represented as X*, where X represents X₁₂ and (*) indicates that X₁₃ is absent. The DNA binding domain comprises several repeats of TALE monomers and this may be represented as (X₁₋₁₁-(X₁₂X₁₃)-X₁₄₋₃₃ or 34 or 35)_{z}, where in an advantageous embodiment, z is at least 5 to 40. In a further advantageous embodiment, z is at least 10 to 26.

The TALE monomers can have a nucleotide binding affinity that is determined by the identity of the amino acids in its RVD. For example, polypeptide monomers with an RVD of NI can preferentially bind to adenine (A), monomers with an RVD of NG can preferentially bind to thymine (T), monomers with an RVD of HD can preferentially bind to cytosine (C) and monomers with an RVD of NN can preferentially bind to both adenine (A) and guanine (G). In some embodiments, monomers with an RVD of IG can preferentially bind to T. Thus, the number and order of the polypeptide monomer repeats in the nucleic acid binding domain of a TALE determines its nucleic acid target specificity. In some embodiments, monomers with an RVD of NS can recognize all four base pairs and can bind to A, T, G or C. The structure and function of TALEs is further described in, for example, Moscou et al., Science 326:1501 (2009); Boch et al., Science 326:1509-1512 (2009); and Zhang et al., Nature Biotechnology 29:149-153 (2011).

The polypeptides used in methods of the invention can be isolated, non-naturally occurring, recombinant or engineered nucleic acid-binding proteins that have nucleic acid or DNA binding regions containing polypeptide monomer repeats that are designed to target specific nucleic acid sequences.

As described herein, polypeptide monomers having an RVD of HN or NH preferentially bind to guanine and thereby allow the generation of TALE polypeptides with high binding specificity for guanine containing target nucleic acid sequences. In some embodiments, polypeptide monomers having RVDs RN, NN, NK, SN, NH, KN, HN, NQ, HH, RG, KH, RH and SS can preferentially bind to guanine. In some embodiments, polypeptide monomers having RVDs RN, NK, NQ, HH, KH, RH, SS and SN can preferentially bind to guanine and can thus allow the generation of TALE polypeptides with high binding specificity for guanine containing target nucleic acid sequences. In some embodiments, polypeptide monomers having RVDs HH, KH, NH, NK, NQ, RH, RN and SS can preferentially bind to guanine and thereby allow the generation of TALE polypeptides with high binding specificity for guanine containing target nucleic acid sequences. In some embodiments, the RVDs that have high binding specificity for guanine are RN, NH RH and KH. Furthermore, polypeptide monomers having an RVD of NV can preferentially bind to adenine and guanine. In some embodiments, monomers having RVDs of H*, HA, KA, N*, NA, NC, NS, RA, and S* bind to adenine, guanine, cytosine and thymine with comparable affinity.

The predetermined N-terminal to C-terminal order of the one or more polypeptide monomers of the nucleic acid or DNA binding domain determines the corresponding predetermined target nucleic acid sequence to which the polypeptides of the invention will bind. As used herein the monomers and at least one or more half monomers are “specifically ordered to target” the genomic locus or gene of interest. In plant genomes, the natural TALE-binding sites always begin with a thymine (T), which may be specified by a cryptic signal within the non-repetitive N-terminus of the TALE polypeptide; in some cases, this region may be referred to as repeat 0. In animal genomes, TALE binding sites do not necessarily have to begin with a thymine (T) and polypeptides of the invention may target DNA sequences that begin with T, A, G or C. The tandem repeat of TALE monomers always ends with a half-length repeat or a stretch of sequence that may share identity with only the first 20 amino acids of a repetitive full-length TALE monomer and this half repeat may be referred to as a half-monomer. Therefore, it follows that the length of the nucleic acid or DNA being targeted is equal to the number of full monomers plus two.

As described in Zhang et al., Nature Biotechnology 29:149-153 (2011), TALE polypeptide binding efficiency may be increased by including amino acid sequences from the “capping regions” that are directly N-terminal or C-terminal of the DNA binding region of naturally occurring TALEs into the engineered TALEs at positions N-terminal or C-terminal of the engineered TALE DNA binding region. Thus, in certain embodiments, the TALE polypeptides described herein further comprise an N-terminal capping region and/or a C-terminal capping region.

An exemplary amino acid sequence of a N-terminal capping region is:

(SEQ ID NO: 15)
M D P I R S R T P S P A R E L L S G P Q P D G V Q
P T A D R G V S P P A G G P L D G L P A R R T M S
R T R L P S P P A P S P A F S A D S F S D L L R Q
F D P S L F N T S L F D S L P P F G A H H T E A A
T G E W D E V Q S G L R A A D A P P P T M R V A V
T A A R P P R A K P A P R R R A A Q P S D A S P A
A Q V D L R T L G Y S Q Q Q Q E K I K P K V R S T
V A Q H H E A L V G H G F T H A H I V A L S Q H P
A A L G T V A V K Y Q D M I A A L P E A T H E A I
V G V G K Q W S G A R A L E A L L T V A G E L R G
P P L Q L D T G Q L L K I A K R G G V T A V E A V
H A W R N A L T G A P L N

An exemplary amino acid sequence of a C-terminal capping region is:

(SEQ ID NO: 16)
R P A L E S I V A Q L S R P D P A L A A L T N D H
L V A L A C L G G R P A L D A V K K G L P H A P A
L I K R T N R R I P E R T S H R V A D H A Q V V R
V L G F F Q C H S H P A Q A F D D A M T Q F G M S
R H G L L Q L F R R V G V T E L E A R S G T L P P
A S Q R W D R I L Q A S G M K R A K P S P T S T Q
T P D Q A S L H A F A D S L E R D L D A P S P M H
E G D Q T R A S

As used herein the predetermined “N-terminus” to “C terminus” orientation of the N-terminal capping region, the DNA binding domain comprising the repeat TALE monomers and the C-terminal capping region provide structural basis for the organization of different domains in the d-TALEs or polypeptides of the invention.

The entire N-terminal and/or C-terminal capping regions are not necessary to enhance the binding activity of the DNA binding region. Therefore, in certain embodiments, fragments of the N-terminal and/or C-terminal capping regions are included in the TALE polypeptides described herein.

In certain embodiments, the TALE polypeptides described herein contain a N-terminal capping region fragment that included at least 10, 20, 30, 40, 50, 54, 60, 70, 80, 87, 90, 94, 100, 102, 110, 117, 120, 130, 140, 147, 150, 160, 170, 180, 190, 200, 210, 220, 230, 240, 250, 260 or 270 amino acids of an N-terminal capping region. In certain embodiments, the N-terminal capping region fragment amino acids are of the C-terminus (the DNA-binding region proximal end) of an N-terminal capping region. As described in Zhang et al., Nature Biotechnology 29:149-153 (2011), N-terminal capping region fragments that include the C-terminal 240 amino acids enhance binding activity equal to the full length capping region, while fragments that include the C-terminal 147 amino acids retain greater than 80% of the efficacy of the full length capping region, and fragments that include the C-terminal 117 amino acids retain greater than 50% of the activity of the full-length capping region.

In some embodiments, the TALE polypeptides described herein contain a C-terminal capping region fragment that included at least 6, 10, 20, 30, 37, 40, 50, 60, 68, 70, 80, 90, 100, 110, 120, 127, 130, 140, 150, 155, 160, 170, 180 amino acids of a C-terminal capping region. In certain embodiments, the C-terminal capping region fragment amino acids are of the N-terminus (the DNA-binding region proximal end) of a C-terminal capping region. As described in Zhang et al., Nature Biotechnology 29:149-153 (2011), C-terminal capping region fragments that include the C-terminal 68 amino acids enhance binding activity equal to the full-length capping region, while fragments that include the C-terminal 20 amino acids retain greater than 50% of the efficacy of the full-length capping region.

In certain embodiments, the capping regions of the TALE polypeptides described herein do not need to have identical sequences to the capping region sequences provided herein. Thus, in some embodiments, the capping region of the TALE polypeptides described herein have sequences that are at least 50%, 60%, 70%, 80%, 85%, 90%, 91%, 92%, 93%, 94%, 95%, 96%, 97%, 98% or 99% identical or share identity to the capping region amino acid sequences provided herein. Sequence identity is related to sequence homology. Homology comparisons may be conducted by eye, or more usually, with the aid of readily available sequence comparison programs. These commercially available computer programs may calculate percent (%) homology between two or more sequences and may also calculate the sequence identity shared by two or more amino acid or nucleic acid sequences. In some preferred embodiments, the capping region of the TALE polypeptides described herein have sequences that are at least 95% identical or share identity to the capping region amino acid sequences provided herein.

Sequence homologies can be generated by any of a number of computer programs known in the art, which include but are not limited to BLAST or FASTA. Suitable computer programs for carrying out alignments like the GCG Wisconsin Bestfit package may also be used. Once the software has produced an optimal alignment, it is possible to calculate % homology, preferably % sequence identity. The software typically does this as part of the sequence comparison and generates a numerical result.

In some embodiments described herein, the TALE polypeptides of the invention include a nucleic acid binding domain linked to the one or more effector domains. The terms “effector domain” or “regulatory and functional domain” refer to a polypeptide sequence that has an activity other than binding to the nucleic acid sequence recognized by the nucleic acid binding domain. By combining a nucleic acid binding domain with one or more effector domains, the polypeptides of the invention may be used to target the one or more functions or activities mediated by the effector domain to a particular target DNA sequence to which the nucleic acid binding domain specifically binds.

In some embodiments of the TALE polypeptides described herein, the activity mediated by the effector domain is a biological activity. For example, in some embodiments the effector domain is a transcriptional inhibitor (i.e., a repressor domain), such as an mSin interaction domain (SID). SID4X domain or a Krüppel-associated box (KRAB) or fragments of the KRAB domain. In some embodiments the effector domain is an enhancer of transcription (i.e. an activation domain), such as the VP16, VP64 or p65 activation domain. In some embodiments, the nucleic acid binding is linked, for example, with an effector domain that includes but is not limited to a transposase, integrase, recombinase, resolvase, invertase, protease, DNA methyltransferase, DNA demethylase, histone acetylase, histone deacetylase, nuclease, transcriptional repressor, transcriptional activator, transcription factor recruiting, protein nuclear-localization signal or cellular uptake signal.

In some embodiments, the effector domain is a protein domain which exhibits activities which include but are not limited to transposase activity, integrase activity, recombinase activity, resolvase activity, invertase activity, protease activity, DNA methyltransferase activity, DNA demethylase activity, histone acetylase activity, histone deacetylase activity, nuclease activity, nuclear-localization signaling activity, transcriptional repressor activity, transcriptional activator activity, transcription factor recruiting activity, or cellular uptake signaling activity. Other preferred embodiments of the invention may include any combination of the activities described herein.

### Meganucleases

In some embodiments, a meganuclease or system thereof can be used to modify a polynucleotide. Meganucleases, which are endodeoxyribonucleases characterized by a large recognition site (double-stranded DNA sequences of 12 to 40 base pairs). Exemplary methods for using meganucleases can be found in U.S. Pat. Nos. 8,163,514, 8,133,697, 8,021,867, 8,119,361, 8,119,381, 8,124,369, and 8,129,134, which are specifically incorporated by reference.

### Sequences Related to Nucleus Targeting and Transportation

In some embodiments, one or more components (e.g., the Cas protein and/or deaminase, Zn Finger protein, TALE, or meganuclease) in the composition for engineering cells may comprise one or more sequences related to nucleus targeting and transportation. Such sequence may facilitate the one or more components in the composition for targeting a sequence within a cell. In order to improve targeting of the CRISPR-Cas protein and/or the nucleotide deaminase protein or catalytic domain thereof used in the methods of the present disclosure to the nucleus, it may be advantageous to provide one or both of these components with one or more nuclear localization sequences (NLSs).

In some embodiments, the NLSs used in the context of the present disclosure are heterologous to the proteins. Non-limiting examples of NLSs include an NLS sequence derived from: the NLS of the SV40 virus large T-antigen, having the amino acid sequence PKKKRKV (SEQ ID No. 17) or PKKKRKVEAS (SEQ ID No. 18); the NLS from nucleoplasmin (e.g., the nucleoplasmin bipartite NLS with the sequence KRPAATKKAGQAKKKK (SEQ ID No. 19)); the c-myc NLS having the amino acid sequence PAAKRVKLD (SEQ ID No. 20) or RQRRNELKRSP (SEQ ID No. 21); the hRNPA1 M9 NLS having the sequence NQSSNFGPMKGGNFGGRSSGPYGGGGQYFAKPRNQGGY (SEQ ID No. 22); the sequence RMRIZFKNKGKDTAELRRRRVEVSVELRKAKKDEQILKRRNV (SEQ ID No. 23) of the IBB domain from importin-alpha; the sequences VSRKRPRP (SEQ ID No. 24) and PPKKARED (SEQ ID No. 25) of the myoma T protein; the sequence PQPKKKPL (SEQ ID No. 26) of human p53; the sequence SALIKKKKKMAP (SEQ ID No. 27) of mouse c-abl IV; the sequences DRLRR (SEQ ID No. 28) and PKQKKRK (SEQ ID No. 29) of the influenza virus NS1; the sequence RKLKKKIKKL (SEQ ID No. 30) of the Hepatitis virus delta antigen; the sequence REKKKFLKRR (SEQ ID No. 31) of the mouse Mx1 protein; the sequence KRKGDEVDGVDEVAKKKSKK (SEQ ID No. 32) of the human poly(ADP-ribose) polymerase; and the sequence RKCLQAGMNLEARKTKK (SEQ ID No. 33) of the steroid hormone receptors (human) glucocorticoid. In general, the one or more NLSs are of sufficient strength to drive accumulation of the DNA-targeting Cas protein in a detectable amount in the nucleus of a eukaryotic cell. In general, strength of nuclear localization activity may derive from the number of NLSs in the CRISPR-Cas protein, the particular NLS(s) used, or a combination of these factors. Detection of accumulation in the nucleus may be performed by any suitable technique. For example, a detectable marker may be fused to the nucleic acid-targeting protein, such that location within a cell may be visualized, such as in combination with a means for detecting the location of the nucleus (e.g., a stain specific for the nucleus such as DAPI). Cell nuclei may also be isolated from cells, the contents of which may then be analyzed by any suitable process for detecting protein, such as immunohistochemistry, Western blot, or enzyme activity assay. Accumulation in the nucleus may also be determined indirectly, such as by an assay for the effect of nucleic acid-targeting complex formation (e.g., assay for deaminase activity) at the target sequence, or assay for altered gene expression activity affected by DNA-targeting complex formation and/or DNA-targeting), as compared to a control not exposed to the CRISPR-Cas protein and deaminase protein, or exposed to a CRISPR-Cas and/or deaminase protein lacking the one or more NLSs.

The CRISPR-Cas and/or nucleotide deaminase proteins may be provided with 1 or more, such as with, 2, 3, 4, 5, 6, 7, 8, 9, 10, or more heterologous NLSs. In some embodiments, the proteins comprises about or more than about 1, 2, 3, 4, 5, 6, 7, 8, 9, 10, or more NLSs at or near the amino-terminus, about or more than about 1, 2, 3, 4, 5, 6, 7, 8, 9, 10, or more NLSs at or near the carboxy-terminus, or a combination of these (e.g., zero or at least one or more NLS at the amino-terminus and zero or at one or more NLS at the carboxy terminus). When more than one NLS is present, each may be selected independently of the others, such that a single NLS may be present in more than one copy and/or in combination with one or more other NLSs present in one or more copies. In some embodiments, an NLS is considered near the N- or C-terminus when the nearest amino acid of the NLS is within about 1, 2, 3, 4, 5, 10, 15, 20, 25, 30, 40, 50, or more amino acids along the polypeptide chain from the N- or C-terminus. In preferred embodiments of the CRISPR-Cas proteins, an NLS attached to the C-terminal of the protein.

In certain embodiments, the CRISPR-Cas protein and the deaminase protein are delivered to the cell or expressed within the cell as separate proteins. In these embodiments, each of the CRISPR-Cas and deaminase protein can be provided with one or more NLSs as described herein. In certain embodiments, the CRISPR-Cas and deaminase proteins are delivered to the cell or expressed with the cell as a fusion protein. In these embodiments one or both of the CRISPR-Cas and deaminase protein is provided with one or more NLSs. Where the nucleotide deaminase is fused to an adaptor protein (such as MS2) as described above, the one or more NLS can be provided on the adaptor protein, provided that this does not interfere with aptamer binding. In particular embodiments, the one or more NLS sequences may also function as linker sequences between the nucleotide deaminase and the CRISPR-Cas protein.

In certain embodiments, guides of the disclosure comprise specific binding sites (e.g. aptamers) for adapter proteins, which may be linked to or fused to an nucleotide deaminase or catalytic domain thereof. When such a guide forms a CRISPR complex (e.g., CRISPR-Cas protein binding to guide and target) the adapter proteins bind and, the nucleotide deaminase or catalytic domain thereof associated with the adapter protein is positioned in a spatial orientation which is advantageous for the attributed function to be effective.

The skilled person will understand that modifications to the guide which allow for binding of the adapter+nucleotide deaminase, but not proper positioning of the adapter+nucleotide deaminase (e.g. due to steric hindrance within the three dimensional structure of the CRISPR complex) are modifications which are not intended. The one or more modified guide may be modified at the tetra loop, the stem loop 1, stem loop 2, or stem loop 3, as described herein, preferably at either the tetra loop or stem loop 2, and in some cases at both the tetra loop and stem loop 2.

In some embodiments, a component (e.g., the dead Cas protein, the nucleotide deaminase protein or catalytic domain thereof, or a combination thereof) in the systems may comprise one or more nuclear export signals (NES), one or more nuclear localization signals (NLS), or any combinations thereof. In some cases, the NES may be an HIV Rev NES. In certain cases, the NES may be MAPK NES. When the component is a protein, the NES or NLS may be at the C terminus of component. Alternatively or additionally, the NES or NLS may be at the N terminus of component. In some examples, the Cas protein and optionally said nucleotide deaminase protein or catalytic domain thereof comprise one or more heterologous nuclear export signal(s) (NES(s)) or nuclear localization signal(s) (NLS(s)), preferably an HIV Rev NES or MAPK NES, preferably C-terminal.

### Templates

In some embodiments, the composition for engineering cells comprise a template, e.g., a recombination template. A template may be a component of another vector as described herein, contained in a separate vector, or provided as a separate polynucleotide. In some embodiments, a recombination template is designed to serve as a template in homologous recombination, such as within or near a target sequence nicked or cleaved by a nucleic acid-targeting effector protein as a part of a nucleic acid-targeting complex.

In an embodiment, the template nucleic acid alters the sequence of the target position. In an embodiment, the template nucleic acid results in the incorporation of a modified, or non-naturally occurring base into the target nucleic acid.

The template sequence may undergo a breakage mediated or catalyzed recombination with the target sequence. In an embodiment, the template nucleic acid may include sequence that corresponds to a site on the target sequence that is cleaved by a Cas protein mediated cleavage event. In an embodiment, the template nucleic acid may include sequence that corresponds to both, a first site on the target sequence that is cleaved in a first Cas protein mediated event, and a second site on the target sequence that is cleaved in a second Cas protein mediated event.

In certain embodiments, the template nucleic acid can include sequence which results in an alteration in the coding sequence of a translated sequence, e.g., one which results in the substitution of one amino acid for another in a protein product, e.g., transforming a mutant allele into a wild type allele, transforming a wild type allele into a mutant allele, and/or introducing a stop codon, insertion of an amino acid residue, deletion of an amino acid residue, or a nonsense mutation. In certain embodiments, the template nucleic acid can include sequence which results in an alteration in a non-coding sequence, e.g., an alteration in an exon or in a 5′ or 3′ non-translated or non-transcribed region. Such alterations include an alteration in a control element, e.g., a promoter, enhancer, and an alteration in a cis-acting or trans-acting control element.

A template nucleic acid having homology with a target position in a target gene may be used to alter the structure of a target sequence. The template sequence may be used to alter an unwanted structure, e.g., an unwanted or mutant nucleotide. The template nucleic acid may include sequence which, when integrated, results in: decreasing the activity of a positive control element; increasing the activity of a positive control element; decreasing the activity of a negative control element; increasing the activity of a negative control element; decreasing the expression of a gene; increasing the expression of a gene; increasing resistance to a disorder or disease; increasing resistance to viral entry; correcting a mutation or altering an unwanted amino acid residue conferring, increasing, abolishing or decreasing a biological property of a gene product, e.g., increasing the enzymatic activity of an enzyme, or increasing the ability of a gene product to interact with another molecule.

The template nucleic acid may include sequence which results in: a change in sequence of 1, 2, 3, 4, 5, 6, 7, 8, 9, 10, 11, 12 or more nucleotides of the target sequence.

A template polynucleotide may be of any suitable length, such as about or more than about 10, 15, 20, 25, 50, 75, 100, 150, 200, 500, 1000, or more nucleotides in length. In an embodiment, the template nucleic acid may be 20+/−10, 30+/−10, 40+/−10, 50+/−10, 60+/−10, 70+/−10, 80+/−10, 90+/−10, 100+/−10, 110+/−10, 120+/−10, 130+/−10, 140+/−10, 150+/−10, 160+/−10, 170+/−10, 180+/−10, 190+/−10, 200+/−10, 210+/−10, of 220+/−10 nucleotides in length. In an embodiment, the template nucleic acid may be 30+/−20, 40+/−20, 50+/−20, 60+/−20, 70+/−20, 80+/−20, 90+/−20, 100+/−20, 110+/−20, 120+/−20, 130+/−20, 140+/−20, 150+/−20, 160+/−20, 170+/−20, 180+/−20, 190+/−20, 200+/−20, 210+/−20, of 220+/−20 nucleotides in length. In an embodiment, the template nucleic acid is 10 to 1,000, 20 to 900, 30 to 800, 40 to 700, 50 to 600, 50 to 500, 50 to 400, 50 to 300, 50 to 200, or 50 to 100 nucleotides in length.

In some embodiments, the template polynucleotide is complementary to a portion of a polynucleotide comprising the target sequence. When optimally aligned, a template polynucleotide might overlap with one or more nucleotides of a target sequences (e.g. about or more than about 1, 5, 10, 15, 20, 25, 30, 35, 40, 45, 50, 60, 70, 80, 90, 100 or more nucleotides). In some embodiments, when a template sequence and a polynucleotide comprising a target sequence are optimally aligned, the nearest nucleotide of the template polynucleotide is within about 1, 5, 10, 15, 20, 25, 50, 75, 100, 200, 300, 400, 500, 1000, 5000, 10000, or more nucleotides from the target sequence.

The exogenous polynucleotide template comprises a sequence to be integrated (e.g., a mutated gene). The sequence for integration may be a sequence endogenous or exogenous to the cell. Examples of a sequence to be integrated include polynucleotides encoding a protein or a non-coding RNA (e.g., a microRNA). Thus, the sequence for integration may be operably linked to an appropriate control sequence or sequences. Alternatively, the sequence to be integrated may provide a regulatory function.

An upstream or downstream sequence may comprise from about 20 bp to about 2500 bp, for example, about 50, 100, 200, 300, 400, 500, 600, 700, 800, 900, 1000, 1100, 1200, 1300, 1400, 1500, 1600, 1700, 1800, 1900, 2000, 2100, 2200, 2300, 2400, or 2500 bp. In some methods, the exemplary upstream or downstream sequence have about 200 bp to about 2000 bp, about 600 bp to about 1000 bp, or more particularly about 700 bp to about 1000.

An upstream or downstream sequence may comprise from about 20 bp to about 2500 bp, for example, about 50, 100, 200, 300, 400, 500, 600, 700, 800, 900, 1000, 1100, 1200, 1300, 1400, 1500, 1600, 1700, 1800, 1900, 2000, 2100, 2200, 2300, 2400, or 2500 bp. In some methods, the exemplary upstream or downstream sequence have about 200 bp to about 2000 bp, about 600 bp to about 1000 bp, or more particularly about 700 bp to about 1000

In certain embodiments, one or both homology arms may be shortened to avoid including certain sequence repeat elements. For example, a 5′ homology arm may be shortened to avoid a sequence repeat element. In other embodiments, a 3′ homology arm may be shortened to avoid a sequence repeat element. In some embodiments, both the 5′ and the 3′ homology arms may be shortened to avoid including certain sequence repeat elements.

In some methods, the exogenous polynucleotide template may further comprise a marker. Such a marker may make it easy to screen for targeted integrations. Examples of suitable markers include restriction sites, fluorescent proteins, or selectable markers. The exogenous polynucleotide template of the disclosure can be constructed using recombinant techniques (see, for example, Sambrook et al., 2001 and Ausubel et al., 1996).

In certain embodiments, a template nucleic acid for correcting a mutation may be designed for use as a single-stranded oligonucleotide. When using a single-stranded oligonucleotide, 5′ and 3′ homology arms may range up to about 200 base pairs (bp) in length, e.g., at least 25, 50, 75, 100, 125, 150, 175, or 200 bp in length.

In certain embodiments, a template nucleic acid for correcting a mutation may be designed for use with a homology-independent targeted integration system. Suzuki et al. describe in vivo genome editing via CRISPR/Cas9 mediated homology-independent targeted integration (2016, Nature 540:144-149). Schmid-Burgk, et al. describe use of the CRISPR-Cas9 system to introduce a double-strand break (DSB) at a user-defined genomic location and insertion of a universal donor DNA (Nat Commun. 2016 Jul. 28; 7:12338). Gao, et al. describe “Plug-and-Play Protein Modification Using Homology-Independent Universal Genome Engineering” (Neuron. 2019 Aug. 21; 103(4):583-597).

### RNAi

In some embodiments, the genetic modulating agents may be interfering RNAs. In certain embodiments, diseases caused by a dominant mutation in a gene is targeted by silencing the mutated gene using RNAi. In some cases, the nucleotide sequence may comprise coding sequence for one or more interfering RNAs. In certain examples, the nucleotide sequence may be interfering RNA (RNAi). As used herein, the term “RNAi” refers to any type of interfering RNA, including but not limited to, siRNAi, shRNAi, endogenous microRNA and artificial microRNA. For instance, it includes sequences previously identified as siRNA, regardless of the mechanism of down-stream processing of the RNA (i.e. although siRNAs are believed to have a specific method of in vivo processing resulting in the cleavage of mRNA, such sequences can be incorporated into the vectors in the context of the flanking sequences described herein). The term “RNAi” can include both gene silencing RNAi molecules, and also RNAi effector molecules which activate the expression of a gene.

In certain embodiments, a modulating agent may comprise silencing one or more endogenous genes. As used herein, “gene silencing” or “gene silenced” in reference to an activity of an RNAi molecule, for example a siRNA or miRNA refers to a decrease in the mRNA level in a cell for a target gene by at least about 5%, about 10%, about 20%, about 30%, about 40%, about 50%, about 60%, about 70%, about 80%, about 90%, about 95%, about 99%, about 100% of the mRNA level found in the cell without the presence of the miRNA or RNA interference molecule. In one preferred embodiment, the mRNA levels are decreased by at least about 70%, about 80%, about 90%, about 95%, about 99%, about 100%.

As used herein, a “siRNA” refers to a nucleic acid that forms a double stranded RNA, which double stranded RNA has the ability to reduce or inhibit expression of a gene or target gene when the siRNA is present or expressed in the same cell as the target gene. The double stranded RNA siRNA can be formed by the complementary strands. In one embodiment, a siRNA refers to a nucleic acid that can form a double stranded siRNA. The sequence of the siRNA can correspond to the full-length target gene, or a subsequence thereof. Typically, the siRNA is at least about 15-50 nucleotides in length (e.g., each complementary sequence of the double stranded siRNA is about 15-50 nucleotides in length, and the double stranded siRNA is about 15-50 base pairs in length, preferably about 19-30 base nucleotides, preferably about 20-25 nucleotides in length, e.g., 20, 21, 22, 23, 24, 25, 26, 27, 28, 29, or 30 nucleotides in length).

As used herein “shRNA” or “small hairpin RNA” (also called stem loop) is a type of siRNA. In one embodiment, these shRNAs are composed of a short, e.g. about 19 to about 25 nucleotide, antisense strand, followed by a nucleotide loop of about 5 to about 9 nucleotides, and the analogous sense strand. Alternatively, the sense strand can precede the nucleotide loop structure and the antisense strand can follow.

The terms “microRNA” or “miRNA” are used interchangeably herein are endogenous RNAs, some of which are known to regulate the expression of protein-coding genes at the posttranscriptional level. Endogenous microRNAs are small RNAs naturally present in the genome that are capable of modulating the productive utilization of mRNA. The term artificial microRNA includes any type of RNA sequence, other than endogenous microRNA, which is capable of modulating the productive utilization of mRNA. MicroRNA sequences have been described in publications such as Lim, et al., Genes & Development, 17, p. 991-1008 (2003), Lim et al Science 299, 1540 (2003), Lee and Ambros Science, 294, 862 (2001), Lau et al., Science 294, 858-861 (2001), Lagos-Quintana et al, Current Biology, 12, 735-739 (2002), Lagos Quintana et al, Science 294, 853-857 (2001), and Lagos-Quintana et al, RNA, 9, 175-179 (2003), which are incorporated by reference. Multiple microRNAs can also be incorporated into a precursor molecule. Furthermore, miRNA-like stem-loops can be expressed in cells as a vehicle to deliver artificial miRNAs and short interfering RNAs (siRNAs) for the purpose of modulating the expression of endogenous genes through the miRNA and or RNAi pathways.

As used herein, “double stranded RNA” or “dsRNA” refers to RNA molecules that are comprised of two strands. Double-stranded molecules include those comprised of a single RNA molecule that doubles back on itself to form a two-stranded structure. For example, the stem loop structure of the progenitor molecules from which the single-stranded miRNA is derived, called the pre-miRNA (Bartel et al. 2004. Cell 1 16:281-297), comprises a dsRNA molecule.

### Antibodies

In certain embodiments, the one or more agents is an antibody. The term “antibody” is used interchangeably with the term “immunoglobulin” herein, and includes intact antibodies, fragments of antibodies, e.g., Fab, F(ab′)2 fragments, and intact antibodies and fragments that have been mutated either in their constant and/or variable region (e.g., mutations to produce chimeric, partially humanized, or fully humanized antibodies, as well as to produce antibodies with a desired trait, e.g., enhanced binding and/or reduced FcR binding). The term “fragment” refers to a part or portion of an antibody or antibody chain comprising fewer amino acid residues than an intact or complete antibody or antibody chain. Fragments can be obtained via chemical or enzymatic treatment of an intact or complete antibody or antibody chain. Fragments can also be obtained by recombinant means. Exemplary fragments include Fab, Fab′, F(ab′)2, Fabc, Fd, dAb, V_{HH} and scFv and/or Fv fragments.

As used herein, a preparation of antibody protein having less than about 50% of non-antibody protein (also referred to herein as a “contaminating protein”), or of chemical precursors, is considered to be “substantially free.” 40%, 30%, 20%, 10% and more preferably 5% (by dry weight), of non-antibody protein, or of chemical precursors is considered to be substantially free. When the antibody protein or biologically active portion thereof is recombinantly produced, it is also preferably substantially free of culture medium, i.e., culture medium represents less than about 30%, preferably less than about 20%, more preferably less than about 10%, and most preferably less than about 5% of the volume or mass of the protein preparation.

The term “antigen-binding fragment” refers to a polypeptide fragment of an immunoglobulin or antibody that binds antigen or competes with intact antibody (i.e., with the intact antibody from which they were derived) for antigen binding (i.e., specific binding). As such these antibodies or fragments thereof are included in the scope of the invention, provided that the antibody or fragment binds specifically to a target molecule.

It is intended that the term “antibody” encompass any Ig class or any Ig subclass (e.g. the IgG1, IgG2, IgG3, and IgG4 subclasses of IgG) obtained from any source (e.g., humans and non-human primates, and in rodents, lagomorphs, caprines, bovines, equines, ovines, etc.).

The term “Ig class” or “immunoglobulin class”, as used herein, refers to the five classes of immunoglobulin that have been identified in humans and higher mammals, IgG, IgM, IgA, IgD, and IgE. The term “Ig subclass” refers to the two subclasses of IgM (H and L), three subclasses of IgA (IgA1, IgA2, and secretory IgA), and four subclasses of IgG (IgG1, IgG2, IgG3, and IgG4) that have been identified in humans and higher mammals. The antibodies can exist in monomeric or polymeric form; for example, IgM antibodies exist in pentameric form, and IgA antibodies exist in monomeric, dimeric or multimeric form.

The term “IgG subclass” refers to the four subclasses of immunoglobulin class IgG-IgG1, IgG2, IgG3, and IgG4 that have been identified in humans and higher mammals by the heavy chains of the immunoglobulins, V1-γ4, respectively. The term “single-chain immunoglobulin” or “single-chain antibody” (used interchangeably herein) refers to a protein having a two-polypeptide chain structure consisting of a heavy and a light chain, said chains being stabilized, for example, by interchain peptide linkers, which has the ability to specifically bind antigen. The term “domain” refers to a globular region of a heavy or light chain polypeptide comprising peptide loops (e.g., comprising 3 to 4 peptide loops) stabilized, for example, by β pleated sheet and/or intrachain disulfide bond. Domains are further referred to herein as “constant” or “variable”, based on the relative lack of sequence variation within the domains of various class members in the case of a “constant” domain, or the significant variation within the domains of various class members in the case of a “variable” domain. Antibody or polypeptide “domains” are often referred to interchangeably in the art as antibody or polypeptide “regions”. The “constant” domains of an antibody light chain are referred to interchangeably as “light chain constant regions”, “light chain constant domains”, “CL” regions or “CL” domains. The “constant” domains of an antibody heavy chain are referred to interchangeably as “heavy chain constant regions”, “heavy chain constant domains”, “CH” regions or “CH” domains). The “variable” domains of an antibody light chain are referred to interchangeably as “light chain variable regions”, “light chain variable domains”, “VL” regions or “VL” domains). The “variable” domains of an antibody heavy chain are referred to interchangeably as “heavy chain constant regions”, “heavy chain constant domains”, “VH” regions or “VH” domains).

The term “region” can also refer to a part or portion of an antibody chain or antibody chain domain (e.g., a part or portion of a heavy or light chain or a part or portion of a constant or variable domain, as defined herein), as well as more discrete parts or portions of said chains or domains. For example, light and heavy chains or light and heavy chain variable domains include “complementarity determining regions” or “CDRs” interspersed among “framework regions” or “FRs”, as defined herein.

The term “conformation” refers to the tertiary structure of a protein or polypeptide (e.g., an antibody, antibody chain, domain or region thereof). For example, the phrase “light (or heavy) chain conformation” refers to the tertiary structure of a light (or heavy) chain variable region, and the phrase “antibody conformation” or “antibody fragment conformation” refers to the tertiary structure of an antibody or fragment thereof.

The term “antibody-like protein scaffolds” or “engineered protein scaffolds” broadly encompasses proteinaceous non-immunoglobulin specific-binding agents, typically obtained by combinatorial engineering (such as site-directed random mutagenesis in combination with phage display or other molecular selection techniques). Usually, such scaffolds are derived from robust and small soluble monomeric proteins (such as Kunitz inhibitors or lipocalins) or from a stably folded extra-membrane domain of a cell surface receptor (such as protein A, fibronectin or the ankyrin repeat).

Such scaffolds have been extensively reviewed in Binz et al. (Engineering novel binding proteins from nonimmunoglobulin domains. Nat Biotechnol 2005, 23:1257-1268), Gebauer and Skerra (Engineered protein scaffolds as next-generation antibody therapeutics. Curr Opin Chem Biol. 2009, 13:245-55), Gill and Damle (Biopharmaceutical drug discovery using novel protein scaffolds. Curr Opin Biotechnol 2006, 17:653-658), Skerra (Engineered protein scaffolds for molecular recognition. J Mol Recognit 2000, 13:167-187), and Skerra (Alternative non-antibody scaffolds for molecular recognition. Curr Opin Biotechnol 2007, 18:295-304), and include without limitation affibodies, based on the Z-domain of staphylococcal protein A, a three-helix bundle of 58 residues providing an interface on two of its alpha-helices (Nygren, Alternative binding proteins: Affibody binding proteins developed from a small three-helix bundle scaffold. FEBS J 2008, 275:2668-2676); engineered Kunitz domains based on a small (ca. 58 residues) and robust, disulphide-crosslinked serine protease inhibitor, typically of human origin (e.g. LACI-D1), which can be engineered for different protease specificities (Nixon and Wood, Engineered protein inhibitors of proteases. Curr Opin Drug Discov Dev 2006, 9:261-268); monobodies or adnectins based on the 10th extracellular domain of human fibronectin III (10Fn3), which adopts an Ig-like beta-sandwich fold (94 residues) with 2-3 exposed loops, but lacks the central disulphide bridge (Koide and Koide, Monobodies: antibody mimics based on the scaffold of the fibronectin type III domain. Methods Mol Biol 2007, 352:95-109); anticalins derived from the lipocalins, a diverse family of eight-stranded beta-barrel proteins (ca. 180 residues) that naturally form binding sites for small ligands by means of four structurally variable loops at the open end, which are abundant in humans, insects, and many other organisms (Skerra, Alternative binding proteins: Anticalins—harnessing the structural plasticity of the lipocalin ligand pocket to engineer novel binding activities. FEBS J 2008, 275:2677-2683); DARPins, designed ankyrin repeat domains (166 residues), which provide a rigid interface arising from typically three repeated beta-turns (Stumpp et al., DARPins: a new generation of protein therapeutics. Drug Discov Today 2008, 13:695-701); avimers (multimerized LDLR-A module) (Silverman et al., Multivalent avimer proteins evolved by exon shuffling of a family of human receptor domains. Nat Biotechnol 2005, 23:1556-1561); and cysteine-rich knottin peptides (Kolmar, Alternative binding proteins: biological activity and therapeutic potential of cystine-knot miniproteins. FEBS J 2008, 275:2684-2690).

“Specific binding” of an antibody means that the antibody exhibits appreciable affinity for a particular antigen or epitope and, generally, does not exhibit significant cross reactivity. “Appreciable” binding includes binding with an affinity of at least 25 μM. Antibodies with affinities greater than 1×10⁷ M⁻¹ (or a dissociation coefficient of 1 μM or less or a dissociation coefficient of 1 nm or less) typically bind with correspondingly greater specificity. Values intermediate of those set forth herein are also intended to be within the scope of the present invention and antibodies of the invention bind with a range of affinities, for example, 100 nM or less, 75 nM or less, 50 nM or less, 25 nM or less, for example 10 nM or less, 5 nM or less, 1 nM or less, or in embodiments 500 pM or less, 100 pM or less, 50 pM or less or 25 pM or less. An antibody that “does not exhibit significant crossreactivity” is one that will not appreciably bind to an entity other than its target (e.g., a different epitope or a different molecule). For example, an antibody that specifically binds to a target molecule will appreciably bind the target molecule but will not significantly react with non-target molecules or peptides. An antibody specific for a particular epitope will, for example, not significantly crossreact with remote epitopes on the same protein or peptide. Specific binding can be determined according to any art-recognized means for determining such binding. Preferably, specific binding is determined according to Scatchard analysis and/or competitive binding assays.

As used herein, the term “affinity” refers to the strength of the binding of a single antigen-combining site with an antigenic determinant. Affinity depends on the closeness of stereochemical fit between antibody combining sites and antigen determinants, on the size of the area of contact between them, on the distribution of charged and hydrophobic groups, etc. Antibody affinity can be measured by equilibrium dialysis or by the kinetic BIACORE™ method. The dissociation constant, Kd, and the association constant, Ka, are quantitative measures of affinity.

As used herein, the term “monoclonal antibody” refers to an antibody derived from a clonal population of antibody-producing cells (e.g., B lymphocytes or B cells) which is homogeneous in structure and antigen specificity. The term “polyclonal antibody” refers to a plurality of antibodies originating from different clonal populations of antibody-producing cells which are heterogeneous in their structure and epitope specificity but which recognize a common antigen. Monoclonal and polyclonal antibodies may exist within bodily fluids, as crude preparations, or may be purified, as described herein.

The term “binding portion” of an antibody (or “antibody portion”) includes one or more complete domains, e.g., a pair of complete domains, as well as fragments of an antibody that retain the ability to specifically bind to a target molecule. It has been shown that the binding function of an antibody can be performed by fragments of a full-length antibody. Binding fragments are produced by recombinant DNA techniques, or by enzymatic or chemical cleavage of intact immunoglobulins. Binding fragments include Fab, Fab′, F(ab′)2, Fabc, Fd, dAb, Fv, single chains, single-chain antibodies, e.g., scFv, and single domain antibodies.

“Humanized” forms of non-human (e.g., murine) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, FR residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable regions correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin.

Examples of portions of antibodies or epitope-binding proteins encompassed by the present definition include: (i) the Fab fragment, having V_{L}, C_{L}, V_{H} and C_{H}1 domains; (ii) the Fab′ fragment, which is a Fab fragment having one or more cysteine residues at the C-terminus of the C_{H}1 domain; (iii) the Fd fragment having V_{H} and C_{H}1 domains; (iv) the Fd′ fragment having V_{H} and C_{H}1 domains and one or more cysteine residues at the C-terminus of the CHI domain; (v) the Fv fragment having the V_{L} and V_{H} domains of a single arm of an antibody; (vi) the dAb fragment (Ward et al., 341 Nature 544 (1989)) which consists of a V_{H} domain or a V_{L} domain that binds antigen; (vii) isolated CDR regions or isolated CDR regions presented in a functional framework; (viii) F(ab′)₂ fragments which are bivalent fragments including two Fab′ fragments linked by a disulphide bridge at the hinge region; (ix) single chain antibody molecules (e.g., single chain Fv; scFv) (Bird et al., 242 Science 423 (1988); and Huston et al., 85 PNAS 5879 (1988)); (x) “diabodies” with two antigen binding sites, comprising a heavy chain variable domain (V_{H}) connected to a light chain variable domain (V_{L}) in the same polypeptide chain (see, e.g., EP 404,097; WO 93/11161; Hollinger et al., 90 PNAS 6444 (1993)); (xi) “linear antibodies” comprising a pair of tandem Fd segments (V_{H}-Cₕ1-V_{H}-Cₕ1) which, together with complementary light chain polypeptides, form a pair of antigen binding regions (Zapata et al., Protein Eng. 8(10):1057-62 (1995); and U.S. Pat. No. 5,641,870).

As used herein, a “blocking” antibody or an antibody “antagonist” is one which inhibits or reduces biological activity of the antigen(s) it binds. In certain embodiments, the blocking antibodies or antagonist antibodies or portions thereof described herein completely inhibit the biological activity of the antigen(s).

Antibodies may act as agonists or antagonists of the recognized polypeptides. For example, the present invention includes antibodies which disrupt receptor/ligand interactions either partially or fully. The invention features both receptor-specific antibodies and ligand-specific antibodies. The invention also features receptor-specific antibodies which do not prevent ligand binding but prevent receptor activation. Receptor activation (i.e., signaling) may be determined by techniques described herein or otherwise known in the art. For example, receptor activation can be determined by detecting the phosphorylation (e.g., tyrosine or serine/threonine) of the receptor or of one of its down-stream substrates by immunoprecipitation followed by western blot analysis. In specific embodiments, antibodies are provided that inhibit ligand activity or receptor activity by at least 95%, at least 90%, at least 85%, at least 80%, at least 75%, at least 70%, at least 60%, or at least 50% of the activity in absence of the antibody.

The invention also features receptor-specific antibodies which both prevent ligand binding and receptor activation as well as antibodies that recognize the receptor-ligand complex. Likewise, encompassed by the invention are neutralizing antibodies which bind the ligand and prevent binding of the ligand to the receptor, as well as antibodies which bind the ligand, thereby preventing receptor activation, but do not prevent the ligand from binding the receptor. Further included in the invention are antibodies which activate the receptor. These antibodies may act as receptor agonists, i.e., potentiate or activate either all or a subset of the biological activities of the ligand-mediated receptor activation, for example, by inducing dimerization of the receptor. The antibodies may be specified as agonists, antagonists or inverse agonists for biological activities comprising the specific biological activities of the peptides disclosed herein. The antibody agonists and antagonists can be made using methods known in the art. See, e.g., PCT publication WO 96/40281; U.S. Pat. No. 5,811,097; Deng et al., Blood 92(6):1981-1988 (1998); Chen et al., Cancer Res. 58(16):3668-3678 (1998); Harrop et al., J. Immunol. 161(4):1786-1794 (1998); Zhu et al., Cancer Res. 58(15):3209-3214 (1998); Yoon et al., J. Immunol. 160(7):3170-3179 (1998); Prat et al., J. Cell. Sci. III (Pt2):237-247 (1998); Pitard et al., J. Immunol. Methods 205(2):177-190 (1997); Liautard et al., Cytokine 9(4):233-241 (1997); Carlson et al., J. Biol. Chem. 272(17):11295-11301 (1997); Taryman et al., Neuron 14(4):755-762 (1995); Muller et al., Structure 6(9):1153-1167 (1998); Bartunek et al., Cytokine 8(1):14-20 (1996).

The antibodies as defined for the present invention include derivatives that are modified, i.e., by the covalent attachment of any type of molecule to the antibody such that covalent attachment does not prevent the antibody from generating an anti-idiotypic response. For example, but not by way of limitation, the antibody derivatives include antibodies that have been modified, e.g., by glycosylation, acetylation, pegylation, phosphylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, metabolic synthesis of tunicamycin, etc. Additionally, the derivative may contain one or more non-classical amino acids.

Simple binding assays can be used to screen for or detect agents that bind to a target protein, or disrupt the interaction between proteins (e.g., a receptor and a ligand). Because certain targets of the present invention are transmembrane proteins, assays that use the soluble forms of these proteins rather than full-length protein can be used, in some embodiments. Soluble forms include, for example, those lacking the transmembrane domain and/or those comprising the IgV domain or fragments thereof which retain their ability to bind their cognate binding partners. Further, agents that inhibit or enhance protein interactions for use in the compositions and methods described herein, can include recombinant peptido-mimetics.

Detection methods useful in screening assays include antibody-based methods, detection of a reporter moiety, detection of cytokines as described herein, and detection of a gene signature as described herein.

Another variation of assays to determine binding of a receptor protein to a ligand protein is through the use of affinity biosensor methods. Such methods may be based on the piezoelectric effect, electrochemistry, or optical methods, such as ellipsometry, optical wave guidance, and surface plasmon resonance (SPR).

### Aptamers

In certain embodiments, the one or more agents is an aptamer. Nucleic acid aptamers are nucleic acid species that have been engineered through repeated rounds of in vitro selection or equivalently, SELEX (systematic evolution of ligands by exponential enrichment) to bind to various molecular targets such as small molecules, proteins, nucleic acids, cells, tissues and organisms. Nucleic acid aptamers have specific binding affinity to molecules through interactions other than classic Watson-Crick base pairing. Aptamers are useful in biotechnological and therapeutic applications as they offer molecular recognition properties similar to antibodies. In addition to their discriminate recognition, aptamers offer advantages over antibodies as they can be engineered completely in a test tube, are readily produced by chemical synthesis, possess desirable storage properties, and elicit little or no immunogenicity in therapeutic applications. In certain embodiments, RNA aptamers may be expressed from a DNA construct. In other embodiments, a nucleic acid aptamer may be linked to another polynucleotide sequence. The polynucleotide sequence may be a double stranded DNA polynucleotide sequence. The aptamer may be covalently linked to one strand of the polynucleotide sequence. The aptamer may be ligated to the polynucleotide sequence. The polynucleotide sequence may be configured, such that the polynucleotide sequence may be linked to a solid support or ligated to another polynucleotide sequence.

Aptamers, like peptides generated by phage display or monoclonal antibodies (“mAbs”), are capable of specifically binding to selected targets and modulating the target's activity, e.g., through binding, aptamers may block their target's ability to function. A typical aptamer is 10-15 kDa in size (30-45 nucleotides), binds its target with sub-nanomolar affinity, and discriminates against closely related targets (e.g., aptamers will typically not bind other proteins from the same gene family). Structural studies have shown that aptamers are capable of using the same types of binding interactions (e.g., hydrogen bonding, electrostatic complementarity, hydrophobic contacts, steric exclusion) that drives affinity and specificity in antibody-antigen complexes.

Aptamers have a number of desirable characteristics for use in research and as therapeutics and diagnostics including high specificity and affinity, biological efficacy, and excellent pharmacokinetic properties. In addition, they offer specific competitive advantages over antibodies and other protein biologics. Aptamers are chemically synthesized and are readily scaled as needed to meet production demand for research, diagnostic or therapeutic applications. Aptamers are chemically robust. They are intrinsically adapted to regain activity following exposure to factors such as heat and denaturants and can be stored for extended periods (>1 yr) at room temperature as lyophilized powders. Not being bound by a theory, aptamers bound to a solid support or beads may be stored for extended periods.

Oligonucleotides in their phosphodiester form may be quickly degraded by intracellular and extracellular enzymes such as endonucleases and exonucleases. Aptamers can include modified nucleotides conferring improved characteristics on the ligand, such as improved in vivo stability or improved delivery characteristics. Examples of such modifications include chemical substitutions at the ribose and/or phosphate and/or base positions. SELEX identified nucleic acid ligands containing modified nucleotides are described, e.g., in U.S. Pat. No. 5,660,985, which describes oligonucleotides containing nucleotide derivatives chemically modified at the 2′ position of ribose, 5 position of pyrimidines, and 8 position of purines, U.S. Pat. No. 5,756,703 which describes oligonucleotides containing various 2′-modified pyrimidines, and U.S. Pat. No. 5,580,737 which describes highly specific nucleic acid ligands containing one or more nucleotides modified with 2′-amino (2′-NH₂), 2′-fluoro (2′-F), and/or 2′-0-methyl (2′-OMe) substituents. Modifications of aptamers may also include, modifications at exocyclic amines, substitution of 4-thiouridine, substitution of 5-bromo or 5-iodo-uracil; backbone modifications, phosphorothioate or allyl phosphate modifications, methylations, and unusual base-pairing combinations such as the isobases isocytidine and isoguanosine. Modifications can also include 3′ and 5′ modifications such as capping. As used herein, the term phosphorothioate encompasses one or more non-bridging oxygen atoms in a phosphodiester bond replaced by one or more sulfur atoms. In further embodiments, the oligonucleotides comprise modified sugar groups, for example, one or more of the hydroxyl groups is replaced with halogen, aliphatic groups, or functionalized as ethers or amines. In one embodiment, the 2′-position of the furanose residue is substituted by any of an O-methyl, O-alkyl, O-allyl, S-alkyl, S-allyl, or halo group. Methods of synthesis of 2′-modified sugars are described, e.g., in Sproat, et al., Nucl. Acid Res. 19:733-738 (1991); Cotten, et al, Nucl. Acid Res. 19:2629-2635 (1991); and Hobbs, et al, Biochemistry 12:5138-5145 (1973). Other modifications are known to one of ordinary skill in the art. In certain embodiments, aptamers include aptamers with improved off-rates as described in International Patent Publication No. WO 2009012418, “Method for generating aptamers with improved off-rates,” incorporated herein by reference in its entirety. In certain embodiments aptamers are chosen from a library of aptamers. Such libraries include, but are not limited to those described in Rohloff et al., “Nucleic Acid Ligands With Protein-like Side Chains: Modified Aptamers and Their Use as Diagnostic and Therapeutic Agents,” Molecular Therapy Nucleic Acids (2014) 3, e201. Aptamers are also commercially available (see, e.g., SomaLogic, Inc., Boulder, Colo.). In certain embodiments, the present invention may utilize any aptamer containing any modification as described herein.

### Adoptive Cell Transfer

In certain embodiments, the methods of the present invention may be used to predict a response to adoptive cell transfer methods. In certain embodiments, modulating gene program activity or treating with one or more agents capable of modulating one or more identified therapeutic targets (e.g., a gene in a gene module comprising an interacting genetic variant) shifts an immune cell to be resistant to dysfunction or have increased effector function. Such immune cells may be used to increase the effectiveness of adoptive cell transfer. In certain embodiments, immune cells are shifted to be more suppressive to treat diseases requiring a decreased immune response (e.g., autoimmune diseases). As used herein, “ACT”, “adoptive cell therapy” and “adoptive cell transfer” may be used interchangeably. In certain embodiments, Adoptive Cell Therapy (ACT) can refer to the transfer of cells to a patient with the goal of transferring the functionality and characteristics into the new host by engraftment of the cells (see, e.g., Mettananda et al., Editing an a-globin enhancer in primary human hematopoietic stem cells as a treatment for β-thalassemia, Nat Commun. 2017 Sep. 4; 8(1):424). As used herein, the term “engraft” or “engraftment” refers to the process of cell incorporation into a tissue of interest in vivo through contact with existing cells of the tissue. Adoptive Cell Therapy (ACT) can refer to the transfer of cells, most commonly immune-derived cells, back into the same patient or into a new recipient host with the goal of transferring the immunologic functionality and characteristics into the new host. If possible, use of autologous cells helps the recipient by minimizing GVHD issues. The adoptive transfer of autologous tumor infiltrating lymphocytes (TIL) (Zacharakis et al., (2018) Nat Med. 2018 June; 24(6):724-730; Besser et al., (2010) Clin. Cancer Res 16 (9) 2646-55; Dudley et al., (2002) Science 298 (5594): 850-4; and Dudley et al., (2005) Journal of Clinical Oncology 23 (10): 2346-57.) or genetically re-directed peripheral blood mononuclear cells (Johnson et al., (2009) Blood 114 (3): 535-46; and Morgan et al., (2006) Science 314(5796) 126-9) has been used to successfully treat patients with advanced solid tumors, including melanoma, metastatic breast cancer and colorectal carcinoma, as well as patients with CD19-expressing hematologic malignancies (Kalos et al., (2011) Science Translational Medicine 3 (95): 95ra73). In certain embodiments, allogenic cells immune cells are transferred (see, e.g., Ren et al., (2017) Clin Cancer Res 23 (9) 2255-2266). As described further herein, allogenic cells can be edited to reduce alloreactivity and prevent graft-versus-host disease. Thus, use of allogenic cells allows for cells to be obtained from healthy donors and prepared for use in patients as opposed to preparing autologous cells from a patient after diagnosis.

Aspects of the invention involve the adoptive transfer of immune system cells, such as T cells, specific for selected antigens, such as tumor associated antigens or tumor specific neoantigens (see, e.g., Maus et al., 2014, Adoptive Immunotherapy for Cancer or Viruses, Annual Review of Immunology, Vol. 32: 189-225; Rosenberg and Restifo, 2015, Adoptive cell transfer as personalized immunotherapy for human cancer, Science Vol. 348 no. 6230 pp. 62-68; Restifo et al., 2015, Adoptive immunotherapy for cancer: harnessing the T cell response. Nat. Rev. Immunol. 12(4): 269-281; and Jenson and Riddell, 2014, Design and implementation of adoptive therapy with chimeric antigen receptor-modified T cells. Immunol Rev. 257(1): 127-144; and Rajasagi et al., 2014, Systematic identification of personal tumor-specific neoantigens in chronic lymphocytic leukemia. Blood. 2014 Jul. 17; 124(3):453-62).

In certain embodiments, an antigen (such as a tumor antigen) to be targeted in adoptive cell therapy (such as particularly CAR or TCR T-cell therapy) of a disease (such as particularly of tumor or cancer) may be selected from a group consisting of: B cell maturation antigen (BCMA) (see, e.g., Friedman et al., Effective Targeting of Multiple BCMA-Expressing Hematological Malignancies by Anti-BCMA CAR T Cells, Hum Gene Ther. 2018 Mar. 8; Berdeja J G, et al. Durable clinical responses in heavily pretreated patients with relapsed/refractory multiple myeloma: updated results from a multicenter study of bb2121 anti-Bcma CAR T cell therapy. Blood. 2017; 130:740; and Mouhieddine and Ghobrial, Immunotherapy in Multiple Myeloma: The Era of CAR T Cell Therapy, Hematologist, May-June 2018, Volume 15, issue 3); PSA (prostate-specific antigen); prostate-specific membrane antigen (PSMA); PSCA (Prostate stem cell antigen); Tyrosine-protein kinase transmembrane receptor ROR1; fibroblast activation protein (FAP); Tumor-associated glycoprotein 72 (TAG72); Carcinoembryonic antigen (CEA); Epithelial cell adhesion molecule (EPCAM); Mesothelin; Human Epidermal growth factor Receptor 2 (ERBB2 (Her2/neu)); Prostase; Prostatic acid phosphatase (PAP); elongation factor 2 mutant (ELF2M); Insulin-like growth factor 1 receptor (IGF-1R); gp100; BCR-ABL (breakpoint cluster region-Abelson); tyrosinase; New York esophageal squamous cell carcinoma 1 (NY-ESO-1); κ-light chain, LAGE (L antigen); MAGE (melanoma antigen); Melanoma-associated antigen 1 (MAGE-A1); MAGE A3; MAGE A6; legumain; Human papillomavirus (HPV) E6; HPV E7; prostein; survivin; PCTA1 (Galectin 8); Melan-A/MART-1; Ras mutant; TRP-1 (tyrosinase related protein 1, or gp75); Tyrosinase-related Protein 2 (TRP2); TRP-2/INT2 (TRP-2/intron 2); RAGE (renal antigen); receptor for advanced glycation end products 1 (RAGE1); Renal ubiquitous 1, 2 (RU1, RU2); intestinal carboxyl esterase (iCE); Heat shock protein 70-2 (HSP70-2) mutant; thyroid stimulating hormone receptor (TSHR); CD123; CD171; CD19; CD20; CD22; CD26; CD30; CD33; CD44v7/8 (cluster of differentiation 44, exons 7/8); CD53; CD92; CD100; CD148; CD150; CD200; CD261; CD262; CD362; CS-1 (CD2 subset 1, CRACC, SLAMF7, CD319, and 19A24); C-type lectin-like molecule-1 (CLL-1); ganglioside GD3 (aNeu5Ac(2-8)aNeu5Ac(2-3)bDGalp(1-4)bDGlcp(1-1)Cer); Tn antigen (Tn Ag); Fms-Like Tyrosine Kinase 3 (FLT3); CD38; CD138; CD44v6; B7H3 (CD276); KIT (CD117); Interleukin-13 receptor subunit alpha-2 (IL-13Ra2); Interleukin 11 receptor alpha (IL-11Ra); prostate stem cell antigen (PSCA); Protease Serine 21 (PRSS21); vascular endothelial growth factor receptor 2 (VEGFR2); Lewis(Y) antigen; CD24; Platelet-derived growth factor receptor beta (PDGFR-beta); stage-specific embryonic antigen-4 (SSEA-4); Mucin 1, cell surface associated (MUC1); mucin 16 (MUC16); epidermal growth factor receptor (EGFR); epidermal growth factor receptor variant III (EGFRvIII); neural cell adhesion molecule (NCAM); carbonic anhydrase IX (CAIX); Proteasome (Prosome, Macropain) Subunit, Beta Type, 9 (LMP2); ephrin type-A receptor 2 (EphA2); Ephrin B2; Fucosyl GM1; sialyl Lewis adhesion molecule (sLe); ganglioside GM3 (aNeu5Ac(2-3)bDGalp(1-4)bDGlcp(1-1)Cer); TGS5; high molecular weight-melanoma-associated antigen (HMWMAA); o-acetyl-GD2 ganglioside (OAcGD2); Folate receptor alpha; Folate receptor beta; tumor endothelial marker 1 (TEM1/CD248); tumor endothelial marker 7-related (TEM7R); claudin 6 (CLDN6); G protein-coupled receptor class C group 5, member D (GPRC5D); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (OR51E2); TCR Gamma Alternate Reading Frame Protein (TARP); Wilms tumor protein (WT1); ETS translocation-variant gene 6, located on chromosome 12p (ETV6-AML); sperm protein 17 (SPA17); X Antigen Family, Member 1A (XAGE1); angiopoietin-binding cell surface receptor 2 (Tie 2); CT (cancer/testis (antigen)); melanoma cancer testis antigen-1 (MAD-CT-1); melanoma cancer testis antigen-2 (MAD-CT-2); Fos-related antigen 1; p53; p53 mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin B 1; Cyclin D1; v-myc avian myelocytomatosis viral oncogene neuroblastoma derived homolog (MYCN); Ras Homolog Family Member C (RhoC); Cytochrome P450 1B1 (CYP1B1); CCCTC-Binding Factor (Zinc Finger Protein)-Like (BORIS); Squamous Cell Carcinoma Antigen Recognized By T Cells-1 or 3 (SART1, SART3); Paired box protein Pax-5 (PAX5); proacrosin binding protein sp32 (OY-TES1); lymphocyte-specific protein tyrosine kinase (LCK); A kinase anchor protein 4 (AKAP-4); synovial sarcoma, X breakpoint-1, -2, -3 or -4 (SSX1, SSX2, SSX3, SSX4); CD79a; CD79b; CD72; Leukocyte-associated immunoglobulin-like receptor 1 (LAIR1); Fc fragment of IgA receptor (FCAR); Leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRL5); mouse double minute 2 homolog (MDM2); livin; alphafetoprotein (AFP); transmembrane activator and CAML Interactor (TACI); B-cell activating factor receptor (BAFF-R); V-Ki-ras2 Kirsten rat sarcoma viral oncogene homolog (KRAS); immunoglobulin lambda-like polypeptide 1 (IGLL1); 707-AP (707 alanine proline); ART-4 (adenocarcinoma antigen recognized by T4 cells); BAGE (B antigen; b-catenin/m, b-catenin/mutated); CAMEL (CTL-recognized antigen on melanoma); CAP1 (carcinoembryonic antigen peptide 1); CASP-8 (caspase-8); CDC27m (cell-division cycle 27 mutated); CDK4/m (cycline-dependent kinase 4 mutated); Cyp-B (cyclophilin B); DAM (differentiation antigen melanoma); EGP-2 (epithelial glycoprotein 2); EGP-40 (epithelial glycoprotein 40); Erbb2, 3, 4 (erythroblastic leukemia viral oncogene homolog-2, -3, 4); FBP (folate binding protein); fAchR (Fetal acetylcholine receptor); G250 (glycoprotein 250); GAGE (G antigen); GnT-V (N-acetylglucosaminyltransferase V); HAGE (helicose antigen); ULA-A (human leukocyte antigen-A); HST2 (human signet ring tumor 2); KIAA0205; KDR (kinase insert domain receptor); LDLR/FUT (low density lipid receptor/GDP L-fucose: b-D-galactosidase 2-a-L fucosyltransferase); L1CAM (L1 cell adhesion molecule); MC1R (melanocortin 1 receptor); Myosin/m (myosin mutated); MUM-1, -2, -3 (melanoma ubiquitous mutated 1, 2, 3); NA88-A (NA cDNA clone of patient M88); KG2D (Natural killer group 2, member D) ligands; oncofetal antigen (h5T4); p190 minor bcr-abl (protein of 190KD bcr-abl); Pml/RARa (promyelocytic leukemia/retinoic acid receptor a); PRAME (preferentially expressed antigen of melanoma); SAGE (sarcoma antigen); TEL/AML1 (translocation Ets-family leukemia/acute myeloid leukemia 1); TPI/m (triosephosphate isomerase mutated); CD70; and any combination thereof.

In certain embodiments, an antigen to be targeted in adoptive cell therapy (such as particularly CAR or TCR T-cell therapy) of a disease (such as particularly of tumor or cancer) is a tumor-specific antigen (TSA).

In certain embodiments, an antigen to be targeted in adoptive cell therapy (such as particularly CAR or TCR T-cell therapy) of a disease (such as particularly of tumor or cancer) is a neoantigen.

In certain embodiments, an antigen to be targeted in adoptive cell therapy (such as particularly CAR or TCR T-cell therapy) of a disease (such as particularly of tumor or cancer) is a tumor-associated antigen (TAA).

In certain embodiments, an antigen to be targeted in adoptive cell therapy (such as particularly CAR or TCR T-cell therapy) of a disease (such as particularly of tumor or cancer) is a universal tumor antigen. In certain preferred embodiments, the universal tumor antigen is selected from the group consisting of: a human telomerase reverse transcriptase (hTERT), survivin, mouse double minute 2 homolog (MDM2), cytochrome P450 1B 1 (CYP1B), HER2/neu, Wilms' tumor gene 1 (WT1), livin, alphafetoprotein (AFP), carcinoembryonic antigen (CEA), mucin 16 (MUC16), MUC1, prostate-specific membrane antigen (PSMA), p53, cyclin (Dl), and any combinations thereof.

In certain embodiments, an antigen (such as a tumor antigen) to be targeted in adoptive cell therapy (such as particularly CAR or TCR T-cell therapy) of a disease (such as particularly of tumor or cancer) may be selected from a group consisting of: CD19, BCMA, CD70, CLL-1, MAGE A3, MAGE A6, HPV E6, HPV E7, WT1, CD22, CD171, ROR1, MUC16, and SSX2. In certain preferred embodiments, the antigen may be CD19. For example, CD19 may be targeted in hematologic malignancies, such as in lymphomas, more particularly in B-cell lymphomas, such as without limitation in diffuse large B-cell lymphoma, primary mediastinal b-cell lymphoma, transformed follicular lymphoma, marginal zone lymphoma, mantle cell lymphoma, acute lymphoblastic leukemia including adult and pediatric ALL, non-Hodgkin lymphoma, indolent non-Hodgkin lymphoma, or chronic lymphocytic leukemia. For example, BCMA may be targeted in multiple myeloma or plasma cell leukemia (see, e.g., 2018 American Association for Cancer Research (AACR) Annual meeting Poster: Allogeneic Chimeric Antigen Receptor T Cells Targeting B Cell Maturation Antigen). For example, CLL1 may be targeted in acute myeloid leukemia. For example, MAGE A3, MAGE A6, SSX2, and/or KRAS may be targeted in solid tumors. For example, HPV E6 and/or HPV E7 may be targeted in cervical cancer or head and neck cancer. For example, WT1 may be targeted in acute myeloid leukemia (AML), myelodysplastic syndromes (MDS), chronic myeloid leukemia (CIVIL), non-small cell lung cancer, breast, pancreatic, ovarian or colorectal cancers, or mesothelioma. For example, CD22 may be targeted in B cell malignancies, including non-Hodgkin lymphoma, diffuse large B-cell lymphoma, or acute lymphoblastic leukemia. For example, CD171 may be targeted in neuroblastoma, glioblastoma, or lung, pancreatic, or ovarian cancers. For example, ROR1 may be targeted in ROR1+ malignancies, including non-small cell lung cancer, triple negative breast cancer, pancreatic cancer, prostate cancer, ALL, chronic lymphocytic leukemia, or mantle cell lymphoma. For example, MUC16 may be targeted in MUC16ecto+epithelial ovarian, fallopian tube or primary peritoneal cancer. For example, CD70 may be targeted in both hematologic malignancies as well as in solid cancers such as renal cell carcinoma (RCC), gliomas (e.g., GBM), and head and neck cancers (HNSCC). CD70 is expressed in both hematologic malignancies as well as in solid cancers, while its expression in normal tissues is restricted to a subset of lymphoid cell types (see, e.g., 2018 American Association for Cancer Research (AACR) Annual meeting Poster: Allogeneic CRISPR Engineered Anti-CD70 CAR-T Cells Demonstrate Potent Preclinical Activity Against Both Solid and Hematological Cancer Cells).

Various strategies may for example be employed to genetically modify T cells by altering the specificity of the T cell receptor (TCR) for example by introducing new TCR α and β chains with selected peptide specificity (see U.S. Pat. No. 8,697,854; PCT Patent Publications: WO2003020763, WO2004033685, WO2004044004, WO2005114215, WO2006000830, WO2008038002, WO2008039818, WO2004074322, WO2005113595, WO2006125962, WO2013166321, WO2013039889, WO2014018863, WO2014083173; U.S. Pat. No. 8,088,379).

As an alternative to, or addition to, TCR modifications, chimeric antigen receptors (CARs) may be used in order to generate immunoresponsive cells, such as T cells, specific for selected targets, such as malignant cells, with a wide variety of receptor chimera constructs having been described (see U.S. Pat. Nos. 5,843,728; 5,851,828; 5,912,170; 6,004,811; 6,284,240; 6,392,013; 6,410,014; 6,753,162; 8,211,422; and PCT Publication WO9215322).

In general, CARs are comprised of an extracellular domain, a transmembrane domain, and an intracellular domain, wherein the extracellular domain comprises an antigen-binding domain that is specific for a predetermined target. While the antigen-binding domain of a CAR is often an antibody or antibody fragment (e.g., a single chain variable fragment, scFv), the binding domain is not particularly limited so long as it results in specific recognition of a target. For example, in some embodiments, the antigen-binding domain may comprise a receptor, such that the CAR is capable of binding to the ligand of the receptor. Alternatively, the antigen-binding domain may comprise a ligand, such that the CAR is capable of binding the endogenous receptor of that ligand.

The antigen-binding domain of a CAR is generally separated from the transmembrane domain by a hinge or spacer. The spacer is also not particularly limited, and it is designed to provide the CAR with flexibility. For example, a spacer domain may comprise a portion of a human Fc domain, including a portion of the CH3 domain, or the hinge region of any immunoglobulin, such as IgA, IgD, IgE, IgG, or IgM, or variants thereof. Furthermore, the hinge region may be modified so as to prevent off-target binding by FcRs or other potential interfering objects. For example, the hinge may comprise an IgG4 Fc domain with or without a S228P, L235E, and/or N297Q mutation (according to Kabat numbering) in order to decrease binding to FcRs. Additional spacers/hinges include, but are not limited to, CD4, CD8, and CD28 hinge regions.

The transmembrane domain of a CAR may be derived either from a natural or from a synthetic source. Where the source is natural, the domain may be derived from any membrane bound or transmembrane protein. Transmembrane regions of particular use in this disclosure may be derived from CD8, CD28, CD3, CD45, CD4, CD5, CDS, CD9, CD 16, CD22, CD33, CD37, CD64, CD80, CD86, CD 134, CD137, CD 154, TCR. Alternatively, the transmembrane domain may be synthetic, in which case it will comprise predominantly hydrophobic residues such as leucine and valine. Preferably a triplet of phenylalanine, tryptophan and valine will be found at each end of a synthetic transmembrane domain. Optionally, a short oligo- or polypeptide linker, preferably between 2 and 10 amino acids in length may form the linkage between the transmembrane domain and the cytoplasmic signaling domain of the CAR. A glycine-serine doublet provides a particularly suitable linker.

Alternative CAR constructs may be characterized as belonging to successive generations. First-generation CARs typically consist of a single-chain variable fragment of an antibody specific for an antigen, for example comprising a VL linked to a VH of a specific antibody, linked by a flexible linker, for example by a CD8a hinge domain and a CD8a transmembrane domain, to the transmembrane and intracellular signaling domains of either CD3 or FcRγ (scFv-CD3t or scFv-FcRγ; see U.S. Pat. Nos. 7,741,465; 5,912,172; 5,906,936). Second-generation CARs incorporate the intracellular domains of one or more costimulatory molecules, such as CD28, OX40 (CD134), or 4-1BB (CD137) within the endodomain (for example scFv-CD28/OX40/4-1BB-CD3ζ; see U.S. Pat. Nos. 8,911,993; 8,916,381; 8,975,071; 9,101,584; 9,102,760; 9,102,761). Third-generation CARs include a combination of costimulatory endodomains, such a CD3ζ-chain, CD97, GDI 1a-CD18, CD2, ICOS, CD27, CD154, CDS, OX40, 4-1BB, CD2, CD7, LIGHT, LFA-1, NKG2C, B7-H3, CD30, CD40, PD-1, or CD28 signaling domains (for example scFv-CD28-4-1BB-CD3ζ or scFv-CD28-OX40-CD3ζ; see U.S. Pat. Nos. 8,906,682; 8,399,645; 5,686,281; PCT Publication No. WO2014134165; PCT Publication No. WO2012079000). In certain embodiments, the primary signaling domain comprises a functional signaling domain of a protein selected from the group consisting of CD3 zeta, CD3 gamma, CD3 delta, CD3 epsilon, common FcR gamma (FCERIG), FcR beta (Fc Epsilon Rib), CD79a, CD79b, Fc gamma RIIa, DAP10, and DAP12. In certain preferred embodiments, the primary signaling domain comprises a functional signaling domain of CD3t or FcRγ. In certain embodiments, the one or more costimulatory signaling domains comprise a functional signaling domain of a protein selected, each independently, from the group consisting of: CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, a ligand that specifically binds with CD83, CDS, ICAM-1, GITR, BAFFR, HVEM (LIGHTR), SLAMF7, NKp80 (KLRF1), CD160, CD19, CD4, CD8 alpha, CD8 beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11 a, LFA-1, ITGAM, CD11b, ITGAX, CD11c, ITGB1, CD29, ITGB2, CD18, ITGB7, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, NKp44, NKp30, NKp46, and NKG2D. In certain embodiments, the one or more costimulatory signaling domains comprise a functional signaling domain of a protein selected, each independently, from the group consisting of: 4-1BB, CD27, and CD28. In certain embodiments, a chimeric antigen receptor may have the design as described in U.S. Pat. No. 7,446,190, comprising an intracellular domain of CD3 chain (such as amino acid residues 52-163 of the human CD3 zeta chain, as shown in SEQ ID NO: 14 of U.S. Pat. No. 7,446,190), a signaling region from CD28 and an antigen-binding element (or portion or domain; such as scFv). The CD28 portion, when between the zeta chain portion and the antigen-binding element, may suitably include the transmembrane and signaling domains of CD28 (such as amino acid residues 114-220 of SEQ ID NO: 10, full sequence shown in SEQ ID NO: 6 of U.S. Pat. No. 7,446,190; these can include the following portion of CD28 as set forth in Genbank identifier NM_006139 (sequence version 1, 2 or 3): IEVMYPPPYLDNEKSNGTIIHVKGKHLCPSPLFPGPSKPFWVLVVVGGVLACYSLLVTVA FIIFWVRSKRSRLLHSDYMNMTPRRPGPTRKHYQPYAPPRDFAAYRS)) (SEQ. I.D. No. 3). Alternatively, when the zeta sequence lies between the CD28 sequence and the antigen-binding element, intracellular domain of CD28 can be used alone (such as amino sequence set forth in SEQ ID NO: 9 of U.S. Pat. No. 7,446,190). Hence, certain embodiments employ a CAR comprising (a) a zeta chain portion comprising the intracellular domain of human CD3t chain, (b) a costimulatory signaling region, and (c) an antigen-binding element (or portion or domain), wherein the costimulatory signaling region comprises the amino acid sequence encoded by SEQ ID NO: 6 of U.S. Pat. No. 7,446,190.

Alternatively, costimulation may be orchestrated by expressing CARs in antigen-specific T cells, chosen so as to be activated and expanded following engagement of their native αβTCR, for example by antigen on professional antigen-presenting cells, with attendant costimulation. In addition, additional engineered receptors may be provided on the immunoresponsive cells, for example to improve targeting of a T-cell attack and/or minimize side effects

By means of an example and without limitation, Kochenderfer et al., (2009) J Immunother. 32 (7): 689-702 described anti-CD19 chimeric antigen receptors (CAR). FMC63-28Z CAR contained a single chain variable region moiety (scFv) recognizing CD19 derived from the FMC63 mouse hybridoma (described in Nicholson et al., (1997) Molecular Immunology 34: 1157-1165), a portion of the human CD28 molecule, and the intracellular component of the human TCR-ζ molecule. FMC63-CD828BBZ CAR contained the FMC63 scFv, the hinge and transmembrane regions of the CD8 molecule, the cytoplasmic portions of CD28 and 4-1BB, and the cytoplasmic component of the TCR-ζ molecule. The exact sequence of the CD28 molecule included in the FMC63-28Z CAR corresponded to Genbank identifier NM_006139; the sequence included all amino acids starting with the amino acid sequence IEVMYPPPY (SEQ. I.D. No. 2) and continuing all the way to the carboxy-terminus of the protein. To encode the anti-CD19 scFv component of the vector, the authors designed a DNA sequence which was based on a portion of a previously published CAR (Cooper et al., (2003) Blood 101: 1637-1644). This sequence encoded the following components in frame from the 5′ end to the 3′ end: an XhoI site, the human granulocyte-macrophage colony-stimulating factor (GM-CSF) receptor a-chain signal sequence, the FMC63 light chain variable region (as in Nicholson et al., supra), a linker peptide (as in Cooper et al., supra), the FMC63 heavy chain variable region (as in Nicholson et al., supra), and a NotI site. A plasmid encoding this sequence was digested with XhoI and NotI. To form the MSGV-FMC63-28Z retroviral vector, the XhoI and NotI-digested fragment encoding the FMC63 scFv was ligated into a second XhoI and NotI-digested fragment that encoded the MSGV retroviral backbone (as in Hughes et al., (2005) Human Gene Therapy 16: 457-472) as well as part of the extracellular portion of human CD28, the entire transmembrane and cytoplasmic portion of human CD28, and the cytoplasmic portion of the human TCR-ζ molecule (as in Maher et al., 2002) Nature Biotechnology 20: 70-75). The FMC63-28Z CAR is included in the KTE-C19 (axicabtagene ciloleucel) anti-CD19 CAR-T therapy product in development by Kite Pharma, Inc. for the treatment of inter alia patients with relapsed/refractory aggressive B-cell non-Hodgkin lymphoma (NHL). Accordingly, in certain embodiments, cells intended for adoptive cell therapies, more particularly immunoresponsive cells such as T cells, may express the FMC63-28Z CAR as described by Kochenderfer et al. (supra). Hence, in certain embodiments, cells intended for adoptive cell therapies, more particularly immunoresponsive cells such as T cells, may comprise a CAR comprising an extracellular antigen-binding element (or portion or domain; such as scFv) that specifically binds to an antigen, an intracellular signaling domain comprising an intracellular domain of a CD3t chain, and a costimulatory signaling region comprising a signaling domain of CD28. Preferably, the CD28 amino acid sequence is as set forth in Genbank identifier NM_006139 (sequence version 1, 2 or 3) starting with the amino acid sequence IEVMYPPPY (SEQ ID NO: 4) and continuing all the way to the carboxy-terminus of the protein. The sequence is reproduced herein:

(SEQ ID NO: 5)
IEVMYPPPYLDNEKSNGTIIHVKGKHLCPSPLFPGPSKPFWVLVVVGGV
LACYSLLVTVAFIIFWVRSKRSRLLHSDYMNMTPRRPGPTRKHYQPYAP
PRDFAAYRS.

Preferably, the antigen is CD19, more preferably the antigen-binding element is an anti-CD19 scFv, even more preferably the anti-CD19 scFv as described by Kochenderfer et al. (supra).

Additional anti-CD19 CARs are further described in WO2015187528. More particularly Example 1 and Table 1 of International Patent Publication No. WO2015187528, incorporated by reference herein, demonstrate the generation of anti-CD19 CARs based on a fully human anti-CD19 monoclonal antibody (47G4, as described in US20100104509) and murine anti-CD19 monoclonal antibody (as described in Nicholson et al. and explained above). Various combinations of a signal sequence (human CD8-alpha or GM-CSF receptor), extracellular and transmembrane regions (human CD8-alpha) and intracellular T-cell signaling domains (CD28-CD3ζ; 4-1BB-CD3ζ; CD27-CD3ζ; CD28-CD27-CD3ζ, 4-1BB-CD27-CD3ζ; CD27-4-1BB-CD3ζ; CD28-CD27-FcεRT gamma chain; or CD28-FcεRT gamma chain) were disclosed. Hence, in certain embodiments, cells intended for adoptive cell therapies, more particularly immunoresponsive cells such as T cells, may comprise a CAR comprising an extracellular antigen-binding element that specifically binds to an antigen, an extracellular and transmembrane region as set forth in Table 1 of WO2015187528 and an intracellular T-cell signaling domain as set forth in Table 1 of WO2015187528. Preferably, the antigen is CD19, more preferably the antigen-binding element is an anti-CD19 scFv, even more preferably the mouse or human anti-CD19 scFv as described in Example 1 of WO2015187528. In certain embodiments, the CAR comprises, consists essentially of or consists of an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, or SEQ ID NO: 13 as set forth in Table 1 of WO2015187528.

By means of an example and without limitation, chimeric antigen receptor that recognizes the CD70 antigen is described in International Patent Publication No. WO2012058460A2 (see also, Park et al., CD70 as a target for chimeric antigen receptor T cells in head and neck squamous cell carcinoma, Oral Oncol. 2018 March; 78:145-150; and Jin et al., CD70, a novel target of CAR T-cell therapy for gliomas, Neuro Oncol. 2018 Jan. 10; 20(1):55-65). CD70 is expressed by diffuse large B-cell and follicular lymphoma and also by the malignant cells of Hodgkins lymphoma, Waldenstrom's macroglobulinemia and multiple myeloma, and by HTLV-1- and EBV-associated malignancies. (Agathanggelou et al. Am. J. Pathol. 1995; 147: 1152-1160; Hunter et al., Blood 2004; 104:4881. 26; Lens et al., J Immunol. 2005; 174:6212-6219; Baba et al., J Virol. 2008; 82:3843-3852.) In addition, CD70 is expressed by non-hematological malignancies such as renal cell carcinoma and glioblastoma. (Junker et al., J Urol. 2005; 173:2150-2153; Chahlavi et al., Cancer Res 2005; 65:5428-5438) Physiologically, CD70 expression is transient and restricted to a subset of highly activated T, B, and dendritic cells.

By means of an example and without limitation, chimeric antigen receptor that recognizes BCMA has been described (see, e.g., US20160046724A1; WO2016014789A2; WO2017211900A1; WO2015158671A1; US20180085444A1; WO2018028647A1; US20170283504A1; and WO2013154760A1).

In certain embodiments, the immune cell may, in addition to a CAR or exogenous TCR as described herein, further comprise a chimeric inhibitory receptor (inhibitory CAR) that specifically binds to a second target antigen and is capable of inducing an inhibitory or immunosuppressive or repressive signal to the cell upon recognition of the second target antigen. In certain embodiments, the chimeric inhibitory receptor comprises an extracellular antigen-binding element (or portion or domain) configured to specifically bind to a target antigen, a transmembrane domain, and an intracellular immunosuppressive or repressive signaling domain. In certain embodiments, the second target antigen is an antigen that is not expressed on the surface of a cancer cell or infected cell or the expression of which is downregulated on a cancer cell or an infected cell. In certain embodiments, the second target antigen is an MHC-class I molecule. In certain embodiments, the intracellular signaling domain comprises a functional signaling portion of an immune checkpoint molecule, such as for example PD-1 or CTLA4. Advantageously, the inclusion of such inhibitory CAR reduces the chance of the engineered immune cells attacking non-target (e.g., non-cancer) tissues.

Alternatively, T-cells expressing CARs may be further modified to reduce or eliminate expression of endogenous TCRs in order to reduce off-target effects. Reduction or elimination of endogenous TCRs can reduce off-target effects and increase the effectiveness of the T cells (U.S. Pat. No. 9,181,527). T cells stably lacking expression of a functional TCR may be produced using a variety of approaches. T cells internalize, sort, and degrade the entire T cell receptor as a complex, with a half-life of about 10 hours in resting T cells and 3 hours in stimulated T cells (von Essen, M. et al. 2004. J. Immunol. 173:384-393). Proper functioning of the TCR complex requires the proper stoichiometric ratio of the proteins that compose the TCR complex. TCR function also requires two functioning TCR zeta proteins with ITAM motifs. The activation of the TCR upon engagement of its MHC-peptide ligand requires the engagement of several TCRs on the same T cell, which all must signal properly. Thus, if a TCR complex is destabilized with proteins that do not associate properly or cannot signal optimally, the T cell will not become activated sufficiently to begin a cellular response.

Accordingly, in some embodiments, TCR expression may eliminated using RNA interference (e.g., shRNA, siRNA, miRNA, etc.), CRISPR, or other methods that target the nucleic acids encoding specific TCRs (e.g., TCR-α and TCR-β) and/or CD3 chains in primary T cells. By blocking expression of one or more of these proteins, the T cell will no longer produce one or more of the key components of the TCR complex, thereby destabilizing the TCR complex and preventing cell surface expression of a functional TCR.

In some instances, CAR may also comprise a switch mechanism for controlling expression and/or activation of the CAR. For example, a CAR may comprise an extracellular, transmembrane, and intracellular domain, in which the extracellular domain comprises a target-specific binding element that comprises a label, binding domain, or tag that is specific for a molecule other than the target antigen that is expressed on or by a target cell. In such embodiments, the specificity of the CAR is provided by a second construct that comprises a target antigen binding domain (e.g., an scFv or a bispecific antibody that is specific for both the target antigen and the label or tag on the CAR) and a domain that is recognized by or binds to the label, binding domain, or tag on the CAR. See, e.g., International Patent Publication Nos. WO 2013/044225, WO 2016/000304, WO 2015/057834, WO 2015/057852, and WO 2016/070061, U.S. Pat. No. 9,233,125, US Patent Publication No. 2016/0129109. In this way, a T-cell that expresses the CAR can be administered to a subject, but the CAR cannot bind its target antigen until the second composition comprising an antigen-specific binding domain is administered.

Alternative switch mechanisms include CARs that require multimerization in order to activate their signaling function (see, e.g., US Patent Publication Nos. 2015/0368342, US 2016/0175359, US 2015/0368360) and/or an exogenous signal, such as a small molecule drug (US Patent Publication No. 2016/0166613, Yung et al., Science, 2015), in order to elicit a T-cell response. Some CARs may also comprise a “suicide switch” to induce cell death of the CAR T-cells following treatment (Buddee et al., PLoS One, 2013) or to downregulate expression of the CAR following binding to the target antigen (WO 2016/011210).

Alternative techniques may be used to transform target immunoresponsive cells, such as protoplast fusion, lipofection, transfection or electroporation. A wide variety of vectors may be used, such as retroviral vectors, lentiviral vectors, adenoviral vectors, adeno-associated viral vectors, plasmids or transposons, such as a Sleeping Beauty transposon (see U.S. Pat. Nos. 6,489,458; 7,148,203; 7,160,682; 7,985,739; 8,227,432), may be used to introduce CARs, for example using 2nd generation antigen-specific CARs signaling through CD3ζ and either CD28 or CD137. Viral vectors may for example include vectors based on HIV, SV40, EBV, HSV or BPV.

Cells that are targeted for transformation may for example include T cells, Natural Killer (NK) cells, cytotoxic T lymphocytes (CTL), regulatory T cells, human embryonic stem cells, tumor-infiltrating lymphocytes (TIL) or a pluripotent stem cell from which lymphoid cells may be differentiated. T cells expressing a desired CAR may for example be selected through co-culture with γ-irradiated activating and propagating cells (AaPC), which co-express the cancer antigen and co-stimulatory molecules. The engineered CAR T-cells may be expanded, for example by co-culture on AaPC in presence of soluble factors, such as IL-2 and IL-21. This expansion may for example be carried out so as to provide memory CAR+ T cells (which may for example be assayed by non-enzymatic digital array and/or multi-panel flow cytometry). In this way, CAR T cells may be provided that have specific cytotoxic activity against antigen-bearing tumors (optionally in conjunction with production of desired chemokines such as interferon-y). CART cells of this kind may for example be used in animal models, for example to treat tumor xenografts.

In certain embodiments, ACT includes co-transferring CD4+Th1 cells and CD8+ CTLs to induce a synergistic antitumour response (see, e.g., Li et al., Adoptive cell therapy with CD4+T helper 1 cells and CD8+ cytotoxic T cells enhances complete rejection of an established tumor, leading to generation of endogenous memory responses to non-targeted tumor epitopes. Clin Transl Immunology. 2017 October; 6(10): e160).

In certain embodiments, Th17 cells are transferred to a subject in need thereof. Th17 cells have been reported to directly eradicate melanoma tumors in mice to a greater extent than Th1 cells (Muranski P, et al., Tumor-specific Th17-polarized cells eradicate large established melanoma. Blood. 2008 Jul. 15; 112(2):362-73; and Martin-Orozco N, et al., T helper 17 cells promote cytotoxic T cell activation in tumor immunity. Immunity. 2009 Nov. 20; 31(5):787-98). Those studies involved an adoptive T cell transfer (ACT) therapy approach, which takes advantage of CD4⁺ T cells that express a TCR recognizing tyrosinase tumor antigen. Exploitation of the TCR leads to rapid expansion of Th17 populations to large numbers ex vivo for reinfusion into the autologous tumor-bearing hosts.

In certain embodiments, ACT may include autologous iPSC-based vaccines, such as irradiated iPSCs in autologous anti-tumor vaccines (see e.g., Kooreman, Nigel G. et al., Autologous iPSC-Based Vaccines Elicit Anti-tumor Responses In Vivo, Cell Stem Cell 22, 1-13, 2018, doi.org/10.1016/j.stem.2018.01.016).

Unlike T-cell receptors (TCRs) that are MHC restricted, CARs can potentially bind any cell surface-expressed antigen and can thus be more universally used to treat patients (see Irving et al., Engineering Chimeric Antigen Receptor T-Cells for Racing in Solid Tumors: Don't Forget the Fuel, Front. Immunol., 3 Apr. 2017, doi.org/10.3389/fimmu.2017.00267). In certain embodiments, in the absence of endogenous T-cell infiltrate (e.g., due to aberrant antigen processing and presentation), which precludes the use of TIL therapy and immune checkpoint blockade, the transfer of CAR T-cells may be used to treat patients (see, e.g., Hinrichs C S, Rosenberg S A. Exploiting the curative potential of adoptive T-cell therapy for cancer. Immunol Rev (2014) 257(1):56-71. doi:10.1111/imr.12132).

Approaches such as the foregoing may be adapted to provide methods of treating and/or increasing survival of a subject having a disease, such as a neoplasia, for example by administering an effective amount of an immunoresponsive cell comprising an antigen recognizing receptor that binds a selected antigen, wherein the binding activates the immunoresponsive cell, thereby treating or preventing the disease (such as a neoplasia, a pathogen infection, an autoimmune disorder, or an allogeneic transplant reaction).

In certain embodiments, the treatment can be administered after lymphodepleting pretreatment in the form of chemotherapy (typically a combination of cyclophosphamide and fludarabine) or radiation therapy. Initial studies in ACT had short lived responses and the transferred cells did not persist in vivo for very long (Houot et al., T-cell-based immunotherapy: adoptive cell transfer and checkpoint inhibition. Cancer Immunol Res (2015) 3(10):1115-22; and Kamta et al., Advancing Cancer Therapy with Present and Emerging Immuno-Oncology Approaches. Front. Oncol. (2017) 7:64). Immune suppressor cells like Tregs and MDSCs may attenuate the activity of transferred cells by outcompeting them for the necessary cytokines. Not being bound by a theory lymphodepleting pretreatment may eliminate the suppressor cells allowing the TILs to persist.

In one embodiment, the treatment can be administrated into patients undergoing an immunosuppressive treatment (e.g., glucocorticoid treatment). The cells or population of cells may be made resistant to at least one immunosuppressive agent due to the inactivation of a gene encoding a receptor for such immunosuppressive agent. In certain embodiments, the immunosuppressive treatment provides for the selection and expansion of the immunoresponsive T cells within the patient.

In certain embodiments, the treatment can be administered before primary treatment (e.g., surgery or radiation therapy) to shrink a tumor before the primary treatment. In another embodiment, the treatment can be administered after primary treatment to remove any remaining cancer cells.

In certain embodiments, immunometabolic barriers can be targeted therapeutically prior to and/or during ACT to enhance responses to ACT or CAR T-cell therapy and to support endogenous immunity (see, e.g., Irving et al., Engineering Chimeric Antigen Receptor T-Cells for Racing in Solid Tumors: Don't Forget the Fuel, Front. Immunol., 3 Apr. 2017, doi.org/10.3389/fimmu.2017.00267).

The administration of cells or population of cells, such as immune system cells or cell populations, such as more particularly immunoresponsive cells or cell populations, as disclosed herein may be carried out in any convenient manner, including by aerosol inhalation, injection, ingestion, transfusion, implantation or transplantation. The cells or population of cells may be administered to a patient subcutaneously, intradermally, intratumorally, intranodally, intramedullary, intramuscularly, intrathecally, by intravenous or intralymphatic injection, or intraperitoneally. In some embodiments, the disclosed CARs may be delivered or administered into a cavity formed by the resection of tumor tissue (i.e. intracavity delivery) or directly into a tumor prior to resection (i.e. intratumoral delivery). In one embodiment, the cell compositions of the present invention are preferably administered by intravenous injection.

The administration of the cells or population of cells can consist of the administration of 10⁴-10⁹ cells per kg body weight, preferably 10⁵ to 10⁶ cells/kg body weight including all integer values of cell numbers within those ranges. Dosing in CAR T cell therapies may for example involve administration of from 10⁶ to 10⁹ cells/kg, with or without a course of lymphodepletion, for example with cyclophosphamide. The cells or population of cells can be administrated in one or more doses. In another embodiment, the effective amount of cells are administrated as a single dose. In another embodiment, the effective amount of cells are administrated as more than one dose over a period time. Timing of administration is within the judgment of managing physician and depends on the clinical condition of the patient. The cells or population of cells may be obtained from any source, such as a blood bank or a donor. While individual needs vary, determination of optimal ranges of effective amounts of a given cell type for a particular disease or conditions are within the skill of one in the art. An effective amount means an amount which provides a therapeutic or prophylactic benefit. The dosage administrated will be dependent upon the age, health and weight of the recipient, kind of concurrent treatment, if any, frequency of treatment and the nature of the effect desired.

In another embodiment, the effective amount of cells or composition comprising those cells are administrated parenterally. The administration can be an intravenous administration. The administration can be directly done by injection within a tumor.

To guard against possible adverse reactions, engineered immunoresponsive cells may be equipped with a transgenic safety switch, in the form of a transgene that renders the cells vulnerable to exposure to a specific signal. For example, the herpes simplex viral thymidine kinase (TK) gene may be used in this way, for example by introduction into allogeneic T lymphocytes used as donor lymphocyte infusions following stem cell transplantation (Greco, et al., Improving the safety of cell therapy with the TK-suicide gene. Front. Pharmacol. 2015; 6: 95). In such cells, administration of a nucleoside prodrug such as ganciclovir or acyclovir causes cell death. Alternative safety switch constructs include inducible caspase 9, for example triggered by administration of a small-molecule dimerizer that brings together two nonfunctional icasp9 molecules to form the active enzyme. A wide variety of alternative approaches to implementing cellular proliferation controls have been described (see U.S. Patent Publication No. 20130071414; PCT Patent Publication WO2011146862; PCT Patent Publication WO2014011987; PCT Patent Publication WO2013040371; Zhou et al. BLOOD, 2014, 123/25:3895-3905; Di Stasi et al., The New England Journal of Medicine 2011; 365:1673-1683; Sadelain M, The New England Journal of Medicine 2011; 365:1735-173; Ramos et al., Stem Cells 28(6):1107-15 (2010)).

In a further refinement of adoptive therapies, genome editing may be used to tailor immunoresponsive cells to alternative implementations, for example providing edited CAR T cells (see Poirot et al., 2015, Multiplex genome edited T-cell manufacturing platform for “off-the-shelf” adoptive T-cell immunotherapies, Cancer Res 75 (18): 3853; Ren et al., 2017, Multiplex genome editing to generate universal CAR T cells resistant to PD1 inhibition, Clin Cancer Res. 2017 May 1; 23(9):2255-2266. doi: 10.1158/1078-0432.CCR-16-1300. Epub 2016 Nov. 4; Qasim et al., 2017, Molecular remission of infant B-ALL after infusion of universal TALEN gene-edited CART cells, Sci Transl Med. 2017 Jan. 25; 9(374); Legut, et al., 2018, CRISPR-mediated TCR replacement generates superior anticancer transgenic T cells. Blood, 131(3), 311-322; and Georgiadis et al., Long Terminal Repeat CRISPR-CAR-Coupled “Universal” T Cells Mediate Potent Anti-leukemic Effects, Molecular Therapy, In Press, Corrected Proof, Available online 6 Mar. 2018). Cells may be edited using any CRISPR system and method of use thereof as described herein. CRISPR systems may be delivered to an immune cell by any method described herein. In preferred embodiments, cells are edited ex vivo and transferred to a subject in need thereof. Immunoresponsive cells, CAR T cells or any cells used for adoptive cell transfer may be edited. Editing may be performed for example to insert or knock-in an exogenous gene, such as an exogenous gene encoding a CAR or a TCR, at a preselected locus in a cell (e.g. TRAC locus); to eliminate potential alloreactive T-cell receptors (TCR) or to prevent inappropriate pairing between endogenous and exogenous TCR chains, such as to knock-out or knock-down expression of an endogenous TCR in a cell; to disrupt the target of a chemotherapeutic agent in a cell; to block an immune checkpoint, such as to knock-out or knock-down expression of an immune checkpoint protein or receptor in a cell; to knock-out or knock-down expression of other gene or genes in a cell, the reduced expression or lack of expression of which can enhance the efficacy of adoptive therapies using the cell; to knock-out or knock-down expression of an endogenous gene in a cell, said endogenous gene encoding an antigen targeted by an exogenous CAR or TCR; to knock-out or knock-down expression of one or more MHC constituent proteins in a cell; to activate a T cell; to modulate cells such that the cells are resistant to exhaustion or dysfunction; and/or increase the differentiation and/or proliferation of functionally exhausted or dysfunctional CD8+ T-cells (see PCT Patent Publications: WO2013176915, WO2014059173, WO2014172606, WO2014184744, and WO2014191128).

In certain embodiments, editing may result in inactivation of a gene. By inactivating a gene, it is intended that the gene of interest is not expressed in a functional protein form. In a particular embodiment, the CRISPR system specifically catalyzes cleavage in one targeted gene thereby inactivating said targeted gene. The nucleic acid strand breaks caused are commonly repaired through the distinct mechanisms of homologous recombination or non-homologous end joining (NHEJ). However, NHEJ is an imperfect repair process that often results in changes to the DNA sequence at the site of the cleavage. Repair via non-homologous end joining (NHEJ) often results in small insertions or deletions (Indel) and can be used for the creation of specific gene knockouts. Cells in which a cleavage induced mutagenesis event has occurred can be identified and/or selected by well-known methods in the art. In certain embodiments, homology directed repair (HDR) is used to concurrently inactivate a gene (e.g., TRAC) and insert an endogenous TCR or CAR into the inactivated locus.

Hence, in certain embodiments, editing of cells (such as by CRISPR/Cas), particularly cells intended for adoptive cell therapies, more particularly immunoresponsive cells such as T cells, may be performed to insert or knock-in an exogenous gene, such as an exogenous gene encoding a CAR or a TCR, at a preselected locus in a cell. Conventionally, nucleic acid molecules encoding CARs or TCRs are transfected or transduced to cells using randomly integrating vectors, which, depending on the site of integration, may lead to clonal expansion, oncogenic transformation, variegated transgene expression and/or transcriptional silencing of the transgene. Directing of transgene(s) to a specific locus in a cell can minimize or avoid such risks and advantageously provide for uniform expression of the transgene(s) by the cells. Without limitation, suitable ‘safe harbor’ loci for directed transgene integration include CCR5 or AAVS1. Homology-directed repair (HDR) strategies are known and described elsewhere in this specification allowing to insert transgenes into desired loci (e.g., TRAC locus).

Further suitable loci for insertion of transgenes, in particular CAR or exogenous TCR transgenes, include without limitation loci comprising genes coding for constituents of endogenous T-cell receptor, such as T-cell receptor alpha locus (TRA) or T-cell receptor beta locus (TRB), for example T-cell receptor alpha constant (TRAC) locus, T-cell receptor beta constant 1 (TRBC1) locus or T-cell receptor beta constant 2 (TRBC1) locus. Advantageously, insertion of a transgene into such locus can simultaneously achieve expression of the transgene, potentially controlled by the endogenous promoter, and knock-out expression of the endogenous TCR. This approach has been exemplified in Eyquem et al., (2017) Nature 543: 113-117, wherein the authors used CRISPR/Cas9 gene editing to knock-in a DNA molecule encoding a CD19-specific CAR into the TRAC locus downstream of the endogenous promoter; the CAR-T cells obtained by CRISPR were significantly superior in terms of reduced tonic CAR signaling and exhaustion.

T cell receptors (TCR) are cell surface receptors that participate in the activation of T cells in response to the presentation of antigen. The TCR is generally made from two chains, α and β, which assemble to form a heterodimer and associates with the CD3-transducing subunits to form the T cell receptor complex present on the cell surface. Each α and β chain of the TCR consists of an immunoglobulin-like N-terminal variable (V) and constant (C) region, a hydrophobic transmembrane domain, and a short cytoplasmic region. As for immunoglobulin molecules, the variable region of the α and β chains are generated by V(D)J recombination, creating a large diversity of antigen specificities within the population of T cells. However, in contrast to immunoglobulins that recognize intact antigen, T cells are activated by processed peptide fragments in association with an MHC molecule, introducing an extra dimension to antigen recognition by T cells, known as MHC restriction. Recognition of MHC disparities between the donor and recipient through the T cell receptor leads to T cell proliferation and the potential development of graft versus host disease (GVHD). The inactivation of TCRα or TCRβ can result in the elimination of the TCR from the surface of T cells preventing recognition of alloantigen and thus GVHD. However, TCR disruption generally results in the elimination of the CD3 signaling component and alters the means of further T cell expansion.

Hence, in certain embodiments, editing of cells (such as by CRISPR/Cas), particularly cells intended for adoptive cell therapies, more particularly immunoresponsive cells such as T cells, may be performed to knock-out or knock-down expression of an endogenous TCR in a cell. For example, NHEJ-based or HDR-based gene editing approaches can be employed to disrupt the endogenous TCR alpha and/or beta chain genes. For example, gene editing system or systems, such as CRISPR/Cas system or systems, can be designed to target a sequence found within the TCR beta chain conserved between the beta 1 and beta 2 constant region genes (TRBC1 and TRBC2) and/or to target the constant region of the TCR alpha chain (TRAC) gene.

Allogeneic cells are rapidly rejected by the host immune system. It has been demonstrated that, allogeneic leukocytes present in non-irradiated blood products will persist for no more than 5 to 6 days (Boni, Muranski et al. 2008 Blood 1; 112(12):4746-54). Thus, to prevent rejection of allogeneic cells, the host's immune system usually has to be suppressed to some extent. However, in the case of adoptive cell transfer the use of immunosuppressive drugs also have a detrimental effect on the introduced therapeutic T cells. Therefore, to effectively use an adoptive immunotherapy approach in these conditions, the introduced cells would need to be resistant to the immunosuppressive treatment. Thus, in a particular embodiment, the present invention further comprises a step of modifying T cells to make them resistant to an immunosuppressive agent, preferably by inactivating at least one gene encoding a target for an immunosuppressive agent. An immunosuppressive agent is an agent that suppresses immune function by one of several mechanisms of action. An immunosuppressive agent can be, but is not limited to a calcineurin inhibitor, a target of rapamycin, an interleukin-2 receptor a-chain blocker, an inhibitor of inosine monophosphate dehydrogenase, an inhibitor of dihydrofolic acid reductase, a corticosteroid or an immunosuppressive antimetabolite. The present invention allows conferring immunosuppressive resistance to T cells for immunotherapy by inactivating the target of the immunosuppressive agent in T cells. As non-limiting examples, targets for an immunosuppressive agent can be a receptor for an immunosuppressive agent such as: CD52, glucocorticoid receptor (GR), a FKBP family gene member and a cyclophilin family gene member.

In certain embodiments, editing of cells (such as by CRISPR/Cas), particularly cells intended for adoptive cell therapies, more particularly immunoresponsive cells such as T cells, may be performed to block an immune checkpoint, such as to knock-out or knock-down expression of an immune checkpoint protein or receptor in a cell. Immune checkpoints are inhibitory pathways that slow down or stop immune reactions and prevent excessive tissue damage from uncontrolled activity of immune cells. In certain embodiments, the immune checkpoint targeted is the programmed death-1 (PD-1 or CD279) gene (PDCD1). In other embodiments, the immune checkpoint targeted is cytotoxic T-lymphocyte-associated antigen (CTLA-4). In additional embodiments, the immune checkpoint targeted is another member of the CD28 and CTLA4 Ig superfamily such as BTLA, LAG3, ICOS, PDL1 or KIR. In further additional embodiments, the immune checkpoint targeted is a member of the TNFR superfamily such as CD40, OX40, CD137, GITR, CD27 or TIM-3.

Additional immune checkpoints include Src homology 2 domain-containing protein tyrosine phosphatase 1 (SHP-1) (Watson H A, et al., SHP-1: the next checkpoint target for cancer immunotherapy? Biochem Soc Trans. 2016 Apr. 15; 44(2):356-62). SHP-1 is a widely expressed inhibitory protein tyrosine phosphatase (PTP). In T-cells, it is a negative regulator of antigen-dependent activation and proliferation. It is a cytosolic protein, and therefore not amenable to antibody-mediated therapies, but its role in activation and proliferation makes it an attractive target for genetic manipulation in adoptive transfer strategies, such as chimeric antigen receptor (CAR) T cells. Immune checkpoints may also include T cell immunoreceptor with Ig and ITIM domains (TIGIT/Vstm3/WUCAM/VSIG9) and VISTA (Le Mercier I, et al., (2015) Beyond CTLA-4 and PD-1, the generation Z of negative checkpoint regulators. Front. Immunol. 6:418).

International Patent Publication No. WO 2014172606 relates to the use of MT1 and/or MT2 inhibitors to increase proliferation and/or activity of exhausted CD8+ T-cells and to decrease CD8+ T-cell exhaustion (e.g., decrease functionally exhausted or unresponsive CD8+ immune cells). In certain embodiments, metallothioneins are targeted by gene editing in adoptively transferred T cells.

In certain embodiments, targets of gene editing may be at least one targeted locus involved in the expression of an immune checkpoint protein. Such targets may include, but are not limited to CTLA4, PPP2CA, PPP2CB, PTPN6, PTPN22, PDCD1, ICOS (CD278), PDL1, KIR, LAG3, HAVCR2, BTLA, CD160, TIGIT, CD96, CRTAM, LAIR1, SIGLEC7, SIGLEC9, CD244 (2B4), TNFRSF10B, TNFRSF10A, CASP8, CASP10, CASP3, CASP6, CASP7, FADD, FAS, TGFBRII, TGFRBRI, SMAD2, SMAD3, SMAD4, SMAD10, SKI, SKIL, TGIF1, IL10RA, IL10RB, HMOX2, IL6R, IL6ST, EIF2AK4, CSK, PAG1, SIT1, FOXP3, PRDM1, BATF, VISTA, GUCY1A2, GUCY1A3, GUCY1B2, GUCY1B3, MT1, MT2, CD40, OX40, CD137, GITR, CD27, SHP-1, TIM-3, CEACAM-1, CEACAM-3, or CEACAM-5. In preferred embodiments, the gene locus involved in the expression of PD-1 or CTLA-4 genes is targeted. In other preferred embodiments, combinations of genes are targeted, such as but not limited to PD-1 and TIGIT.

By means of an example and without limitation, International Patent Publication No. WO 2016196388 concerns an engineered T cell comprising (a) a genetically engineered antigen receptor that specifically binds to an antigen, which receptor may be a CAR; and (b) a disrupted gene encoding a PD-L1, an agent for disruption of a gene encoding a PD-L1, and/or disruption of a gene encoding PD-L1, wherein the disruption of the gene may be mediated by a gene editing nuclease, a zinc finger nuclease (ZFN), CRISPR/Cas9 and/or TALEN. WO2015142675 relates to immune effector cells comprising a CAR in combination with an agent (such as CRISPR, TALEN or ZFN) that increases the efficacy of the immune effector cells in the treatment of cancer, wherein the agent may inhibit an immune inhibitory molecule, such as PD1, PD-L1, CTLA-4, TIM-3, LAG-3, VISTA, BTLA, TIGIT, LAIR1, CD160, 2B4, TGFR beta, CEACAM-1, CEACAM-3, or CEACAM-5. Ren et al., (2017) Clin Cancer Res 23 (9) 2255-2266 performed lentiviral delivery of CAR and electro-transfer of Cas9 mRNA and gRNAs targeting endogenous TCR, β-2 microglobulin (B2M) and PD1 simultaneously, to generate gene-disrupted allogeneic CART cells deficient of TCR, HLA class I molecule and PD1.

In certain embodiments, cells may be engineered to express a CAR, wherein expression and/or function of methylcytosine dioxygenase genes (TET1, TET2 and/or TET3) in the cells has been reduced or eliminated, such as by CRISPR, ZNF or TALEN (for example, as described in International Patent Publication No. WO 201704916).

In certain embodiments, editing of cells (such as by CRISPR/Cas), particularly cells intended for adoptive cell therapies, more particularly immunoresponsive cells such as T cells, may be performed to knock-out or knock-down expression of an endogenous gene in a cell, said endogenous gene encoding an antigen targeted by an exogenous CAR or TCR, thereby reducing the likelihood of targeting of the engineered cells. In certain embodiments, the targeted antigen may be one or more antigen selected from the group consisting of CD38, CD138, CS-1, CD33, CD26, CD30, CD53, CD92, CD100, CD148, CD150, CD200, CD261, CD262, CD362, human telomerase reverse transcriptase (hTERT), survivin, mouse double minute 2 homolog (MDM2), cytochrome P450 1B1 (CYP1B), HER2/neu, Wilms' tumor gene 1 (WT1), livin, alphafetoprotein (AFP), carcinoembryonic antigen (CEA), mucin 16 (MUC16), MUC1, prostate-specific membrane antigen (PSMA), p53, cyclin (D1), B cell maturation antigen (BCMA), transmembrane activator and CAML Interactor (TACI), and B-cell activating factor receptor (BAFF-R) (for example, as described in International Patent Publication Nos. WO 2016011210 and WO 2017011804).

In certain embodiments, editing of cells (such as by CRISPR/Cas), particularly cells intended for adoptive cell therapies, more particularly immunoresponsive cells such as T cells, may be performed to knock-out or knock-down expression of one or more MHC constituent proteins, such as one or more HLA proteins and/or beta-2 microglobulin (B2M), in a cell, whereby rejection of non-autologous (e.g., allogeneic) cells by the recipient's immune system can be reduced or avoided. In preferred embodiments, one or more HLA class I proteins, such as HLA-A, B and/or C, and/or B2M may be knocked-out or knocked-down. Preferably, B2M may be knocked-out or knocked-down. By means of an example, Ren et al., (2017) Clin Cancer Res 23 (9) 2255-2266 performed lentiviral delivery of CAR and electro-transfer of Cas9 mRNA and gRNAs targeting endogenous TCR, β-2 microglobulin (B2M) and PD1 simultaneously, to generate gene-disrupted allogeneic CAR T cells deficient of TCR, HLA class I molecule and PD1.

In other embodiments, at least two genes are edited. Pairs of genes may include, but are not limited to PD1 and TCRα, PD1 and TCRβ, CTLA-4 and TCRα, CTLA-4 and TCRβ, LAG3 and TCRα, LAG3 and TCRβ, Tim3 and TCRα, Tim3 and TCRβ, BTLA and TCRα, BTLA and TCRβ, BY55 and TCRα, BY55 and TCRβ, TIGIT and TCRα, TIGIT and TCRβ, B7H5 and TCRα, B7H5 and TCRβ, LAIR1 and TCRα, LAIR1 and TCRβ, SIGLEC10 and TCRα, SIGLEC10 and TCR(3, 2B4 and TCRα, 2B4 and TCRβ, B2M and TCRα, B2M and TCR(3.

In certain embodiments, a cell may be multiply edited (multiplex genome editing) as taught herein to (1) knock-out or knock-down expression of an endogenous TCR (for example, TRBC1, TRBC2 and/or TRAC), (2) knock-out or knock-down expression of an immune checkpoint protein or receptor (for example PD1, PD-L1 and/or CTLA4); and (3) knock-out or knock-down expression of one or more MHC constituent proteins (for example, HLA-A, B and/or C, and/or B2M, preferably B2M).

Whether prior to or after genetic modification of the T cells, the T cells can be activated and expanded generally using methods as described, for example, in U.S. Pat. Nos. 6,352,694; 6,534,055; 6,905,680; 5,858,358; 6,887,466; 6,905,681; 7,144,575; 7,232,566; 7,175,843; 5,883,223; 6,905,874; 6,797,514; 6,867,041; and 7,572,631. T cells can be expanded in vitro or in vivo.

Immune cells may be obtained using any method known in the art. In one embodiment, allogenic T cells may be obtained from healthy subjects. In one embodiment T cells that have infiltrated a tumor are isolated. T cells may be removed during surgery. T cells may be isolated after removal of tumor tissue by biopsy. T cells may be isolated by any means known in the art. In one embodiment, T cells are obtained by apheresis. In one embodiment, the method may comprise obtaining a bulk population of T cells from a tumor sample by any suitable method known in the art. For example, a bulk population of T cells can be obtained from a tumor sample by dissociating the tumor sample into a cell suspension from which specific cell populations can be selected. Suitable methods of obtaining a bulk population of T cells may include, but are not limited to, any one or more of mechanically dissociating (e.g., mincing) the tumor, enzymatically dissociating (e.g., digesting) the tumor, and aspiration (e.g., as with a needle).

The bulk population of T cells obtained from a tumor sample may comprise any suitable type of T cell. Preferably, the bulk population of T cells obtained from a tumor sample comprises tumor infiltrating lymphocytes (TILs).

The tumor sample may be obtained from any mammal. Unless stated otherwise, as used herein, the term “mammal” refers to any mammal including, but not limited to, mammals of the order Logomorpha, such as rabbits; the order Carnivora, including Felines (cats) and Canines (dogs); the order Artiodactyla, including Bovines (cows) and Swine (pigs); or of the order Perssodactyla, including Equines (horses). The mammals may be non-human primates, e.g., of the order Primates, Ceboids, or Simoids (monkeys) or of the order Anthropoids (humans and apes). In some embodiments, the mammal may be a mammal of the order Rodentia, such as mice and hamsters. Preferably, the mammal is a non-human primate or a human. An especially preferred mammal is the human.

T cells can be obtained from a number of sources, including peripheral blood mononuclear cells (PBMC), bone marrow, lymph node tissue, spleen tissue, and tumors. In certain embodiments of the present invention, T cells can be obtained from a unit of blood collected from a subject using any number of techniques known to the skilled artisan, such as Ficoll separation. In one preferred embodiment, cells from the circulating blood of an individual are obtained by apheresis or leukapheresis. The apheresis product typically contains lymphocytes, including T cells, monocytes, granulocytes, B cells, other nucleated white blood cells, red blood cells, and platelets. In one embodiment, the cells collected by apheresis may be washed to remove the plasma fraction and to place the cells in an appropriate buffer or media for subsequent processing steps. In one embodiment of the invention, the cells are washed with phosphate buffered saline (PBS). In an alternative embodiment, the wash solution lacks calcium and may lack magnesium or may lack many if not all divalent cations. Initial activation steps in the absence of calcium lead to magnified activation. As those of ordinary skill in the art would readily appreciate a washing step may be accomplished by methods known to those in the art, such as by using a semi-automated “flow-through” centrifuge (for example, the Cobe 2991 cell processor) according to the manufacturer's instructions. After washing, the cells may be resuspended in a variety of biocompatible buffers, such as, for example, Ca-free, Mg-free PBS. Alternatively, the undesirable components of the apheresis sample may be removed and the cells directly resuspended in culture media.

In another embodiment, T cells are isolated from peripheral blood lymphocytes by lysing the red blood cells and depleting the monocytes, for example, by centrifugation through a PERCOLL™ gradient. A specific subpopulation of T cells, such as CD28+, CD4+, CDC, CD45RA+, and CD45RO+ T cells can be further isolated by positive or negative selection techniques. For example, in one preferred embodiment, T cells are isolated by incubation with anti-CD3/anti-CD28 (i.e., 3×28)-conjugated beads, such as DYNABEADS® M-450 CD3/CD28 T, or XCYTE DYNABEADS™ for a time period sufficient for positive selection of the desired T cells. In one embodiment, the time period is about 30 minutes. In a further embodiment, the time period ranges from 30 minutes to 36 hours or longer and all integer values there between. In a further embodiment, the time period is at least 1, 2, 3, 4, 5, or 6 hours. In yet another preferred embodiment, the time period is 10 to 24 hours. In one preferred embodiment, the incubation time period is 24 hours. For isolation of T cells from patients with leukemia, use of longer incubation times, such as 24 hours can increase cell yield. Longer incubation times may be used to isolate T cells in any situation where there are few T cells as compared to other cell types, such in isolating tumor infiltrating lymphocytes (TIL) from tumor tissue or from immunocompromised individuals. Further, use of longer incubation times can increase the efficiency of capture of CD8+ T cells.

Enrichment of a T cell population by negative selection can be accomplished with a combination of antibodies directed to surface markers unique to the negatively selected cells. A preferred method is cell sorting and/or selection via negative magnetic immunoadherence or flow cytometry that uses a cocktail of monoclonal antibodies directed to cell surface markers present on the cells negatively selected. For example, to enrich for CD4+ cells by negative selection, a monoclonal antibody cocktail typically includes antibodies to CD14, CD20, CD11b, CD16, HLA-DR, and CD8.

Further, monocyte populations (i.e., CD14+ cells) may be depleted from blood preparations by a variety of methodologies, including anti-CD14 coated beads or columns, or utilization of the phagocytotic activity of these cells to facilitate removal. Accordingly, in one embodiment, the invention uses paramagnetic particles of a size sufficient to be engulfed by phagocytotic monocytes. In certain embodiments, the paramagnetic particles are commercially available beads, for example, those produced by Life Technologies under the trade name Dynabeads™. In one embodiment, other non-specific cells are removed by coating the paramagnetic particles with “irrelevant” proteins (e.g., serum proteins or antibodies). Irrelevant proteins and antibodies include those proteins and antibodies or fragments thereof that do not specifically target the T cells to be isolated. In certain embodiments, the irrelevant beads include beads coated with sheep anti-mouse antibodies, goat anti-mouse antibodies, and human serum albumin.

In brief, such depletion of monocytes is performed by preincubating T cells isolated from whole blood, apheresed peripheral blood, or tumors with one or more varieties of irrelevant or non-antibody coupled paramagnetic particles at any amount that allows for removal of monocytes (approximately a 20:1 bead:cell ratio) for about 30 minutes to 2 hours at 22 to 37 degrees C., followed by magnetic removal of cells which have attached to or engulfed the paramagnetic particles. Such separation can be performed using standard methods available in the art. For example, any magnetic separation methodology may be used including a variety of which are commercially available, (e.g., DYNAL® Magnetic Particle Concentrator (DYNAL MPC®)). Assurance of requisite depletion can be monitored by a variety of methodologies known to those of ordinary skill in the art, including flow cytometric analysis of CD14 positive cells, before and after depletion.

For isolation of a desired population of cells by positive or negative selection, the concentration of cells and surface (e.g., particles such as beads) can be varied. In certain embodiments, it may be desirable to significantly decrease the volume in which beads and cells are mixed together (i.e., increase the concentration of cells) to ensure maximum contact of cells and beads. For example, in one embodiment, a concentration of 2 billion cells/ml is used. In one embodiment, a concentration of 1 billion cells/ml is used. In a further embodiment, greater than 100 million cells/ml is used. In a further embodiment, a concentration of cells of 10, 15, 20, 25, 30, 35, 40, 45, or 50 million cells/ml is used. In yet another embodiment, a concentration of cells from 75, 80, 85, 90, 95, or 100 million cells/ml is used. In further embodiments, concentrations of 125 or 150 million cells/ml can be used. Using high concentrations can result in increased cell yield, cell activation, and cell expansion. Further, use of high cell concentrations allows more efficient capture of cells that may weakly express target antigens of interest, such as CD28-negative T cells, or from samples where there are many tumor cells present (i.e., leukemic blood, tumor tissue, etc). Such populations of cells may have therapeutic value and would be desirable to obtain. For example, using high concentration of cells allows more efficient selection of CD8+ T cells that normally have weaker CD28 expression.

In a related embodiment, it may be desirable to use lower concentrations of cells. By significantly diluting the mixture of T cells and surface (e.g., particles such as beads), interactions between the particles and cells is minimized. This selects for cells that express high amounts of desired antigens to be bound to the particles. For example, CD4+ T cells express higher levels of CD28 and are more efficiently captured than CD8+ T cells in dilute concentrations. In one embodiment, the concentration of cells used is 5×10⁶/ml. In other embodiments, the concentration used can be from about 1×10⁵/ml to 1×10⁶/ml, and any integer value in between.

T cells can also be frozen. Wishing not to be bound by theory, the freeze and subsequent thaw step provides a more uniform product by removing granulocytes and to some extent monocytes in the cell population. After a washing step to remove plasma and platelets, the cells may be suspended in a freezing solution. While many freezing solutions and parameters are known in the art and will be useful in this context, one method involves using PBS containing 20% DMSO and 8% human serum albumin, or other suitable cell freezing media, the cells then are frozen to −80° C. at a rate of 1° per minute and stored in the vapor phase of a liquid nitrogen storage tank. Other methods of controlled freezing may be used as well as uncontrolled freezing immediately at −20° C. or in liquid nitrogen.

T cells for use in the present invention may also be antigen-specific T cells. For example, tumor-specific T cells can be used. In certain embodiments, antigen-specific T cells can be isolated from a patient of interest, such as a patient afflicted with a cancer or an infectious disease. In one embodiment, neoepitopes are determined for a subject and T cells specific to these antigens are isolated. Antigen-specific cells for use in expansion may also be generated in vitro using any number of methods known in the art, for example, as described in U.S. Patent Publication No. US 20040224402 entitled, Generation and Isolation of Antigen-Specific T Cells, or in U.S. Pat. No. 6,040,177. Antigen-specific cells for use in the present invention may also be generated using any number of methods known in the art, for example, as described in Current Protocols in Immunology, or Current Protocols in Cell Biology, both published by John Wiley & Sons, Inc., Boston, Mass.

In a related embodiment, it may be desirable to sort or otherwise positively select (e.g. via magnetic selection) the antigen specific cells prior to or following one or two rounds of expansion. Sorting or positively selecting antigen-specific cells can be carried out using peptide-MEW tetramers (Altman, et al., Science. 1996 Oct. 4; 274(5284):94-6). In another embodiment, the adaptable tetramer technology approach is used (Andersen et al., 2012 Nat Protoc. 7:891-902). Tetramers are limited by the need to utilize predicted binding peptides based on prior hypotheses, and the restriction to specific HLAs. Peptide-MHC tetramers can be generated using techniques known in the art and can be made with any MEW molecule of interest and any antigen of interest as described herein. Specific epitopes to be used in this context can be identified using numerous assays known in the art. For example, the ability of a polypeptide to bind to MEW class I may be evaluated indirectly by monitoring the ability to promote incorporation of ¹²⁵I labeled β2-microglobulin (β2m) into MHC class I/β2m/peptide heterotrimeric complexes (see Parker et al., J. Immunol. 152:163, 1994).

In one embodiment cells are directly labeled with an epitope-specific reagent for isolation by flow cytometry followed by characterization of phenotype and TCRs. In one embodiment, T cells are isolated by contacting with T cell specific antibodies. Sorting of antigen-specific T cells, or generally any cells of the present invention, can be carried out using any of a variety of commercially available cell sorters, including, but not limited to, MoFlo sorter (DakoCytomation, Fort Collins, Colo.), FACSAria™, FACSArray™, FACSVantage™, BD™ LSR II, and FACSCalibur™ (BD Biosciences, San Jose, Calif.).

In a preferred embodiment, the method comprises selecting cells that also express CD3. The method may comprise specifically selecting the cells in any suitable manner. Preferably, the selecting is carried out using flow cytometry. The flow cytometry may be carried out using any suitable method known in the art. The flow cytometry may employ any suitable antibodies and stains. Preferably, the antibody is chosen such that it specifically recognizes and binds to the particular biomarker being selected. For example, the specific selection of CD3, CD8, TIM-3, LAG-3, 4-1BB, or PD-1 may be carried out using anti-CD3, anti-CD8, anti-TIM-3, anti-LAG-3, anti-4-1BB, or anti-PD-1 antibodies, respectively. The antibody or antibodies may be conjugated to a bead (e.g., a magnetic bead) or to a fluorochrome. Preferably, the flow cytometry is fluorescence-activated cell sorting (FACS). TCRs expressed on T cells can be selected based on reactivity to autologous tumors. Additionally, T cells that are reactive to tumors can be selected for based on markers using the methods described in International Patent Publication Nos. WO 2014133567 and WO 2014133568, herein incorporated by reference in their entirety. Additionally, activated T cells can be selected for based on surface expression of CD107a.

In one embodiment of the invention, the method further comprises expanding the numbers of T cells in the enriched cell population. Such methods are described in U.S. Pat. No. 8,637,307 and is herein incorporated by reference in its entirety. The numbers of T cells may be increased at least about 3-fold (or 4-, 5-, 6-, 7-, 8-, or 9-fold), more preferably at least about 10-fold (or 20-, 30-, 40-, 50-, 60-, 70-, 80-, or 90-fold), more preferably at least about 100-fold, more preferably at least about 1,000 fold, or most preferably at least about 100,000-fold. The numbers of T cells may be expanded using any suitable method known in the art. Exemplary methods of expanding the numbers of cells are described in International Patent Publication No. WO 2003057171, U.S. Pat. No. 8,034,334, and U.S. Patent Publication No. 2012/0244133, each of which is incorporated herein by reference.

In one embodiment, ex vivo T cell expansion can be performed by isolation of T cells and subsequent stimulation or activation followed by further expansion. In one embodiment of the invention, the T cells may be stimulated or activated by a single agent. In another embodiment, T cells are stimulated or activated with two agents, one that induces a primary signal and a second that is a co-stimulatory signal. Ligands useful for stimulating a single signal or stimulating a primary signal and an accessory molecule that stimulates a second signal may be used in soluble form. Ligands may be attached to the surface of a cell, to an Engineered Multivalent Signaling Platform (EMSP), or immobilized on a surface. In a preferred embodiment both primary and secondary agents are co-immobilized on a surface, for example a bead or a cell. In one embodiment, the molecule providing the primary activation signal may be a CD3 ligand, and the co-stimulatory molecule may be a CD28 ligand or 4-1BB ligand.

In certain embodiments, T cells comprising a CAR or an exogenous TCR may be manufactured as described in International Patent Publication No. WO2015120096 by a method comprising enriching a population of lymphocytes obtained from a donor subject; stimulating the population of lymphocytes with one or more T-cell stimulating agents to produce a population of activated T cells, wherein the stimulation is performed in a closed system using serum-free culture medium; transducing the population of activated T cells with a viral vector comprising a nucleic acid molecule which encodes the CAR or TCR, using a single cycle transduction to produce a population of transduced T cells, wherein the transduction is performed in a closed system using serum-free culture medium; and expanding the population of transduced T cells for a predetermined time to produce a population of engineered T cells, wherein the expansion is performed in a closed system using serum-free culture medium. In certain embodiments, T cells comprising a CAR or an exogenous TCR, may be manufactured as described in WO2015120096, by a method comprising obtaining a population of lymphocytes; stimulating the population of lymphocytes with one or more stimulating agents to produce a population of activated T cells, wherein the stimulation is performed in a closed system using serum-free culture medium; transducing the population of activated T cells with a viral vector comprising a nucleic acid molecule which encodes the CAR or TCR, using at least one cycle transduction to produce a population of transduced T cells, wherein the transduction is performed in a closed system using serum-free culture medium; and expanding the population of transduced T cells to produce a population of engineered T cells, wherein the expansion is performed in a closed system using serum-free culture medium. The predetermined time for expanding the population of transduced T cells may be 3 days. The time from enriching the population of lymphocytes to producing the engineered T cells may be 6 days. The closed system may be a closed bag system. Further provided is population of T cells comprising a CAR or an exogenous TCR obtainable or obtained by said method, and a pharmaceutical composition comprising such cells.

In certain embodiments, T cell maturation or differentiation in vitro may be delayed or inhibited by the method as described in International Patent Publication No. WO2017070395, comprising contacting one or more T cells from a subject in need of a T cell therapy with an AKT inhibitor (such as, e.g., one or a combination of two or more AKT inhibitors disclosed in claim 8 of WO2017070395) and at least one of exogenous Interleukin-7 (IL-7) and exogenous Interleukin-15 (IL-15), wherein the resulting T cells exhibit delayed maturation or differentiation, and/or wherein the resulting T cells exhibit improved T cell function (such as, e.g., increased T cell proliferation; increased cytokine production; and/or increased cytolytic activity) relative to a T cell function of a T cell cultured in the absence of an AKT inhibitor.

In certain embodiments, a patient in need of a T cell therapy may be conditioned by a method as described in International Patent Publication No. WO2016191756 comprising administering to the patient a dose of cyclophosphamide between 200 mg/m2/day and 2000 mg/m2/day and a dose of fludarabine between 20 mg/m2/day and 900 mg/m²/day.

### Screening for Modulating Agents

In certain embodiments, biomarkers are used to screen for therapeutic agents capable of shifting a phenotype. In certain embodiments, the method comprises: a) applying a candidate agent to a cell or cell population; b) detecting modulation of one or more phenotypic aspects of the cell or cell population by the candidate agent (e.g., modulation of expression of one or more genes in a gene module comprising a genetic variant or modulation of an identified pathway or gene program), thereby identifying the agent. The phenotypic aspects of the cell or cell population that is modulated may be a gene signature or biological program specific to a cell type or cell phenotype or phenotype specific to a population of cells (e.g., a responder phenotype). In certain embodiments, steps can include administering candidate modulating agents to cells, detecting identified cell (sub)populations for changes in signatures, or identifying relative changes in cell (sub) populations which may comprise detecting relative abundance of particular gene signatures.

The term “modulate” broadly denotes a qualitative and/or quantitative alteration, change or variation in that which is being modulated. Where modulation can be assessed quantitatively—for example, where modulation comprises or consists of a change in a quantifiable variable such as a quantifiable property of a cell or where a quantifiable variable provides a suitable surrogate for the modulation—modulation specifically encompasses both increase (e.g., activation) or decrease (e.g., inhibition) in the measured variable. The term encompasses any extent of such modulation, e.g., any extent of such increase or decrease, and may more particularly refer to statistically significant increase or decrease in the measured variable. By means of example, modulation may encompass an increase in the value of the measured variable by at least about 10%, e.g., by at least about 20%, preferably by at least about 30%, e.g., by at least about 40%, more preferably by at least about 50%, e.g., by at least about 75%, even more preferably by at least about 100%, e.g., by at least about 150%, 200%, 250%, 300%, 400% or by at least about 500%, compared to a reference situation without said modulation; or modulation may encompass a decrease or reduction in the value of the measured variable by at least about 10%, e.g., by at least about 20%, by at least about 30%, e.g., by at least about 40%, by at least about 50%, e.g., by at least about 60%, by at least about 70%, e.g., by at least about 80%, by at least about 90%, e.g., by at least about 95%, such as by at least about 96%, 97%, 98%, 99% or even by 100%, compared to a reference situation without said modulation. Preferably, modulation may be specific or selective, hence, one or more desired phenotypic aspects of an immune cell or immune cell population may be modulated without substantially altering other (unintended, undesired) phenotypic aspect(s).

The term “agent” broadly encompasses any condition, substance or agent capable of modulating one or more phenotypic aspects of a cell or cell population as disclosed herein. Such conditions, substances or agents may be of physical, chemical, biochemical and/or biological nature. The term “candidate agent” refers to any condition, substance or agent that is being examined for the ability to modulate one or more phenotypic aspects of a cell or cell population as disclosed herein in a method comprising applying the candidate agent to the cell or cell population (e.g., exposing the cell or cell population to the candidate agent or contacting the cell or cell population with the candidate agent) and observing whether the desired modulation takes place.

Agents may include any potential class of biologically active conditions, substances or agents, such as for instance antibodies, proteins, peptides, nucleic acids, oligonucleotides, small molecules, or combinations thereof, as described herein.

The methods of phenotypic analysis can be utilized for evaluating environmental stress and/or state, for screening of chemical libraries, and to screen or identify structural, syntenic, genomic, and/or organism and species variations. For example, a culture of cells, can be exposed to an environmental stress, such as but not limited to heat shock, osmolarity, hypoxia, cold, oxidative stress, radiation, starvation, a chemical (for example a therapeutic agent or potential therapeutic agent) and the like. After the stress is applied, a representative sample can be subjected to analysis, for example at various time points, and compared to a control, such as a sample from an organism or cell, for example a cell from an organism, or a standard value. By exposing cells, or fractions thereof, tissues, or even whole animals, to different members of the chemical libraries, and performing the methods described herein, different members of a chemical library can be screened for their effect on immune phenotypes thereof simultaneously in a relatively short amount of time, for example using a high throughput method.

Aspects of the present disclosure relate to the correlation of an agent with the spatial proximity and/or epigenetic profile of the nucleic acids in a sample of cells. In some embodiments, the disclosed methods can be used to screen chemical libraries for agents that modulate chromatin architecture epigenetic profiles, and/or relationships thereof.

In some embodiments, screening of test agents involves testing a combinatorial library containing a large number of potential modulator compounds. A combinatorial chemical library may be a collection of diverse chemical compounds generated by either chemical synthesis or biological synthesis, by combining a number of chemical “building blocks” such as reagents. For example, a linear combinatorial chemical library, such as a polypeptide library, is formed by combining a set of chemical building blocks (amino acids) in every possible way for a given compound length (for example the number of amino acids in a polypeptide compound). Millions of chemical compounds can be synthesized through such combinatorial mixing of chemical building blocks.

In certain embodiments, the present invention provides for gene signature screening. The concept of signature screening was introduced by Stegmaier et al. (Gene expression-based high-throughput screening (GE-HTS) and application to leukemia differentiation. Nature Genet. 36, 257-263 (2004)), who realized that if a gene-expression signature was the proxy for a phenotype of interest, it could be used to find small molecules that effect that phenotype without knowledge of a validated drug target. The signatures or biological programs of the present invention may be used to screen for drugs that reduce the signature or biological program in cells as described herein. The signature or biological program may be used for GE-HTS. In certain embodiments, pharmacological screens may be used to identify drugs that are selectively toxic to cells having a signature.

The Connectivity Map (cmap) is a collection of genome-wide transcriptional expression data from cultured human cells treated with bioactive small molecules and simple pattern-matching algorithms that together enable the discovery of functional connections between drugs, genes and diseases through the transitory feature of common gene-expression changes (see, Lamb et al., The Connectivity Map: Using Gene-Expression Signatures to Connect Small Molecules, Genes, and Disease. Science 29 Sep. 2006: Vol. 313, Issue 5795, pp. 1929-1935, DOI: 10.1126/science.1132939; and Lamb, J., The Connectivity Map: a new tool for biomedical research. Nature Reviews Cancer January 2007: Vol. 7, pp. 54-60). In certain embodiments, Cmap can be used to screen for small molecules capable of modulating a signature or biological program of the present invention in silico.

The invention is further described in the following examples, which do not limit the scope of the invention described in the claims.

### EXAMPLES

### Example 1—Identify Disease Genes Through Exome Wide Association Analysis

Genome wide association studies (GWAS) can be used to determine structure underlying polygenic traits using single loci (FIG. 1). Statistically significant genomic variants can be identified by comparing frequencies of the variants in disease cases and control cases (FIG. 1A). Genetic risk genes organize into gene programs and each gene program can represent a risk module (FIG. 1B,C) (see, e.g., Smillie, Biton, Ordovas-Montanes et al., Cell 2019). Disease loci can be used to identify gene programs related to biological pathways, identify therapeutic targets, and detection of high risk individuals (FIG. 1D). Applicants identified single variants associated with IBD through exome sequencing. For each variant identified through exome sequencing, Applicants performed a statistical test to measure the association of the variant with a cohort of 50K healthy and IBD individuals. The exome wide association study uncovers dozens of novel disease-associated variants in known IBD related genes such as NOD2, CARDS, IL23R (FIG. 2).

### Example 2—Building Modules of Disease Relevant Genes Using UKBBK and UC Single Cell Atlas

The UK Biobank (UKBBK) phenotypes helps to identify IBD substructure. The UKKBK dataset enables Applicants to discover a substructure within the set of IBD associated variants using clustering (see, e.g., Udler et al., 2018). Applicants measured the association of each of the IBD variants with a range of more granular symptoms such as: blood platelet counts, fatigue, fever. This requires building a matrix consisting of GWAS associations for each SNP and phenotype combination and resulted in 4 groupings of the IBD variants each significantly enriched for increasing risk and likelihood for separate IBD related symptoms/phenotypes (FIG. 3).

A Single cell UC atlas helps to identify IBD substructure. The UC single cell atlas highlights over 60 cell types across 300,000 cells consisting of healthy, inflamed and uninflamed tissues (Smillie C S. et al., Intra- and Inter-cellular Rewiring of the Human Colon during Ulcerative Colitis. Cell. 2019 Jul. 25; 178(3):714-730.e22). Each of the disease genes identified through association analysis is projected on the single cells resulting in 5 groupings of disease genes based on the cell types where they are expressed (FIG. 4). To further narrow down the set of relevant cell types Applicants can determine which cell types the disease genes are differentially expressed in.

The methods described herein can be used for connecting disease symptoms/phenotypes to the relevant molecular phenotypes. Applicants apply machine learning techniques (e.g., multi-domain translation) to map between the space of disease relevant phenotypes/symptoms and the space of molecular phenotypes. Having a common latent space between phenotypes and cell types will help to elucidate the relevant cell types affecting the progression of specific IBD related symptoms.

Applicants asked if UC variants synergize to increase disease risk (FIG. 5). Logistic regression identifies a linear combination of SNPs that best separate the two classes. A deep neural network models nonlinear combinations of SNPs to capture SNP-SNP interactions missed previously. Thus, modeling nonlinear interactions improves predictive power.

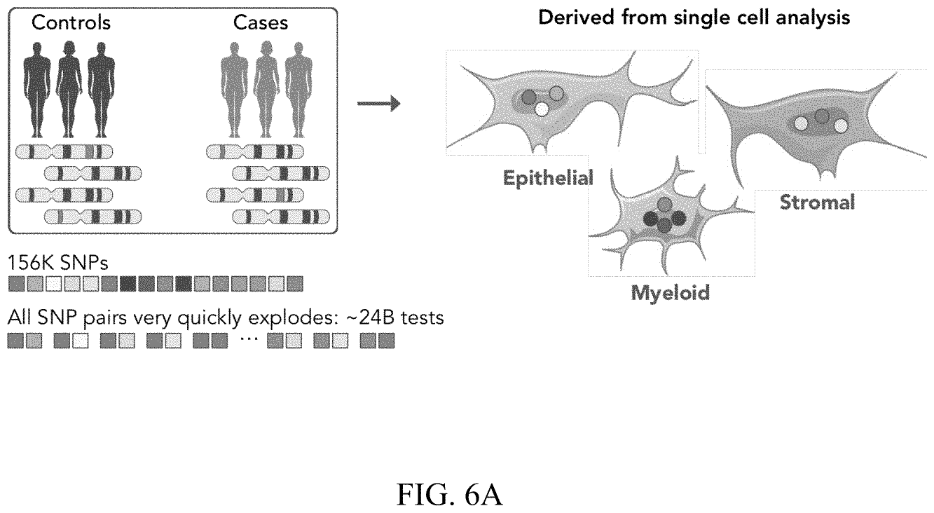

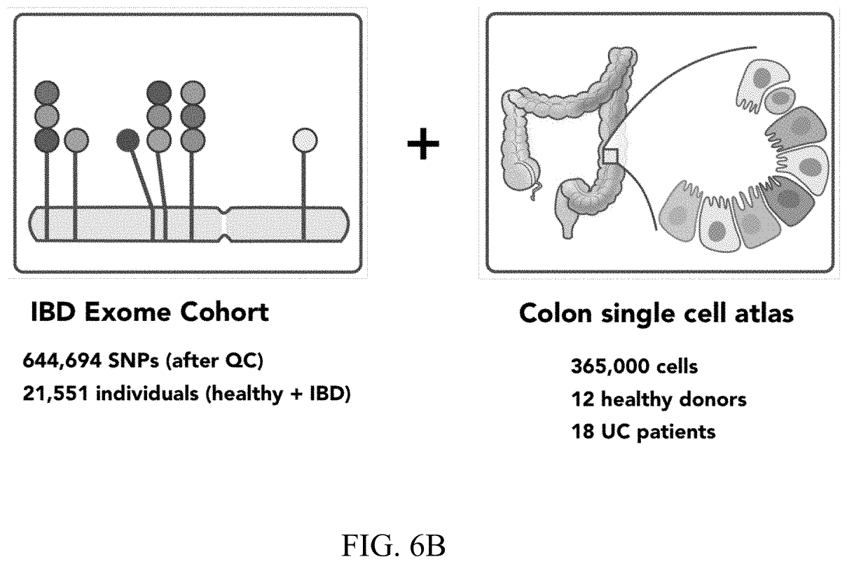

Applicants asked if they can test for genome-wide SNP interactions (FIG. 6A). Using an IBD exome cohort that included 53 thousand samples 2.5 million SNPs were identified. After sample quality control the cohort had 41 thousand samples and 1.8 million SNPs. After variant quality control and using a frequency filter the cohort had 41 thousand samples and 156 thousand SNPs (156,000 SNPs*156,000 SNPs=>˜24 billion interactions that need to be tested). Single cell RNA-seq provides a prior for which genes are likely to interact. Applicants combined a full colon single cell atlas (Smillie, et al., 2019) with the IBD exome (FIG. 6B).

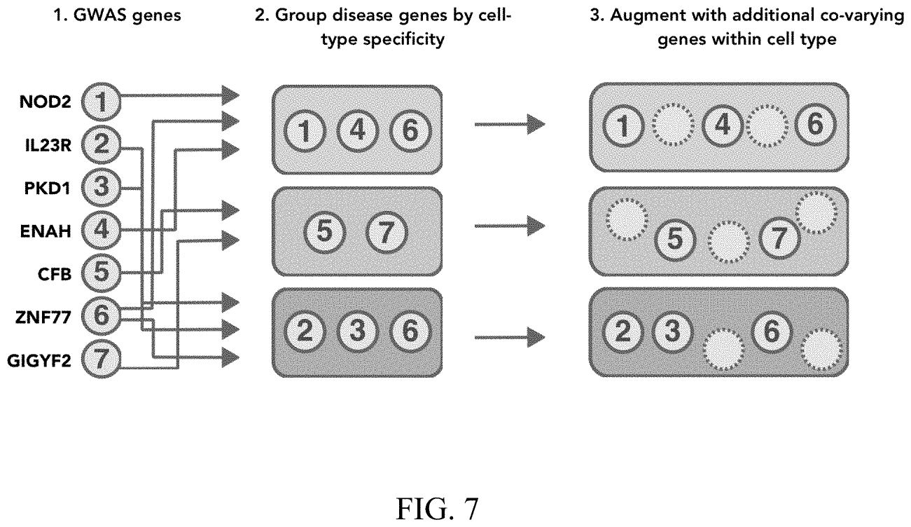

Applicants re-built modules in two ways: (1) cell type specific modules only of GWAS genes, using variation across all cell types and (2) program modules, based on co-variation within a cell type, using the GWAS genes as seeds (FIG. 7). Covariance across single cells and UKBBK phenotypes expands disease genes to modules. Applicants extend beyond the known IBD disease genes to other possible IBD relevant genes by incorporating signals from the UKBBK phenotypes and the single cell expression profiles. Specifically, Applicants identify communities of disease enriched genes in each cell type based on gene covariance within each cell type in the single cell data (FIG. 7). Similarly, the set of genes with significant associations with the UKBBK phenotypes may also be IBD related. Currently, Applicants are developing an EM algorithm to go back and forth between these UKBBK gene modules and single cell gene modules to finalize a high-quality module of genes. Applicants can run enrichment tests to see how well these modules overlap with gene sets that represent ER stress, inflammation and other IBD related disease pathways. Assays for testing the phenotypes are known in the art (e.g., cell based assays for autophagy or ER stress).

### Example 3—Gene Modules Increase Interpretability of the Disease

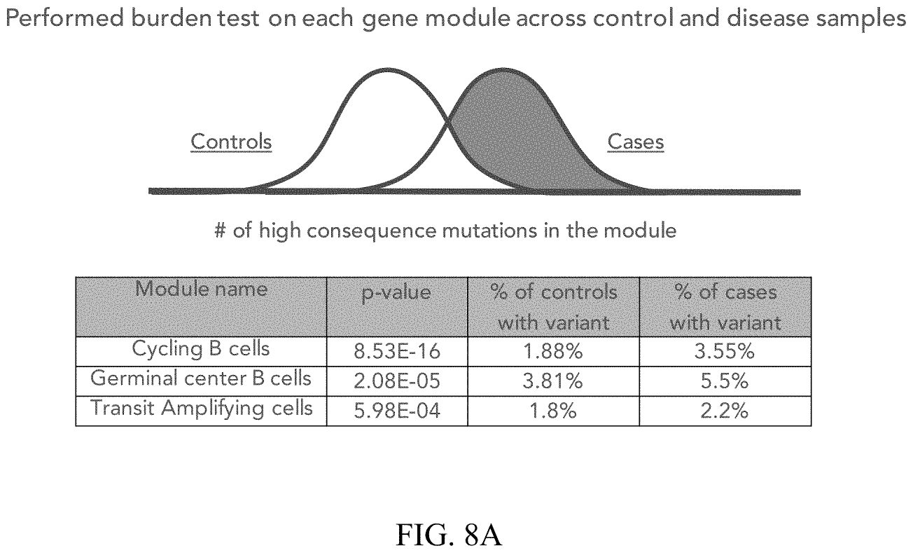

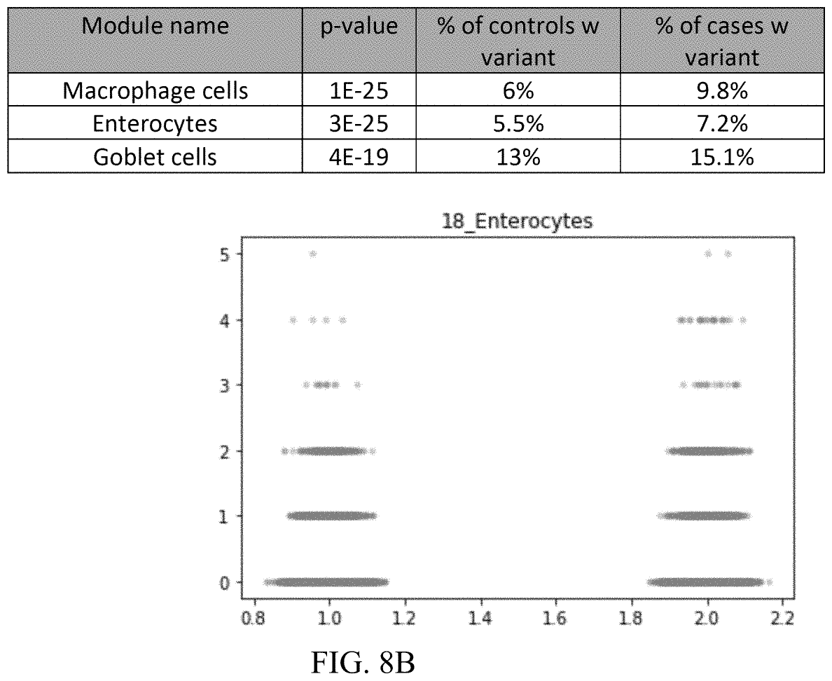

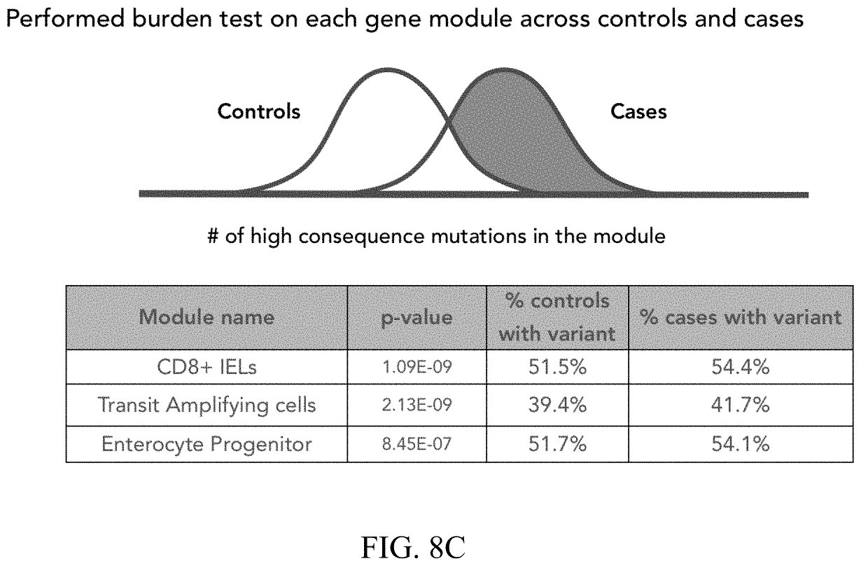

Applicants looked for ways to use the modules for subtle signals. A rare variant burden test measures the contribution of subtle signals and picks up subtler effects (FIG. 8). GWAS style association tests are highly effective at identifying disease variants from population level genetic data but fall short at effectively measuring the impact of rare variants. Many disease related variants will not reach high enough frequency in the population, especially severe variants. Applicants developed a burden test over gene modules combining signals across the low frequency variants in the same module to highlight the most disease relevant cell types. For example, to look for implicated cells, Applicants performed a burden test on each gene module across control and disease samples, looking at a number of high consequence coding mutations in the module. The Cycling B cells module has close to a 2 fold increase in mutations in cases compared to controls (FIG. 8A). Applicants find that gene modules in Macrophages, Enterocytes and Goblet cells have increased mutational burden across the IBD patients (FIG. 8B). This also identified significant differences in modules related to CD8 IEL or enterocyte progenitors (FIG. 8C).

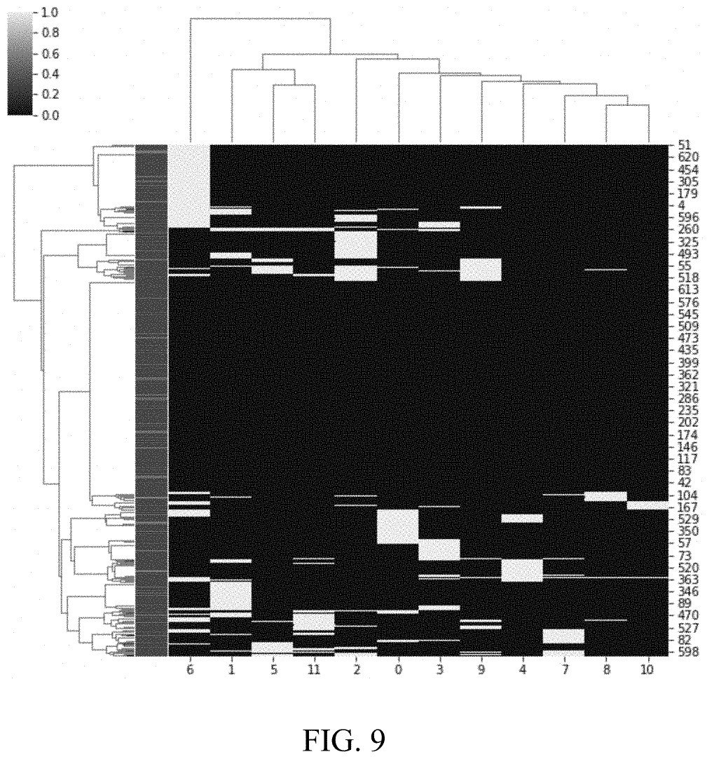

Disease associated modules stratify patients into subtypes. Applicants can use the gene modules built in the previous step to better categorize/stratify patients by reducing the space from 200K variants to 60 meaningful gene modules. Applicants aggregated counts of (high impact) mutations in each gene module for each patient. Clustering this resulting 50K×60 matrix results in 5 groups of patients (FIG. 9). The groups are enriched for disease severity and patient treatments.

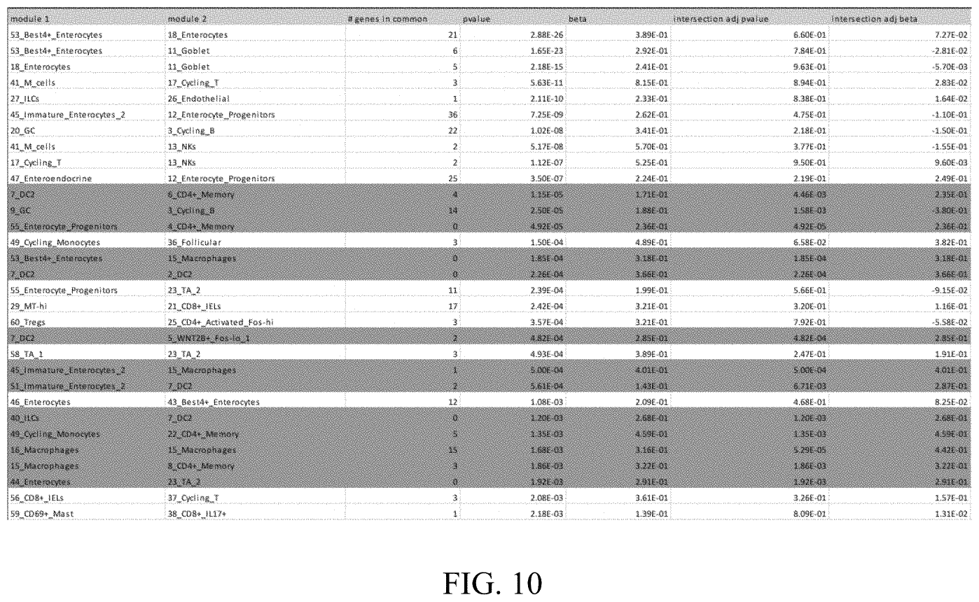

Module-module interactions increase the risk of IBD. Applicants can only capture interactions between pathways through a combined single cell+human genetics approach by testing all pairs of modules and the mutational burden observed in each module. Applicants find significant interactions between modules in Enterocyte progenitors and CD4 memory cells, Best4 Enterocytes and Macrophages and 2 separate modules both in Macrophage cells (FIG. 10, Table 5).

TABLE 5
Modules with the highest burden

| Module name | pvalue | beta | ethnicity group |
|---|---|---|---|
| 56_CD8+_IELs | 1.09E−09 | 8.89E−02 | NFE_IBD_celltype |
| 57_TA_1 | 2.13E−09 | 1.04E−01 | NFE_IBD_celltype |
| 55_Enterocyte_Progeni- | 8.45E−07 | 7.19E−02 | NFE_IBD_celltype |
| tors | | | |
| 43_Best4+_Enterocytes | 1.28E−04 | 7.88E−02 | NFE_IBD_celltype |
| 42_CD8+_IL17+ | 4.60E−04 | 6.95E−02 | NFE_IBD_celltype |
| 57_TA_1 | 4.79E−04 | 1.12E−01 | AJ_IBD_celltype |
| 2_DC2 | 1.10E−03 | 2.68E−01 | FIN_IBD_celltype |
| 37_Cycling_T | 4.27E−03 | 5.23E−02 | NFE_IBD_celltype |
| 40_ILCs | 5.13E−03 | 6.52E−02 | NFE_IBD_celltype |
| 60_Tregs | 6.69E−03 | 4.33E−02 | NFE_IBD_celltype |

### Example 4—Identifying Significant Interactions Between IBD SNPs

Enumerating all possible pairwise and high order SNP interactions quickly explodes and is not feasible. As a proof of concept, Applicants further used the gene modules to reduce the search space over which SNP interactions are tested. Applicants looked into genetic interactions, exploring three kinds of situations and finding statistically significant examples in all.

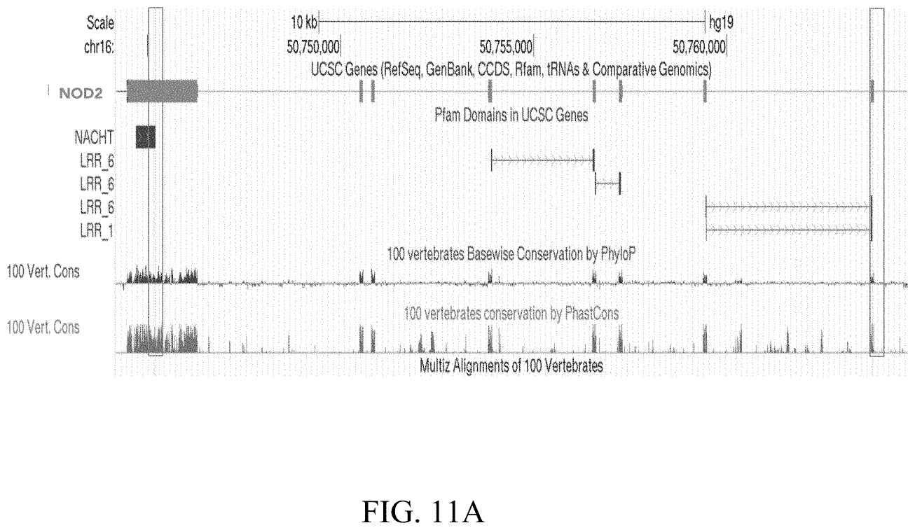

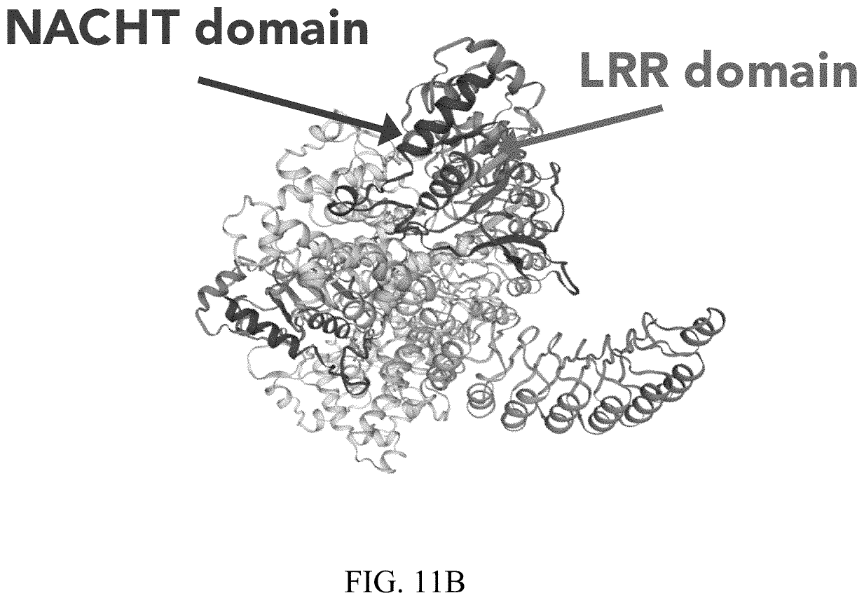

Applicants tested SNP interactions within genes. The simplest approach is to limit all SNP pairs to be within the same gene. Variants can be breaking two different regions of the same gene resulting in incorrect gene function and further downstream effects. Applicants find a significant interaction between two SNPs in the NOD2 gene locus (FIG. 11A). The SNPs are also overlapping two different functionally related annotated protein domains giving increased confidence in the prediction (FIG. 11B).

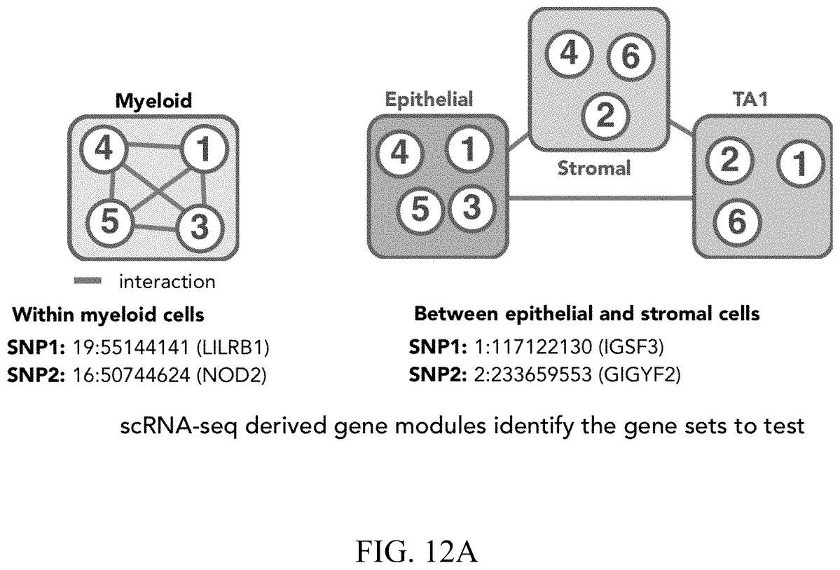

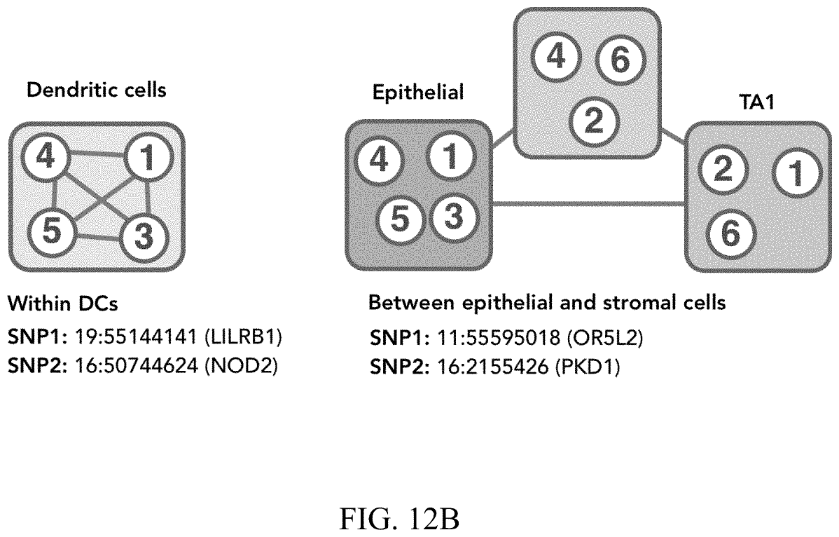

Applicants tested SNP interactions within the same gene module. Beyond SNPs within the same gene, traditionally there is no apparent way to limit SNP pairs to be tested. Here, Applicants use the gene modules to only test SNP pairs where both SNPs are in genes that are part of the same module. This greatly reduces the search space of SNP pairs and in the process, Applicants identified a significant interaction between LILRB1 and NOD2 in neutrophils (FIG. 12A,B). Both these genes are found to be expressed in myeloid cells (e.g., dendritic cells).

Applicants tested SNP interactions between modules in UC (Table 4), first identifying modules that as a whole interact by their aggregate signal and then look at pairs of genes between them. SNP interactions increasing disease risk may not be limited to within the same gene or module but may also be between two SNPs in genes expressed in different cell types and modules. To systematically test all of these interactions would be infeasible as previously described, but Applicants identified interacting modules in above. Applicants can instead enumerate all SNP pairs between the interacting modules identified and test these SNP pairs for significance. This highlights a significant SNP interaction between IGSFR (expressed in epithelial cells) and GIGYF2 (expressed in stromal cells) (FIG. 12A). Additionally, Applicants identified a significant SNP interaction between epithelial and stromal cells, and then specifically between OR5L2 and PKD1 (FIG. 12B).

Applicants identified a list of module interactions (Table 6).

TABLE 6

| | | | | inter- | |
|---|---|---|---|---|---|
| | | | | section | |
| | | # genes in | | adjusted | |
| Module 1 | Module 2 | common | pvalue | pvalue | ethnicity |
| 62_T.CD8_IELs | 30_F.Crypt_loFos_1 | 9 | 2.23E−05 | 3.64E−05 | NFE_IBD_celltype |
| 36_Follicular | 3_Cycling_B | 10 | 6.63E−05 | 6.92E−05 | NFE_IBD_orig |
| 36_Follicular | 3_Cycling_B | 10 | 6.63E−05 | 6.92E−05 | NFE_IBD_orig |
| 71_T.Tcells | 30_F.Crypt_loFos_1 | 6 | 9.66E−05 | 1.08E−04 | NFE_IBD_celltype |
| 61_T.CD8 | 30_F.Crypt_loFos_1 | 8 | 6.06E−05 | 1.29E−04 | NFE_IBD_celltype |
| 55_Enterocyte_Progenitors | 4_CD4+_Memory | 0 | 1.33E−04 | 1.33E−04 | NFE_IBD_orig |
| 47_Enteroendocrine | 23_TA_2 | 1 | 1.82E−04 | 1.82E−04 | NFE_IBD_orig |
| 44_M.Macrophages.uc.dca.LILRA6.UC | 35_E.Goblet.healthy.dca.PRAMEF4.Healthy | 1 | 2.30E−04 | 2.30E−04 | NFE_IBD_dca |
| 71_T.Tcells | 31_F.Crypt_loFos_2 | 0 | 2.95E−04 | 2.95E−04 | NFE_IBD_celltype |
| 39_F.Glia.healthy.dca.CD28.Healthy | 32_E. Enteroendocrine.uc.dca.CCL20.UC | 0 | 5.45E−04 | 5.45E−04 | NFE_IBD_dca |
| 57_TA_1 | 19_CD8+_IELs | 1 | 5.90E−04 | 5.90E−04 | AJ_IBD_orig |
| 54_Secretory_TA | 37_Cycling_T | 1 | 5.50E−04 | 6.03E−04 | NFE_IBD_orig |
| 38_CD8+_IL17+ | 11_Goblet | 2 | 5.92E−01 | 6.74E−04 | NFE_IBD_orig |
| 38_CD8+_IL17+ | 11_Goblet | 2 | 5.92E−01 | 6.74E−04 | NFE_IBD_orig |
| 22_E.Secretory | 4_B.GC | 11 | 4.03E−03 | 7.00E−04 | FIN_IBD_celltype |
| 68_T.NK.uc.dca.IL2RA.UC | 3_T.Cycling_T.uc.dca.TNFAIP3.UC | 8 | 1.10E−03 | 7.16E−04 | AJ_IBD_dca |
| 31_F.Crypt_loFos_2 | 25_E.Stem | 4 | 7.42E−04 | 7.42E−04 | FIN_IBD_celltype |
| 45_I.Immune | 37_F.Microvascular | 3 | 7.79E−04 | 7.79E−04 | AJ_IBD_celltype |
| 41_M.CD69neg_Mast.uc.dca.C5orf66.UC | 5_E.Enterocyte_Progenitor.uc.dca.ENAH.UC | 0 | 8.17E−04 | 8.17E−04 | NFE_IBD_dca |
| 33_Cycling_TA | 17_Cycling_T | 0 | 9.08E−04 | 9.08E−04 | NFE_IBD_orig |
| 33_Cycling_TA | 17_Cycling_T | 0 | 9.08E−04 | 9.08E−04 | NFE_IBD_orig |
| 71_T.Tcells | 27_F.Crypt | 0 | 9.50E−04 | 9.50E−04 | NFE_IBD_celltype |
| 64_T.NK.healthy.dca.PRAMEF4.Healthy | 8_M.Neutrophils.healthy.dca.PRKCB.Healthy | 2 | 8.85E−04 | 9.94E−04 | NFE_IBD_dca |

Applicants also identified a list of SNP interactions (Table 7).

TABLE 7

| SNP1 | SNP2 | pvalue |
|---|---|---|
| 11:55111118[“A”,“G”] | 11:55111057[”G”,“A”] | 2.0197968221E−08 |
| 17:39340812[“T”,“C”] | 5:140476396[“G”,“T”] | 7.9606242699E−08 |
| 11:1265450[“A”,“C”] | 11:55595018[“A”,“G”] | 8.5811831296E−07 |
| 11:1265450[“A”,“C”] | 11:55595017[“G”,“T”] | 9.0111602671E−07 |
| 11:1265450[“A”,“C”] | 11:55595012[“A”,“T”] | 1.0432732592E−06 |
| 11:1265481[“C”,“T”] | 11:55595018[“A”,“G”] | 1.1018565806E−06 |
| 11:55595017[“G”,“T”] | 11:1265481[“C”,“T”] | 1.1542153208E−06 |
| 1:248458419[“G”,“C”] | 19:55148043[“T”,“C”] | 1.3201072181E−06 |
| 11:1265481[“C”,“T”] | 11:55595012[“A”,“T”] | 1.3436862727E−06 |
| 1:248458419[“G”,“C”] | 19:55148045[“G”,“A”] | 1.5098471857E−06 |
| 16:2155426[“T”,“C”] | 17:55183813[“A”,“G”] | 1.3668527490E−05 |
| 16:14958514[“A”,“G”] | 18:44561379[“C”,“T”] | 1.5330616269E−05 |
| 16:14958514[“A”,“G”] | 18:44561375[“T“,“C”] | 1.6795622741E−05 |
| 16:2155426[“T”,“C”] | 11:55595018[“A”,“G”] | 2.0984084931E−05 |
| 16:50763778[“G”,“G”,“C”] | 16:50745926[“C”,“T”] | 2.2579383247E−05 |
| 16:2155426[“T”,“C”] | 11:55595017[“G”,“T”] | 2.2772767022E−05 |
| 16:2155426[“T”,“C”] | 17:55183792[“G”,“A”] | 2.4763857652E−05 |
| 16:2155426[“T”,“C”] | 11:55595012[“A”,“T”] | 3.7328205934E−05 |
| 5:140481841[“T”,“C”] | 5:140476396[“G”,“T”] | 5.1603100002E−05 |
| 16:2155426[“T”,“C”] | 19:55494612[“A”,“G”] | 5.4822337186E−05 |
| 19:20807133[“GGCTTTGCCACATTCTTCACA | 17:55183813[“A”,“G”] | 9.1170822968E−05 |
| TTTGTAGAATTTCTCTCCAGTATGATTCTCTCA | | |
| TGTGTAGTAAGGATTGAGGACTGGTTGAAGG | | |
| CTTTGCCACATTCTTCACATTTGTAGGGTCTCT | | |
| CTCCAGTATGAATTTTCTTATGTGTAGTAAGG | | |
| TTAGAGGAGCACTTAAAA”,“G”] (SEQ ID NO: | | |
| 34) | | |
| 19:2939267[“CACCACCCTTACCCAAGGAGG | 18:44561379[“C”,“T”] | 1.5587633578E−04 |
| CA”,“C”] (SEQ ID NO: 35) | | |
| 5:140476396[“G”,“T”] | 2:233273011[“C”,“G”] | 1.5848137054E−04 |
| 19:2939267[“CACCACCCTTACCCAAGGAGG | 18:44561375[“T”,“C”] | 1.6495790617E−04 |
| CA”,“C”] (SEQ ID NO: 36) | | |
| 17:55183792[“G”,“A”] | 19:20807133[“GGCTTTGCCACATTCTTCACA | 1.6613857473 E−04 |
| | TTTGTAGAATTTCTCTCCAGTATGATTCTCTCA | |
| | TGTGTAGTAAGGATTGAGGACTGGTTGAAGG | |
| | CTTTGCCACATTCTTCACATTTGTAGGGTCTCT | |
| | CTCCAGTATGAATTTTCTTATGTGTAGTAAGG | |
| | TTAGAGGAGCACTTAAAA”,“G”] (SEQ ID NO: | |
| | 37) | |
| 11:55595018[“A”,“G”] | 20:55108506[“C”,“CAATA”] | 1.6917082313 E−04 |
| 11:55595018[“A”,“G”] | 20:55108507[“CGTGT”,“C”] | 1.6917082313 E−04 |
| 11:55595017[“G”,“T”] | 20:55108506[“C”,“CAATA”] | 1.7861698734 E−04 |
| 11:55595017[“G”,“T”] | 20:55108507[“CGTGT”,“C”] | 1.7861698734 E−04 |
| 19:2939267[“CACCACCCTTACCCAAGGAGG | 19:22939464[“GGGTCGAGAAATTGTTAAAA | 1.8122011635 E−04 |
| CA”,“C”](SEQ ID NO: 38) | CCTTTGCCACATTCTTCACATTTGTACGGTTTC | |
| | TCCCCAGTATGAATTATCTTATGT”,“G”] (SEQ | |
| | ID NO: 39) | |

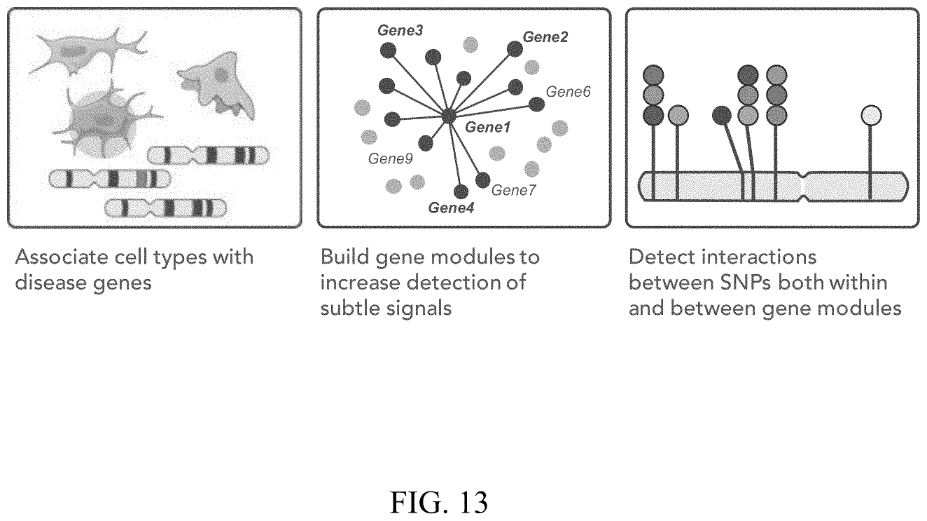

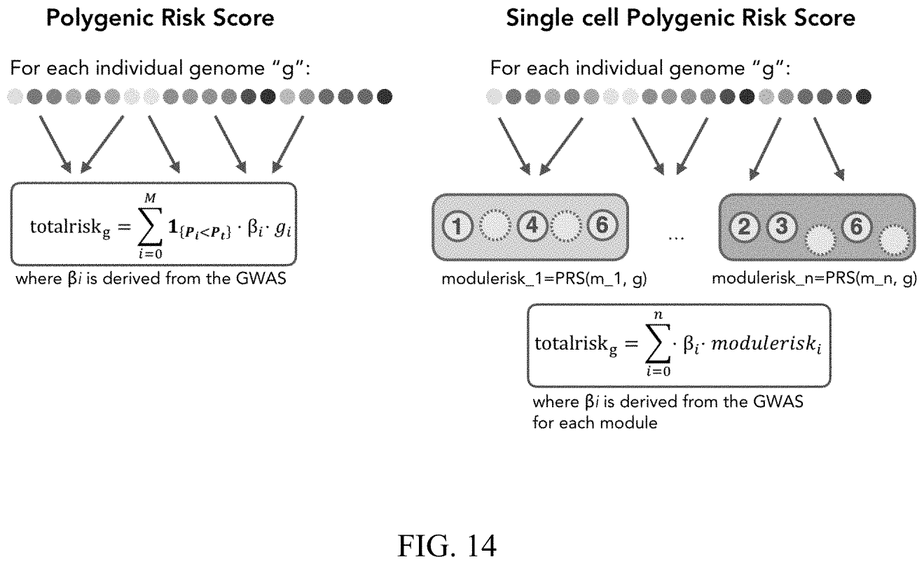

In summary, combining single cell atlases with human genetics allows for (1) associating cell types with disease genes, (2) building gene modules to increase detection of subtle signals, and (3) detect interactions between SNPs both within and between gene modules (FIG. 13). Further, applicants can use the single cell module approach to calculate polygenic risk scores (PRS), such that the PRS can be structured with modular information (FIG. 14). The gene modules allowed Applicants to predict GWAS gene function, and improved the prediction of causal genes in a multi gene region. Applicants incorporated the module structure to identify subtle signals, and map interactions. Applicants can use the present invention for developing a “modular” PRS, patient stratification, and sc-QTLs (quantitative trait loci).

### Example 5—Methods

Statistical Tests for Computing Association Analysis

### Single Variant Test

For a given variant, Applicants define xᵢ∈{0, 1, 2} to be 0 if the variant is homozygous for the reference allele, 1 if the variant is heterozygous and 2 if the variant is homozygous for the alternate allele. For all variants with allele frequency between 5% and 0.05%, Applicants performed a statistical test to determine a beta and p-value quantifying the significance of the variant association with disease over 50K healthy and disease exomes.

∀xᵢ∈Exome:y=β₀+β₁·xᵢ+Σ_{k=1 . . . 20}βₖ₊₁·PCₖ

### Burden Test

The burden test is performed by aggregating variants at the gene module level and testing the significance of the module. The module is represented as a set of genes such as mᵢ={g₁, g₂, . . . , gₙ} and each gene consists of many variants such that gᵢ={x₁, . . . ,xₙ}. The burden of a module is then measured by:

∀mᵢ∈Modules:y=β₀+β₁·Σ_{g}_{ᵢ}_{∈m}_{ᵢ}Σₓ_{ᵢ}_{∈g}_{ᵢ}xᵢ+Σ_{k=1 . . . 20}βₖ₊₁·PCₖ

### Module Interaction Test

Based on the above definitions, Applicants can then test for the significance of two modules interacting to increase disease risk with the following interaction test:

∀ pairs of modules(mᵢ,mⱼ)∈Modules: y=β₀+β₁·Σ_{g}_{ᵢ}_{∈m}_{ᵢ}Σₓ_{ᵢ}_{∈g}_{ᵢ}xᵢ+β₂·Σ_{g}_{ⱼ}_{∈m}_{ⱼ}Σₓ_{ⱼ}_{∈g}_{ⱼ}xⱼ+β₃·Σ_{g}_{ᵢ}_{∈m}_{ᵢ}Σₓ_{ᵢ}_{∈g}_{ᵢ}xᵢ·Σ_{g}_{ⱼ}_{∈m}_{ⱼ}Σₓ_{ⱼ}_{∈g}_{ⱼ}xⱼ+Σ_{k=1 . . . 20}βₖ₊₃·PCₖ

### SNP Interaction Tests

For any two SNPs the significance of the interaction between the two SNPs is measured with the following test:

y=β₀+β₁·xᵢ+β₂·xⱼ+β₃·xᵢ·xⱼ+Σ_{k=1 . . . 20}βₖ₊₃·PCₖ

50K+ exomes used for analysis. 25K healthy exomes and 20K IBD exomes were assembled by the Daly lab. Data processing was then performed to remove low quality samples and low quality genotypes were performed.

UK Biobank. GWAS statistics were pre-computed by the Neale Lab for all 1000 phenotypes in the UKBBK across the 500K genotyped individuals.

UC single cell atlas. 300K single cells from healthy, uninflamed and inflamed tissues from 20+ individuals were processed by the Regev lab (Smillie et al., Cell 2019).

### Example 6—Identifying Disease-Critical Cell Types and Programs Using Single-Cell RNA-Seq and Enhancer-Gene Architectures

### Overview of Methods

Applicants curated scRNAseq data from 10 healthy human tissues and 5 disease human tissues consisting of in total 226 samples, 1.8 million cells and 281 different annotated cell subsets (i.e., identified cell types in each tissue). For each healthy dataset, Applicants constructed cell type specific, differentially disease specific and intra-cellular gene programs (as used in this example “gene program” is used to refer to gene modules). For each disease dataset, Applicants constructed cell type specific gene programs, disease specific gene programs and cell state/intra-cellular gene programs. Details for constructing each class of programs are written in the beginning of the respective analysis sections. Applicants define a gene score as an assignment of a numeric value between 0 and 1 to each gene. Each gene program was converted into a SNP annotation by linking the gene weight to the set of SNPs identified from the SNP to gene mapping strategy.

Applicants define an annotation as an assignment of a numeric value to each SNP with minor allele count≥5 in a 1000 Genomes Project European reference panel¹, as in their previous work²; Applicants primarily focus on annotations with values between 0 and 1. Applicants define a SNP-to-gene (S2G) linking strategy as an assignment of 0, 1 or more linked genes to each SNP. Here Applicants use a distal S2G strategy defined as the union of Roadmap^{3,4} and Activity-by-Contact maps linking Enhancers to genes (Roadmap-U-ABC-tissue). For each gene score X and S2G strategy Y, Applicants define a corresponding combined annotation X×Y by assigning to each SNP the maximum gene score among genes linked to that SNP (or 0 for SNPs with no linked genes); this generalizes the standard approach of constructing annotations from gene scores using window-based strategies^{5,6} and is shown to outperform the latter in pinpointing disease signal⁷. Applicants have publicly released all gene scores and annotations analyzed in this study along with codes to reproduce the analyses (see URLs).

Applicants assessed the informativeness of the resulting combined annotations for disease heritability by applying stratified LD score regression (S-LDSC)² to a set of 127, relatively independent traits. Applicants conditioned the analysis on 86 coding, conserved, regulatory and LD-related annotations from the baseline-LD model (v2.1)^{8,9} (see URLs). S-LDSC uses two metrics to evaluate informativeness for disease heritability: enrichment score and standardized effect size (τ*). Enrichment score is defined as the proportion of heritability explained by SNPs in an annotation divided by the proportion of SNPs in the annotation relative to the corresponding unweighted S2G strategy; and generalizes to annotations with values between 0 and 1¹⁰. Standardized effect size (τ*) is defined as the proportionate change in per-SNP heritability associated with a 1 standard deviation increase in the value of the annotation, conditional on other annotations included in the model⁸. Enrichment score is used as the primary metric of interest here as τ* signal tends to miss significance cut-off for small annotations when conditioned on many annotations. The significance cut-off was determined using the False Discovery Rate (FDR) correction (qvalue<0.05).

### Healthy Blood and Brain Analysis

### Constructing Cell Type Specific Programs

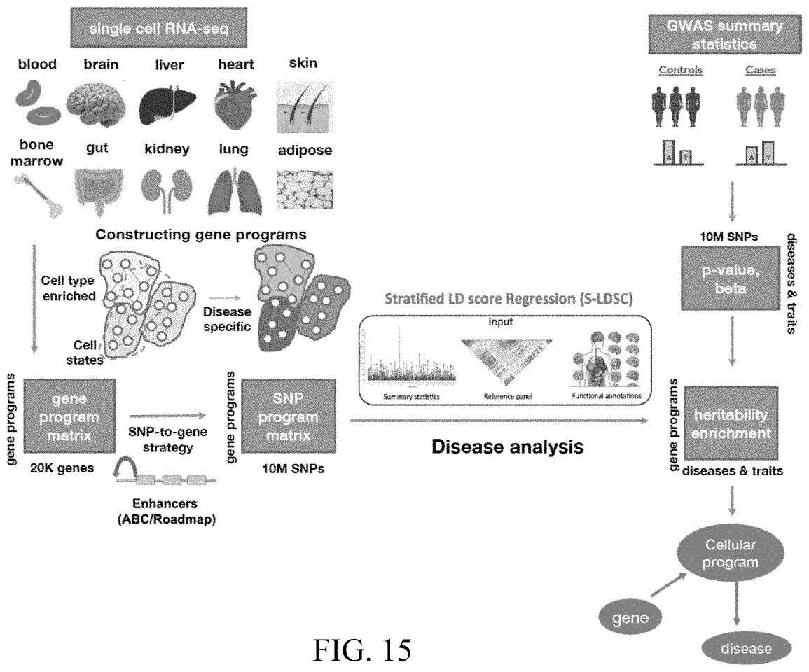

To generate cell type enriched (cell type specific) gene programs from a single cell RNA-seq (scRNA-seq) data, Applicants first cluster and annotate the cells into cell subsets using known cell type specific marker genes (see Methods). Next, a gene-level non-parametric differential expression (DE) analysis is performed between cells in a cell-type versus all other cells and each gene is assigned a probabilistic grade based on the Z score from the DE analysis (Methods). A schematic of this approach is presented in FIG. 15.

### Blood Cell Types and Traits

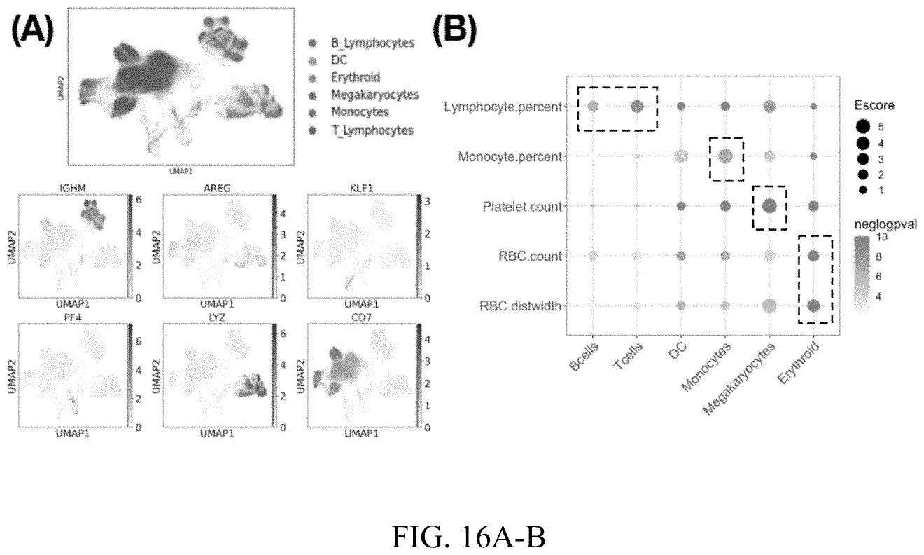

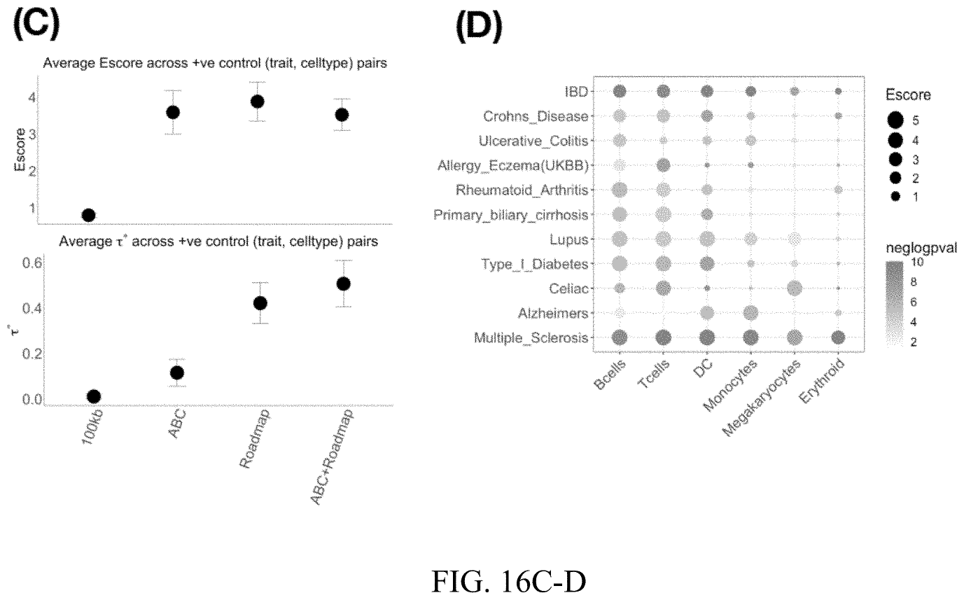

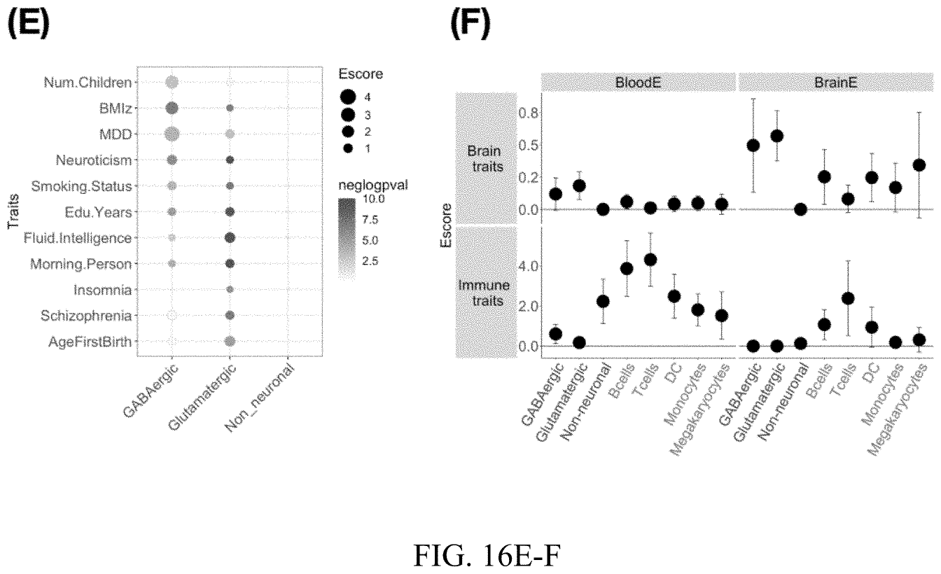

Applicants analyzed four blood related scRNAseq datasets from peripheral blood mononuclear cells (PBMC) (n=73,191 cells across 10 individuals), cord blood (n=263,828 cells across 8 individuals) and bone marrow (n=283894 cells across 8 individuals). Applicants focused the initial analysis on 6 core cell type specific programs derived from this single cell data and 6 blood biomarkers collected in the UK Biobank. Applicants identified pairs of blood biomarkers and cell type enriched programs with expected high cell type specificity as positive controls to validate the results (for e.g. red blood cell counts and volume matched with the Erythroid cell types, Monocyte percentage matched with Monocytes, Lymphocyte percentage matched with T and B Lymphocytes). First, Applicants looked to identify an optimal SNP to gene (S2G) strategy by evaluating a standard 100 kilobase window approach, Activity by Contact (ABC) mapping, Roadmap enhancer mapping and a custom Roadmap union ABC (Roadmap-U-ABC) approach. The Roadmap-U-ABC S2G strategy outperformed all the other methods including the standard 100 kilobase window based S2G strategy both in terms of average Enrichment score and average τ* across these positive controls (FIG. 16C). Additionally, Applicants observed high specificity in enrichment score across positive control blood biomarkers and cell type pairs (FIG. 16B). The Roadmap-U-ABC S2G strategy was used for all following analyses.

Next, the same cell type specific programs from the blood data were evaluated for 10 independent autoimmune traits spanning IBD, Alzheimers, Multiple Sclerosis and more (FIG. 16D). Applicants recapitulated many of the prior signals⁵ such as Allergy-Eczema enrichment in T Lymphocytes and Multiple sclerosis enrichment broadly across all immune cells. Additionally, Applicants identified several novel associations, such as Celiac disease heritability in T Lymphocytes, Ulcerative Colitis heritability in B Lymphocytes and Rheumatoid Arthritis heritability in T and B Lymphocytes. Genes driving the heritability signals were identified by integrating signals from the cell type specific program weight and the GWAS summary statistic significance values (see Methods). Applicants find the T Lymphocyte signal in Celiac disease is driven by CD247 and LBH suggesting a connection with immunodeficiency and cell growth.

### Brain Traits and Cell Types

Applicants analyzed a brain scRNAseq dataset from Allen Brain Atlas (n=47,509 cells across 3 individuals). From this data, Applicants identified 3 core cell type specific programs—GABA-ergic neurons, glutamatergic neurons and non-neuronal programs. Applicants evaluated these programs for 13 brain-related traits. First, Applicants performed a comparison of blood and brain cell types and traits to evaluate the impact of tissue specific S2G strategies. Applicants observed that >2× enrichment score in brain related traits is contributed by both the brain specificity of the cell type specific program and the brain specificity of the S2G strategy (Roadmap-U-ABC-brain) (FIG. 16E). Applicants also observed a >2× enrichment score in blood related traits and blood cell type specific and blood specific Enhancer-to-gene strategy (Roadmap-U-ABC-blood) (FIG. 16F); these two results may reflect the presence of a “blood brain barrier” in disease signal. All following analyses utilized a tissue specific enhancer strategy while linking SNPs to genes.

Applicants observed specificity of enrichment score of brain related traits in GABA-ergic and glutamatergic neuron cell type specific programs when linked to Roadmap-U-ABC-brain S2G strategy (FIG. 16E). GABA-ergic neuron cell type specific program showed high disease signal for Major Depressive Disorder (MDD) and BMI. Top genes driving the signal for MDD and GABA-ergic cell type specific program include genes critical to neurological development (TCF4, PCLO etc) (Methods, Table 12). Glutamatergic neuron cell type specific program showed high disease signal for Intelligence, Education years and Schizophrenia. Non-neuronal cell type specific program did not show any significant disease signal across brain traits.

### Generalizing to Many Healthy Tissues

### Urine Biomarkers and Kidney/Liver Cell Types

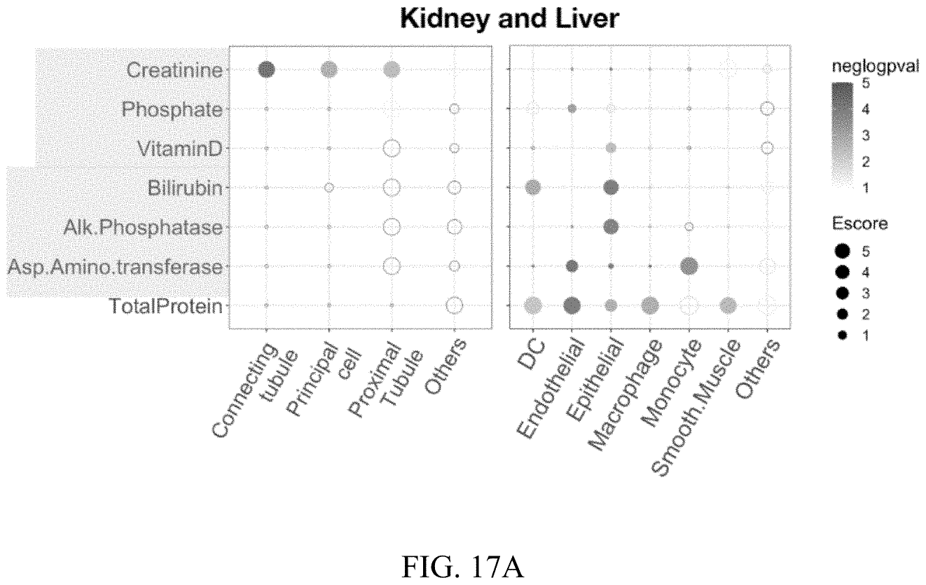

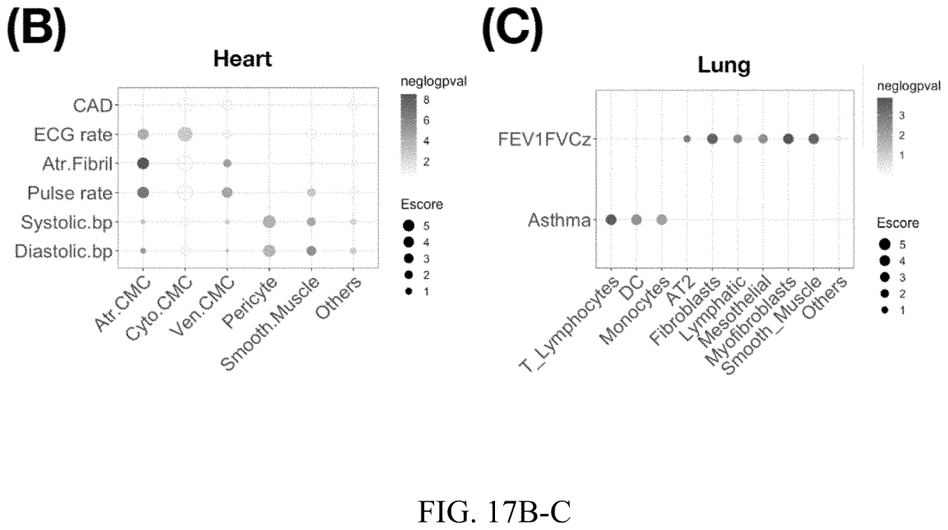

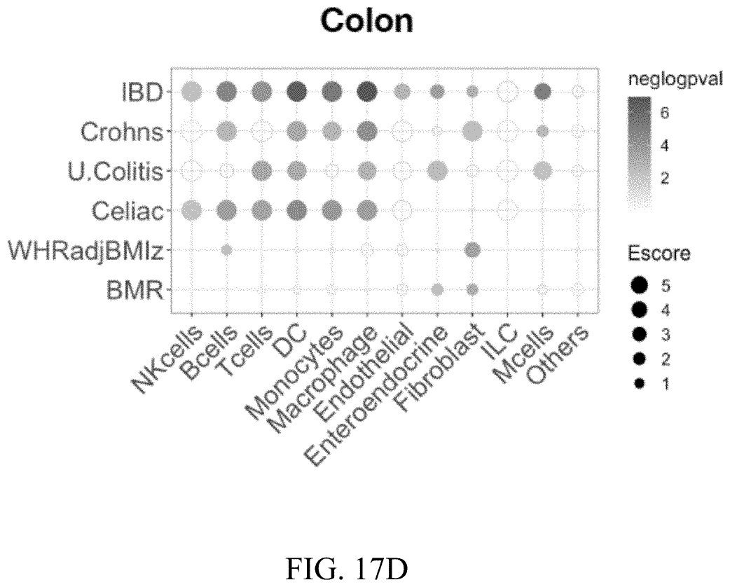

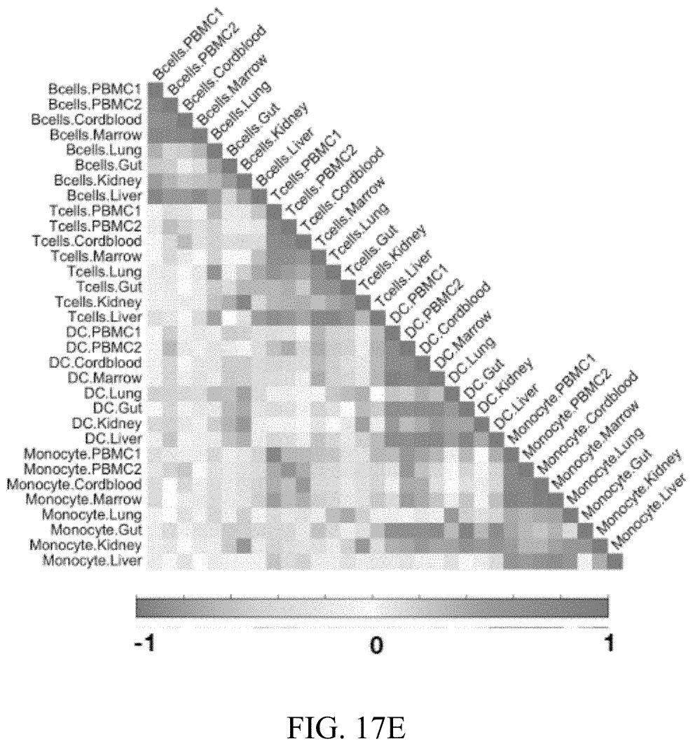

To better understand the genetic basis of 7 urine biomarkers from the UK Biobank evaluated over 500K individuals, Applicants analyzed a kidney scRNAseq dataset (n=40268 cells across 13 individuals) and a liver scRNAseq dataset (n=13340 cells across 4 individuals). Applicants identified 12 core cell type specific programs for kidney and 24 core cell type specific programs for liver tissues. The 7 urine biomarker traits were categorized into 3 related to kidney function and 4 related to liver function. The kidney related urine biomarker enrichment signal was specific to kidney cell type specific programs linked to SNPs using the Roadmap-U-ABC-kidney S2G strategy. Likewise, the liver related urine biomarker enrichment signal was specific to liver cell type specific programs using the Roadmap-U-ABC-kidney S2G strategy (FIG. 17A). Creatinine, a waste product of muscles which is removed from the body through the kidney displays the highest heritability in kidney cell types specifically the proximal tubule, principal cell and connecting tubule. Bilirubin and Alkaline-Phosphatase, both associated with liver damage and function, showed strongest signal in the liver epithelial cells while aspartate amino transferase had highest signal in the Monocyte cells.

### Lung Traits and Lung Cell Types

To examine the genetic basis of lung-related traits Applicants analyzed scRNAseq dataset from the lower lung lobes (n=31,644 cells across 10 individuals). From this data, Applicants identified 19 core cell type specific programs including cell subsets from epithelial, stromal, immune and endothelial compartments. These programs from the lung data were evaluated for 2 lung related traits—lung capacity (Forced Expiratory Volume: FEV1) and Childhood Onset Asthma.

FEV1 is a standard metric of lung capacity measuring the amount of air an individual can force from the lung within one second. FEV1 showed the highest enrichment in connective tissue cells such as Fibroblasts and Myofibroblast cell type specific programs linked using a Roadmap-U-ABC-lung S2G strategy. Fibroblast and myofibroblasts are both highly relevant cell types for lung capacity since their differentiation and production of extracellular matrix (ECM) is a hallmark of Fibrosis and COPD, and both diseases are characterized by reduction in lung capacity. Applicants identified several genes contributing to the heritability signal in Fibroblasts through the scV2F gene analysis and performed a pathway analysis on them identifying significant enrichment in the ‘TGF-beta regulation of extracellular matrix’ and ‘ECM-receptor interaction’ pathways. ITGA1 and LOX maintain ECM production which can determine the tissue architecture, stability and elastic recoil. Additionally, TGFBR3 affects the pool of available TGFB, a master regulator of lung fibrosis, and mutations in TGFBR3 may change lung capacity by altering the regulation of lung fibrotic pathways (FIG. 17C). Furthermore, myofibroblasts represent what is thought of as a disease state of fibroblasts during fibrosis and the scV2F gene analysis identifies the same ECM and TGFB signaling pathways in myofibroblasts. There are additional genes including COL8A1, BAMBI, VCL driving the heritability specific to myofibroblasts that add increased burden to the modulation of ECM and TGF signaling pathway beyond what Applicants found in Fibroblasts.

### Heart Traits and Heart Cell Types

To interrogate the genetic basis of heart-related traits, Applicants curated a scRNAseq dataset of heart tissue consisting of 4 chambers (n=287269 cells across 7 individuals). From these data, Applicants identified 12 core cell type specific programs (Table 12). These programs from the heart data were evaluated for 6 heart-related traits that were categorized into coronary artery disease, blood pressure (Systolic and Diastolic) and cardiac rhythm (ECG rate, pulse rate, Atrial fibrillation).

Systolic and diastolic blood pressure showed high heritability enrichment in pericyte and vascular smooth muscle gene programs, linked using a Roadmap-U-ABC-heart S2G strategy, but showed no signal in cardiomyocytes (FIG. 17B). Consistent with this pattern of cellular heritability, pericytes and vascular smooth muscle cells both are closely associated with blood vessels and can affect blood pressure by modulating vascular tone. Applicants identified several genes contributing to the heritability signal through the scV2F gene analysis and performed a pathway analysis on them identifying ‘Nitric Oxide stimulation of guanylate cyclase’, ‘Vasucular smooth muscle contraction’ and ‘Adrenergic pathway’ as significantly enriched for genes contributing to the heritability signal (Table 12). GUCY1A3 is a well-established nitric acid receptor in the heart and affects vasodilation and blood pressure by relaxing the vascular smooth muscle cells lining blood vessels. Additionally, CACNA1C and EDNRA are important for the function of vascular contraction and maintaining vascular tone, which are mechanisms for regulating blood pressure, and are carried out by pericytes and vascular smooth muscle cells. Finally, PLCE1, PDE8A and CACNA1C are associated with the adrenergic pathway and modulate the blood pressure response to adrenaline (FIG. 17B).

Atrial fibrillation and other cardiac rhythm traits showed highest heritability enrichment in the atrial cardiomyocyte gene program linked using Roadmap-U-ABC-heart S2G strategy (FIG. 17B). Consistent with this pattern of heritability, cardiomyoctes determine heart rhythm through their coordinated electrical activity. Applicants identified several genes contributing to the heritability through the scV2F gene analysis and performed a pathway analysis identifying ‘Potassium channels’ as the top pathway enriched. PKD2L2, CASQ2 and KCNN2 are some of the largest signals driving the heritability indicating that mutations in ion channel genes, which are essential for generating action potentials in cardiomyocytes, may contribute to atrial fibrillation.

Cell Types from Additional Tissues

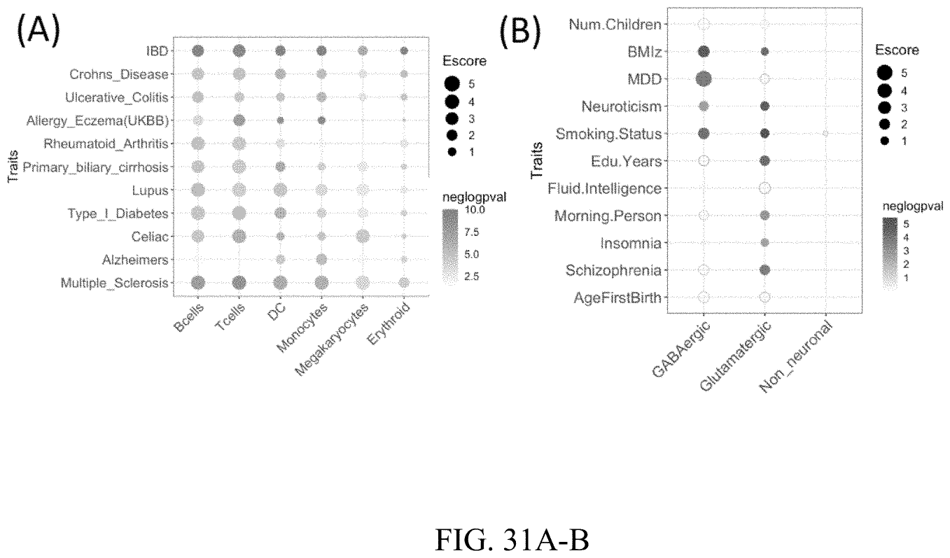

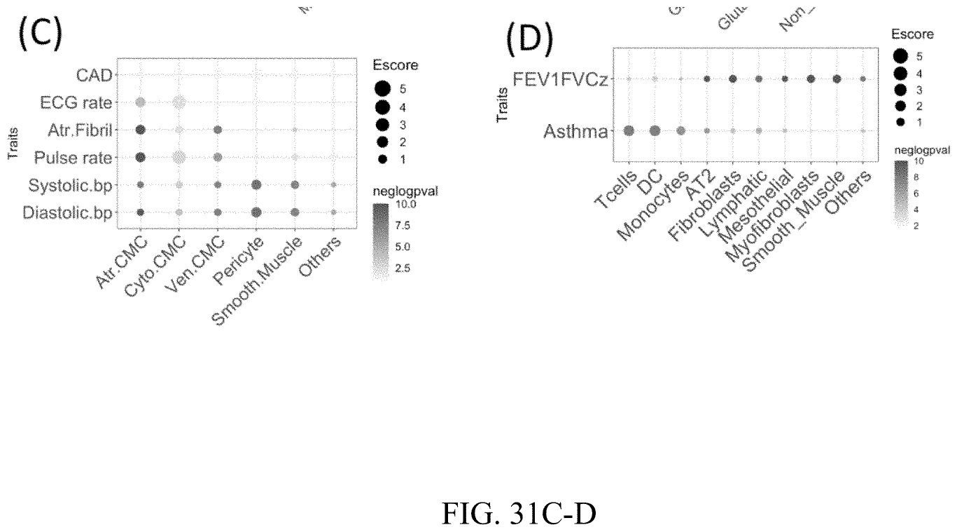

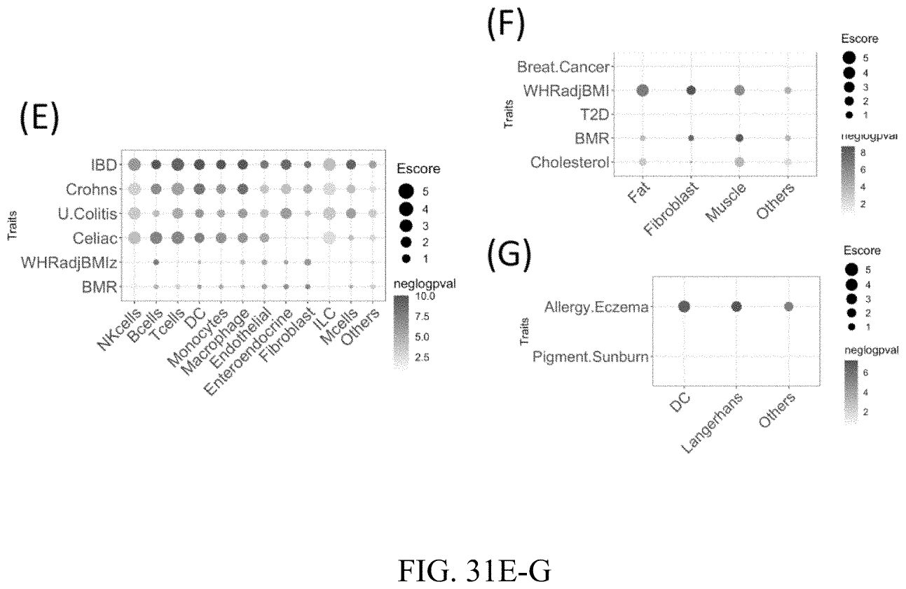

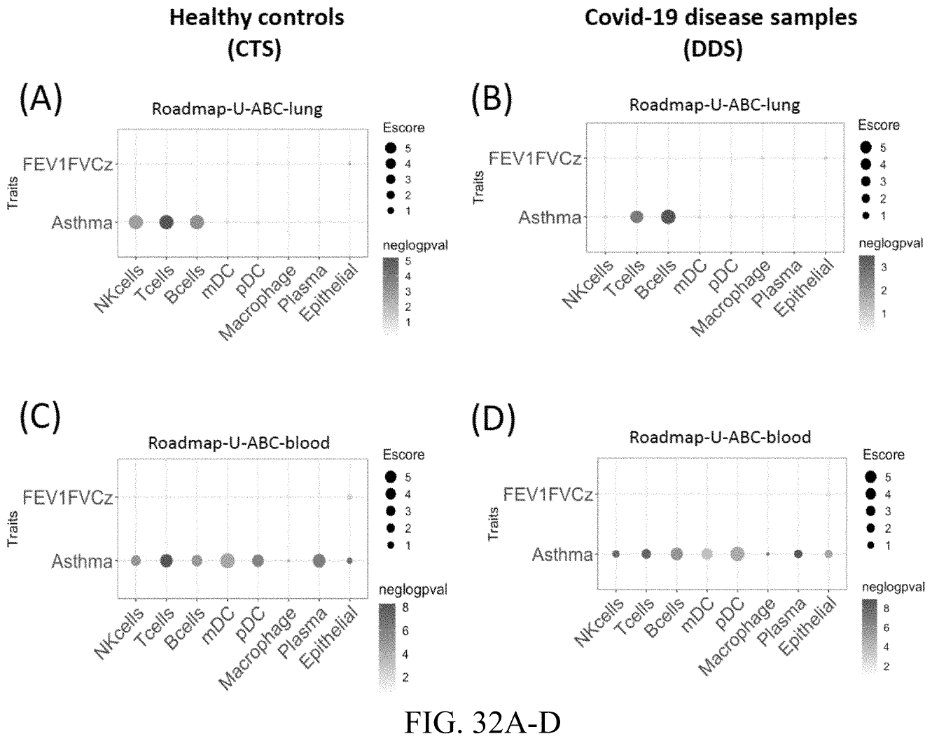

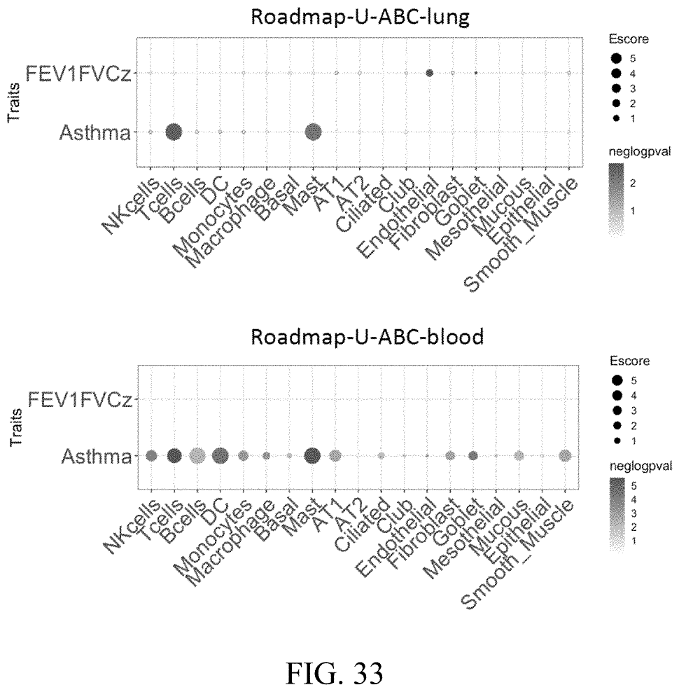

Applicants also analyzed additional scRNAseq data from the human colon (n=110373 cells across 12 individuals), skin (n=71864 cells across 9 individuals) and adipose tissue (n=11184 cells across 3 individuals). Applicants identified 20 cell type specific programs for gut, 13 cell type specific programs for skin and 13 cell type specific programs for adipose data. The Waist-to-Hip Ratio adjusted for BMI and Basal Metabolic traits both exhibited high heritability enrichment in colon resident fibroblast cells (FIG. 31E). The Lymphoma and Dendritic cells in skin showed high enrichment signal for Allergy-Eczema (FIG. 31G). Finally, the strongest signal in adipose tissues data was observed for the Fat cells for the Waist-to-Hip Ratio adjusted for BMI trait (FIG. 31F).

### Analysis of Immune Cells Across 7 Tissue Contexts

Analyzing resident immune cells from varying tissue contexts, Applicants found high similarity between cell type specific programs of the same broad cell types. For this analysis, Applicants looked across the 2 pbmc datasets, as well as bone marrow, cord blood, lung, gut, kidney and liver tissues. B Lymphocytes, T Lymphocytes, DC and Monocytes had correlation within their respective groups (FIG. 17E). Applicants find the resulting heritability enrichment of each cell type specific program to be largely similar and not varying based on the tissue source.

Identifying Disease Specific Programs from Paired Healthy and Disease Single Cell Data

### Constructing Disease Specifically Enriched Gene Programs

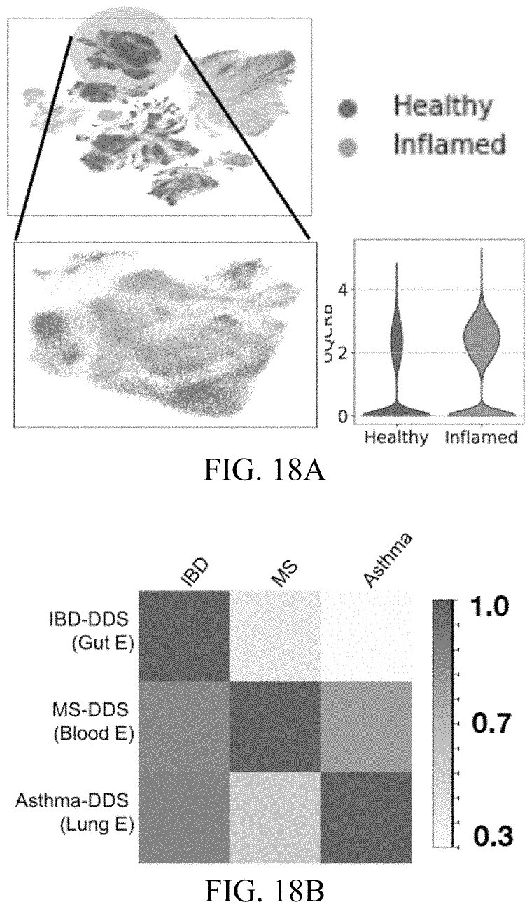

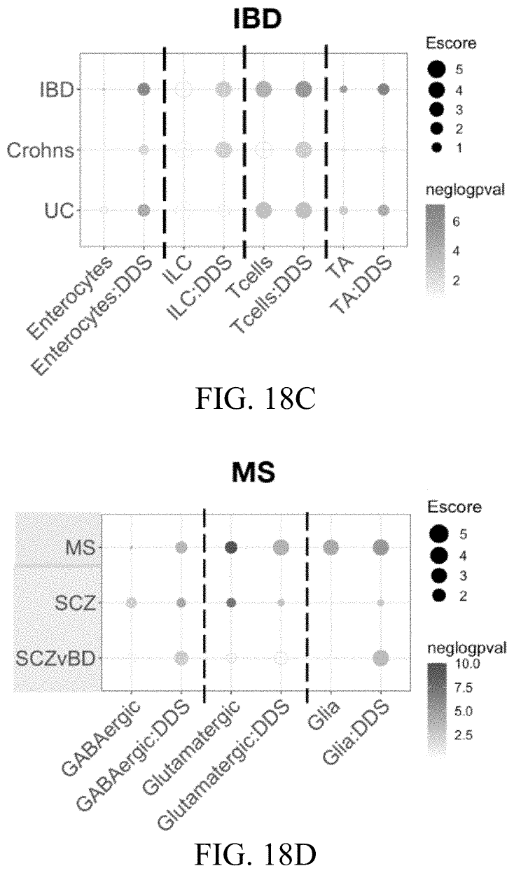

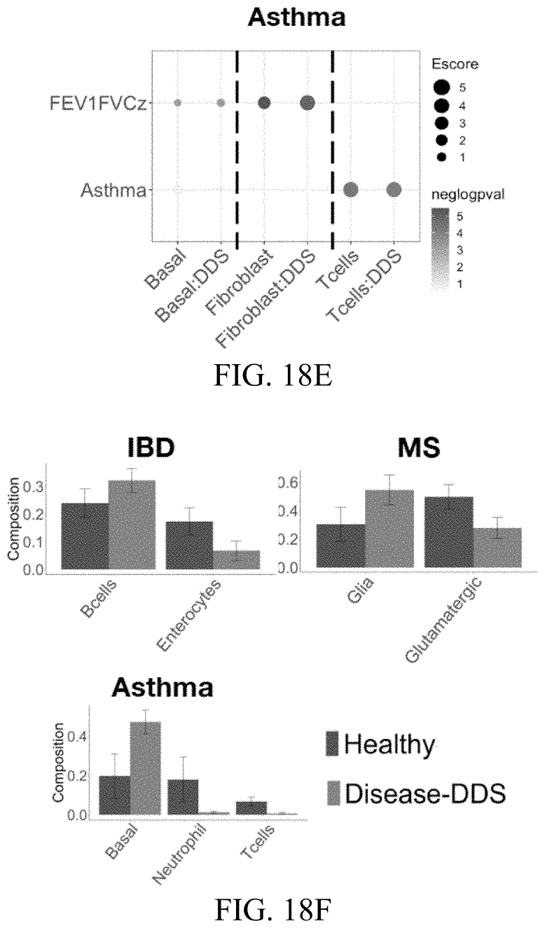

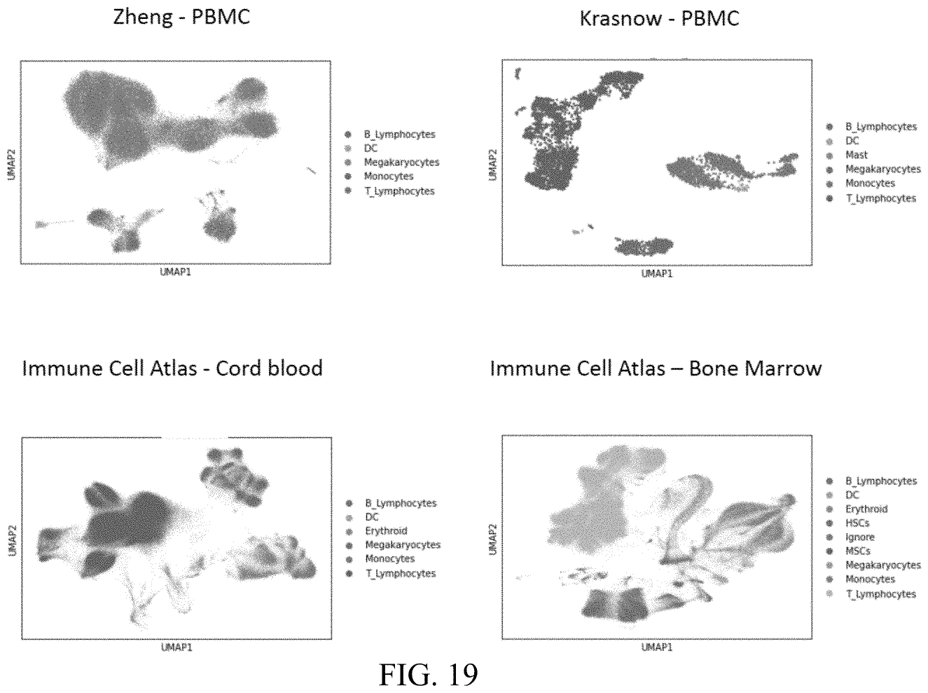

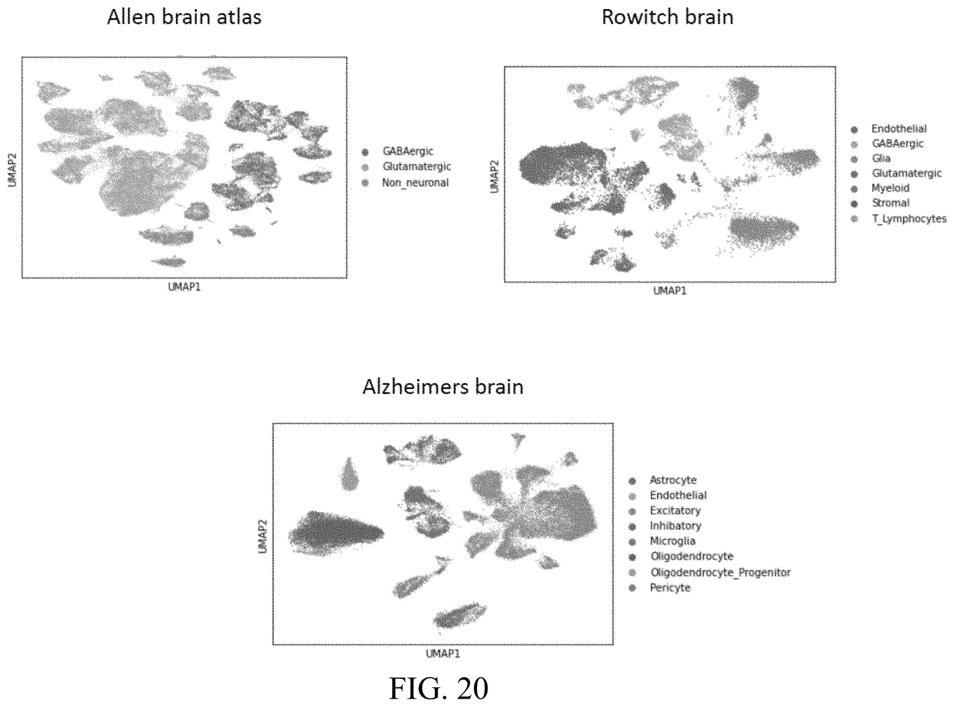

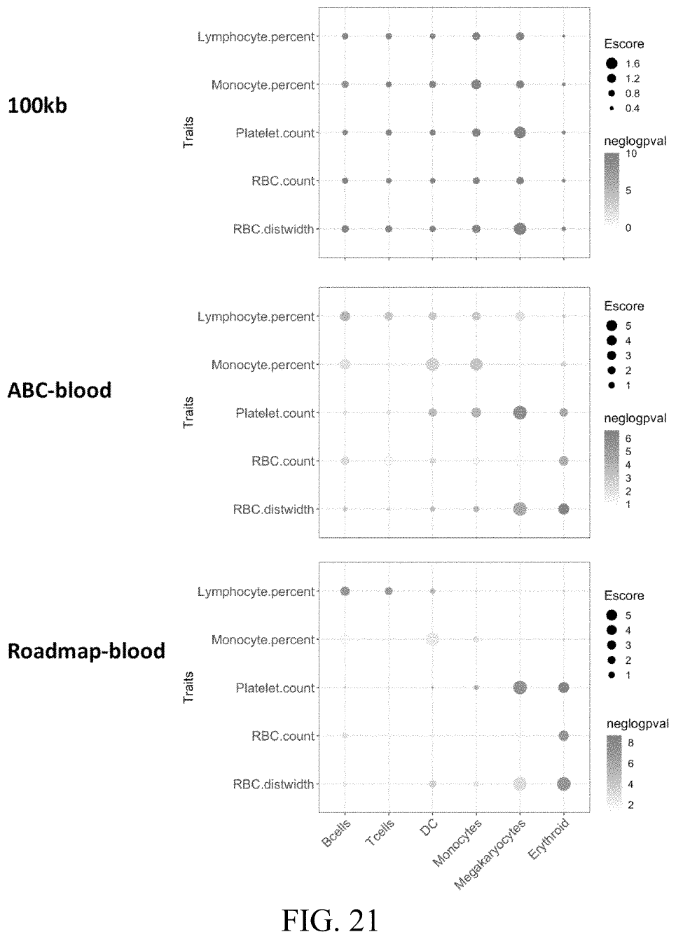

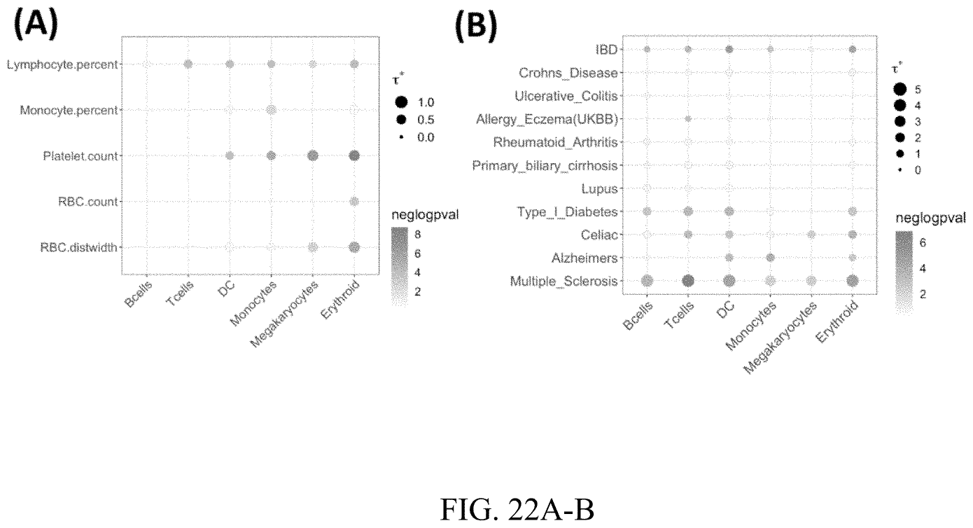

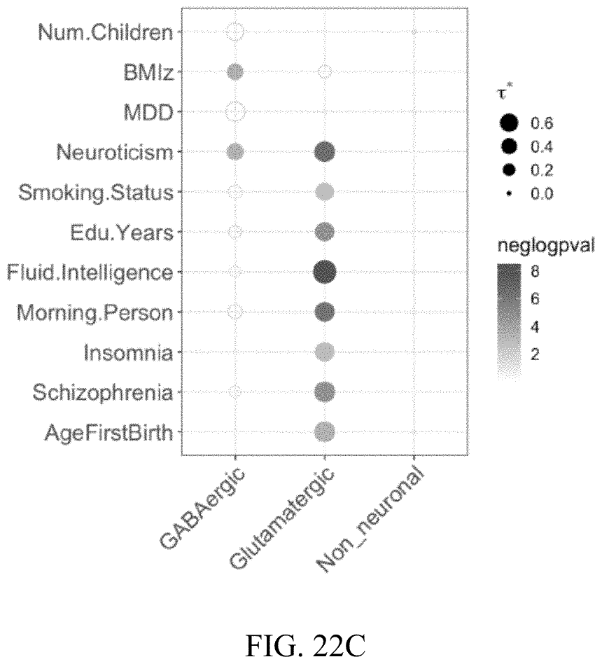

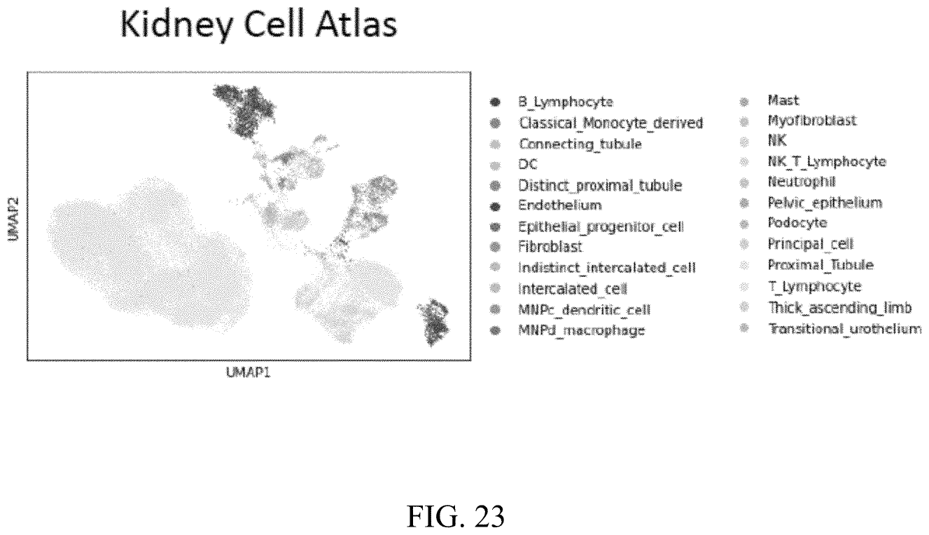

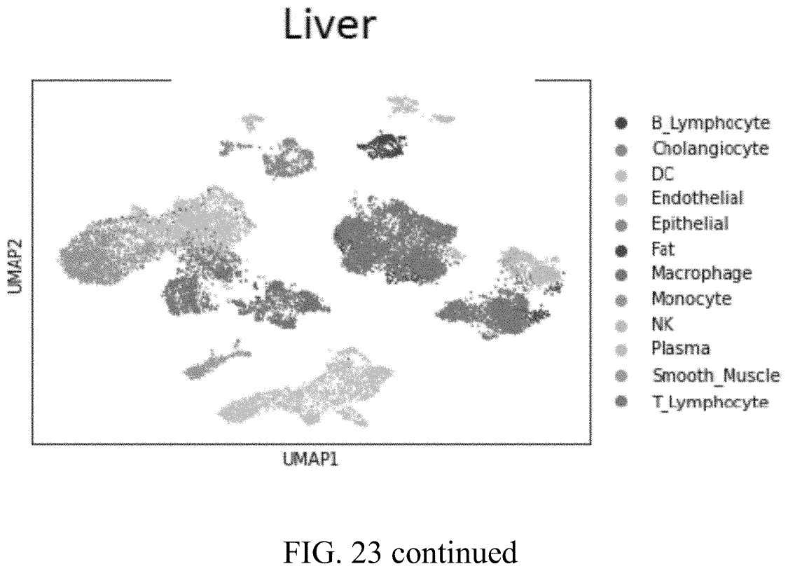

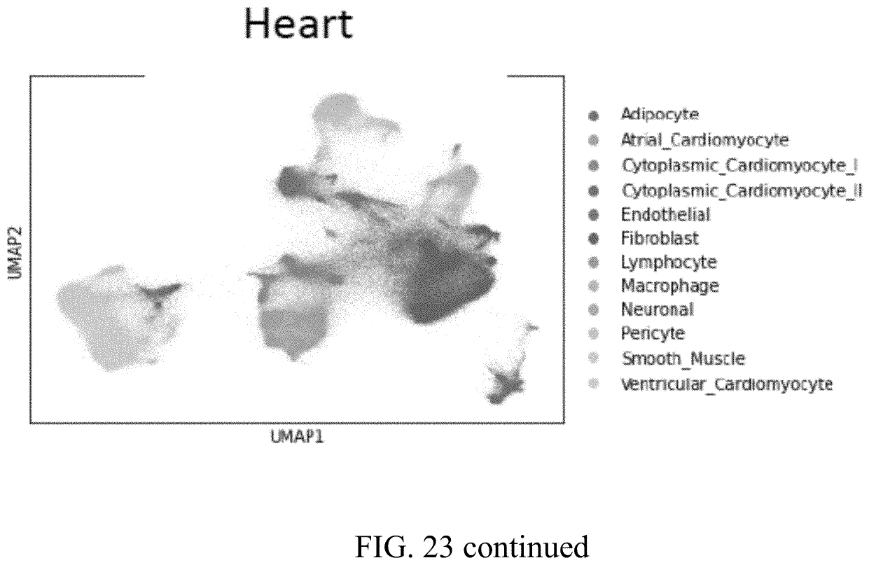

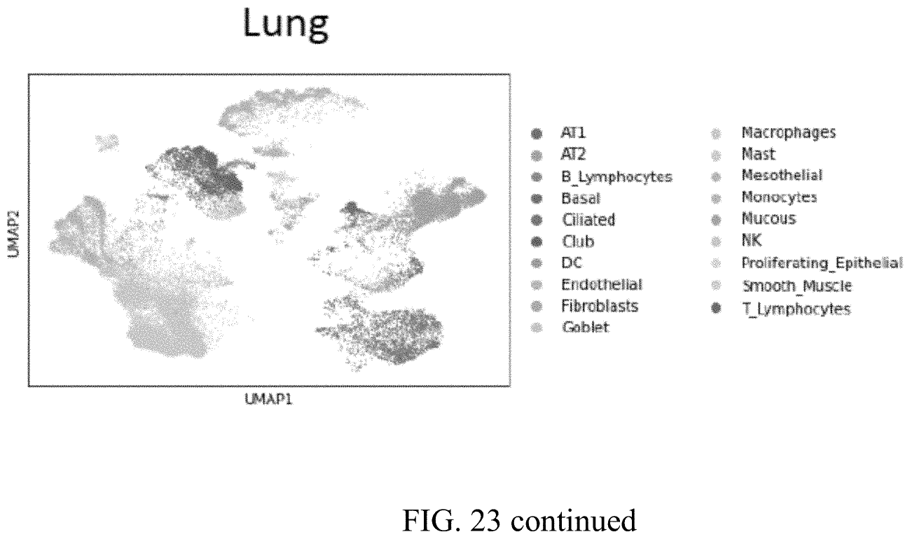

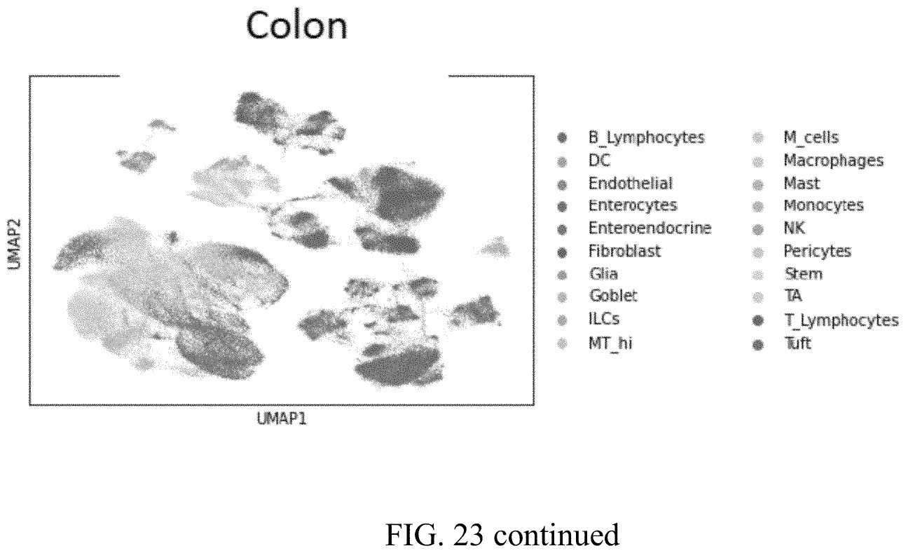

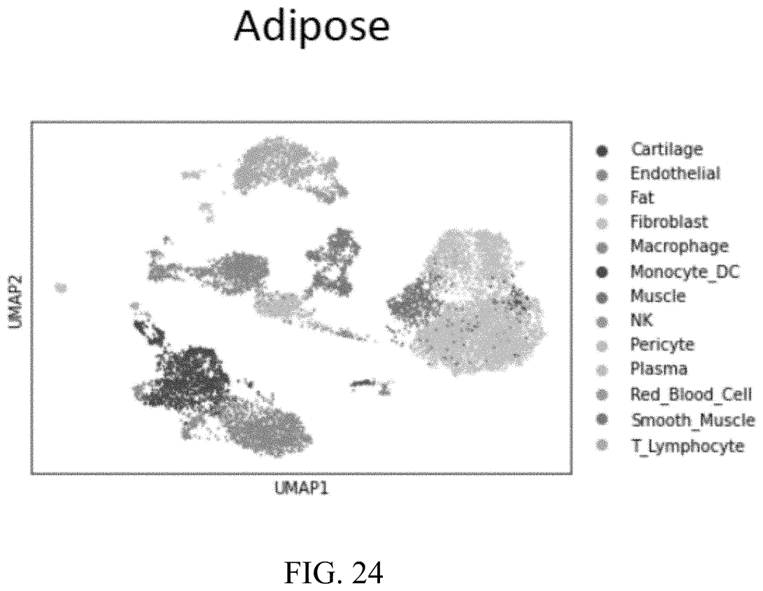

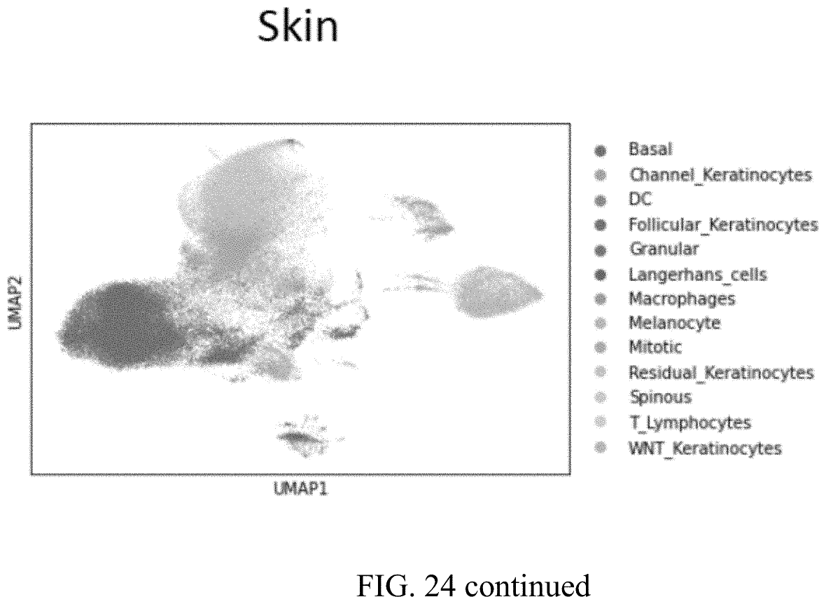

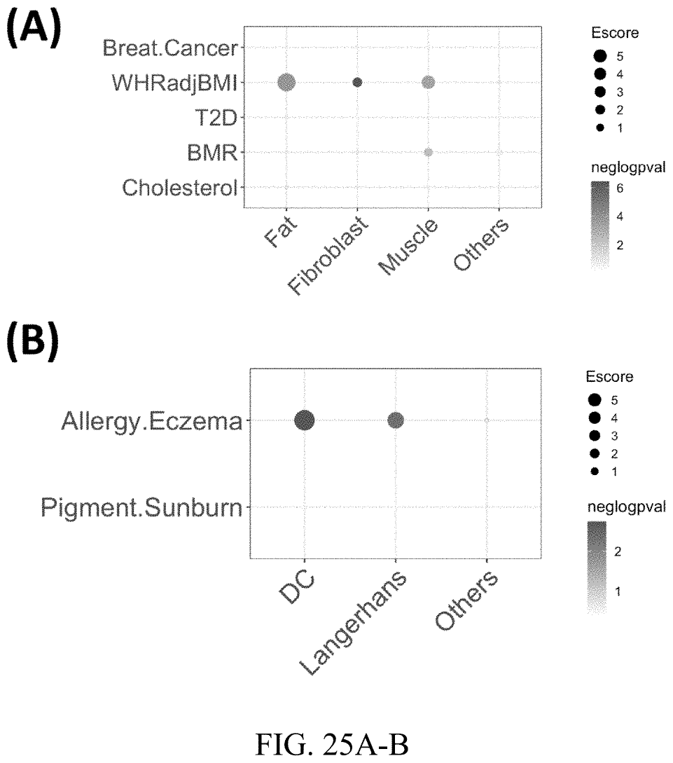

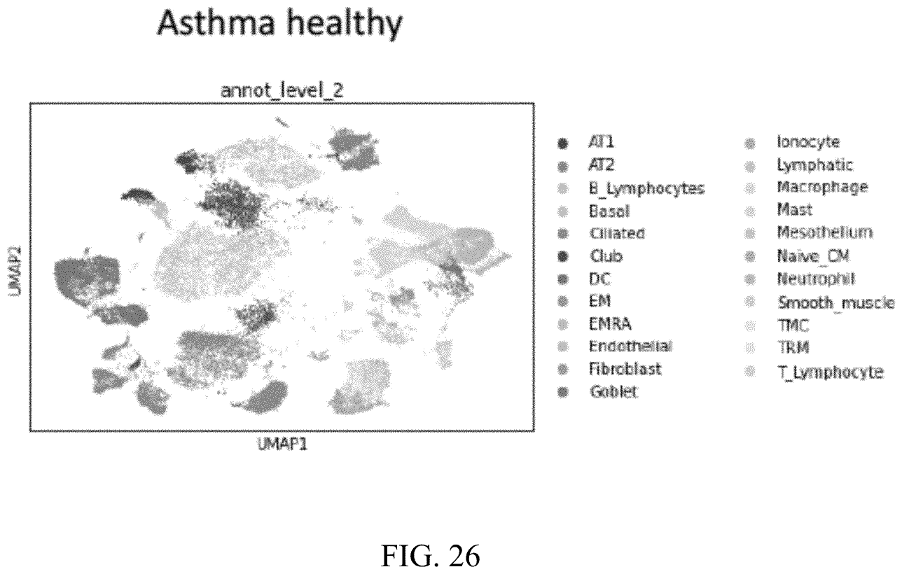

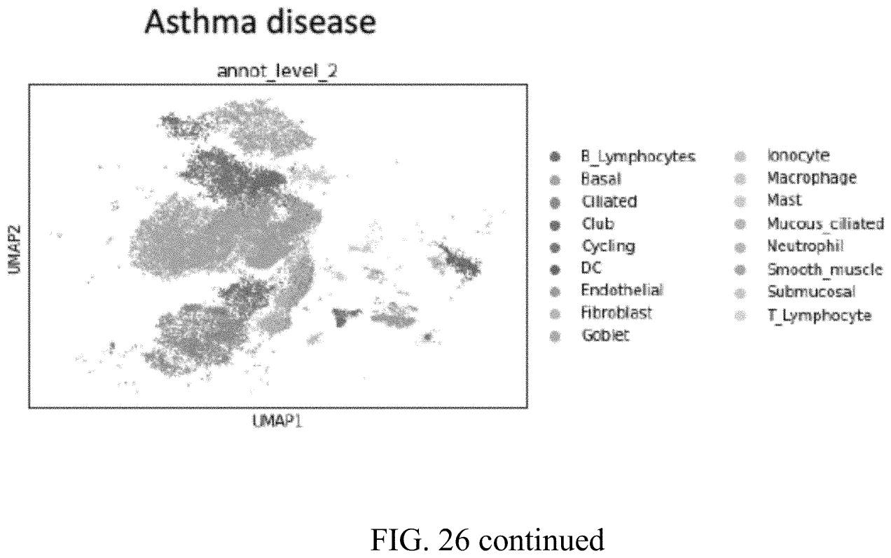

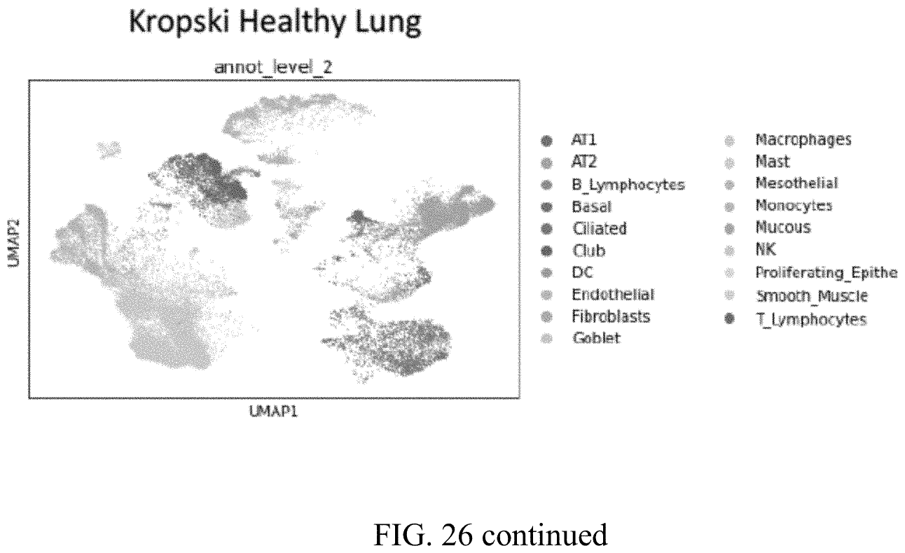

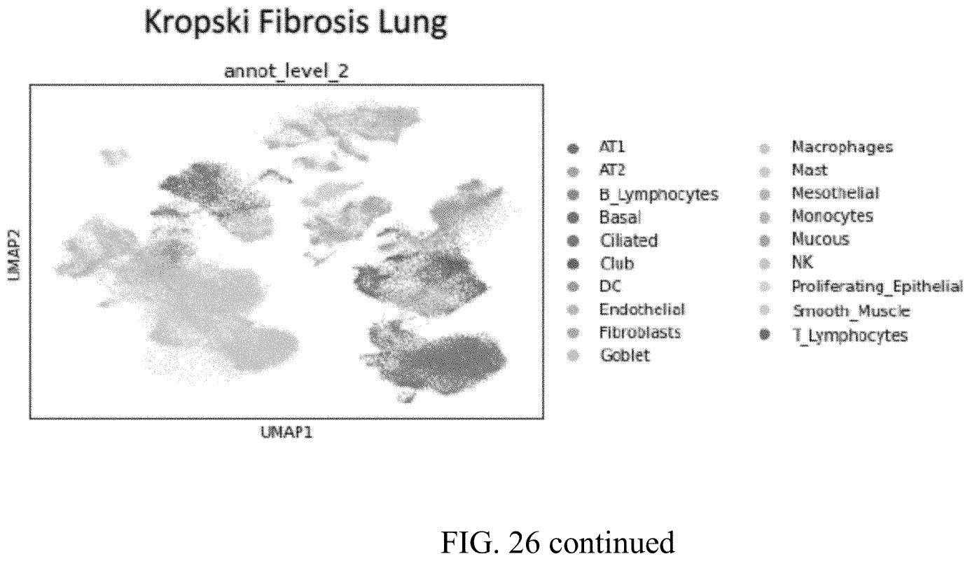

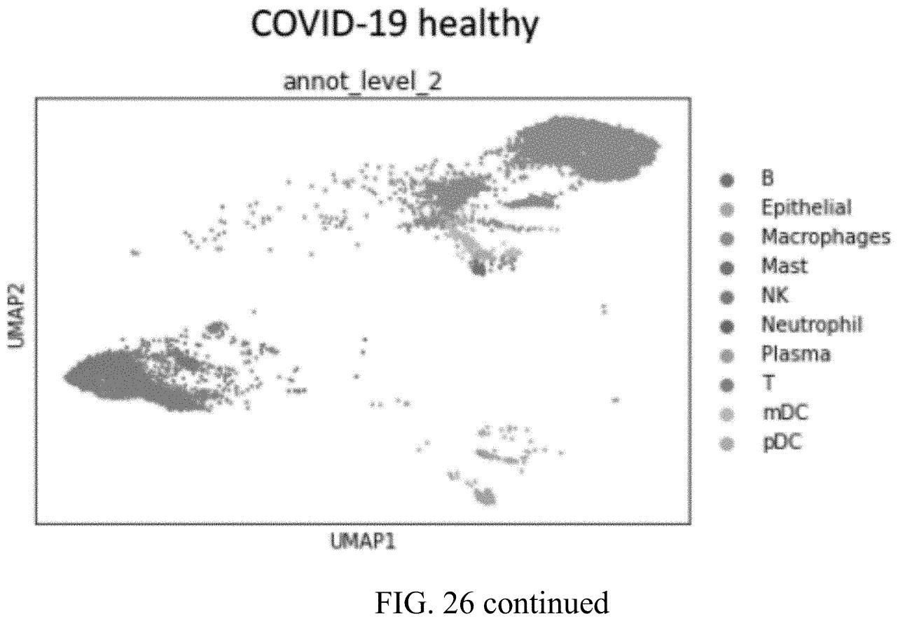

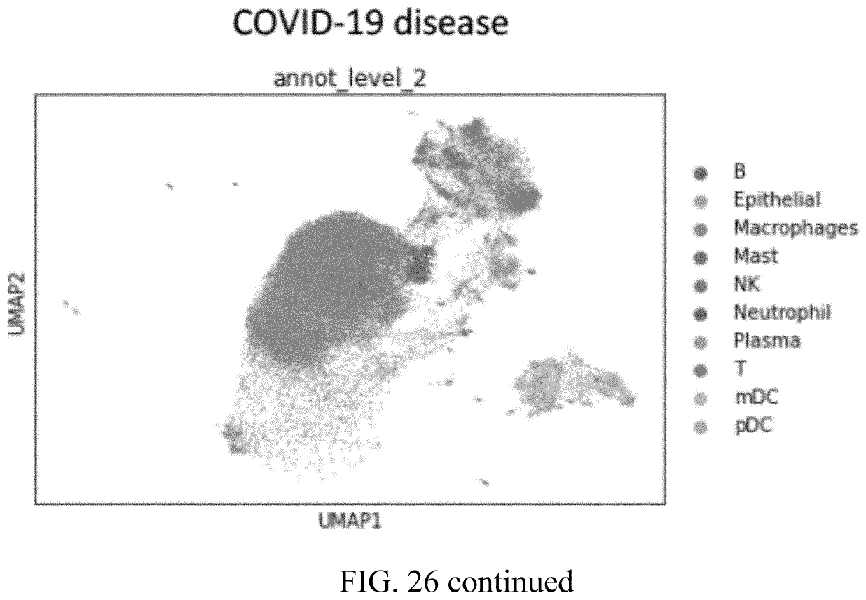

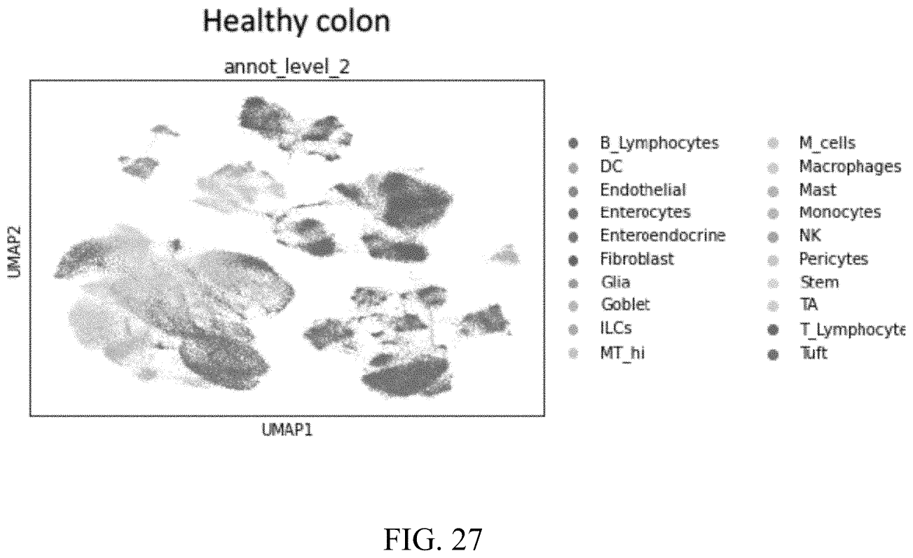

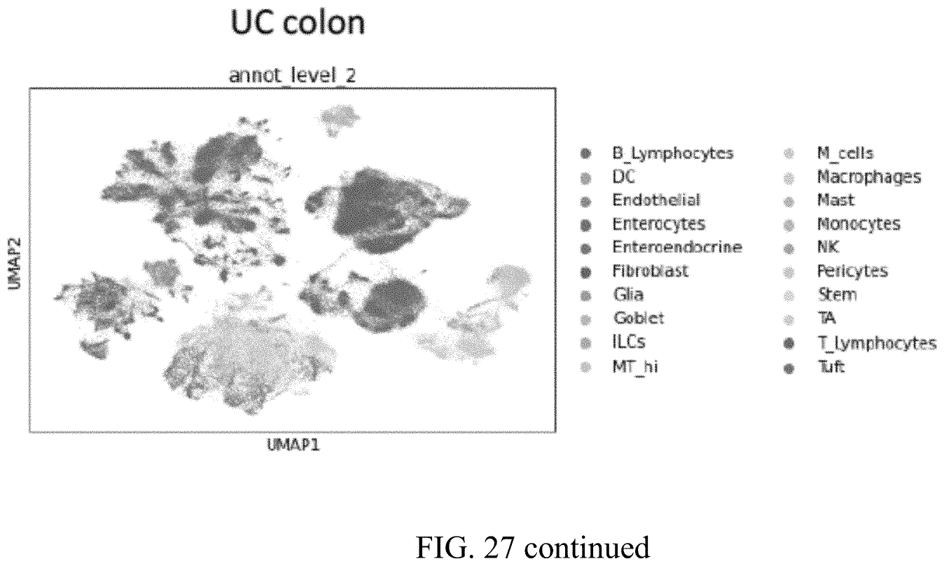

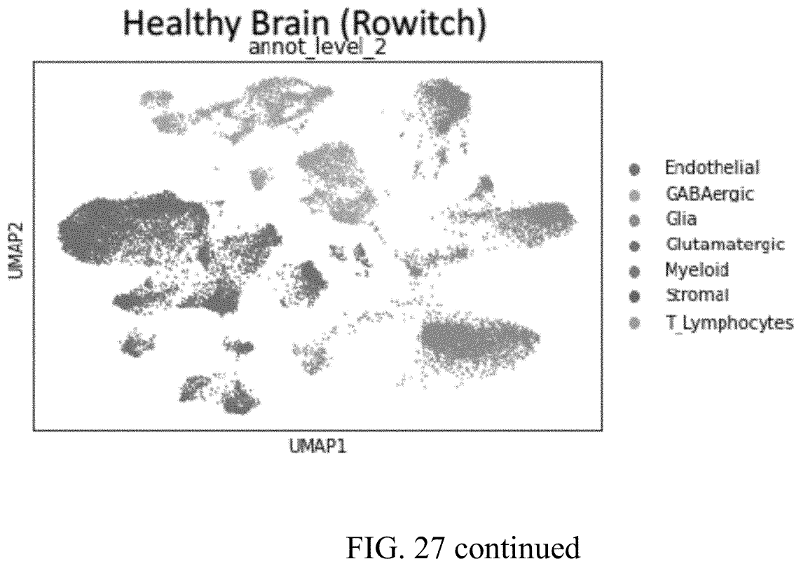

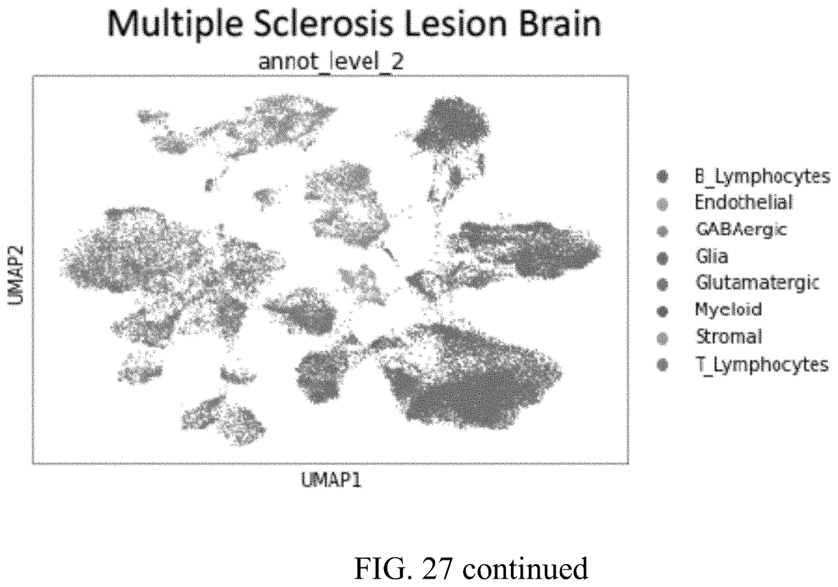

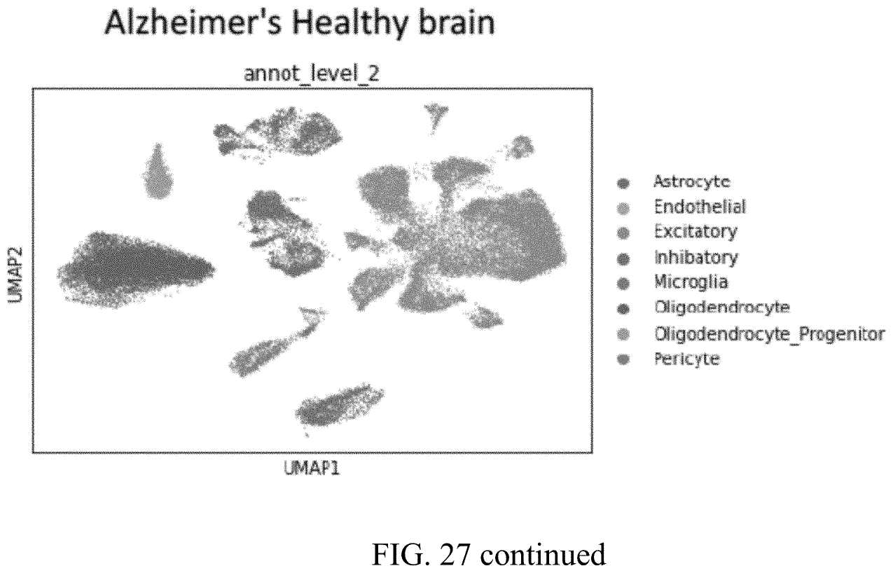

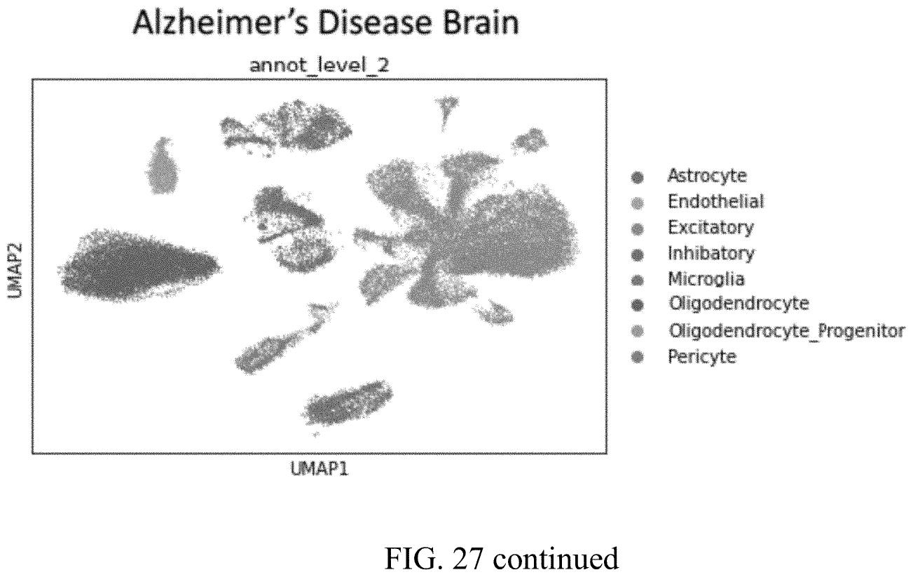

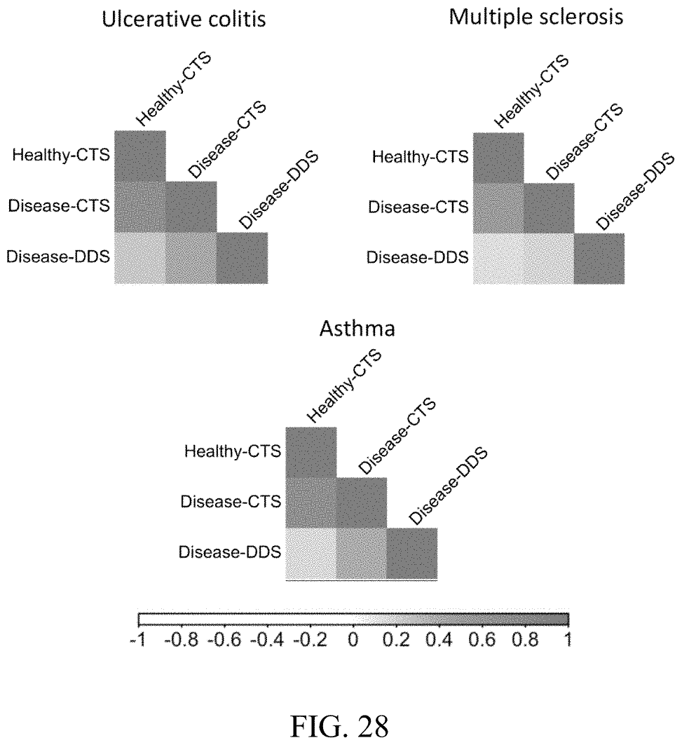

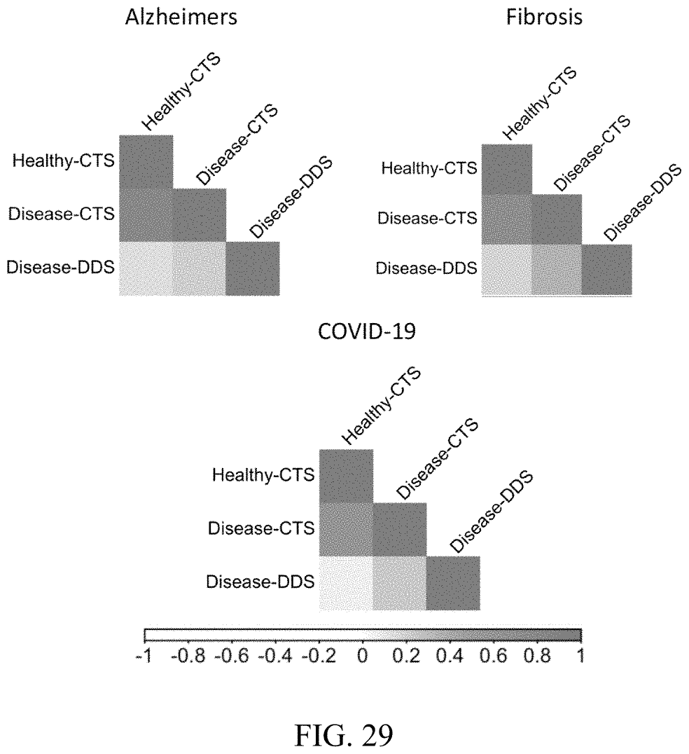

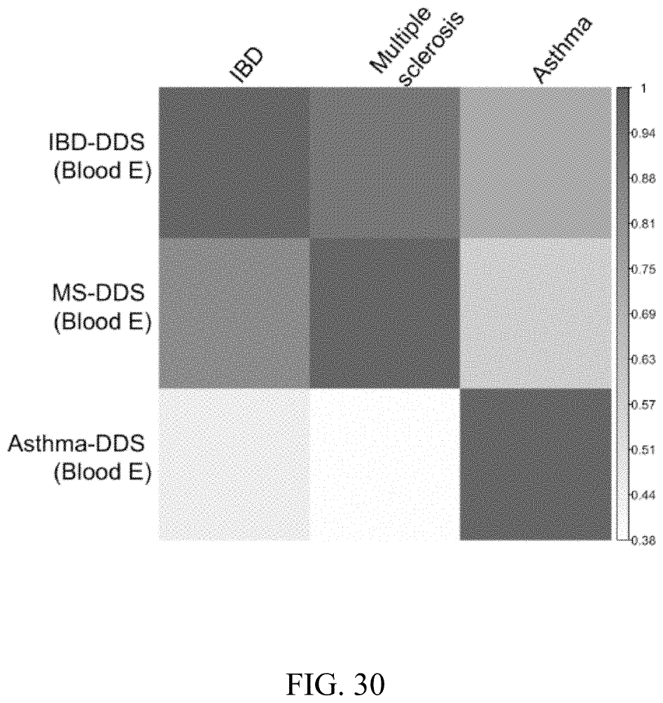

Each disease tissue Applicants analyzed consisted of matched healthy and disease samples. Applicants first constructed cell type specific gene programs across the disease cells alone. Healthy and disease cell type specific programs of the same cell type were predominantly similar (FIG. 18B) so Applicants did not separately perform a heritability analysis over the disease cell type specific programs. Applicants then constructed disease specifically enriched gene programs for each cell type to highlight genes specifically expressed in disease state. To generate disease specifically enriched gene programs from a single cell RNA-seq (scRNA-seq) data, Applicants first cluster and annotate the cells into cell types using marker genes in both the healthy and disease tissues (Methods). Next, a gene-level non-parametric differential expression (DE) analysis is performed between cells from healthy tissue and cells from disease tissue annotated with the same cell-type label and each gene is assigned a probabilistic grade based on the Z score from the DE analysis (Methods). Example of a result from this approach is presented in FIG. 18A.

### IBD Relevant Ulcerative Colitis Disease Specific Programs

Applicants analyzed Ulcerative Colitis scRNAseq consisting of 25 cell types and over 100K cells from each of the healthy and disease contexts and constructed disease differentially specific gene programs for each cell type. Applicants find a strong disease specific signal in T Lymphocyte, Enterocyte and ILC disease specific programs (FIG. 18C). The T Lymphocyte program is enriched for activation genes with much of the heritability signal found in IL2RA, a Treg specific cell type marker, to be driving this signal. IL2RA is a critical gene for Treg function which regulates surrounding T cell response to disease. There is a larger number of Tregs in the disease state which may be due to the overcompensation in product due to the mutations in IL2RA affecting Treg function. Additionally, in Enterocytes disease specific programs Applicants find genes driving this signal. Applicants found these genes are part of the pathway affecting the nutrient absorption function of Enterocytes in disease state.

### Multiple Sclerosis Relevant Disease Specific Programs

Applicants also looked at multiple sclerosis a debilitating autoimmune disorder. Applicants worked with an MS dataset consisting of 10 cell types and over 60K cells from healthy and disease contexts. There is a strong signal in Endothelial cells and Glia cells in the brain (FIG. 18D). In endothelial cells Applicants see that genes driving this signal (Table 9). Mutations in these genes may be inhibiting endothelial cell function in disease states to properly respond to MS disease phenotype in the brain. Additionally, glia cells are critical and known component in MS.

### Lung Capacity Relevant Fibrosis Disease Specific Programs

Applicants also looked at Fibrosis a common lung related disease phenotype and its relationship with lung capacity. Applicants looked at the Fibrosis dataset consisting of 10 cell types and over 60K cells from healthy and Fibrosis disease contexts. There is a strong signal in Endothelial cells in the lung. In myofibroblast cells Applicants see genes driving this signal Table 9). Mutations in these genes may be inhibiting endothelial cell function in disease states to properly respond to fibrosis disease phenotype in the brain.

### Enrichment of Gene Programs and Pathways in Health and Disease

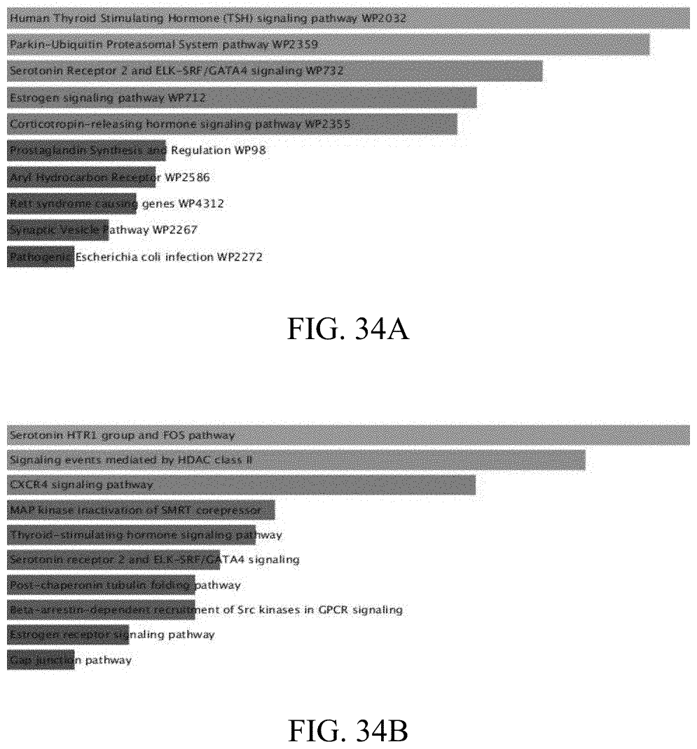

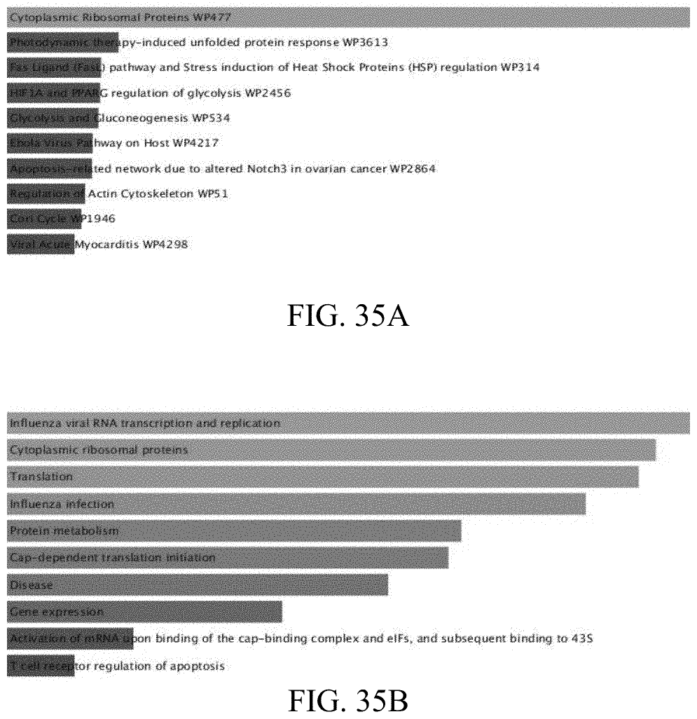

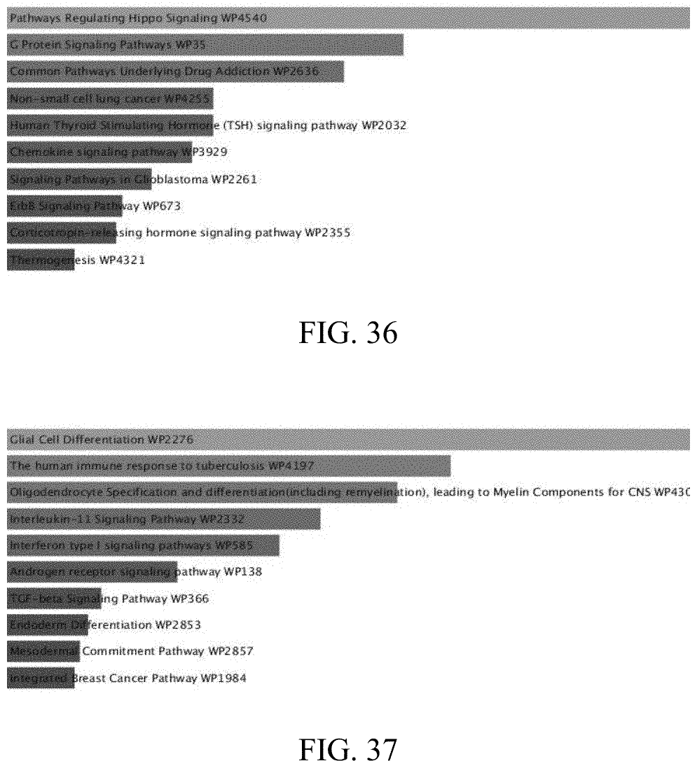

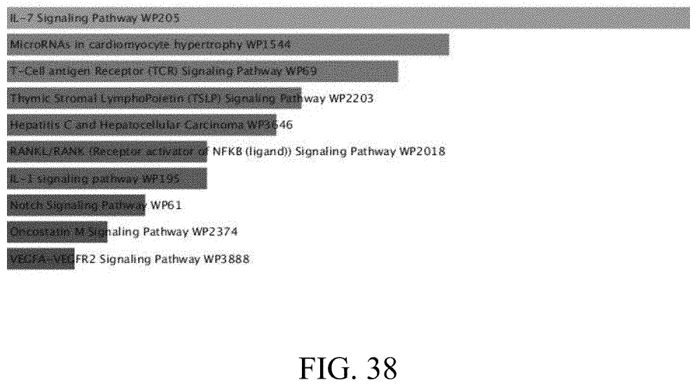

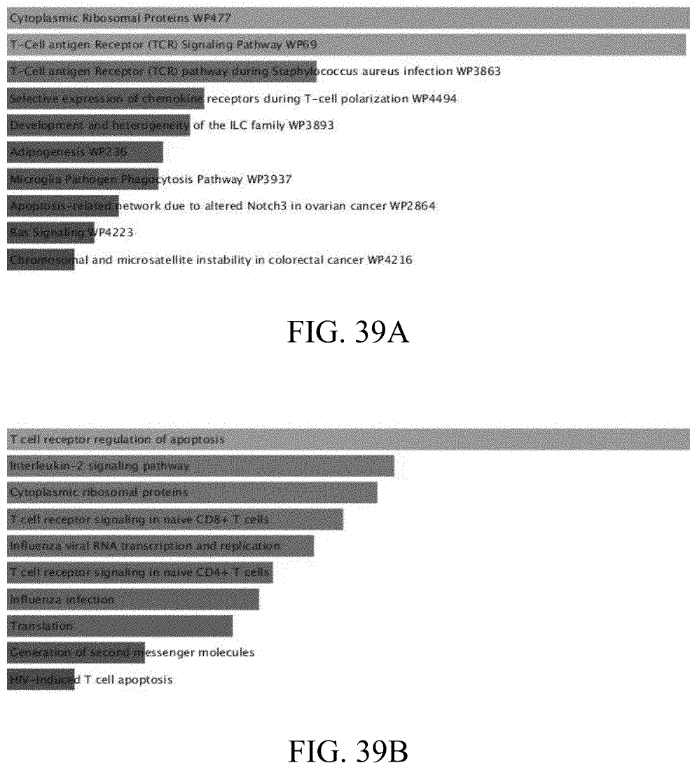

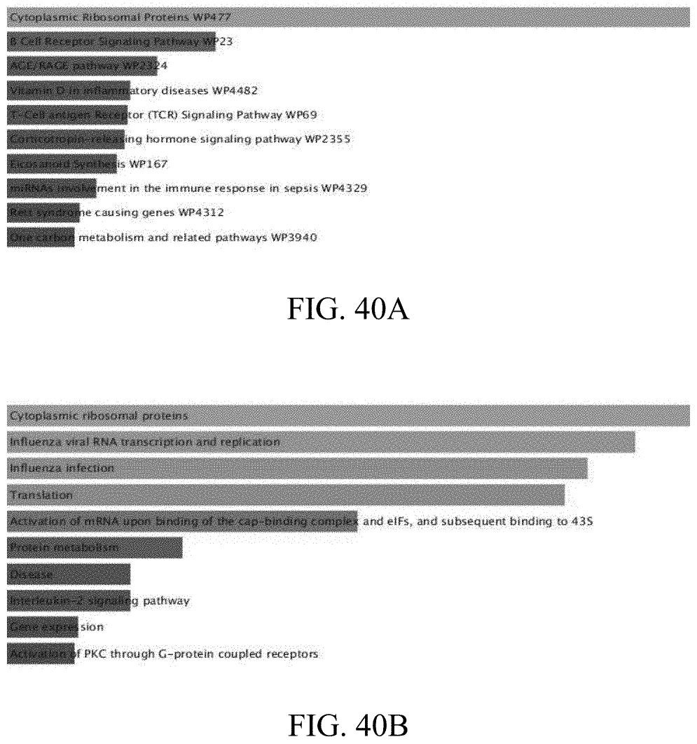

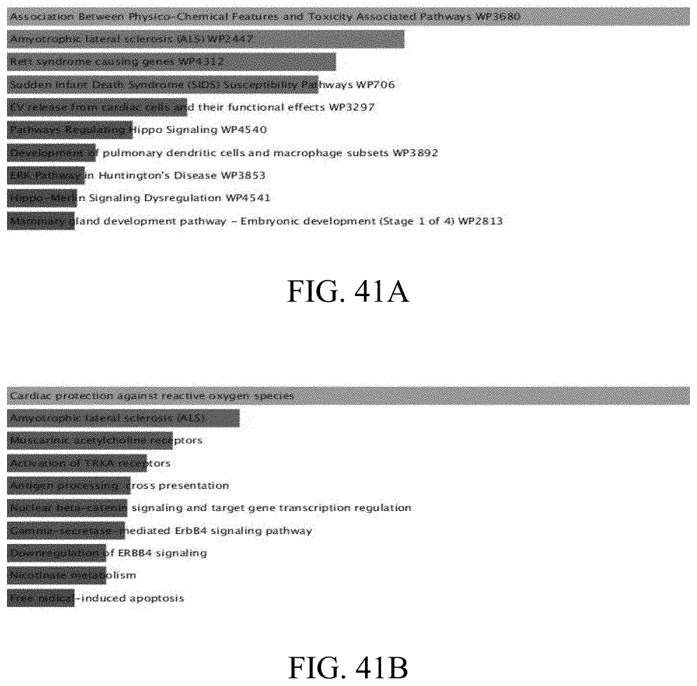

Applicants identified gene programs and pathways in healthy and diseased cells (Tables 8-12 and FIGS. 34-41). Detection of altered gene expression of the programs or altered signaling by the pathways may be used to predict risk for a phenotype. The genes and pathways may also be therapeutic targets to treat or modify disease (e.g., UC) or traits (e.g., depression).

TABLE 8
Gene Signals for Disease

| | | | |
|---|---|---|---|
| PASS_Ulcerative_Colitis | UC | Disease_Enterocytes | LAMB1, RNF186, APEH, DLD, C1orf106, PSMG1, |
| | | | JAK2, TCTA, GPX1, REL, RHOA, ARFRP1, |
| | | | SLC26A6, TNFRSF14, REXO2, TNFSF15, GSDMB, |
| | | | DAG1, STAT3, UBA7, CREM, TMBIM1, MST1R, |
| | | | FAM213B, SLC2A4RG, RBM5, MMEL1, NUCB2, |
| | | | RBM6, GPR35, MAML2, ERRFI1, LPP, ORMDL3, |
| | | | NXPE1, KIAA1109, MAPKAPK2, PHC2, TACC1, |
| | | | PEX13, ACTR1A, SERBP1, SEC16A, ITPKA, |
| | | | ZFP91, P4HA2, CDKN1A, RTF1, MED24, TMEM170A |
| PASS_IBD_deLange2017 | UC | Disease_ILCs | REL, CREM, RPL37, GPR65, CTNNB1, CDKN1A, |
| | | | NFKBIZ, RPS29, RPS21, RPLP2, DYNLL1, |
| | | | RPL23, RPS12, RNF168, PFKFB3, TNFAIP3, |
| | | | PRRC2C, RPS28, C15orf48, RPL28, TIPARP, |
| | | | RPL38, FUS, TOMM7, YWHAZ, ARGLU1, RPS11, |
| | | | RPL34, SFPQ, UBE2S, RPL37A, NFE2L2, NCL, |
| | | | ARL5B, RPLP1, FOSB, TPT1, JUND, PNRC1, |
| | | | RPS20, CHMP1B, DDX5, POLR2K, BIRC3, RPS24, |
| | | | RPS15A, RPL41, UQCRB, YME1L1, C14orf2 |
| PASS_Ulcerative_Colitis | UC | Disease_T_Lymphocytes | GPX1, REL, STAT3, CREM, RBM6, RTF1, |
| | | | BRD7, NFKB1, CHP1, ITLN1, ARAP2, GLCCI1, |
| | | | THADA, SLC30A7, HDAC7, GNB1, CYTH1, RPL23A, |
| | | | USP34, NFATC1, PRDM1, PIK3R1, HSPE1, CAPZA1, |
| | | | IL2RA, CD28, CD44, PRKCB, ADAM17, LEF1, |
| | | | NUCKS1, ANP32E, RBM39, HSPD1, LIMS1, |
| | | | ZC3H12D, ZNF644, TRIM28, CD7, EIF3D, TAB2, |
| | | | SF3B1, EIF3E, IL7R, SMARCE1, ABI1, ELMSAN1, |
| | | | TMEM63A, DDX6, VPS51 |
| PASS_Ulcerative_Colitis | UC | Disease_TA | OTUD3, LAMB1, RNF186, APEH, SNAPC4, |
| | | | DLD, C1orf106, PSMG1, JAK2, SDCCAG3, |
| | | | TCTA, GPX1, REL, RNF123, RHOA, ARFRP1, |
| | | | SLC26A6, TNFRSF14, REXO2, PMPCA, |
| | | | STMN3, TNFSF15, GNA12, GSDMB, DAG1, |
| | | | C21orf33, GRB7, STAT3, TNPO3, IP6K1, |
| | | | UBA7, CUL2, CREM, CAMSAP2, TMBIM1, |
| | | | MST1R, FAM213B, SLC2A4RG, ARPC2, |
| | | | RBM5, MON1A, AAMP, NUCB2, USP4, NOTCH1, |
| | | | PARK7, RBM6, C3orf62, ZFP90, GPR35 |
| PASS_Multiple_sclerosis | MS | Disease_Glutamatergic | FAM213B, RPL5, JUND, RAB3A, LMAN2, |
| | | | OS9, SAE1, KIF5A, MAPK1, SKP1, PRDX5, |
| | | | DEXI, C1orf52, CDC37, SUMF2, B4GALNT1, |
| | | | SF3B6, KPNB1, FKBP1B, MAPK3, SLC12A5, |
| | | | DDX6, NDFIP1, SOX15, CAMK2G, SF3B2, |
| | | | MPI, BANF1, CISD2, EIF3B, ZNHIT3, |
| | | | SYNPR, SRP9, PREX1, EIF2AK3, FXR2, |
| | | | ATP6V0A1, UBE4A, COX5A, CCT6A, ICAM5, |
| | | | PIP4K2C, EXOC7, CHCHD2, PSMA3, RAB18, |
| | | | PRELID1, PARP2, TRMT112, GDI2 |
| PASS_Multiple_sclerosis | MS | Disease_GABAergic | RPL5, PDE4A, JUND, RAB3A, OS9, |
| | | | SAE1, KIF5A, MAPK1, SKP1, PRDX5, |
| | | | C1orf52, CDC37, SF3B6, KPNB1, |
| | | | MAPK3, SLC12A5, DDX6, NDFIP1, |
| | | | CAMK2G, NPEPPS, EPS15L1, SF3B2, |
| | | | ZBTB38, BANF1, CISD2, ZNHIT3, HNRNPM, |
| | | | IFNGR1, SRP9, PREX1, EIF2AK3, ATP6V0A1, |
| | | | SGSM2, UBE4A, CCT6A, UBE2D3, EXOC7, |
| | | | CHCHD2, RAB18, CSGALNACT2, PRELID1, |
| | | | SCAF11, TRMT112, GDI2, TMEM160, |
| | | | C2orf47, SDHA, MARK3, PPHLN1, FKBP2 |
| PASS_Multiple_sclerosis | MS | Disease_Glia | RPL5, RAB3A, OS9, MANBA, SKP1, |
| | | | PRDX5, C3, NDFIP1, SF3B2, BANF1, |
| | | | IFNGR1, SRP9, PREXI, UBE2D3, RGCC, |
| | | | CHCHD2, RNF213, SCAF11, TRMT112, |
| | | | GDI2, DPYD, SYK, FKBP2, STMN3, RPL24, |
| | | | RPS9, RPS13, FCHSD2, MRPL51, HSPB1, |
| | | | RPS6, GNAI2, RNF19A, YPEL3, RAMP1, |
| | | | RNF111, NDRG4, ABCA1, CKB, DRAP1, |
| | | | LGI3, HINT1, IRS2, PTPRC, IFI16, |
| | | | NDUFA12, MEF2A, NUDC, ABCA2, MYL6 |
| UKB_460K.lung_FEV1FVCzSMOKE | asthma_disease | Fibroblast | ITGA1, MFAP2, PTCH1, BMP4, LOX, |
| | | | RBMS3, NTM, DLC1, NTN4, TGFBR3, |
| | | | HTRA1, ADAMTS2, CALD1, COL4A2, |
| | | | DNAJB4, NEXN, LTBP1, MRC2, LMCD1, |
| | | | PEAK1, RERG, MACF1, LRP1, FOXO3, |
| | | | DTWD1, COPS6, PLXDC2, FGF7, PDZRN3, |
| | | | RHOBTB3, NR1D1, DST, FNDC3B, LTBP2, |
| | | | LTBP4, NUCKS1, PAPPA, IL1R1, CAPZB, |
| | | | SEPT2, ANTXR1, NR3C1, STARD13, HMCN1, |
| | | | JMJD1C, P4HA2, ZFP36L2, PLAC9, |
| | | | ARF4, IFITM2 |
| UKB_460K.lung_FEV1FVCzSMOKE | asthma_disease | Basal | THSD4, CDC123, SNRPF, MFAP2, |
| | | | SDHB, NSRP1, BMP4, TNS1, RBMS3, |
| | | | VGLL4, TSHZ3, EML4, ABCE1, COX7A2L, |
| | | | EFEMP1, SMG6, FAM213A, MTUS1, AKR1A1, |
| | | | KLHL21, CALD1, SCAPER, BLMH, TGFB2, |
| | | | SH3PXD2A, DEF6, LRP1, ITGA2, COPS6, |
| | | | PABPC4, PHB, PLXDC2, FAF1, TP53I13, |
| | | | ITGAV, RHOBTB3, NR1D1, DST, ADRB2, |
| | | | LTBP4, NUCKS1, IL1R1, DSP, EIF3E, |
| | | | COPS2, PRSS23, NIPSNAP1, ANTXR1, |
| | | | NDUFA12, AJUBA |
| PASS_ChildOnsetAsthma_Ferreira2019 | asthma_disease | T_Lymphocyte | CAMK4, FMNL1, GPR183, RORA, IRF1, |
| | | | DEF6, THEMIS, CD52, BCL2, RFTN1, |
| | | | CFL1, CD247, NFKBIA, SLFN5, CCDC85B, |
| | | | IQGAP2, GRB2, PRKCB, DIAPH1, SH3BGRL3, |
| | | | FXYD5, TAGAP, SLAMF1, MYCBP2, CREM, |
| | | | AKAP13, ETS1, STK4, OSTF1, UBE2B, CELF2, |
| | | | RUNX3, SNRPF, AKNA, RCSD1, SCML4, BATF, |
| | | | CXCR6, CTSW, PRKCH, CALM3, SNRPD2, |
| | | | SPOCK2, CHMP4A, SEPT1, ENO1, NEDD8, |
| | | | LSM14A, TNFRSF1B, SSR2 |

TABLE 9
Disease Genes

| | MS Disease | MS Disease | MS Disease | MS Disease | Asthma T | Lung capacity | Lung capacity |
|---|---|---|---|---|---|---|---|
| UC | Myeloid | Stromal | Endothelial | Glutamatergic | cells | Basal | Fibroblast |
| GPX1 | PIAS1 | NSD1 | IFITM2 | NMT1 | CAMK4 | THSD4 | ITGA1 |
| REL | ITM2B | UBE2D3 | HSPB1 | CNIH2 | FMNL1 | CDC123 | MFAP2 |
| STAT3 | DSCAM | CBLB | WARS | TMEM151A | GPR183 | SNRPF | PTCH1 |
| CREM | NIPBL | PDSS2 | IQGAP1 | RAB1B | RORA | MFAP2 | BMP4 |
| RBM6 | CHSY3 | PEAK1 | PDIA6 | NFE2L1 | IRF1 | SDHB | LOX |
| RTF1 | PLP1 | MYL6 | RPL7A | RASGRP1 | DEF6 | NSRP1 | RBMS3 |
| BRD7 | | PTPN13 | ALDOA | PTGER3 | THEMIS | BMP4 | NTM |
| NFKB1 | | CAMSAP2 | NEDD4 | IPO9 | CD52 | TNS1 | DLC1 |
| CHP1 | | SSH2 | GAPDH | MNT | BCL2 | RBMS3 | NTN4 |
| ITLN1 | | TOP1 | HSPA5 | DNM1 | RFTN1 | VGLL4 | TGFBR3 |
| ARAP2 | | PACS1 | LPP | HEXIM1 | CFL1 | TSHZ3 | HTRA1 |
| GLCCI1 | | EGFR | RPL19 | CBX1 | CD247 | EML4 | ADAMTS2 |
| THADA | | YTHDC1 | FUT8 | GNB1 | NFKBIA | ABCE1 | CALD1 |
| SLC30A7 | | MYCBP2 | OOEP | CSE1L | SLFN5 | COX7A2L | COL4A2 |
| HDAC7 | | HSPA5 | RPL38 | NCAM1 | CCDC85B | EFEMP1 | DNAJB4 |
| GNB1 | | RPL19 | RPS15 | GNAO1 | IQGAP2 | SMG6 | NEXN |
| CYTH1 | | PTGDS | SLC26A3 | KCNAB2 | GRB2 | FAM213A | LTBP1 |
| RPL23A | | GNAS | RPL6 | PSMC3 | PRKCB | MTUS1 | MRC2 |
| USP34 | | SLC26A3 | AFF3 | P2RY14 | DIAPH1 | AKR1A1 | LMCD1 |
| NFATC1 | | SEC31A | TPT1 | GPRC5B | SH3BGRL3 | KLHL21 | PEAK1 |
| PRDM1 | | TRPC1 | ACTG1 | C10orf11 | FXYD5 | CALD1 | RERG |
| PIK3R1 | | AFF3 | ANXA5 | ADRM1 | TAGAP | SCAPER | MACF1 |
| HSPE1 | | HOOK3 | KCTD8 | ZCRB1 | SLAMF1 | BLMH | LRP1 |
| CAPZA1 | | EHBP1 | S100A6 | TUBA1A | MYCBP2 | TGFB2 | FOXO3 |
| IL2RA | | RAP1B | LDHA | CCDC148 | CREM | SH3PXD2A | DTWD1 |
| CD28 | | PRKCA | WASF2 | TCEB1 | AKAP13 | DEF6 | COPS6 |
| CD44 | | HIPK3 | S100A11 | TUBA1B | ETS1 | LRP1 | PLXDC2 |
| PRKCB | | ADCY3 | PRSS23 | LUZP2 | STK4 | ITGA2 | FGF7 |
| ADAM17 | | TBC1D5 | PABPC1 | C1orf95 | OSTF1 | COPS6 | PDZRN3 |
| LEF1 | | PLEKHA5 | RPL36 | SYT1 | UBE2B | PABPC4 | RHOBTB3 |
| NUCKS1 | | ASH1L | ACTB | TCAF1 | CELF2 | PHB | NR1D1 |
| ANP32E | | ARHGAP21 | PTMA | MAP2K1 | RUNX3 | PLXDC2 | DST |
| RBM39 | | CLASP1 | RPS14 | CALB2 | SNRPF | FAF1 | FNDC3B |
| HSPD1 | | CDH11 | RPLP1 | CBR1 | AKNA | TP53I13 | LTBP2 |
| LIMS1 | | CFLAR | RPL28 | KIF5A | RCSD1 | ITGAV | LTBP4 |
| ZC3H12D | | CREB3L2 | RPSA | C16orf72 | SCML4 | RHOBTB3 | NUCKS1 |
| ZNF644 | | LTBP1 | SPARCL1 | PPP4R2 | BATF | NR1D1 | PAPPA |
| TRIM28 | | MSI2 | BCL2L1 | GSTP1 | CXCR6 | DST | IL1R1 |
| CD7 | | FBXO11 | CST3 | CGGBP1 | CTSW | ADRB2 | CAPZB |
| EIF3D | | GAPVD1 | KALRN | SNAPC5 | PRKCH | LTBP4 | SEPT2 |
| TAB2 | | ITM2B | RPS29 | KCTD10 | CALM3 | NUCKS1 | ANTXR1 |
| SF3B1 | | SYT1 | TMSB10 | NR2F2 | SNRPD2 | IL1R1 | NR3C1 |
| EIF3E | | FOXO3 | RPS20 | TMEM70 | SPOCK2 | DSP | STARD13 |
| IL7R | | FNDC3B | TPST2 | UBXN1 | CHMP4A | EIF3E | HMCN1 |
| SMARCE1 | | NOVA1 | RPL31 | SAR1B | SEPT1 | COPS2 | JMJD1C |
| ABU | | PDE1A | RPL15 | CNTN5 | ENO1 | PRSS23 | P4HA2 |
| ELMSAN1 | | TMEM132C | RPL35 | NCOR2 | NEDD8 | NIPSNAP1 | ZFP36L2 |
| TMEM63A | | LRP4 | ZBTB16 | L3MBTL2 | LSM14A | ANTXR1 | PLAC9 |
| DDX6 | | PLCB4 | APOD | TIMM17A | TNFRSF1B | NDUFA12 | ARF4 |
| VPS51 | | NR2F1 | HSPA8 | DNAJC18 | SSR2 | AJUBA | IFITM2 |

TABLE 10
Disease MS glutamatergic

| | | | Adjusted | Odds | Combined | |
|---|---|---|---|---|---|---|
| Term | Overlap | P-value | P-value | Ratio | Score | Genes |
| Serotonin HTR1 group and FOS | 11749 | 6.93E−05 | 1.05E−01 | 37.50 | 359.15 | GNAO1; MAP2K1; RASGRP1 |
| pathway | | | | | | |
| Signaling events mediated by HDAC | 13940 | 1.17E−04 | 8.80E−02 | 31.58 | 286.00 | NCOR2; TUBA1B; GNB1 |
| class II | | | | | | |
| CXCR4 signaling pathway | 4/116 | 2.01E−04 | 1.01E−01 | 13.79 | 117.38 | GNAO1; MAP2K1; GNB1; DNM1 |
| MAP kinase inactivation of SMRT | 43875 | 5.47E−04 | 2.06E−01 | 57.14 | 429.23 | NCOR2; MAP2K1 |
| corepressor | | | | | | |
| Thyroid-stimulating hormone | 24167 | 6.02E−04 | 1.82E−01 | 18.18 | 134.82 | GNAO1; MAP2K1; GNB1 |
| signaling pathway | | | | | | |
| Serotonin receptor 2 and ELK- | 43877 | 7.19E−04 | 1.81E−01 | 50.00 | 361.90 | MAP2K1; RASGRP1 |
| SRF/GATA4 signaling | | | | | | |
| Post-chaperonin tubulin folding | 43878 | 8.13E−04 | 1.75E−01 | 47.06 | 334.80 | TUBA1B; TUBA1A |
| pathway | | | | | | |
| Beta-arrestin-dependent | 43878 | 8.13E−04 | 1.54E−01 | 47.06 | 334.80 | MAP2K1; DNM1 |
| recruitment of Src kinases in GPCR | | | | | | |
| signaling | | | | | | |
| Estrogen receptor signaling pathway | 43881 | 1.13E−03 | 1.90E−01 | 40.00 | 271.39 | MAP2K1; GNB1 |
| Gap junction pathway | 32933 | 1.48E−03 | 2.24E−01 | 13.33 | 86.86 | TUBA1B; MAP2K1; TUBA1A |
| L1CAM interactions | 34394 | 1.68E−03 | 2.31E−01 | 12.77 | 81.57 | MAP2K1; NCAM1; DNM1 |
| Cooperation of prefoldin and | 43888 | 2.07E−03 | 2.60E−01 | 29.63 | 183.18 | TUBA1B; TUBA1A |
| TriC/CCT in actin and tubulin folding | | | | | | |
| MHC class II antigen presentation | 3/103 | 2.18E−03 | 2.53E−01 | 11.65 | 71.39 | SAR1B; KIF5A; DNM1 |
| G-protein activation | 43889 | 2.22E−03 | 2.40E−01 | 28.57 | 174.56 | GNAO1; GNB1 |
| Inhibition of insulin secretion by | 43890 | 2.38E−03 | 2.40E−01 | 27.59 | 166.62 | GNAO1; GNB1 |
| adrenaline/noradrenaline | | | | | | |
| Immune system | 8/998 | 3.11E−03 | 2.93E−01 | 3.21 | 18.52 | MAP2K1; PSMC3; SAR1B; KIF5A; NCAM1; |
| | | | | | | TCEB1; RASGRP1; DNM1 |
| EGF receptor transactivation by | 12451 | 3.27E−03 | 2.90E−01 | 23.53 | 134.69 | MAP2K1; GNB1 |
| GPCRs in cardiac hypertrophy | | | | | | |
| Prion diseases | 12816 | 3.46E−03 | 2.90E−01 | 22.86 | 129.54 | MAP2K1; NCAM1 |
| Signal transduction by L1 | 12816 | 3.46E−03 | 2.75E−01 | 22.86 | 129.54 | MAP2K1; NCAM1 |
| Phospholipids as signaling | 13181 | 3.65E−03 | 2.76E−01 | 22.22 | 124.70 | MAP2K1; GNB1 |
| intermediaries | | | | | | |
| Adaptive immune system | 6/606 | 3.87E−03 | 2.78E−01 | 3.96 | 22.00 | PSMC3; SAR1B; KIF5A; TCEB1; RASGRP1; |
| | | | | | | DNM1 |
| Developmental biology | 5/420 | 3.89E−03 | 2.67E−01 | 4.76 | 26.43 | NCOR2; MAP2K1; NCAM1; NR2F2; DNM1 |
| FSH regulation of apoptosis | 4/263 | 4.19E−03 | 2.75E−01 | 6.08 | 33.31 | MAP2K1; GPRC5B; TUBA1A; GNB1 |
| Plasma membrane estrogen | 15008 | 4.72E−03 | 2.97E−01 | 19.51 | 104.51 | GNAO1; GNB1 |
| receptor signaling | | | | | | |
| Bioactive peptide-induced signaling | 15008 | 4.72E−03 | 2.85E−01 | 19.51 | 104.51 | MAP2K1; GNB1 |
| pathway | | | | | | |
| Cell differentiation by G alpha (i/o) | 15738 | 5.18E−03 | 3.01E−01 | 18.60 | 97.91 | MAP2K1; RASGRP1 |
| pathway inferred from mouse | | | | | | |
| Neuro2A model | | | | | | |
| Protein folding | 19391 | 7.78E−03 | 4.35E−01 | 15.09 | 73.30 | TUBA1B; TUBA1A |
| Thromboxane A2 receptor signaling | 20486 | 8.65E−03 | 4.67E−01 | 14.29 | 67.85 | GNB1; DNM1 |
| Pathogenic Escherichia coli infection | 20852 | 8.96E−03 | 4.66E−01 | 14.04 | 66.18 | TUBA1B; TUBA1A |
| Neurotrophic factor-mediated Trk | 21947 | 9.88E−03 | 4.98E−01 | 13.33 | 61.56 | MAP2K1; DNM1 |
| receptor signaling | | | | | | |
| Ephrin receptor B forward pathway | 21947 | 9.88E−03 | 4.81E−01 | 13.33 | 61.56 | MAP2K1; DNM1 |
| Endothelins | 23408 | 1.12E−02 | 5.28E−01 | 12.50 | 56.16 | GNAO1; MAP2K1 |
| LPA receptor mediated events | 23774 | 1.15E−02 | 5.27E−01 | 12.31 | 54.93 | GNAO1; GNB1 |
| Destabilization of mRNA by AUF1 | 24139 | 1.19E−02 | 5.27E−01 | 12.12 | 53.75 | PSMC3; TCEB1 |
| (hnRNP D0) | | | | | | |
| Activation of RAS in B cells | 43835 | 1.24E−02 | 5.37E−01 | 80.00 | 350.95 | RASGRP1 |
| ERK activation | 43835 | 1.24E−02 | 5.22E−01 | 80.00 | 350.95 | MAP2K1 |
| Nifedipine activity | 43835 | 1.24E−02 | 5.08E−01 | 80.00 | 350.95 | MAP2K1 |
| Renal cell carcinoma | 25600 | 1.33E−02 | 5.27E−01 | 11.43 | 49.39 | MAP2K1; TCEB1 |
| Long-term depression | 25600 | 1.33E−02 | 5.14E−01 | 11.43 | 49.39 | GNAO1; MAP2K1 |
| NCAM signaling for neurite out- | 25600 | 1.33E−02 | 5.01E−01 | 11.43 | 49.39 | MAP2K1; NCAM1 |
| growth | | | | | | |
| G alpha (i) signaling events | 3/199 | 1.35E−02 | 4.97E−01 | 6.03 | 25.96 | P2RY14; PTGER3; GNB1 |
| HIV infection | 3/200 | 1.37E−02 | 4.92E−01 | 6.00 | 25.75 | PSMC3; NMT1; TCEB1 |
| Gastrin-CREB signaling pathway via | 3/206 | 1.48E−02 | 5.20E−01 | 5.83 | 24.54 | MAP2K1; GNB1; RASGRP1 |
| PKC and MAPK | | | | | | |
| ADP signalling through P2Y | 43836 | 1.49E−02 | 5.12E−01 | 66.67 | 280.39 | GNB1 |
| purinoceptor 1 | | | | | | |
| G beta-gamma signaling through | 43836 | 1.49E−02 | 5.00E−01 | 66.67 | 280.39 | GNB1 |
| P13K gamma | | | | | | |
| Multi-drug resistance factors | 43836 | 1.49E−02 | 4.89E−01 | 66.67 | 280.39 | GSTP1 |
| PIK3C1/B pathway | 43836 | 1.49E−02 | 4.79E−01 | 66.67 | 280.39 | MAP2K1 |
| Transcriptional regulation of white | 28157 | 1.59E−02 | 5.00E−01 | 10.39 | 43.02 | NCOR2; NR2F2 |
| adipocyte differentiation | | | | | | |
| Opioid signaling | 29252 | 1.71E−02 | 5.27E−01 | 10.00 | 40.69 | GNAO1; GNB1 |
| Arachidonate epoxygenase/epoxide | 43837 | 1.74E−02 | 5.25E−01 | 57.14 | 231.59 | GSTP1 |
| hydrolase pathway | | | | | | |
| MEK activation | 43837 | 1.74E−02 | 5.14E−01 | 57.14 | 231.59 | MAP2K1 |
| T cell signal transduction | 30348 | 1.83E−02 | 5.32E−01 | 9.64 | 38.55 | MAP2K1; RASGRP1 |
| Interleukin-2 signaling pathway | 6/847 | 1.84E−02 | 5.25E−01 | 2.83 | 11.31 | MAP2K1; GPRC5B; MNT; PTGER3; TCEB1; |
| | | | | | | RASGRP1 |
| Chromatin remodeling by nuclear | 43838 | 1.98E−02 | 5.54E−01 | 50.00 | 196.03 | NCOR2 |
| receptors to facilitate initiation of | | | | | | |
| transcription in carcinoma cells | | | | | | |
| HIF-1 degradation in normoxia | 32174 | 2.05E−02 | 5.61E−01 | 9.09 | 35.36 | PSMC3; TCEB1 |
| Prostate cancer | 32540 | 2.09E−02 | 5.63E−01 | 8.99 | 34.77 | MAP2K1; GSTP1 |
| Prostanoid ligand receptors | 43839 | 2.23E−02 | 5.90E−01 | 44.44 | 169.07 | PTGER3 |
| Rapid glucocorticoid receptor | 43839 | 2.23E−02 | 5.80E−01 | 44.44 | 169.07 | GNB1 |
| pathway | | | | | | |
| COPII-mediated vesicle transport | 43839 | 2.23E−02 | 5.70E−01 | 44.44 | 169.07 | SAR1B |
| Fc gamma receptor-mediated | 34366 | 2.31E−02 | 5.82E−01 | 8.51 | 32.06 | MAP2K1; DNM1 |
| phagocytosis | | | | | | |
| G-protein signaling pathways | 34731 | 2.36E−02 | 5.84E−01 | 8.42 | 31.55 | GNAO1; GNB1 |
| Protein metabolism | 4/442 | 2.44E−02 | 5.94E−01 | 3.62 | 13.45 | TUBA1B; TUBA1A; SAR1B; TIMM17A |
| Interferon-gamma signaling | 35462 | 2.45E−02 | 5.88E−01 | 8.25 | 30.58 | MAP2K1; NCAM1 |
| pathway | | | | | | |
| Gap junction degradation | 43840 | 2.47E−02 | 5.83E−01 | 40.00 | 148.00 | DNM1 |
| Splicing regulation through Sam68 | 43840 | 2.47E−02 | 5.74E−01 | 40.00 | 148.00 | MAP2K1 |
| Downstream signaling events Of B | 35827 | 2.50E−02 | 5.72E−01 | 8.16 | 30.11 | PSMC3; RASGRP1 |
| cell receptor (BCR) | | | | | | |
| Potassium channels | 36192 | 2.55E−02 | 5.74E−01 | 8.08 | 29.66 | GNB1; KCNAB2 |
| Antigen presentation: folding, | | | | | | |
| assembly, and peptide loading of | 3/255 | 2.59E−02 | 5.74E−01 | 4.71 | 17.20 | PSMC3; SAR1B; TCEB1 |
| class I MHC proteins | | | | | | |
| Disease | 5/674 | 2.61E−02 | 5.70E−01 | 2.97 | 10.82 | MAP2K1; SYT1; PSMC3; NMT1; TCEB1 |
| Melanogenesis | 2/101 | 2.64E−02 | 5.70E−01 | 7.92 | 28.78 | GNAO1; MAP2K1 |
| Acetylcholine neurotransmitter | 43841 | 2.72E−02 | 5.78E−01 | 36.36 | 131.12 | SYT1 |
| release cycle | | | | | | |
| Norepinephrine neurotransmitter | 43841 | 2.72E−02 | 5.70E−01 | 36.36 | 131.12 | SYT1 |
| release cycle | | | | | | |
| Osteopontin signaling | 43841 | 2.72E−02 | 5.62E−01 | 36.36 | 131.12 | MAP2K1 |
| Gamma-aminobutyric acid receptor | 43842 | 2.96E−02 | 6.04E−01 | 33.33 | 117.33 | DNM1 |
| life cycle | | | | | | |
| Assembly of HIV virion | 43842 | 2.96E−02 | 5.96E−01 | 33.33 | 117.33 | NMT1 |
| Beta-arrestins in GPCR | 43842 | 2.96E−02 | 5.88E−01 | 33.33 | 117.33 | DNM1 |
| desensitization | | | | | | |
| Dopamine neurotransmitter release | 43842 | 2.96E−02 | 5.80E−01 | 33.33 | 117.33 | SYT1 |
| cycle | | | | | | |
| MAP kinase downregulation by | 43843 | 3.20E−02 | 6.20E−01 | 30.77 | 105.88 | MAP2K1 |
| phosphorylation of MEK1 by | | | | | | |
| Cdk5/p35 | | | | | | |
| Melanocyte development and | 43843 | 3.20E−02 | 6.12E−01 | 30.77 | 105.88 | MAP2K1 |
| pigmentation pathway | | | | | | |
| Neuronal system | 3/283 | 3.37E−02 | 6.37E−01 | 4.24 | 14.37 | SYT1; GNB1; KCNAB2 |
| Signaling by GPCR | 6/977 | 3.41E−02 | 6.36E−01 | 2.46 | 8.30 | GNAO1; MAP2K1; P2RY14; PTGER3; GNB1; |
| | | | | | | RASGRP1 |
| Retrograde neurotrophin signaling | 43844 | 3.44E−02 | 6.34E−01 | 28.57 | 96.24 | DNM1 |
| S1P/S1P4 pathway | 43844 | 3.44E−02 | 6.27E−01 | 28.57 | 96.24 | GNAO1 |
| T cell receptor/Ras pathway | 43844 | 3.44E−02 | 6.19E−01 | 28.57 | 96.24 | MAP2K1 |
| Visual signal transduction | 43844 | 3.44E−02 | 6.12E−01 | 28.57 | 96.24 | GNB1 |
| HIV life cycle | 2/118 | 3.52E−02 | 6.18E−01 | 6.78 | 22.69 | NMT1; TCEB1 |
| Notch signaling pathway | 2/121 | 3.68E−02 | 6.39E−01 | 6.61 | 21.83 | NCOR2; DNM1 |
| Calcium signaling by HBx of hepatitis | 43845 | 3.69E−02 | 6.33E−01 | 26.67 | 88.01 | MAP2K1 |
| B virus | | | | | | |
| Signaling to p38 via RIT and RIN | 43845 | 3.69E−02 | 6.25E−01 | 26.67 | 88.01 | MAP2K1 |
| Eicosanoid ligand-binding G-protein | 43845 | 3.69E−02 | 6.18E−01 | 26.67 | 88.01 | PTGER3 |
| coupled receptors | | | | | | |
| Integration of energy metabolism | 2/125 | 3.91E−02 | 6.48E−01 | 6.40 | 20.75 | GNAO1; GNB1 |
| Glutamate neurotransmitter release | 43846 | 3.93E−02 | 6.45E−01 | 25.00 | 80.93 | SYT1 |
| cycle | | | | | | |
| Rap1 signaling | 43846 | 3.93E−02 | 6.38E−01 | 25.00 | 80.93 | RASGRP1 |
| Inhibition of platelet activation by | 43846 | 3.93E−02 | 6.31E−01 | 25.00 | 80.93 | MAP2K1 |
| aspirin | | | | | | |
| Interleukin-9 signaling pathway | 43846 | 3.93E−02 | 6.24E−01 | 25.00 | 80.93 | MAP2K1 |
| Nucleotide-like (purinergic) G- | 43846 | 3.93E−02 | 6.18E−01 | 25.00 | 80.93 | P2RY14 |
| protein coupled receptors | | | | | | |
| HIV factor interactions with host | 2/128 | 4.08E−02 | 6.35E−01 | 6.25 | 20.00 | PSMC3; TCEB1 |
| SHC-related events | 43847 | 4.17E−02 | 6.42E−01 | 23.53 | 74.77 | MAP2K1 |
| SHC1 events in EGFR signaling | 43847 | 4.17E−02 | 6.36E−01 | 23.53 | 74.77 | MAP2K1 |
| Cadmium-induced DNA biosynthesis | 43847 | 4.17E−02 | 6.29E−01 | 23.53 | 74.77 | MAP2K1 |
| and proliferation in macrophages | | | | | | |
| Chylomicron-mediated lipid | 43847 | 4.17E−02 | 6.23E−01 | 23.53 | 74.77 | SAR1B |
| transport | | | | | | |
| Synaptic proteins at the synaptic | 43847 | 4.17E−02 | 6.17E−01 | 23.53 | 74.77 | NCAM1 |
| junction | | | | | | |
| Endocytotic role of NDK, phosphins | 43847 | 4.17E−02 | 6.11E−01 | 23.53 | 74.77 | DNM1 |
| and dynamin | | | | | | |
| Membrane trafficking | 2/133 | 4.37E−02 | 6.34E−01 | 6.02 | 18.83 | SAR1B; DNM1 |
| Human cytomegalovirus and MAP | 43848 | 4.41E−02 | 6.34E−01 | 22.22 | 69.37 | MAP2K1 |
| kinase pathways | | | | | | |
| Serotonin receptor 4/6/7 and NR3C | 43848 | 4.41E−02 | 6.28E−01 | 22.22 | 69.37 | MAP2K1 |
| signaling | | | | | | |
| Botulinum neurotoxicity | 43848 | 4.41E−02 | 6.22E−01 | 22.22 | 69.37 | SYT1 |
| Downregulation of MTA-3 in ER- | 43848 | 4.41E−02 | 6.16E−01 | 22.22 | 69.37 | TUBA1A |
| negative breast tumors | | | | | | |
| Effect of METS on macrophage | 43848 | 4.41E−02 | 6.11E−01 | 22.22 | 69.37 | NCOR2 |
| differentiation | | | | | | |
| NGF signaling via TRKA from the | 2/136 | 4.55E−02 | 6.24E−01 | 5.88 | 18.18 | MAP2K1; DNM1 |
| plasma membrane | | | | | | |
| GABA biosynthesis, release, | 43849 | 4.65E−02 | 6.32E−01 | 21.05 | 64.61 | SYT1 |
| reuptake and degradation | | | | | | |
| Hypoxic and oxygen homeostasis | 43849 | 4.65E−02 | 6.26E−01 | 21.05 | 64.61 | TCEB1 |
| regulation of HIF-1-alpha | | | | | | |
| Small ligand GPCRs | 43849 | 4.65E−02 | 6.21E−01 | 21.05 | 64.61 | PTGER3 |
| Class C GPCRs (metabotropic | 43849 | 4.65E−02 | 6.15E−01 | 21.05 | 64.61 | GPRC5B |
| glutamate and pheromone | | | | | | |
| receptors) | | | | | | |
| MAL role in Rho-mediated activation | 43849 | 4.65E−02 | 6.10E−01 | 21.05 | 64.61 | MAP2K1 |
| of SRF | | | | | | |
| FRS2-mediated activation | 43849 | 4.65E−02 | 6.05E−01 | 21.05 | 64.61 | MAP2K1 |
| T cell receptor signaling pathway | 2/139 | 4.73E−02 | 6.10E−01 | 5.76 | 17.56 | MAP2K1; RASGRP1 |
| Pathways in cancer | 3/325 | 4.76E−02 | 6.09E−01 | 3.69 | 11.24 | MAP2K1; GSTP1; TCEB1 |
| Axon guidance | 3/325 | 4.76E−02 | 6.04E−01 | 3.69 | 11.24 | MAP2K1; NCAM1; DNM1 |
| EGF/EGFR signaling pathway | 2/141 | 4.85E−02 | 6.10E−01 | 5.67 | 17.17 | MAP2K1; DNM1 |
| Sprouty regulation of tyrosine | 43850 | 4.89E−02 | 6.10E−01 | 20.00 | 60.38 | MAP2K1 |
| kinase signals | | | | | | |
| Nerve growth factor (NGF) pathway | 43850 | 4.89E−02 | 6.05E−01 | 20.00 | 60.38 | MAP2K1 |
| Presynaptic function of kainate | 43851 | 5.12E−02 | 6.29E−01 | 19.05 | 56.60 | GNB1 |
| receptors | | | | | | |
| IGF1 signaling pathway | 43851 | 5.12E−02 | 6.24E−01 | 19.05 | 56.60 | MAP2K1 |
| S1P/S1P1 pathway | 43851 | 5.12E−02 | 6.19E−01 | 19.05 | 56.60 | GNAO1 |
| Nicotine activity on dopaminergic | 43851 | 5.12E−02 | 6.14E−01 | 19.05 | 56.60 | GNB1 |
| neurons | | | | | | |
| PKC-catalyzed phosphorylation of | 43851 | 5.12E−02 | 6.09E−01 | 19.05 | 56.60 | GNB1 |
| inhibitory phosphoprotein of myosin | | | | | | |
| phosphatase | | | | | | |
| Calcium regulation in the cardiac cell | 2/149 | 5.35E−02 | 6.31E−01 | 5.37 | 15.72 | GNAO1; GNB1 |
| CCR3 signaling in eosinophils | 43852 | 5.36E−02 | 6.27E−01 | 18.18 | 53.20 | MAP2K1 |
| Signaling by the B cell receptor | 2/151 | 5.48E−02 | 6.37E−01 | 5.30 | 15.39 | PSMC3; RASGRP1 |
| (BCR) | | | | | | |
| BAD phosphorylation mediated by | 43853 | 5.60E−02 | 6.45E−01 | 17.39 | 50.14 | MAP2K1 |
| IGF1R signaling | | | | | | |
| Signaling events mediated by PRL | 43853 | 5.60E−02 | 6.40E−01 | 17.39 | 50.14 | TUBA1B |
| Collagen binding in corneal epithelia | 43853 | 5.60E−02 | 6.36E−01 | 17.39 | 50.14 | MAP2K1 |
| mediated by Erk and PI-3 Kinase | | | | | | |
| Downregulation of SMAD2/3- | 43853 | 5.60E−02 | 6.31E−01 | 17.39 | 50.14 | NCOR2 |
| SMAD4 transcriptional activity | | | | | | |
| Eicosanoid metabolism | 43853 | 5.60E−02 | 6.26E−01 | 17.39 | 50.14 | PTGER3 |
| Visual signal transduction: rods | 43853 | 5.60E−02 | 6.21E−01 | 17.39 | 50.14 | GNB1 |
| Phagosome | 2/154 | 5.67E−02 | 6.25E−01 | 5.19 | 14.90 | TUBA1B; TUBA1A |
| Ras-independent pathway in NK | 43854 | 5.83E−02 | 6.38E−01 | 16.67 | 47.36 | MAP2K1 |
| cell-mediated cytotoxicity | | | | | | |
| Inhibition of cellular proliferation by | 43854 | 5.83E−02 | 6.34E−01 | 16.67 | 47.36 | MAP2K1 |
| Gleevec | | | | | | |
| SREBP signaling | 43854 | 5.83E−02 | 6.29E−01 | 16.67 | 47.36 | SAR1B |
| Dorso-ventral axis formation | 43854 | 5.83E−02 | 6.25E−01 | 16.67 | 47.36 | MAP2K1 |
| Toll receptor cascades | 2/159 | 6.00E−02 | 6.38E−01 | 5.03 | 14.15 | MAP2K1; DNM1 |
| Glutathione conjugation | 43855 | 6.07E−02 | 6.41E−01 | 16.00 | 44.83 | GSTP1 |
| SHC1 events in ERBB2 signaling | 43855 | 6.07E−02 | 6.36E−01 | 16.00 | 44.83 | MAP2K1 |
| Cellular response to hypoxia | 43855 | 6.07E−02 | 6.32E−01 | 16.00 | 44.83 | TCEB1 |
| Ck1/Cdk5 regulation by type 1 | 43855 | 6.07E−02 | 6.28E−01 | 16.00 | 44.83 | GNB1 |
| glutamate receptors | | | | | | |
| TPO signaling pathway | 43855 | 6.07E−02 | 6.23E−01 | 16.00 | 44.83 | MAP2K1 |
| PIP2 hydrolysis | 43855 | 6.07E−02 | 6.19E−01 | 16.00 | 44.83 | RASGRP1 |
| RXR/VDR pathway | 43856 | 6.30E−02 | 6.39E−01 | 15.38 | 42.52 | NCOR2 |
| Ras signaling pathway | 43856 | 6.30E−02 | 6.35E−01 | 15.38 | 42.52 | MAP2K1 |
| CARM1 and regulation of the | 43856 | 6.30E−02 | 6.30E−01 | 15.38 | 42.52 | NCOR2 |
| estrogen receptor | | | | | | |
| ERBB2 role in signal transduction | 43856 | 6.30E−02 | 6.26E−01 | 15.38 | 42.52 | MAP2K1 |
| and oncology | | | | | | |
| Estrogen receptor transcription | 43856 | 6.30E−02 | 6.22E−01 | 15.38 | 42.52 | NCOR2 |
| factor targets | | | | | | |
| ADP signalling through P2Y | 43857 | 6.54E−02 | 6.41E−01 | 14.81 | 40.41 | GNB1 |
| purinoceptor 12 | | | | | | |
| Kinesins | 43857 | 6.54E−02 | 6.37E−01 | 14.81 | 40.41 | KIF5A |
| Mammalian calpain regulation of | 43857 | 6.54E−02 | 6.33E−01 | 14.81 | 40.41 | MAP2K1 |
| cell motility | | | | | | |
| ERK5 role in neuronal survival | 43857 | 6.54E−02 | 6.29E−01 | 14.81 | 40.41 | MAP2K1 |
| pathway | | | | | | |
| G-protein beta-gamma signalling | 43858 | 6.77E−02 | 6.47E−01 | 14.29 | 38.46 | GNB1 |
| RNA polymerase III transcription | 43858 | 6.77E−02 | 6.43E−01 | 14.29 | 38.46 | SNAPC5 |
| initiation From type 3 promoter | | | | | | |
| Recycling pathway of cell adhesion | 43858 | 6.77E−02 | 6.39E−01 | 14.29 | 38.46 | DNM1 |
| molecule L1 | | | | | | |
| Phototransduction | 43859 | 7.01E−02 | 6.57E−01 | 13.79 | 36.67 | GNB1 |
| Influence of Ras and Rho proteins | 43859 | 7.01E−02 | 6.53E−01 | 13.79 | 36.67 | MAP2K1 |
| on G1 to S transition | | | | | | |
| S1P/51P3 pathway | 43859 | 7.01E−02 | 6.49E−01 | 13.79 | 36.67 | GNAO1 |
| Lipoprotein metabolism | 43859 | 7.01E−02 | 6.45E−01 | 13.79 | 36.67 | SAR1B |
| Thyroid cancer | 43859 | 7.01E−02 | 6.41E−01 | 13.79 | 36.67 | MAP2K1 |
| Meta pathway biotransformation | 2/174 | 7.03E−02 | 6.39E−01 | 4.60 | 12.21 | GSTP1; KCNAB2 |
| Gap junction trafficking and | 43860 | 7.24E−02 | 6.55E−01 | 13.33 | 35.01 | DNM1 |
| regulation | | | | | | |
| Activation of kainate receptors upon | 43860 | 7.24E−02 | 6.51E−01 | 13.33 | 35.01 | GNB1 |
| glutamate binding | | | | | | |
| Apoptosis intrinsic pathway | 43860 | 7.24E−02 | 6.47E−01 | 13.33 | 35.01 | NMT1 |
| Retinoic acid receptor-mediated | 43860 | 7.24E−02 | 6.43E−01 | 13.33 | 35.01 | NCOR2 |
| signaling | | | | | | |
| Signaling pathway from G-protein | 43860 | 7.24E−02 | 6.39E−01 | 13.33 | 35.01 | MAP2K1 |
| families | | | | | | |
| PDGFA signaling pathway | 43860 | 7.24E−02 | 6.36E−01 | 13.33 | 35.01 | MAP2K1 |
| Prostaglandin biosynthesis and | 43861 | 7.47E−02 | 6.52E−01 | 12.90 | 33.47 | PTGER3 |
| regulation | | | | | | |
| Ras family activation regulation | 43861 | 7.47E−02 | 6.48E−01 | 12.90 | 33.47 | RASGRP1 |
| Signal amplification | 43861 | 7.47E−02 | 6.45E−01 | 12.90 | 33.47 | GNB1 |
| Inwardly rectifying potassium | 43861 | 7.47E−02 | 6.41E−01 | 12.90 | 33.47 | GNB1 |
| channels | | | | | | |
| Stathmin and breast cancer | 43861 | 7.47E−02 | 6.37E−01 | 12.90 | 33.47 | TUBA1A |
| resistance to antimicrotubule agents | | | | | | |
| Transcription | 2/181 | 7.52E−02 | 6.38E−01 | 4.42 | 11.43 | SNAPC5; TCEB1 |
| Hypothetical network for drug | 11689 | 7.70E−02 | 6.50E−01 | 12.50 | 32.04 | MAP2K1 |
| addiction | | | | | | |
| Netrin-mediated signaling events | 11689 | 7.70E−02 | 6.46E−01 | 12.50 | 32.04 | MAP2K1 |
| Glucagon signaling in metabolic | 12055 | 7.93E−02 | 6.62E−01 | 12.12 | 30.71 | GNB1 |
| regulation | | | | | | |
| Glucagon-type ligand receptors | 12055 | 7.93E−02 | 6.58E−01 | 12.12 | 30.71 | GNB1 |
| Chemokine signaling pathway | 2/189 | 8.10E−02 | 6.69E−01 | 4.23 | 10.64 | MAP2K1; GNB1 |
| HIF-2-alpha transcription factor | 12420 | 8.16E−02 | 6.70E−01 | 11.76 | 29.47 | TCEB1 |
| network | | | | | | |
| MAPK/TRK pathway | 12420 | 8.16E−02 | 6.66E−01 | 11.76 | 29.47 | MAP2K1 |
| EPO receptor signaling | 12420 | 8.16E−02 | 6.63E−01 | 11.76 | 29.47 | MAP2K1 |
| Transmission across chemical | 2/190 | 8.18E−02 | 6.60E−01 | 4.21 | 10.54 | SYT1; GNB1 |
| synapses | | | | | | |
| GPCR ligand binding | 3/410 | 8.28E−02 | 6.65E−01 | 2.93 | 7.29 | P2RY14; PTGER3; GNB1 |
| Interleukin-7 signaling pathway | 12785 | 8.39E−02 | 6.71E−01 | 11.43 | 28.31 | MAP2K1 |
| fMLP induced chemokine gene | 12785 | 8.39E−02 | 6.67E−01 | 11.43 | 28.31 | MAP2K1 |
| expression in HMC-1 cells | | | | | | |
| GM-CSF-mediated signaling events | 13150 | 8.62E−02 | 6.82E−01 | 11.11 | 27.23 | MAP2K1 |
| Signaling to ERKs | 13150 | 8.62E−02 | 6.78E−01 | 11.11 | 27.23 | MAP2K1 |
| Transport to the Golgi and | 13150 | 8.62E−02 | 6.75E−01 | 11.11 | 27.23 | SAR1B |
| subsequent modification | | | | | | |
| Neurotransmitter release cycle | 13150 | 8.62E−02 | 6.71E−01 | 11.11 | 27.23 | SYT1 |
| Platelet aggregation (plug | 13516 | 8.85E−02 | 6.86E−01 | 10.81 | 26.21 | RASGRP1 |
| formation) | | | | | | |
| Signaling of hepatocyte growth | 13881 | 9.08E−02 | 7.00E−01 | 10.53 | 25.25 | MAP2K1 |
| factor receptor | | | | | | |
| Trefoil factor initiation of mucosal | 13881 | 9.08E−02 | 6.96E−01 | 10.53 | 25.25 | MAP2K1 |
| healing | | | | | | |
| Nuclear receptors | 13881 | 9.08E−02 | 6.93E−01 | 10.53 | 25.25 | NR2F2 |
| Platelet activation, signaling and | 2/205 | 9.30E−02 | 7.06E−01 | 3.90 | 9.27 | GNB1; RASGRP1 |
| aggregation | | | | | | |
| Angiotensin II-mediated activation | 14246 | 9.31E−02 | 7.03E−01 | 10.26 | 24.35 | MAP2K1 |
| of JNK pathway via Pyk2-dependent | | | | | | |
| signaling | | | | | | |
| FRS2-mediated cascade | 14246 | 9.31E−02 | 6.99E−01 | 10.26 | 24.35 | MAP2K1 |
| Transcriptional activity of | 14977 | 9.76E−02 | 7.30E−01 | 9.76 | 22.70 | NCOR2 |
| SMAD2/SMAD3-SMAD4 | | | | | | |
| heterotrimer | | | | | | |
| ERBB1 internalization pathway | 14977 | 9.76E−02 | 7.26E−01 | 9.76 | 22.70 | DNM1 |
| FOXM1 transcription factor network | 14977 | 9.76E−02 | 7.23E−01 | 9.76 | 22.70 | MAP2K1 |
| Fc epsilon receptor I signaling in | 14977 | 9.76E−02 | 7.19E−01 | 9.76 | 22.70 | MAP2K1 |
| mast cells | | | | | | |
| Bladder cancer | 15342 | 9.99E−02 | 7.32E−01 | 9.52 | 21.94 | MAP2K1 |
| Growth hormone receptor signaling | 15707 | 1.02E−01 | 7.45E−01 | 9.30 | 21.22 | MAP2K1 |
| Insulin secretion regulation by | 15707 | 1.02E−01 | 7.42E−01 | 9.30 | 21.22 | GNB1 |
| glucagon-like peptide-1 | | | | | | |
| Voltage-gated potassium channels | 15707 | 1.02E−01 | 7.38E−01 | 9.30 | 21.22 | KCNAB2 |
| G-protein-mediated events | 16072 | 1.04E−01 | 7.51E−01 | 9.09 | 20.54 | GNAO1 |
| HNF3A pathway | 16072 | 1.04E−01 | 7.47E−01 | 9.09 | 20.54 | NR2F2 |
| NCAM1 interactions | 16072 | 1.04E−01 | 7.44E−01 | 9.09 | 20.54 | NCAM1 |
| ERBB2/ERBB3 signaling events | 16072 | 1.04E−01 | 7.40E−01 | 9.09 | 20.54 | MAP2K1 |
| Signaling by NGF | 2/221 | 1.06E−01 | 7.45E−01 | 3.62 | 8.14 | MAP2K1; DNM1 |
| G alpha (z) signaling events | 16438 | 1.07E−01 | 7.49E−01 | 8.89 | 19.90 | GNB1 |
| RNA polymerase III transcription | 16438 | 1.07E−01 | 7.45E−01 | 8.89 | 19.90 | SNAPC5 |
| Interleukin-3 signaling pathway | 16438 | 1.07E−01 | 7.42E−01 | 8.89 | 19.90 | MAP2K1 |
| Signal transduction | 5/1020 | 1.10E−01 | 7.62E−01 | 1.96 | 4.33 | NCOR2; GNAO1; MAP2K1; GNB1; DNM1 |
| Actions of nitric oxide in the heart | 17168 | 1.11E−01 | 7.66E−01 | 8.51 | 18.70 | GNB1 |
| Regulation of transcription by | 17168 | 1.11E−01 | 7.63E−01 | 8.51 | 18.70 | NCOR2 |
| NOTCH1 intracellular domain | | | | | | |
| Delta Np63 pathway | 17168 | 1.11E−01 | 7.59E−01 | 8.51 | 18.70 | ADRM1 |
| Hemostasis pathway | 3/468 | 1.12E−01 | 7.60E−01 | 2.56 | 5.62 | KIF5A; GNB1; RASGRP1 |
| HES/HEY pathway | 17533 | 1.13E−01 | 7.67E−01 | 8.33 | 18.14 | NCOR2 |
| Lipid digestion, mobilization, and | 17533 | 1.13E−01 | 7.64E−01 | 8.33 | 18.14 | SAR1B |
| transport | | | | | | |
| Diurnally regulated genes with | 17533 | 1.13E−01 | 7.61E−01 | 8.33 | 18.14 | GSTP1 |
| circadian orthologs | | | | | | |
| G alpha 12 pathway | 17899 | 1.16E−01 | 7.72E−01 | 8.16 | 17.62 | MAP2K1 |
| Interleukin-5 signaling pathway | 17899 | 1.16E−01 | 7.69E−01 | 8.16 | 17.62 | MAP2K1 |
| Ceramide signaling pathway | 17899 | 1.16E−01 | 7.65E−01 | 8.16 | 17.62 | MAP2K1 |
| Aquaporin-mediated transport | 18264 | 1.18E−01 | 7.77E−01 | 8.00 | 17.11 | GNB1 |
| Glutathione metabolism | 18629 | 1.20E−01 | 7.88E−01 | 7.84 | 16.63 | GSTP1 |
| Interleukin-2 receptor beta chain in | 18994 | 1.22E−01 | 7.99E−01 | 7.69 | 16.17 | MAP2K1 |
| T cell activation | | | | | | |
| Signaling events mediated by stem | 18994 | 1.22E−01 | 7.95E−01 | 7.69 | 16.17 | MAP2K1 |
| cell factor receptor (c-Kit) | | | | | | |
| Taste transduction | 18994 | 1.22E−01 | 7.92E−01 | 7.69 | 16.17 | GNB1 |
| Mitochondrial protein import | 18994 | 1.22E−01 | 7.89E−01 | 7.69 | 16.17 | TIMM17A |
| Endometrial cancer | 18994 | 1.22E−01 | 7.85E−01 | 7.69 | 16.17 | MAP2K1 |
| Apoptosis | 2/242 | 1.23E−01 | 7.84E−01 | 3.31 | 6.94 | PSMC3; NMT1 |
| GABA A and B receptor activation | 19360 | 1.24E−01 | 7.93E−01 | 7.55 | 15.73 | GNB1 |
| Thrombin signaling through | 19360 | 1.24E−01 | 7.89E−01 | 7.55 | 15.73 | GNB1 |
| protease-activated receptors | | | | | | |
| RANKL signaling pathway | 19725 | 1.27E−01 | 8.00E−01 | 7.41 | 15.31 | MAP2K1 |
| Kit receptor signaling pathway | 19725 | 1.27E−01 | 7.96E−01 | 7.41 | 15.31 | MAP2K1 |
| Non-small cell lung cancer | 19725 | 1.27E−01 | 7.93E−01 | 7.41 | 15.31 | MAP2K1 |
| T cell receptor signaling in naive | 20090 | 1.29E−01 | 8.04E−01 | 7.27 | 14.91 | RASGRP1 |
| CD8+ T cells | | | | | | |
| Class A GPCRs (rhodopsin-like) | 2/253 | 1.32E−01 | 8.19E−01 | 3.16 | 6.41 | P2RY14; PTGER3 |
| Acute myeloid leukemia | 20821 | 1.33E−01 | 8.24E−01 | 7.02 | 14.15 | MAP2K1 |
| SHP2 signaling | 20821 | 1.33E−01 | 8.21E−01 | 7.02 | 14.15 | MAP2K1 |
| Keratinocyte differentiation | 20821 | 1.33E−01 | 8.17E−01 | 7.02 | 14.15 | MAP2K1 |
| Mechanism of gene regulation by | 20821 | 1.33E−01 | 8.14E−01 | 7.02 | 14.15 | NCOR2 |
| peroxisome proliferators via PPAR- | | | | | | |
| alpha | | | | | | |
| Arachidonic acid metabolism | 21186 | 1.35E−01 | 8.24E−01 | 6.90 | 13.79 | CBR1 |
| Autodegradation of Cdh1 by Cdh1- | 21186 | 1.35E−01 | 8.21E−01 | 6.90 | 13.79 | PSMC3 |
| APC/C | | | | | | |
| BDNF signaling pathway | 2/261 | 1.39E−01 | 8.37E−01 | 3.07 | 6.06 | MAP2K1; GPRC5B |
| Natural killer cell receptor signaling | 21916 | 1.40E−01 | 8.40E−01 | 6.67 | 13.12 | MAP2K1 |
| pathway | | | | | | |
| HIV genome transcription | 22282 | 1.42E−01 | 8.50E−01 | 6.56 | 12.81 | TCEB1 |
| Leptin signaling pathway | 22282 | 1.42E−01 | 8.46E−01 | 6.56 | 12.81 | MAP2K1 |
| Licensing factor removal from | 22282 | 1.42E−01 | 8.43E−01 | 6.56 | 12.81 | PSMC3 |
| origins | | | | | | |
| FGF signaling pathway | 22282 | 1.42E−01 | 8.40E−01 | 6.56 | 12.81 | NCAM1 |
| Signaling events mediated by focal | 22647 | 1.44E−01 | 8.49E−01 | 6.45 | 12.50 | MAP2K1 |
| adhesion kinase | | | | | | |
| Colorectal cancer | 22647 | 1.44E−01 | 8.46E−01 | 6.45 | 12.50 | MAP2K1 |
| Proteasome degradation | 23012 | 1.46E−01 | 8.55E−01 | 6.35 | 12.21 | PSMC3 |
| Neuroactive ligand-receptor | 2/272 | 1.48E−01 | 8.63E−01 | 2.94 | 5.62 | P2RY14; PTGER3 |
| interaction | | | | | | |
| Angiotensin II-stimulated signaling | 23377 | 1.48E−01 | 8.61E−01 | 6.25 | 11.93 | MAP2K1 |
| through G-proteins and beta- | | | | | | |
| arrestin | | | | | | |
| MAPK cascade role in angiogenesis | 23377 | 1.48E−01 | 8.58E−01 | 6.25 | 11.93 | MAP2K1 |
| Ubiquitin-mediated degradation of | 23377 | 1.48E−01 | 8.54E−01 | 6.25 | 11.93 | PSMC3 |
| phosphorylated Cdc25A | | | | | | |
| Validated nuclear estrogen receptor | 23377 | 1.48E−01 | 8.51E−01 | 6.25 | 11.93 | NCOR2 |
| alpha network | | | | | | |
| Glioma | 23743 | 1.50E−01 | 8.60E−01 | 6.15 | 11.66 | MAP2K1 |
| ERK1/ERK2 MAPK pathway | 23743 | 1.50E−01 | 8.57E−01 | 6.15 | 11.66 | MAP2K1 |
| Signaling by TGF-beta receptor | 24108 | 1.53E−01 | 8.66E−01 | 6.06 | 11.40 | NCOR2 |
| complex | | | | | | |
| T cell receptor signaling in naive | 24473 | 1.55E−01 | 8.75E−01 | 5.97 | 11.14 | RASGRP1 |
| CD4+ T cells | | | | | | |
| Telomerase regulation | 24473 | 1.55E−01 | 8.71E−01 | 5.97 | 11.14 | NR2F2 |
| cAMP cell motility pathway inferred | 24473 | 1.55E−01 | 8.68E−01 | 5.97 | 11.14 | MAP2K1 |
| from amoeba model | | | | | | |
| Immune system signaling by | 2/280 | 1.55E−01 | 8.66E−01 | 2.86 | 5.33 | MAP2K1; NCAM1 |
| interferons, interleukins, prolactin, | | | | | | |
| and growth hormones | | | | | | |
| Activation of NF-kappaB in B cells | 24838 | 1.57E−01 | 8.73E−01 | 5.88 | 10.90 | PSMC3 |
| CD8/T cell receptor downstream | 24838 | 1.57E−01 | 8.70E−01 | 5.88 | 10.90 | MAP2K1 |
| pathway | | | | | | |
| NEAT involvement in hypertrophy of | 25204 | 1.59E−01 | 8.79E−01 | 5.80 | 10.66 | MAP2K1 |
| the heart | | | | | | |
| Pancreatic cancer | 25569 | 1.61E−01 | 8.87E−01 | 5.71 | 10.44 | MAP2K1 |
| Signaling events mediated by HDAC | 25569 | 1.61E−01 | 8.84E−01 | 5.71 | 10.44 | NCOR2 |
| class I | | | | | | |
| Signaling events mediated by | 25569 | 1.61E−01 | 8.81E−01 | 5.71 | 10.44 | MAP2K1 |
| VEGFR1 and VEGFR2 | | | | | | |
| Long-term potentiation | 25569 | 1.61E−01 | 8.78E−01 | 5.71 | 10.44 | MAP2K1 |
| Phase II of biological oxidations: | 25934 | 1.63E−01 | 8.86E−01 | 5.63 | 10.22 | GSTP1 |
| conjugation | | | | | | |
| Bacterial invasion of epithelial cells | 25934 | 1.63E−01 | 8.83E−01 | 5.63 | 10.22 | DNM1 |
| Interleukin-6 signaling pathway | 25934 | 1.63E−01 | 8.79E−01 | 5.63 | 10.22 | MAP2K1 |
| Melanoma | 25934 | 1.63E−01 | 8.76E−01 | 5.63 | 10.22 | MAP2K1 |
| Cyclin A-Cdk2-associated events at S | 26299 | 1.65E−01 | 8.85E−01 | 5.56 | 10.00 | PSMC3 |
| phase entry | | | | | | |
| TGF-beta regulation of extracellular | 3/565 | 1.67E−01 | 8.92E−01 | 2.12 | 3.80 | MAP2K1; NR2F2; NFE2L1 |
| matrix | | | | | | |
| Signaling by NOTCH1 | 26665 | 1.67E−01 | 8.89E−01 | 5.48 | 9.80 | NCOR2 |
| Chronic myeloid leukemia | 26665 | 1.67E−01 | 8.86E−01 | 5.48 | 9.80 | MAP2K1 |
| Degradation of beta-catenin by the | 26665 | 1.67E−01 | 8.83E−01 | 5.48 | 9.80 | PSMC3 |
| destruction complex | | | | | | |
| Seven transmembrane receptor | 27030 | 1.69E−01 | 8.91E−01 | 5.41 | 9.60 | MAP2K1 |
| signaling through beta-arrestin | | | | | | |
| Prolactin activation of MAPK | 27395 | 1.71E−01 | 8.99E−01 | 5.33 | 9.41 | MAP2K1 |
| signaling | | | | | | |
| VEGF signaling pathway | 27760 | 1.74E−01 | 9.07E−01 | 5.26 | 9.22 | MAP2K1 |
| G alpha (12/13) signaling events | 28126 | 1.76E−01 | 9.14E−01 | 5.19 | 9.04 | GNB1 |
| Apoptosis regulation | 28491 | 1.78E−01 | 9.22E−01 | 5.13 | 8.86 | PSMC3 |
| Signaling by SCF-KIT | 28491 | 1.78E−01 | 9.19E−01 | 5.13 | 8.86 | MAP2K1 |
| Antigen processing: cross | 28856 | 1.80E−01 | 9.26E−01 | 5.06 | 8.69 | PSMC3 |
| presentation | | | | | | |
| Signaling events mediated by | 28856 | 1.80E−01 | 9.23E−01 | 5.06 | 8.69 | MAP2K1 |
| hepatocyte growth factor receptor | | | | | | |
| (c-Met) | | | | | | |
| p73 transcription factor network | 28856 | 1.80E−01 | 9.20E−01 | 5.06 | 8.69 | TUBA1A |
| Fc epsilon receptor I signaling | 28856 | 1.80E−01 | 9.17E−01 | 5.06 | 8.69 | MAP2K1 |
| pathway | | | | | | |
| MAP kinase signaling pathway | 29587 | 1.84E−01 | 9.35E−01 | 4.94 | 8.36 | MAP2K1 |
| MAPK signaling pathway | 2/314 | 1.85E−01 | 9.38E−01 | 2.55 | 4.30 | MAP2K1; RASGRP1 |
| APC/C-mediated degradation of cell | 29952 | 1.86E−01 | 9.39E−01 | 4.88 | 8.21 | PSMC3 |
| cycle proteins | | | | | | |
| Platelet homeostasis | 29952 | 1.86E−01 | 9.36E−01 | 4.88 | 8.21 | GNB1 |
| Drug metabolism: cytochrome P450 | 30317 | 1.88E−01 | 9.43E−01 | 4.82 | 8.06 | GSTP1 |
| Innate immune system | 2/319 | 1.90E−01 | 9.48E−01 | 2.51 | 4.17 | MAP2K1; DNM1 |
| Differentiation pathway in PC12 | 30682 | 1.90E−01 | 9.47E−01 | 4.76 | 7.91 | MAP2K1 |
| cells | | | | | | |
| T cell receptor regulation of | 3/603 | 1.91E−01 | 9.48E−01 | 1.99 | 3.30 | GSTP1; TCEB1; RASGRP1 |
| apoptosis | | | | | | |
| Asparagine N-linked glycosylation | 31048 | 1.92E−01 | 9.51E−01 | 4.71 | 7.77 | SAR1B |
| Integrin cell surface interactions | 31048 | 1.92E−01 | 9.48E−01 | 4.71 | 7.77 | RASGRP1 |
| MicroRNAs in cardiomyocyte | 31048 | 1.92E−01 | 9.44E−01 | 4.71 | 7.77 | MAP2K1 |
| hypertrophy | | | | | | |
| Progesterone-mediated oocyte | 31413 | 1.94E−01 | 9.51E−01 | 4.65 | 7.63 | MAP2K1 |
| maturation | | | | | | |
| mRNA stability regulation by | 31413 | 1.94E−01 | 9.48E−01 | 4.65 | 7.63 | PSMC3 |
| proteins that bind AU-rich elements | | | | | | |
| Mitotic G2-G2/M phases | 31778 | 1.96E−01 | 9.55E−01 | 4.60 | 7.49 | TUBA1A |
| Androgen receptor signaling, | 32143 | 1.98E−01 | 9.62E−01 | 4.55 | 7.36 | NCOR2 |
| proteolysis, and transcription | | | | | | |
| regulation | | | | | | |
| DNA replication pre-Initiation | 32143 | 1.98E−01 | 9.59E−01 | 4.55 | 7.36 | PSMC3 |
| Signaling by ERBB4 | 33970 | 2.08E−01 | 1.00E+00 | 4.30 | 6.75 | MAP2K1 |
| ERBB signaling pathway | 34335 | 2.10E−01 | 1.00E+00 | 4.26 | 6.64 | MAP2K1 |
| RNA polymerase I, RNA polymerase | 34700 | 2.12E−01 | 1.00E+00 | 4.21 | 6.53 | SNAPC5 |
| III, and mitochondrial transcription | | | | | | |
| Class B GPCRs (secretin family | 34700 | 2.12E−01 | 1.00E+00 | 4.21 | 6.53 | GNB1 |
| receptors) | | | | | | |
| Mitochondrial pathway of | 35431 | 2.16E−01 | 1.00E+00 | 4.12 | 6.32 | NMT1 |
| apoptosis: BH3-only Bcl-2 family | | | | | | |
| Granule cell survival pathway | 36161 | 2.20E−01 | 1.00E+00 | 4.04 | 6.12 | MAP2K1 |
| Senescence and autophagy | 36161 | 2.20E−01 | 1.00E+00 | 4.04 | 6.12 | MAP2K1 |
| Integrin-mediated cell adhesion | 1/100 | 2.22E−01 | 1.00E+00 | 4.00 | 6.02 | MAP2K1 |
| Gene expression | 4/968 | 2.22E−01 | 1.00E+00 | 1.65 | 2.49 | SNAPC5; NCOR2; PSMC3; TCEB1 |
| GnRH signaling pathway | 1/101 | 2.24E−01 | 1.00E+00 | 3.96 | 5.93 | MAP2K1 |
| RNA polymerase II transcription | 1/101 | 2.24E−01 | 1.00E+00 | 3.96 | 5.93 | TCEB1 |
| Signaling by ERBB2 | 1/102 | 2.26E−01 | 1.00E+00 | 3.92 | 5.84 | MAP2K1 |
| Chagas disease | 1/104 | 2.30E−01 | 1.00E+00 | 3.85 | 5.66 | GNAO1 |
| Fibroblast growth factor receptor | 1/105 | 2.32E−01 | 1.00E+00 | 3.81 | 5.57 | MAP2K1 |
| pathway | | | | | | |
| ERBB1 downstream pathway | 1/106 | 2.34E−01 | 1.00E+00 | 3.77 | 5.49 | MAP2K1 |
| G alpha i pathway | 1/108 | 2.37E−01 | 1.00E+00 | 3.70 | 5.33 | MAP2K1 |
| Signaling by insulin receptor | 1/109 | 2.39E−01 | 1.00E+00 | 3.67 | 5.25 | MAP2K1 |
| Signaling by interleukins | 1/109 | 2.39E−01 | 1.00E+00 | 3.67 | 5.25 | MAP2K1 |
| Signaling by EGFR in cancer | 1/111 | 2.43E−01 | 1.00E+00 | 3.60 | 5.10 | MAP2K1 |
| Epidermal growth factor receptor | 1/111 | 2.43E−01 | 1.00E+00 | 3.60 | 5.10 | MAP2K1 |
| (EGFR) pathway | | | | | | |
| S phase | 1/112 | 2.45E−01 | 1.00E+00 | 3.57 | 5.02 | PSMC3 |
| Lipid metabolism regulation by | 1/112 | 2.45E−01 | 1.00E+00 | 3.57 | 5.02 | NCOR2 |
| peroxisome proliferator-activated | | | | | | |
| receptor alpha (PPAR-alpha) | | | | | | |
| Oocyte meiosis | 1/113 | 2.47E−01 | 1.00E+00 | 3.54 | 4.95 | MAP2K1 |
| mTOR signaling pathway | 1/113 | 2.47E−01 | 1.00E+00 | 3.54 | 4.95 | MAP2K1 |
| Vascular smooth muscle contraction | 1/116 | 2.53E−01 | 1.00E+00 | 3.45 | 4.74 | MAP2K1 |
| Cell cycle checkpoints | 1/117 | 2.55E−01 | 1.00E+00 | 3.42 | 4.68 | PSMC3 |
| p53 activity regulation | 1/118 | 2.56E−01 | 1.00E+00 | 3.39 | 4.61 | CSE1L |
| Signaling by NOTCH | 1/119 | 2.58E−01 | 1.00E+00 | 3.36 | 4.55 | NCOR2 |
| G alpha s pathway | 1/120 | 2.60E−01 | 1.00E+00 | 3.33 | 4.49 | MAP2K1 |
| Interleukin-1 regulation of | | | | | | |
| extracellular matrix | 1/120 | 2.60E−01 | 1.00E+00 | 3.33 | 4.49 | NR2F2 |
| Signaling by PDGF | 1/122 | 2.64E−01 | 1.00E+00 | 3.28 | 4.37 | MAP2K1 |
| G alpha (s) signaling events | 1/125 | 2.69E−01 | 1.00E+00 | 3.20 | 4.20 | GNB1 |
| Interleukin-1 signaling pathway | 1/125 | 2.69E−01 | 1.00E+00 | 3.20 | 4.20 | MAP2K1 |
| Factors involved in megakaryocyte | 1/125 | 2.69E−01 | 1.00E+00 | 3.20 | 4.20 | KIF5A |
| development and platelet | | | | | | |
| production | | | | | | |
| Neurotrophin signaling pathway | 1/126 | 2.71E−01 | 1.00E+00 | 3.17 | 4.14 | MAP2K1 |
| Signaling by FGFR in disease | 1/128 | 2.75E−01 | 1.00E+00 | 3.13 | 4.04 | MAP2K1 |
| PDGFB signaling pathway | 1/129 | 2.77E−01 | 1.00E+00 | 3.10 | 3.98 | MAP2K1 |
| Adipogenesis | 1/133 | 2.84E−01 | 1.00E+00 | 3.01 | 3.79 | NCOR2 |
| Cell adhesion molecules (CAMs) | 1/133 | 2.84E−01 | 1.00E+00 | 3.01 | 3.79 | NCAM1 |
| Mitotic G1-G1/S phases | 1/135 | 2.88E−01 | 1.00E+00 | 2.96 | 3.69 | PSMC3 |
| Ubiquitin-mediated proteolysis | 1/136 | 2.89E−01 | 1.00E+00 | 2.94 | 3.65 | TCEB1 |
| Natural killer cell-mediated | 1/137 | 2.91E−01 | 1.00E+00 | 2.92 | 3.60 | MAP2K1 |
| cytotoxicity | | | | | | |
| Biological oxidations | 1/139 | 2.95E−01 | 1.00E+00 | 2.88 | 3.52 | GSTP1 |
| p53 signaling pathway | 1/139 | 2.95E−01 | 1.00E+00 | 2.88 | 3.52 | CSE1L |
| Toll-like receptor signaling pathway | 1/142 | 3.00E−01 | 1.00E+00 | 2.82 | 3.39 | MAP2K1 |
| regulation | | | | | | |
| Cell cycle | 2/453 | 3.13E−01 | 1.00E+00 | 1.77 | 2.05 | TUBA1A; PSMC3 |
| Integrin signaling pathway | 1/155 | 3.23E−01 | 1.00E+00 | 2.58 | 2.92 | MAP2K1 |
| Myometrial relaxation and | 1/155 | 3.23E−01 | 1.00E+00 | 2.58 | 2.92 | GNB1 |
| contraction pathways | | | | | | |
| Protein processing in the | 1/166 | 3.41E−01 | 1.00E+00 | 2.41 | 2.59 | SAR1B |
| endoplasmic reticulum | | | | | | |
| Interferon signaling | 1/168 | 3.44E−01 | 1.00E+00 | 2.38 | 2.54 | NCAM1 |
| Lipid and lipoprotein metabolism | 2/489 | 3.47E−01 | 1.00E+00 | 1.64 | 1.73 | NCOR2; SAR1B |
| Fatty acid, triacylglycerol, and | 1/173 | 3.53E−01 | 1.00E+00 | 2.31 | 2.41 | NCOR2 |
| ketone body metabolism | | | | | | |
| Calcium signaling pathway | 1/178 | 3.61E−01 | 1.00E+00 | 2.25 | 2.29 | PTGER3 |
| TGF-beta signaling pathway | 1/185 | 3.72E−01 | 1.00E+00 | 2.16 | 2.14 | MAP2K1 |
| Metabolism | 5/1615 | 3.79E−01 | 1.00E+00 | 1.24 | 1.20 | NCOR2; GNAO1; CBR1; GSTP1; GNB1 |
| Amino acid metabolism | 1/195 | 3.88E−01 | 1.00E+00 | 2.05 | 1.94 | PSMC3 |
| Post-translational protein | | | | | | |
| modification | 1/196 | 3.89E−01 | 1.00E+00 | 2.04 | 1.93 | SAR1B |
| Endocytosis | 1/201 | 3.97E−01 | 1.00E+00 | 1.99 | 1.84 | DNM1 |
| DNA replication | 1/207 | 4.06E−01 | 1.00E+00 | 1.93 | 1.74 | PSMC3 |
| Antigen-activated B-cell receptor | 1/211 | 4.12E−01 | 1.00E+00 | 1.90 | 1.68 | MAP2K1 |
| generation of second messengers | | | | | | |
| Actin cytoskeleton regulation | 1/226 | 4.34E−01 | 1.00E+00 | 1.77 | 1.48 | MAP2K1 |
| Focal adhesion | 1/233 | 4.44E−01 | 1.00E+00 | 1.72 | 1.39 | MAP2K1 |
| Interleukin-4 regulation of apoptosis | 1/267 | 4.90E−01 | 1.00E+00 | 1.50 | 1.07 | RASGRP1 |
| Insulin signaling pathway | 1/277 | 5.03E−01 | 1.00E+00 | 1.44 | 0.99 | MAP2K1 |
| Oncostatin M | 1/311 | 5.44E−01 | 1.00E+00 | 1.29 | 0.78 | CALB2 |
| Generic transcription pathway | 1/377 | 6.14E−01 | 1.00E+00 | 1.06 | 0.52 | NCOR2 |
| Transmembrane transport of small | 1/432 | 6.65E−01 | 1.00E+00 | 0.93 | 0.38 | GNB1 |
| molecules | | | | | | |
| Olfactory transduction | 1/432 | 6.65E−01 | 1.00E+00 | 0.93 | 0.38 | GNB1 |

TABLE 11
Gene signals Healthy

| | | | |
|---|---|---|---|
| PASS_Celiac | zheng_pbmc | T_Lymphocytes | ETS1, CD247, RCAN3, CD28, TXK, |
| | | | ANKRD12, LBH, C12orf75, ANXA6, |
| | | | UBASH3A, GRAP2, PA2G4, NDFIP1, |
| | | | RORA, C11orf58, TNFAIP8, RAC2, |
| | | | PYHIN1, RPL18, DSTN, SOCS3, APRT, |
| | | | RPL6, ARL4C, BCL11B, LAT, TAF7, |
| | | | MIF, PTPRCAP, STMN1, HINT1, LEF1, |
| | | | RPS25, GZMK, RPA2, SOD1, PRR5, |
| | | | C9orf78, SKAP1, RPS12, RPS20, |
| | | | SPOCK2, DGCR6L, ANXA2R, TMEM173, |
| | | | ISG20, CCR7, SLC9A3R1, NPM1, |
| | | | METTL9 |
| PASS_Ulcerative_Colitis | zheng_pbmc | B_Lymphocytes | GPX1, REL, LSP1, FAM26F, IMPDH2, |
| | | | EIF6, BRK1, NFKBIA, SHMT2, LAPTM5, |
| | | | RPL23A, CTSS, PRKCB, BANK1, ALOX5, |
| | | | TCF4, CCDC50, HHEX, MS4A1, RPS5, |
| | | | ENSA, BCAS4, USF2, SLC50A1, SCIMP, |
| | | | ARID5B, RPS13, DUSP1, AFF3, FAU, |
| | | | PNOC, ZFP36L1, SELL, NCF4, DBNL, |
| | | | ADK, RPL28, CD19, EZR, RPSA, RPL23, |
| | | | PLAC8, CCNI, PPAPDC1B, LSM10, PKIG, |
| | | | RPS24, RNASET2, PRR13, LTA4H |
| PASS_MDD_Wray2018 | brain | GABAergic | TCF4, PCLO, BEND4, ZNF462, SEMA6D, |
| | | | TMEM106B, CHRM2, TMX2, MAP7D1, |
| | | | ADARB1, TAOK3, NYAP2, RTN1, ASTN2, |
| | | | GABRA1, ZNF608, SRRM4, NTM, |
| | | | CCDC152, EYS, GRIA1, GPX1, CKAP2, |
| | | | HSBP1L1, C7orf72, SERPINI1, ERBB4, |
| | | | MEGF11, TCAIM, B4GALT6, RAPGEF4, |
| | | | ROBO2, BICD1, C1QTNF7, NMNAT2, |
| | | | SGCZ, NTRK2, CC2D2A, PSME2, |
| | | | PTPRN, CNTNAP5, PER3, SEC61G, |
| | | | OSBPL3, RBMS3, RNF152, |
| | | | CDH9, DLGAP1, SMARCA4, ZPBP |
| PASS_Intelligence_SavageJansen2018 | brain | Glutamatergic | RBFOX1, RNF123, DCC, TRAIP, NEGR1, |
| | | | IP6K1, NICN1, AMT, ATXN2L, TCTA, |
| | | | RBM6, GPX1, RHOA, CAMKV, BSN, |
| | | | CSE1L, TUFM, EXOC4, FOXO3, APEH, |
| | | | SH2B1, CCDC101, RBM5, CALN1, |
| | | | DPP4, SULT1A1, MON1A, SULT1A2, |
| | | | MGAT3, CLN3, ARFGEF2, PRKAG1, DDN, |
| | | | DAG1, GBF1, ZNF638, THRB, LONRF2, |
| | | | AKTIP, FOXP1, MYBPHL, MEF2C, |
| | | | PTPRT, MGEA5, NKIRAS1, RHEBL1, |
| | | | SPNS1, SHISA9, EFTUD1, PPM1E |
| UKB_460K.lung_FEV1FVCzSMOKE | kropski_lung | Fibroblasts | ITGA1, MFAP2, LOX, RBMS3, TGFBR3, |
| | | | HTRA1, EFEMP1, ADAMTS2, CALD1, |
| | | | COL4A2, DNAJB4, NEXN, LTBP1, MRC2, |
| | | | LMCD1, RERG, MACF1, LRP1, DTWD1, |
| | | | PLXDC2, ITGAV, FGF7, PDZRN3, |
| | | | RHOBTB3, DST, LTBP2, TIMP3, LTBP4, |
| | | | IL1R1, ADAMTS5, PRSS23, ANTXR1, |
| | | | COL16A1, SMAD3, PHLDB2, HMCN1, |
| | | | P4HA2, ZFP36L2, MAP1LC3A, PLAC9, |
| | | | ARF4, IFITM2, HSPG2, SFRP2, NID2, |
| | | | HOXB2, COL6A3, IFITM1, PDGFRL, |
| | | | ADD3 |
| UKB_460K.lung_FEV1FVCzSMOKE | kropski_lung | Myofibroblasts | ITGA1, MFAP2, NPNT, LOX, RBMS3, |
| | | | TGFBR3, HTRA1, EFEMP1, ADAMTS2, |
| | | | CALD1, COL4A2, NEXN, LTBP1, MRC2, |
| | | | LMCD1, RERG, MACF1, LRP1, FGF7, |
| | | | RHOBTB3, DST, LTBP2, TIMP3, LTBP4, |
| | | | IL1R1, ANTXR1, COL16A1, HMCN1, |
| | | | PLAC9, IFITM2, HSPG2, COL6A3, |
| | | | TFPI, CYBRD1,TPM1, FBN1, MMP14, |
| | | | SERPING1, MYL9, COL8A1, PDGFRA, |
| | | | RASL12, ENAH, FEZ1, BAMBI, VCL, |
| | | | PARVA, GPX8, FGFR4, |
| | | | ANGPT1 |
| UKB_460K.bp_DIASTOLICadjMEDz | heart | Pericyte | PLCE1, ARHGAP42, AGT, GUCY1A3, |
| | | | PDE1A, ADCY3, TNS1, MKLN1, MRVI1, |
| | | | CACNA1C, SETBP1, GPAT2, JAG1, ABO, |
| | | | EBF1, CDC42BPA, BCAS3, NGF, SEPT9, |
| | | | ENPEP, ZBTB46, EPOR, GUCY1B3, |
| | | | RGL3, EBF2, SOX13, TBX2, WISP1, |
| | | | TRAK1, CENPO, TNS2, ANO1, PRKG1, |
| | | | DENND2A, LMOD1, NOTCH3, TCF4, |
| | | | SOX5, RBPMS, THSD7B, INPP4B, |
| | | | RERG, KALRN, COL5A3, ANKS1A, |
| | | | ARHGEF17, COBLL1, NFASC, SGIP1, |
| | | | GPRIN3 |
| UKB_460K.bp_SYSTOLICadjMEDz | heart | Pericyte | PLCE1, ARHGAP42, MKLN1, AGT, EBF1, |
| | | | TNS1, GUCY1A3, TBX2, SETBP1, EBF2, |
| | | | ADCY3, CACNA1C, SEPT9, BCAS3, |
| | | | DCBLD1, MRVI1, TNS2, NGF, FHL5, |
| | | | ENPEP, EDNRA, ZBTB46, THSD7B, |
| | | | PDE8A, SGIP1, GUCY1B3, EPOR, |
| | | | SOX13, NBEAL1, RGL3, COBLL1, |
| | | | PRKG1, HIGD1B, HIP1, CDC42BPA, |
| | | | JAG1, PDE1A, INPP4B, FAM213A, |
| | | | DENND2A, ANKS1A, GJA4, PTH1R, |
| | | | DOCK6, SLC12A2, NRP1, CENPO, |
| | | | WISP1, DGKH, APOLD1 |
| UKB_460K.bp_DIASTOLICadjMEDz | heart | Smooth_Muscle | CACNB2, CELF1, GUCY1A3, PDE1A, |
| | | | ADCY3, COL4A1, TNS1, MICAL3, |
| | | | MRVI1, PRDM16, MYO9B, CACNA1C, |
| | | | SETBP1, SLMAP, JAG1, TMEM165, |
| | | | LIMA1, EBF1, CNNM2, CLIC4, |
| | | | BCAS3, SLC4A7, SEPT9, |
| | | | ENPEP, SLC8A1, CDKAL1, COL4A2, |
| | | | ARHGEF26, RGL3, TBX2, CFAP69, |
| | | | ACTN4, PHLDB2, PDE5A, FRK, MYOCD, |
| | | | RYR2, FAM13A, GLS, CRIM1, ANO1, |
| | | | PRKG1, SPEG, FERMT2, DENND2A, |
| | | | COL21A1, COL1A1, ZHX3, LMOD1, |
| | | | RSRC1 |
| UKB_460K.bp_SYSTOLICadjMEDz | heart | Smooth_Muscle | CACNB2, CELF1, EBF1, TNS1, SLC4A7, |
| | | | GUCY1A3, TBX2, SETBP1, ADCY3, |
| | | | TCF7L2, CACNA1C, SEPT9, BCAS3, |
| | | | PRDM16, MRVI1, CNNM2, FHL5, ENPEP, |
| | | | MYO9B, FERMT2, JPH2, FN1, COL21A1, |
| | | | CAMK2G, VGLL4, HERC4, VCL, EDNRA, |
| | | | CDKAL1, SGCD, ARID5B, RGS7BP, |
| | | | SGIP1, ARHGEF26, TPM1, FRYL, |
| | | | KIF5B, AFAP1, CCDC6, ITGA9, |
| | | | FAM13A, SLC8A1, PALLD, |
| | | | TMEM165, NBEAL1, TCF7L1, GEM, |
| | | | SYNE1, RGL3, GLS |
| PASS_AtrialFibrillation_Nielsen2018 | heart | Atrial_Cardiomyocyte | CAV2, PPFIA4, TBX5, MYH6, PKD2L2, |
| | | | ASAH1, SPATS2L, CAV1, FAM13B, |
| | | | CASQ2, KCNN2, GBF1, HCN4, CFL2, |
| | | | KCND3, CAMK2D, CPEB4, PCM1, TTN, |
| | | | ATXN1, KCNH2, SSPN, ZNF292, |
| | | | CAND2, DPF3, FRMD4B, AKAP6, |
| | | | SMIM8, KLHL3, IGF1R, CDK6, |
| | | | USP34, FBXO32, SCN5A, ZBTB38, |
| | | | MYOT, SAMD8, CASZ1, NKX2-5, |
| | | | HIP1R, MYO18B, ERBB2, FBN2, |
| | | | C10orf76, SCMH1, TMEM40, |
| | | | NUCKS1, GJA5, LRIG1, MURC |
| PASS_Ulcerative_Colitis | xavier_colon | M_cells | PPP4C, TMSB10, LGALS4, GOLM1, |
| | | | GPX2, EPCAM, NDUFS8, AKR1C3, |
| | | | LGALS3, GMDS, KRT19, KRT18, |
| | | | SPIB, KRT8, S100A14, S100A6 |
| PASS_Ulcerative_Colitis | xavier_colon | Enteroendocrine | PNKD, UQCR10, UQCRC1, CLDN3, |
| | | | DBI, PPP1R1B, CLDN4, KRTCAP3, |
| | | | AURKAIP1, HSPD1, TIMM13, PIGR, |
| | | | FXYD3, GCG, KIF12, SLIRP, TMSB10, |
| | | | S100A10, LGALS4, ROMO1, MDH2, |
| | | | MRPL41, CHCHD10, C15orf48, FABP1, |
| | | | CISD1, C19orf70, MGST1, ATP5G1, |
| | | | PRSS3, H3F3A, COX6A1, CARHSP1, |
| | | | ECH1, HMGCS2, MPC2, NDUFB7, |
| | | | LAMTOR4, NDUFS5, GPX2, PRDX5, |
| | | | GAPDH, SCG5, TXN, EMC10, DCTPP1, |
| | | | CDX1, SNRPB, BAG1, EPCAM |
| UKB_460K.disease_ALLERGY_ECZEMA_DIAGNOSED | skin | Langerhans_cells | IL18R1, IL1R1, RUNX3, NFATC2, |
| | | | NDFIP1, FCER1G, HSPE1, UBE2E2, |
| | | | PLXNC1, RASA2, ARHGAP15, REL, |
| | | | DRAP1, EAF2, HCLS1, APOBR, |
| | | | RIN3, PRKCB, ARL6IP4, LAMTOR2, |
| | | | FPR3, ZMIZ1, GPR183, KYNU, |
| | | | ARRDC2, RILPL2, FNDC4, TMEM156, |
| | | | TMED5, ZFHX3, CFL1, NR4A2, |
| | | | ANKRD44, CNTRL, SCAMP2, |
| | | | CSGALNACT2, RASSF5, SCNM1, |
| | | | TYMP, CIITA, ICAM3, PTPRC, |
| | | | FES, CD52, FAM109A, ATPAF2, |
| | | | DEF6, TNFAIP3, OTULIN, |
| | | | FCGRT |

TABLE 12
Healthy Genes

| | | | | | | | | | | Atrial |
|---|---|---|---|---|---|---|---|---|---|---|
| | | | | | | | | | | Fibril- |
| Celiac | UC PBMC | | Intelli- | | | | | | Allergy | lation |
| PBMC T | B | | gence | UC | FEV1 | | Diastolic | Systolic | Eczema | atrial |
| lympho- | lympho- | MDD | gluta- | colon | lung | Fev1 lung | heart | heart | skin | cardio- |
| cytes | cytes | Gabaergic | matergic | M cell | fibro | myo | pericyte | pericyte | langerhan | myocyte |
| ETS1 | GPX1 | TCF4 | RBFOX1 | PPP4C | ITGA1 | ITGA1 | PLCE1 | PLCE1 | IL18R1 | CAV2 |
| CD247 | REL | PCLO | RNF123 | TMSB10 | MFAP2 | MFAP2 | ARHGAP42 | ARHGAP42 | URI. | PPFIA4 |
| RCAN3 | LSP1 | BEND4 | DCC | LGALS4 | LOX | NPNT | AGT | MKLN1 | RUNX3 | TBX5 |
| CD28 | FAM26F | ZNF462 | TRAIP | GOLM1 | RBMS3 | LOX | GUCY1A3 | AGT | NFATC2 | MYH6 |
| TXK | IMPDH2 | SEMA6D | NEGR1 | GPX2 | TGFBR3 | RBMS3 | PDE1A | EBF1 | NDFIP1 | PKD2L2 |
| ANKRD12 | EIF6 | TMEM10 | IP6K1 | EPCAM | HTRA1 | TGFBR3 | ADCY3 | TNS1 | FCER1G | ASAH1 |
| | | 6B | | | | | | | | |
| LBH | BRK1 | CHRM2 | NICN1 | NDUFS8 | EFEMP1 | HTRA1 | TNS1 | GUCY1A3 | HSPE1 | SPATS2L |
| C12orf75 | NFKBIA | TMX2 | AMT | AKR1C3 | ADAMTS2 | EFEMP1 | MKLN1 | TBX2 | UBE2E2 | CAV1 |
| ANXA6 | SHMT2 | MAP7D1 | ATXN2L | LGALS3 | CALD1 | ADAMTS2 | MRVI1 | SETBP1 | PLXNC1 | FAM13B |
| UBASH3A | LAPTM5 | ADARB1 | TCTA | GMDS | COL4A2 | CALD1 | CACNA1C | EBF2 | RASA2 | CASQ2 |
| GRAP2 | RPL23A | TAOK3 | RBM6 | KRT19 | DNAJB4 | COL4A2 | SETBP1 | ADCY3 | ARHGAP | KCNN2 |
| | | | | | | | | | 15 | |
| PA2G4 | CTSS | NYAP2 | GPX1 | KRT18 | NEXN | NEXN | GPAT2 | CACNA1C | REL | GBF1 |
| NDFIP1 | PRKCB | RTN1 | RHOA | SPIB | LTBP1 | LTBP1 | JAG1 | SEPT9 | DRAP1 | HCN4 |
| RORA | BANK1 | ASTN2 | CAMKV | KRT8 | MRC2 | MRC2 | ABO | BCAS3 | EAF2 | CFL2 |
| C11orf58 | TCF4 | GABRA1 | BSN | S100A14 | LMCD1 | LMCD1 | EBF1 | DCBLD1 | HCLS1 | KCND3 |
| TNFAIP8 | ALOX5 | ZNF608 | CSE1L | S100A6 | RERG | RERG | CDC42BPA | MRVI1 | APOBR | CAMK2D |
| RAC2 | CCDC50 | SRRM4 | TUFM | | MACF1 | MACF1 | BCAS3 | TNS2 | RIN3 | CPEB4 |
| PYHIN1 | HHEX | NTM | EXOC4 | | LRP1 | LRP1 | NGF | NGF | PRKCB | PCM1 |
| RPL18 | MS4A1 | CCDC152 | FOXO3 | | DTWD1 | FGF7 | SEPT9 | FHL5 | ARL6IP4 | TTN |
| DSTN | RPS5 | EYS | APEH | | PLXDC2 | RHOBTB3 | ENPEP | ENPEP | LAMTOR2 | ATXN1 |
| SOCS3 | ENSA | GRIA1 | SH2B1 | | ITGAV | DST | ZBTB46 | EDNRA | FPR3 | KCNH2 |
| APRT | BCAS4 | GPX1 | CCDC101 | | FGF7 | LTBP2 | EPOR | ZBTB46 | ZMIZ1 | SSPN |
| RPL6 | USF2 | CKAP2 | RBM5 | | PDZRN3 | TIMP3 | GUCY1B3 | THSD7B | GPR183 | ZNF292 |
| ARL4C | SLC50A1 | HSBP1L1 | CALN1 | | RHOBTB3 | LTBP4 | RGL3 | PDE8A | KYNU | CAND2 |
| BCL11B | SCIMP | C7orf72 | DPP4 | | DST | IL1R1 | EBF2 | SGIP1 | ARRDC2 | DPF3 |
| LAT | ARID5B | SERPINI1 | SULT1A1 | | LTBP2 | ANTXR1 | SOX13 | GUCY1B3 | RILPL2 | FRMD4B |
| TAF7 | RPS13 | ERBB4 | MON1A | | TIMP3 | COL16A1 | TBX2 | EPOR | FNDC4 | AKAP6 |
| MIF | DUSP1 | MEGF11 | SULT1A2 | | LTBP4 | HMCN1 | WISP1 | SOX13 | TMEM156 | SMIM8 |
| PTPRCAP | AFF3 | TCAIM | MGAT3 | | IL1R1 | PLAC9 | TRAK1 | NBEAL1 | TMED5 | KLHL3 |
| STMN1 | FAU | B4GALT6 | CLN3 | | ADAMTS5 | IFITM2 | CENPO | RGL3 | ZFHX3 | IGF1R |
| HINT1 | PNOC | RAPGEF4 | ARFGEF2 | | PRSS23 | HSPG2 | TNS2 | COBLL1 | CFL1 | CDK6 |
| LEF1 | ZFP36L1 | ROBO2 | PRKAG1 | | ANTXR1 | COL6A3 | ANO1 | PRKG1 | NR4A2 | USP34 |
| RPS25 | SELL | BICD1 | DDN | | COL16A1 | TFPI | PRKG1 | HIGD1B | ANKRD44 | FBXO32 |
| GZMK | NCF4 | C1QTNF7 | DAG1 | | SMAD3 | CYBRD1 | DENND2A | HIP1 | CNTRL | SCN5A |
| RPA2 | DBNL | NMNAT2 | GBF1 | | PHLDB2 | TPM1 | LMOD1 | CDC42BPA | SCAMP2 | ZBTB38 |
| SOD1 | ADK | SGCZ | ZNF638 | | HMCN1 | FBN1 | NOTCH3 | JAG1 | CSGALNA | MYOT |
| | | | | | | | | | CT2 | |
| PRR5 | RPL28 | NTRK2 | THRB | | P4HA2 | MMP14 | TCF4 | PDE1A | RASSF5 | SAMD8 |
| C9orf78 | CD19 | CC2D2A | LONRF2 | | ZFP36L2 | SERPING1 | SOX5 | INPP4B | SCNM1 | CASZ1 |
| SKAP1 | EZR | PSME2 | AKTIP | | MAP1LC3A | MYL9 | RBPMS | FAM213A | TYMP | NKX2-5 |
| RPS12 | RPSA | PTPRN | FOXP1 | | PLAC9 | COL8A1 | THSD7B | DENND2A | CIITA | HIP1R |
| RPS20 | RPL23 | CNTNAP5 | MYBPHL | | ARF4 | PDGFRA | INPP4B | ANKS1A | ICAM3 | MYO18B |
| SPOCK2 | PLAC8 | PER3 | MEF2C | | IFITM2 | RASL12 | RERG | GJA4 | PTPRC | ERBB2 |
| DGCR6L | CCNI | SEC61G | PTPRT | | HSPG2 | ENAH | KALRN | PTH1R | FES | FBN2 |
| ANXA2R | PPAPDC1B | OSBPL3 | MGEA5 | | SFRP2 | FEZ1 | COL5A3 | DOCK6 | CD52 | C10orf76 |
| TMEM173 | LSM10 | RBMS3 | NKIRAS1 | | NID2 | BAMBI | ANKS1A | SLC12A2 | FAM109A | SCMH1 |
| ISG20 | PKIG | RNF152 | RHEBL1 | | HOXB2 | VCL | ARHGEF17 | NRP1 | ATPAF2 | TMEM40 |
| CCR7 | RPS24 | CDH9 | SPNS1 | | COL6A3 | PARVA | COBLL1 | CENPO | DEF6 | NUCKS1 |
| SLC9A3R1 | RNASET2 | DLGAP1 | SHISA9 | | IFITM1 | GPX8 | NFASC | WISP1 | TNFAIP3 | GJA5 |
| NPM1 | PRR13 | SMARCA4 | EFTUD1 | | PDGFRL | FGFR4 | SGIP1 | DGKH | OTULIN | LRIG1 |
| METTL9 | LTA4H | ZPBP | PPM1E | | ADD3 | ANGPT1 | GPRIN3 | APOLD1 | FCGRT | MURC |

### DISCUSSION

Applicants conclude that Enhancer-to-gene strategy (Roadmap-U-ABC) captures highly specific disease signal for cell type enriched programs across multiple healthy tissues and this approach can be used effectively to nominate driving genes specific to a disease.

Applicants further provide a new approach integrating gene level signals from MAGMA and macro (T cells) cell type level information from scLDSC to get intermediate micro (Tregs) cell type level information.

Even though these analyses identify genes and pathways associated with known disease processes, they are not synonymous with the canonical disease markers. For example, smooth muscle actin is an immunohistochemical marker, but it was not identified in the analysis. Instead TGFBR3 was identified. TGFBR3 is the least understood of the genes in the TGFB signaling pathway. However, its role in regulating the available TGFB is a novel finding.

### Methods

### Identifying Genes Driving Heritability Signal

Applicants first subset the full gene list to only consider the top genes enriched in the cell type specific program. Subsequently, Applicants ranked all remaining genes using a MAGMA gene level significance score and considered the top 10 ranked genes to be the genes most highly influencing disease heritability signal.

### Shared NMF Clustering of Healthy and Disease Tissue Gene Expression

Let H_{P×N1} be the observed gene expression data for a tissue T from a healthy individual and D_{P×N2} be the observed gene expression data for the corresponding tissue from a disease individual. P is the number of features(genes) and N₁ and N₂ are the number of single cell samples from the healthy and disease tissue respectively.

Applicants assume a non-negative matrix factorization for H and D as follows

$$\begin{matrix}{H}_{P \times {N}_{1}} \approx [ \underbrace{{L}_{P \times {K}_{C}}^{CH} {L}_{P \times {K}_{H}}^{UH}}_{{L}^{H}} ] {F}_{( {K}_{C} + {K}_{H} ) \times {N}_{1}}^{( H )} {L}^{CH} , \\ {L}^{UH} , {F}^{H} non \text{-} negative & ( 1 ) \\ {D}_{P \times {N}_{2}} \approx [ \underbrace{{L}_{P \times {K}_{C}}^{CD} {L}_{P \times {K}_{D}}^{UD}}_{{L}^{D}} ] {F}_{( {K}_{C} + {K}_{D} ) \times {N}_{2}}^{( D )} {L}^{CD} , \\ {L}^{UD} , {F}^{D} non \text{-} negative & ( 2 )\end{matrix}$$

where K_{C} is the number of shared clusters between the healthy and the disease samples, K_{H} is the number of healthy specific clusters and K_{D} is the number of disease specific clusters. Applicants assume that L^{CH} is very close to L^{CD} but not exact to account for other factors like experimental conditions perturbing the estimates slightly. Applicants frame this in the form of the following optimization problem

$${argmin}_{{L}^{H} , {L}^{D} , {P}^{H} , {P}^{D}} \{ \frac{1}{2} {‖ H - {L}^{H} {F}^{H} ‖}_{F}^{2} + \frac{1}{2} {‖ D - {L}^{D} {F}^{D} ‖}_{F}^{2} + \frac{v}{2} ( {‖ {L}^{H} ‖}_{F}^{2} + {‖ {L}^{D} ‖}_{F}^{2} ) + \frac{γ}{2} {‖ {L}^{CH} - {L}^{CD} ‖}_{F}^{2} \} \qquad ( 3 )$$

γ is a tuning parameter that controls how close L^{CH} is to L^{CD}. μ represents a tuning parameter that controls for the size of the loadings and the factors.

To compute the multiplicative updates of the NMF optimization problem in Equation 3 can be determined by computing the derivatives of the optimizing criterion with respect to each parameter of interest. Applicants call the optimizing criterion as Q

∇Q(L^{H})=−HF^{H}^{^{T}}+L^{H}F^{H}F^{H}^{^{T}}+μL^{H}−γ[L^{CD}0] (4)

∇Q(L^{D})=−HF^{D}^{^{T}}+L^{D}F^{D}F^{D}^{^{T}}+μL^{D}−γ[L^{CH}0] (5)

∇Q(F^{H})=−L^{H}^{^{T}}H+L^{H}^{^{T}}L^{H}F^{H} (6)

∇Q(F^{D})=−L^{D}^{^{T}}D+L^{D}^{^{T}}L^{D}F^{D} (7)

Following the multiplicative update rules of NMF as per Lee and Seung (NIPS 2001), Applicants get the following iterative updates

$$\begin{matrix}{L}_{ij}^{H} ← {L}_{ij}^{H} \frac{{( H {F}^{{H}^{T}} + γ [ {L}^{CD} 0 ] )}_{ij}}{{( {L}^{H} {F}^{H} {F}^{{H}^{T}} + μ {L}^{H} )}_{ij}} & ( 8 ) \\ {L}_{ij}^{D} ← {L}_{ij}^{D} \frac{{( {DF}^{{D}^{T}} + γ [ {L}^{CH} 0 ] )}_{ij}}{{( {L}^{D} {F}^{D} {F}^{{D}^{T}} + μ {L}^{D} )}_{ij}} & ( 9 ) \\ {F}_{ij}^{H} ← {F}_{ij}^{H} \frac{{( {L}^{{H}^{T}} H )}_{ij}}{{( {L}^{{H}^{T}} {L}^{H} {F}^{H} )}_{ij}} & ( 10 ) \\ {F}_{ij}^{D} ← {F}_{ij}^{D} \frac{{( {L}^{{D}^{T}} D )}_{ij}}{{( {L}^{{D}^{T}} {L}^{D} {F}^{D} )}_{ij}} & ( 11 )\end{matrix}$$

### REFERENCES

1. 1000 Genomes Project Consortium. A global reference for human genetic variation. Molecular cell, 526(7571):68-74, 2015.
2. H. K. Finucane, B. Bulik-Sullivan, A. Gusev, G. Trynka, Y. Reshef, P. R. Loh, V. Anttila, H. Xu, C. Zang, K. Farh, and S. Ripke. Partitioning heritability by functional annotation using genome-wide association summary statistics. Nature genetics, 47:1228{1235, 2015.
3. Y. Liu, A. Sarkar, and M. Kellis. Evidence of a recombination rate valley in human regulatory domains. Genome Biology, page 193, 2017.
4. J. Ernst et al. Mapping and analysis of chromatin state dynamics in nine human cell types. Nature, 473:43-49, 2011.
5. H. K. Finucane, Y. A. Reshef, V. Anttila, K. Slowikowski, A. Gusev, A. Byrnes, et al. Heritability enrichment of specifically expressed genes identifies disease-relevant tissues and cell types. Nature genetics, 50:621-629, 2018.
6. X. Zhu and M. Stephens. Large-scale genome-wide enrichment analyses identify new trait-associated genes and pathways across 31 human phenotypes. Nature communications, 9(1):4361, 2018.
7. K. K. Dey et al. Unique contribution of enhancer-driven and master-regulator genes to autoimmune disease revealed using functionally informed SNP-to-gene strategies. bioRxiv, page p. 784439, 2020.
8. S. Gazal et al. Linkage disequilibrium{dependent architecture of human complex traits shows action of negative selection. Nature genetics, 49(10):1421-1427, 2017.
9. S. Gazal, C. Marquez-Luna, H. K. Finucane, and A. L. Price. Reconciling s-ldsc and ldak models and functional enrichment estimates. Nature genetics, 51(8):1202-1204, 2019.
10. F. Hormozdiari et al. Leveraging molecular quantitative trait loci to understand the genetic architecture of diseases and complex traits. Nature genetics, 50(7):1041-1047, 2018.

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

1. A method of identifying genes associated with one or more phenotypes or identifying phenotypes associated with genes comprising:
a. providing one or more gene modules constructed from one or more single cell atlases;
b. linking genetic variants to the one or more gene modules based on enhancer-gene connections, wherein genetic variants located in enhancers predicted to regulate genes in the one or more gene modules are linked to the module; and
c. identifying one or more phenotypes associated with the genetic variants linked to each gene module, thereby identifying genes associated with the phenotypes or phenotypes associated with the genes.

2. The method of claim 1, wherein the method is for identifying genes associated with one or more phenotypes specific to a tissue comprising:
a. providing one or more gene modules constructed from one or more single cell atlases for the tissue;
b. linking genetic variants to the one or more gene modules based on enhancer-gene connections, wherein genetic variants located in enhancers predicted to regulate genes in the one or more gene modules are linked to the module; and
c. identifying one or more phenotypes associated with the genetic variants linked to each gene module, thereby identifying genes associated with the phenotypes

3. The method of claim 2, wherein linking genetic variants to the one or more gene modules comprises: calculating a gene score for genes in each module; and assigning a variant to the gene with the highest score among genes linked to that variant according to both an Activity-by-Contact (ABC) model and an epigenomic model, preferably,
wherein the epigenomic model uses chromatin state, gene expression, regulatory motif enrichment and regulator expression to predict enhancer-gene connections; and/or
wherein gene score is based on the enrichment of each gene in each module and/or a gene level significance score based on GWAS p values of all surrounding SNPs.

4. (canceled)

5. The method of claim 1, wherein the phenotype is a disease phenotype and the gene modules comprise genes differentially expressed between healthy and disease states in the tissue, whereby gene programs associated with the disease phenotype are identified, preferably, wherein the differentially expressed genes are cell type specific, whereby cell types associated with the disease phenotype are identified; or
wherein the gene modules comprise transcriptomes specific for cell types in the tissue, whereby cell types associated with the phenotype are identified; or
wherein the gene modules comprise biological programs indicating cell states in the tissue, whereby cell states associated with the phenotype are identified, preferably, wherein the biological programs are determined by negative matrix factorization (NMF), topic modeling, or word embeddings.

6-9. (canceled)

10. The method of claim 1, wherein the method is for identifying phenotypes associated with genes comprising:
a. providing one or more gene modules comprising one or more genes of interest and one or more covarying genes constructed from one or more single cell atlases for a tissue associated with the genes of interest;
b. linking genetic variants to the one or more gene modules based on enhancer-gene connections, wherein genetic variants located in enhancers predicted to regulate genes in the one or more gene modules are linked to the module; and
c. identifying one or more phenotypes associated with the genetic variants linked to each gene module, thereby identifying phenotypes associated with the genes of interest.

11. The method of claim 10, wherein linking genetic variants to the one or more gene modules comprises: calculating a gene score for genes in each module; and assigning a variant to the gene with the highest score among genes linked to that variant according to both an Activity-by-Contact (ABC) model and an epigenomic model, preferably,
wherein the epigenomic model uses chromatin state, gene expression, regulatory motif enrichment and regulator expression to predict enhancer-gene connections; and/or
wherein gene score is based on the enrichment of each gene in each module and/or a gene level significance score based on GWAS p values of all surrounding SNPs.

12-13. (canceled)

14. The method of claim 10, wherein the one or more genes of interest comprise one or more disease associated genes and wherein the tissue is associated with the disease, whereby phenotypes associated with disease associated genes are identified; or
wherein the gene modules comprise transcriptomes specific for cell types in the tissue, whereby phenotypes associated with cell types are identified; or
wherein the gene modules comprise biological programs indicating cell states in the tissue, whereby phenotypes associated with cell states are identified, preferably, wherein the biological programs are determined by negative matrix factorization (NMF), topic modeling, or word embeddings.

15-17. (canceled)

18. A method of determining a risk score for a disease phenotype comprising detecting in a subject two or more genetic variants associated with the disease phenotype and linked to a common gene module identified according to claim 5; or detecting in a subject one or more gene modules or cells identified according to claim 5.

19. (canceled)

20. The method of claim 1, wherein the gene modules are constructed using single cell RNA-seq data from the single cell atlas; and/or
wherein the gene modules are constructed using single cell epigenetic data from the single cell atlas, preferably, wherein the epigenetic data comprises single cell ChIP-seq data; and/or
wherein the gene modules are constructed using single cell ATAC-seq data from the single cell atlas; and/or
wherein the genetic variants are single nucleotide polymorphisms (SNPs), preferably, wherein the SNPs are associated with phenotypes based on genome wide association studies (GWAS); and/or
wherein the enhancers are specific to the tissue; and/or
wherein identifying one or more phenotypes associated with the genetic variants linked to each gene module comprises stratified LD score regression across a set of phenotypes; and/or
wherein the one or more single cell atlases were generated from a diseased tissue; and/or
wherein the one or more single cell atlases were generated from a healthy tissue.

21-29. (canceled)

30. An unbiased method of identifying interacting genetic variants associated with a phenotype comprising:
a. assigning genetic variants identified in one or more subjects having the phenotype to one or more gene modules,
wherein the gene modules are derived from a single cell atlas specific for a tissue of interest associated with the phenotype,
wherein the atlas comprises one or more single cell analyses of genomic loci comprising the genetic variants, and
wherein a genetic variant is assigned to a gene module where the genomic loci comprising the genetic variant is transcriptionally active in the module; and
b. determining interactions by testing the association of two or more genetic variants within the same module or between associated modules with the phenotype.

31. The method of claim 30, wherein the genetic variant is present in a gene, preferably, wherein the gene is a protein coding gene or a non-protein coding gene, more preferably, wherein the genetic variant is present in an exon or intron in the gene; or wherein the genetic variant is present in a regulatory element controlling expression of a gene.

32-34. (canceled)

35. The method of claim 30, wherein the single cell atlas comprises one or more single cell analyses of tissues having the phenotype and tissues having a control phenotype; and/or
wherein the single cell analyses comprise single cell RNA-seq data; and/or
wherein the single cell analyses comprise epigenetic data, preferably, wherein the epigenetic data comprises single cell ChIP-seq data; and/or
wherein the single cell analyses comprise single cell ATAC-seq data; and/or
wherein the phenotype is a disease state, preferably,
wherein the disease state is classified by severity or subtype; or
wherein the genetic variants tested are present at a higher frequency in subjects having the disease than in control subjects; or
wherein the gene modules are conserved across disease states; or
wherein the gene modules are non-conserved across disease states; and/or
wherein each gene module comprises genes or genomic loci that are transcriptionally active in a specific cell type, whereby the gene modules are cell type specific; or
wherein each gene module comprises a gene program expressed across the single cells; or
wherein associated gene modules comprise cell type specific modules for interacting cell types, preferably, wherein the interacting cell types are selected from the group consisting of immune cells, stromal cells and epithelial cells.

36-45. (canceled)

46. The method of claim 35, wherein the gene modules are constructed by:
a. grouping one or more genes associated with the phenotype by cell type specificity; and
b. adding one or more additional genes to each group that co-vary in each cell type with the genes associated with the phenotype.

47. The method of claim 35, wherein each gene module comprises genes differentially expressed in single cell types between disease and control subjects; or
wherein each gene module comprises genes located in open chromatin in single cells; or
wherein each gene module comprises genes located in chromatin comprising active epigenetic marks in single cells.

48-52. (canceled)

53. The method of claim 30, further comprising identifying genetic variants in the one or more subjects, preferably, wherein the genetic variants are identified by whole exome sequencing (WES); and/or
further comprising identifying pathways associated with the phenotype, said method comprising clustering the identified genetic variants by traits associated with the tissue of interest, preferably, wherein the genetic variants are clustered using Bayesian nonnegative matrix factorization (bNMF); and/or
further comprising identifying cell types associated with the phenotype, said method comprising determining the expression of genomic loci comprising the identified genetic variants in single cells in the tissue; and/or
further comprising determining a risk score for the phenotype for a subject, said method comprising detecting in the subject genetic variants in one or more gene modules comprising an interacting genetic variant, wherein detecting a genetic variant in the gene modules indicates increased risk for the phenotype.

54-58. (canceled)

59. The method of claim 30, wherein the tissue of interest is colon or intestinal tissue.

60. The method of claim 35, wherein the disease is inflammatory bowel disease (IBD), preferably, wherein the IBD is ulcerative colitis (UC); or
wherein the disease is cancer, preferably, wherein the cancer is colorectal cancer (CRC).

61-63. (canceled)

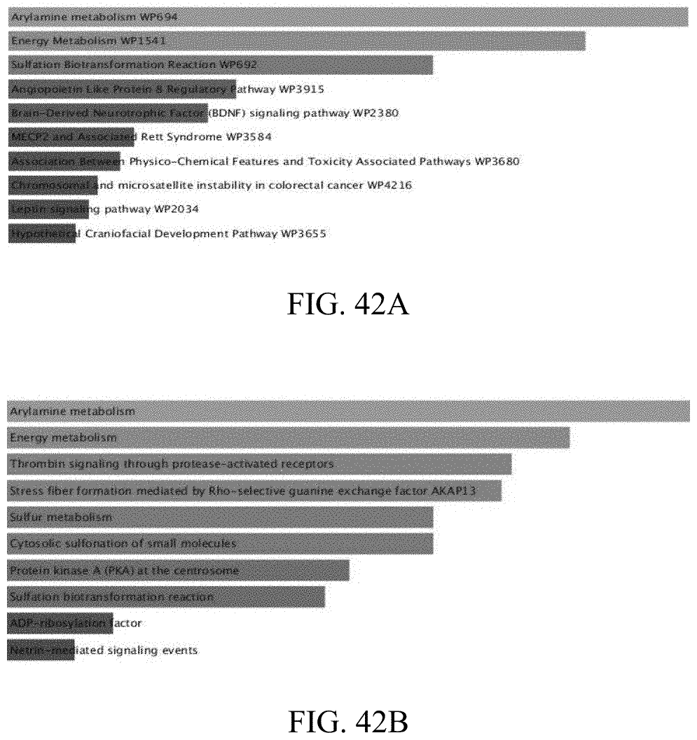

64. A method of determining a risk score for a phenotype comprising:
detecting in a subject genetic variants in one or more cell type specific gene modules, wherein detecting a variant in a gene module indicates increased risk for a disease phenotype, and wherein the one or more gene modules comprise one or more genes associated with the disease phenotype and one or more genes that co-vary with the disease genes in each cell type; or
detecting in a subject altered expression of one or more gene modules in Tables 8 to 12 or altered signaling in a pathway in FIGS. 34 to 42.

65. The method of claim 64, wherein the genes associated with the disease phenotype are determined by genome wide association studies; or
wherein the genes associated with the disease phenotype are determined by the method according to claim 30; or
wherein the cell type specific gene expression is determined by single cell RNA sequencing one or more control and disease tissue samples; and/or
wherein the disease is inflammatory bowel disease (IBD), preferably, wherein the IBD is ulcerative colitis (UC); and/or
wherein the one or more cell type specific gene modules are selected from Table 4, Table 5, Table 6, or the group consisting of myeloid cells, epithelial cells, stromal cells, cycling B cells, germinal center B cells, transit amplifying cells, macrophages, enterocytes, enterocyte progenitors, CD8+ IELs and goblet cells; or
wherein the disease is cancer, preferably, wherein the cancer is colorectal cancer (CRC).

66-72. (canceled)

73. A method of modifying a phenotype comprising:
treating inflammatory bowel disease (IBD) in a subject in need thereof by altering one or more genetic variants, or altering expression, activity and/or function of one or more genes comprising the one or more genetic variants in one or more cell types, wherein the one or more genetic variants are selected from Table 7 or from the group consisting of 16:50763778 (NOD2), 16:50745199 (NOD2), 19:55144141 (LILRB1), 16:50744624 (NOD2), 1:117122130 (IGSF3), 2:233659553 (GIGYF2), 11:55595018 (OR5L2) and 16:2155426 (PKD1), preferably,
wherein the IBD is ulcerative colitis (UC); and/or
wherein two or more genetic variants or genes comprising the genetic variants are altered; or
wherein the one or more genetic variants are in transcriptionally active loci in the same cell type; or
wherein the one or more genetic variants are in transcriptionally active loci in different cell types; or
wherein the one or more genetic variants are within NOD2, more preferably, wherein the one or more genetic variants are 16:50763778 and 16:50745199; and/or
wherein the expression, activity and/or function of the one or more genes comprising the one or more genetic variants is reduced or abolished; and/or
wherein the one or more genetic variants is altered using genome editing; and/or
wherein the one or more genetic variants or genes comprising the one or more genetic variants are altered in one or more cell types in vivo; or
wherein the one or more genetic variants or genes comprising the one or more genetic variants are altered in one or more cell types ex vivo and the cells are transferred to the subject; and/or
wherein the one or more genetic variants or genes comprising the one or more genetic variants are altered in intestinal stem cells; and/or
wherein the one or more genetic variants or genes comprising the one or more genetic variants are altered in transit-amplifying cells (TA cells); or
administering one or more agents to a subject in need thereof capable of altering expression of one or more gene modules in Tables 8 to 12 or altering signaling in a pathway in FIGS. 34 to 42, preferably,
wherein Major Depressive Disorder (MDD) and/or body mass index (BMI) is treated and the one or more agents alter the GABA-ergic neuron cell type program, more preferably, wherein TCF4 and/or PCLO are altered; or
wherein decreased lung capacity and/or asthma is treated and the one or more agents alter the TGF-beta regulation of extracellular matrix and/or ECM-receptor interaction program, more preferably, wherein one or more genes selected from the group consisting of ITGA1, LOX, TGFBR3, COL8A1, BAMBI and VCL are altered; or
wherein abnormal systolic and diastolic blood pressure is treated and the one or more agents alter the pericyte and/or vascular smooth muscle gene program, more preferably, wherein one or more genes selected from the group consisting of GUCY1A3, CACNA1C, PDE8A and EDNRA are altered; or
wherein abnormal atrial fibrillation and cardiac rhythm is treated and the one or more agents alter the atrial cardiomyocyte gene program, more preferably, wherein one or more genes selected from the group consisting of PKD2L2, CASQ2 and KCNN2 are altered; or wherein ‘potassium channel’ pathways are altered; or
wherein ulcerative colitis is treated and the one or more agents alter the T Lymphocyte, enterocyte and/or ILC disease gene program, more preferably, wherein IL2RA is altered.

74-84. (canceled)

85. The method of claim 73, wherein the cells are treated with one or more agents comprising a small molecule, small molecule degrader, genetic modifying agent, antibody, antibody fragment, antibody-like protein scaffold, aptamer, protein, or any combination thereof, preferably,
wherein the genetic modifying agent comprises a CRISPR system, RNAi system, a zinc finger nuclease system, a TALE system, or a meganuclease, more preferably, wherein the CRISPR system may be a CRISPR-Cas base editing system, a prime editor system, or a CAST system.

86-88. (canceled)

89. The method of claim 30, wherein the genetic variants are single-nucleotide polymorphisms (SNPs).

90. The method of claim 64,
wherein an altered GABA-ergic neuron cell type program indicates a risk for Major Depressive Disorder (MDD) and/or body mass index (BMI), preferably, wherein TCF4 and/or PCLO are detected; or
wherein an altered TGF-beta regulation of extracellular matrix and/or ECM-receptor interaction program indicates a risk for decreased lung capacity and/or asthma, preferably, wherein one or more genes selected from the group consisting of ITGA1, LOX, TGFBR3, COL8A1, BAMBI and VCL are detected; or
wherein an altered pericyte and/or vascular smooth muscle gene program indicates a risk for abnormal systolic and diastolic blood pressure, preferably, wherein one or more genes selected from the group consisting of GUCY1A3, CACNA1C, PDE8A and EDNRA are detected; or
wherein an altered atrial cardiomyocyte gene program indicates a risk for abnormal atrial fibrillation and cardiac rhythm, preferably, wherein one or more genes selected from the group consisting of PKD2L2, CASQ2 and KCNN2 are detected; or wherein ‘potassium channel’ pathways are detected; or
wherein an altered T Lymphocyte, enterocyte and/or ILC disease gene program indicates a risk for ulcerative colitis, preferably, wherein IL2RA is detected.

91-113. (canceled)


SEQUENCE LISTING (39 sequences)

SEQ ID NO: 1 - DNA, 19 bp, Artificial Sequence
  Synthetic
atccaggcgttcctgccgc

SEQ ID NO: 2 - DNA, 85 bp, Artificial Sequence
  Synthetic
atgggactcttcagacaatccaaacatccaatggccaatatcacctggatggccaaccacactggatggtcggatttcatcctgt

SEQ ID NO: 3 - DNA, 79 bp, Artificial Sequence
  Synthetic
aagcccgaccaccccagagcccacctcagagcccgcccccagcccgaccaccccggagcccacctcagagcccgccccc

SEQ ID NO: 4 - DNA, 79 bp, Artificial Sequence; identical to SEQ ID NO: 3
  Synthetic

SEQ ID NO: 5 - DNA, 77 bp, Artificial Sequence
  Synthetic
gggtcgagaaattgttaaaacctttgccacattcttcacatttgtacggtttctccccagtatgaattatcttatgt

SEQ ID NO: 6 - DNA, 168 bp, Artificial Sequence
  Synthetic
ggctttgccacattcttcacatttgtagaatttctctccagtatgattctctcatgtgtagtaaggattgaggactggttgaaggctttgccacattcttcacatttgtagggtctctctccagtatgaattttcttatgtgtagtaaggttagaggagcacttaaaa

SEQ ID NO: 7 - DNA, 41 bp, Artificial Sequence
  Synthetic
agccacagcccccacagccagagccacagcccccacagccg

SEQ ID NO: 8 - DNA, 23 bp, Artificial Sequence
  Synthetic
caccacccttacccaaggaggca

SEQ ID NO: 9 - DNA, 19 bp, Artificial Sequence
  Synthetic
atccaggcgttcctgccgc

SEQ ID NO: 10 - DNA, 85 bp, Artificial Sequence; identical to SEQ ID NO: 2
  Synthetic

SEQ ID NO: 11 - DNA, 19 bp, Artificial Sequence
  Synthetic
atccaggcgttcctgccgc

SEQ ID NO: 12 - DNA, 23 bp, Artificial Sequence
  Synthetic
caccacccttacccaaggaggca

SEQ ID NO: 13 - DNA, 41 bp, Artificial Sequence; identical to SEQ ID NO: 7
  Synthetic

SEQ ID NO: 14 - DNA, 77 bp, Artificial Sequence; identical to SEQ ID NO: 5
  Synthetic

SEQ ID NO: 15 - protein, 288 aa, Artificial Sequence
  Synthetic
MDPIRSRTPSPARELLSGPQPDGVQPTADRGVSPPAGGPLDGLPARRTMSRTRLPSPPAPSPAFSADSFSDLLRQFDPSLFNTSLFDSLPPFGAHHTEAATGEWDEVQSGLRAADAPPPTMRVAVTAARPPRAKPAPRRRAAQPSDASPAAQVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLN

SEQ ID NO: 16 - protein, 183 aa, Artificial Sequence
  Synthetic
RPALESIVAQLSRPDPALAALTNDHLVALACLGGRPALDAVKKGLPHAPALIKRTNRRIPERTSHRVADHAQVVRVLGFFQCHSHPAQAFDDAMTQFGMSRHGLLQLFRRVGVTELEARSGTLPPASQRWDRILQASGMKRAKPSPTSTQTPDQASLHAFADSLERDLDAPSPMHEGDQTRAS

SEQ ID NO: 17 - protein, 7 aa, Simian virus 40
PKKKRKV

SEQ ID NO: 18 - protein, 10 aa, Simian virus 40
PKKKRKVEAS

SEQ ID NO: 19 - protein, 16 aa, Artificial Sequence
  Synthetic
KRPAATKKAGQAKKKK

SEQ ID NO: 20 - protein, 9 aa, Homo sapiens
PAAKRVKLD

SEQ ID NO: 21 - protein, 11 aa, Homo sapiens
RQRRNELKRSP

SEQ ID NO: 22 - protein, 38 aa, Homo sapiens
NQSSNFGPMKGGNFGGRSSGPYGGGGQYFAKPRNQGGY

SEQ ID NO: 23 - protein, 42 aa, Homo sapiens
RMRIZFKNKGKDTAELRRRRVEVSVELRKAKKDEQILKRRNV

SEQ ID NO: 24 - protein, 8 aa, Homo sapiens
VSRKRPRP

SEQ ID NO: 25 - protein, 8 aa, Homo sapiens
PPKKARED

SEQ ID NO: 26 - protein, 8 aa, Homo sapiens
PQPKKKPL

SEQ ID NO: 27 - protein, 12 aa, Mus sp.
SALIKKKKKMAP

SEQ ID NO: 28 - protein, 5 aa, Influenza virus
DRLRR

SEQ ID NO: 29 - protein, 7 aa, Influenza virus
PKQKKRK

SEQ ID NO: 30 - protein, 10 aa, Hepatitis D virus
RKLKKKIKKL

SEQ ID NO: 31 - protein, 10 aa, Mus sp.
REKKKFLKRR

SEQ ID NO: 32 - protein, 20 aa, Homo sapiens
KRKGDEVDGVDEVAKKKSKK

SEQ ID NO: 33 - protein, 17 aa, Homo sapiens
RKCLQAGMNLEARKTKK

SEQ ID NO: 34 - DNA, 168 bp, Artificial Sequence; identical to SEQ ID NO: 6
  Synthetic

SEQ ID NO: 35 - DNA, 23 bp, Artificial Sequence
  Synthetic
caccacccttacccaaggaggca

SEQ ID NO: 36 - DNA, 23 bp, Artificial Sequence
  Synthetic
caccacccttacccaaggaggca

SEQ ID NO: 37 - DNA, 168 bp, Artificial Sequence; identical to SEQ ID NO: 6
  Synthetic

SEQ ID NO: 38 - DNA, 23 bp, Artificial Sequence
  Synthetic
caccacccttacccaaggaggca

SEQ ID NO: 39 - DNA, 77 bp, Artificial Sequence; identical to SEQ ID NO: 5
  Synthetic